SEQUENCE LISTING (61 sequences)

SEQ ID NO: 1 - protein, 735 aa, synthetic construct [sequence omitted]

SEQ ID NO: 2 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 3 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 4 - DNA, 2175 bp, synthetic construct
  source 1..2175: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 5 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 6 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 7 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 9 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 10 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 11 - DNA, 2217 bp, synthetic construct
  source 1..2217: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagaggccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgtgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 12 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGEQTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 744 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAIEQTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 744 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALAEITRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 744 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLKNAETARPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLNLAIEQTRPARQAATTDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGMLNEQTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 18 - protein, 746 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRSGNRADSETARPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 19 - protein, 741 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGTGDTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - protein, 744 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLQGETIRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 21 - protein, 746 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQNLANPETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRAPQETTRPARQAAAADVNWQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGANLTTTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGALLAGEQTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 25 - protein, 744 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITCEEEIRTTNPVATEQYGSVSTNLQRGGLRAEQTRPARQAATADVNNQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 26 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKNNVDIEKVMITDEEEIRTTNPVATEQYGSVSTNLQRARLDETARPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 27 - protein, 746 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMLTDEEEIRTTNPVATEQYGSVSTNLQRGTNLARGETARPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 28 - DNA, 2238 bp, synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacttggctttaggggagcaaacaagacctgcaagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 29 - DNA, 2235 bp, synthetic construct
  source 1..2235: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaaccttgctatagaacaaacacgccccgccagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 30 - DNA, 2235 bp, synthetic construct
  source 1..2235: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcctcgcgttagcagagataactcgacccgcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 31 - DNA, 2235 bp, synthetic construct
  source 1..2235: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcctgaagaacgcggaaacagcacgtcccgcaagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 32 - DNA, 2238 bp, synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcctcaaccttgcgatagagcaaaccaggcccgcgagacaagcagctaccaccgatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 33 - DNA, 2232 bp, synthetic construct
  source 1..2232: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcatgttaaatgagcaaactcggccagccagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 34 - DNA, 2241 bp, synthetic construct
  source 1..2241: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagcggagcggcaaccgagcagatagcgagactgcaagacccgctagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 35 - DNA, 2226 bp, synthetic construct
  source 1..2226: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaccggtgatacccggcctgctagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 36 - DNA, 2235 bp, synthetic construct
  source 1..2235: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctacaaggtgagacaattagacccgcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 37 - DNA, 2241 bp, synthetic construct
  source 1..2241: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggccaaaacctcgcgaacccagagacaacacgtcctgcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 38 - DNA, 2238 bp, synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaaccgtgcaccgcaggagacaacccggcccgccagacaagcagctgcagcagatgtcaactggcaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 39 - DNA, 2232 bp, synthetic construct
  source 1..2232: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcgccaacttaactacgactcgtcccgcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 40 - DNA, 2238 bp, synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcgctctgctggcgggcgagcagacaagaccggcaagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 41 - DNA, 2235 bp, synthetic construct
  source 1..2235: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacatgtgaagaggaaatcaggacaaccaatcccgtggctacggagcagtatggttctgtatctaccaacctccagagaggcggtttgagagctgagcaaacccgcccagccagacaagcagctaccgcagatgtcaacaatcaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 42 - DNA, 2232 bp, synthetic construct
  source 1..2232: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaaacaatgtggacattgaaaaggtcatgattacagacgaagaggaaatcaggacaaccaatcccgtggctacggagcagtatggttctgtatctaccaacctccagagggcgcgtcttgacgaaactgccagacctgcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 43 - DNA, 2241 bp, synthetic construct
  source 1..2241: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgttaacagacgaagaggaaatcaggacaaccaatcccgtggctacggagcagtatggttctgtatctaccaacctccagagaggcacaaacctagcgcggggtgaaaccgctagaccagcgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 44 - protein, 10 aa, synthetic construct
LALGEQTRPA

SEQ ID NO: 45 - protein, 9 aa, synthetic construct
LAIEQTRPA

SEQ ID NO: 46 - protein, 9 aa, synthetic construct
LALAEITRP

SEQ ID NO: 47 - protein, 9 aa, synthetic construct
LKNAETARP

SEQ ID NO: 48 - protein, 10 aa, synthetic construct
LNLAIEQTRP

SEQ ID NO: 49 - protein, 8 aa, synthetic construct
MLNEQTRP

SEQ ID NO: 50 - protein, 11 aa, synthetic construct
RSGNRADSETA

SEQ ID NO: 51 - protein, 6 aa, synthetic construct
TGDTRP

SEQ ID NO: 52 - protein, 9 aa, synthetic construct
LQGETIRPA

SEQ ID NO: 53 - protein, 11 aa, synthetic construct
QNLANPETTRP

SEQ ID NO: 54 - protein, 10 aa, synthetic construct
RAPQETTRPA

SEQ ID NO: 55 - protein, 8 aa, synthetic construct
ANLTTTRP

SEQ ID NO: 56 - protein, 10 aa, synthetic construct
ALLAGEQTRP

SEQ ID NO: 57 - protein, 9 aa, synthetic construct
GLRAEQTRP

SEQ ID NO: 58 - protein, 8 aa, synthetic construct
RARLDETA

SEQ ID NO: 59 - protein, 11 aa, synthetic construct
TNLARGETARP

SEQ ID NO: 60 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 61 - DNA, 2238 bp, synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacttggctcttggggaaactacgcgtcctgcaagacaagcagctaccgcagatgtcaacacacaaggcgttctaccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

(19) United States
(12) Patent Application Publication
(10) Pub. No.: US 2025/0250305 A1
(43) Pub. Date: Aug. 7, 2025

(54) CAPSID VARIANTS AND METHODS OF USING THE SAME

(71) Applicant: DYNO THERAPEUTICS, INC., Watertown, MA (US)

(72) Inventors: Hanna LEVITIN, Cambridge, MA (US); Heikki TURUNEN, Sommerville, MA (US)

(73) Assignee: DYNO THERAPEUTICS, INC., Watertown, MA (US)

(21) Appl. No.: 18/856,540

(22) PCT Filed: Apr. 14, 2023
(86) PCT No.: PCT/US2023/065815

§ 371 Date: Oct. 11, 2024

Related U.S. Application Data
(60) Provisional application No. 63/443,262, filed on Feb. 3, 2023.
(60) Provisional application No. 63/331,543, filed on Apr. 15, 2022.

(51) Int. Cl. C07K 14/005; A61K 48/00; C12N 15/86

(52) U.S. Cl. CPC C07K 14/005; A61K 48/0075; C12N 15/86; C12N 2750/14122; C12N 2750/14143

(57) ABSTRACT

The disclosure is directed in part to variant capsid polypeptides that can be used to deliver payloads.

### CROSS-REFERENCE TO RELATED APPLICATIONS

This application claims priority to U.S. Provisional Application No. 63/331,543, filed Apr. 15, 2022, and U.S. Provisional Application No. 63/443,262, filed Feb. 3, 2023, each of which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Apr. 14, 2023, is named “DYO-015WOSEQ.XML” and is 119,720 bytes in size.

### BACKGROUND

Dependoparvoviruses, e.g. adeno-associated dependoparvoviruses, e.g. adeno-associated viruses (AAVs), are of interest as vectors for delivering various payloads to cells, including in human subjects.

### SUMMARY

The present disclosure provides, in part, improved variant dependoparvovirus capsid polypeptides (e.g. variants of AAV2), such as VP1, methods of producing a dependoparvovirus, compositions for use in the same, as well as viral particles comprising such capsid polypeptides. In some embodiments, the viral particles that comprise the capsid polypeptides have increased ocular transduction as compared to viral particles without the mutations in the capsid proteins.

In some embodiments, the disclosure is directed, in part, to a nucleic acid comprising a sequence encoding a variant capsid protein as provided for herein. In some embodiments, the dependoparvovirus is an adeno-associated dependoparvovirus (AAV). In some embodiments, the AAV is an AAV2 variant.

In some embodiments, the disclosure is directed, in part, to a capsid polypeptide described herein.

In some embodiments, the disclosure is directed, in part, to a dependoparvovirus particle comprising a capsid polypeptides described herein.

In some embodiments, the disclosure is directed, in part, to a vector, e.g., a plasmid, comprising a nucleic acid described herein.

In some embodiments, the disclosure is directed, in part, to a dependoparvovirus particle comprising a nucleic acid described herein (e.g., a nucleic acid comprising a sequence encoding a capsid polypeptide, such as VP1, wherein the encoding sequence comprises a change or mutation as provided herein.

In some embodiments, the disclosure is directed, in part, to a dependoparvovirus particle comprising a variant capsid polypeptide described herein, for example, comprising a polypeptide that has at least 85, 90, 91, 92, 93, 94, 95, 96, 97, 98, 99%, or 100% identity to a VP1, VP2, or VP3 sequence of SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

In some embodiments, the disclosure is directed, in part, to a dependoparvovirus particle comprising a variant capsid polypeptide described herein, for example, comprising a polypeptide that is a VP1, VP2, or VP3 sequence of SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

In some embodiments, the capsid polypeptide comprises a mutation selected from a mutation associated with any of VAR-1 to VAR-16. In some embodiments, the capsid polypeptide comprises more than one, for example, 2, 3, 4, 5, 6, 7, 8, 9, 10, 11, 12, or all of the mutations selected from a mutation associated with any of VAR-1 to VAR-16.

In some embodiments, the capsid polypeptide comprises an amino acid sequence that is 95% or more amino acid sequence identity to an amino acid sequence of one of SEQ ID NOs: 12-27 and has at least 80% of the mutations in one of SEQ ID NO: 12-27 as compared to SEQ ID NO: 1.

In some embodiments, the capsid polypeptide comprises an amino acid sequence that is less than 95% amino acid sequence identity to an amino acid sequence of one of SEQ ID NOs: 12-27 and has at least 80% of the mutations in one of SEQ ID NO: 12-27 as compared to SEQ ID NO: 1.

In some embodiments, the capsid polypeptide comprises an amino acid sequence that is 95% or more amino acid sequence identity to an amino acid sequence of one of SEQ ID NOs: 12-27 and has less than 80% of the mutations in one of SEQ ID NO: 12-27 as compared to SEQ ID NO: 1.

In some embodiments, the disclosure is directed, in part, to a nucleic acid molecule comprising SEQ ID NO: 28, 29, 30, 31, 32, 33, 34, 35, 36, 37, 38, 39, 40, 41, 42, 43, a fragment thereof, or a variant thereof having at least 85, 90, 91, 92, 93, 94, 95, 96, 97, 98, 99, or 100% sequence identity thereto.

In some embodiments, the disclosure is directed, in part, to a vector comprising a nucleic acid described herein, e.g., a nucleic acid comprising a sequence encoding a capsid polypeptide, e.g. a VP1 polypeptide, wherein the encoding sequence comprises a change or mutation as provided for herein.

In some embodiments, the disclosure is directed, in part, to a cell, cell-free system, or other translation system comprising a nucleic acid or vector described herein, e.g., comprising a sequence encoding capsid polypeptide, such as VP1, wherein the capsid polypeptide encoding sequence comprises a change or mutation as provided for herein in the encoding sequence. In some embodiments, the cell, cell-free system, or other translation system comprises a dependoparvovirus particle described herein, e.g., wherein the particle comprises a nucleic acid comprising a sequence encoding a capsid polypeptide, such as a VP1 polypeptide, wherein the encoding sequence comprises a change or mutation as provided for herein.

In some embodiments, the disclosure is directed, in part, to a cell, cell-free system, or other translation system comprising a polypeptide described herein, wherein the polypeptide encoding sequence comprises a change or mutation as provided for herein. In some embodiments, the cell, cell-free system, or other translation system comprises a dependoparvovirus particle described herein, e.g., wherein the particle comprises a nucleic acid comprising a sequence encoding a VP1 polypeptide, wherein the VP1 encoding sequence comprises a change or mutation corresponding such as provided for herein.

In some embodiments, the disclosure is directed, in part, to a method of delivering a payload to a cell comprising contacting the cell with a dependoparvovirus particle comprising a nucleic acid described herein. In some embodiments, the disclosure is directed, in part, to a method of delivering a payload to a cell comprising contacting the cell with a dependoparvovirus particle comprising a capsid polypeptide described herein.

In some embodiments, the disclosure is directed, in part, to a method of making a dependoparvovirus particle, comprising providing a cell, cell-free system, or other translation system, comprising a nucleic acid described herein (e.g., a nucleic acid comprising a sequence encoding an AAV2 capsid variant as provided for herein); and cultivating the cell, cell-free system, or other translation system, under conditions suitable for the production of the dependoparvovirus particle, thereby making the dependoparvovirus particle. In some embodiments, the disclosure is directed, in part, to a method of making a dependoparvovirus particle described herein.

In some embodiments, the disclosure is directed, in part, to a method of making a dependoparvovirus particle, comprising providing a cell, cell-free system, or other translation system, comprising a polypeptide described herein; and cultivating the cell, cell-free system, or other translation system, under conditions suitable for the production of the dependoparvovirus particle, thereby making the dependoparvovirus particle. In some embodiments, the disclosure is directed, in part, to a method of making a dependoparvovirus particle described herein.

In some embodiments, the disclosure is directed, in part, to a dependoparvovirus particle made in a cell, cell-free system, or other translation system, wherein the cell, cell-free system, or other translation system comprises a nucleic acid encoding a dependoparvovirus comprising an capsid variant as provided for herein.

In some embodiments, the disclosure is directed, in part, to a method of treating a disease or condition in a subject, comprising administering to the subject a dependoparvovirus particle described herein in an amount effective to treat the disease or condition.

The invention is further described with reference to the following numbered embodiments.

### BRIEF DESCRIPTION OF THE DRAWINGS

FIG. 1. Diagram of tissues collected in each region of the eye. In the retina (left and center figures), peripheral and central retina samples from each of the superior, nasal, inferior and temporal regions of the retina were separately collected, macula was also separately collected. In each region, neural retina and choroid/RPE layers (center figure) were separately collected. In the trabecular meshwork/Schlemm's canal (TM/SC) region (right figure), superior, temporal, nasal and inferior samples were separately collected.

FIG. 2A-2C. Multisequence alignment of representative reference capsid VP1 polypeptides. Such alignment can be used to determine the amino acid positions which correspond to positions within different reference capsid polypeptides.

FIG. 3A-3B. Single nuclear RNA sequencing results for AAV2 wild-type (FIG. 3A) and VAR-1, VAR-2, VAR-4, VAR-5, VAR-6, VAR-7, VAR-8, and VAR-11 (FIG. 3B) from retina tissue samples from intravitreal (“IVT”) administration of the medium throughput study (Example 3), reporting number of unique transduction events for each of AAV2 wild-type and VAR-1, VAR-2, VAR-4, VAR-5, VAR-6, VAR-7, VAR-8, and VAR-11, respectively. All results are normalized to the amount of virus reads.

FIG. 4A-4B. Single nuclear RNA sequencing results for AAV2 wild-type (FIG. 4A) and VAR-1, VAR-2, VAR-4, VAR-5, VAR-6, VAR-7, VAR-8, and VAR-11 (FIG. 4B) from retina tissue samples from intravitreal (“IVT”) administration of the medium throughput study (Example 3), reporting the number of unique transduction events for each of AAV2 wildtype and VAR-1, VAR-2, VAR-4, VAR-5, VAR-6, VAR-7, VAR-8, and VAR-11, respectively. All results are normalized to the amount of vector genome (vg) in the input test article.

FIG. 5. Single nuclear RNA sequencing results for VAR-1, VAR-2, VAR-4, VAR-5, VAR-6, VAR-7, VAR-8, and VAR-11 from macula tissue samples from intravitreal (“IVT”) administration of the medium throughput study (Example 3), reporting the number of unique transduction events for each of the variants. All results are normalized to the amount of vector genome (vg) in the input test article. Results indicate zero transduction of AAV2 wild-type (not shown) for any of the listed cell types.

FIG. 6. Single nuclear RNA sequencing results for VAR-1, VAR-2, VAR-4, VAR-5, VAR-6, VAR-7, VAR-8, and VAR-11, from trabecular meshwork tissue samples from intravitreal (“IVT”) administration of the medium throughput study (Example 3), reporting the number of unique transduction events for each of the variants. All results are normalized to the amount of vector genome (vg) in the input test article. Results indicate zero transduction of AAV2 wild-type (not shown), VAR-5, and VAR-11 for any of the listed cell types.

FIG. 7A-7B. Single nuclear RNA sequencing results for AAV2 wild-type (FIG. 7A) and VAR-2, VAR-3, VAR-13, and VAR-14 (FIG. 7B) from trabecular meshwork tissue samples from intracameral (“IC”) administration of the medium throughput study (Example 3), reporting the number of unique transduction events for each of the variants. All results are normalized to the amount of vector genome (vg) in the input test article. Results indicate zero transduction of VAR-2 for any of the listed cell types.

FIG. 8. Retinal distribution of AAV VAR-8-eGFP from a higher dose (2.26e11vg) intravitreal ocular injection in cynomolgus monkey. The genome packaged in the VAR-8 capsid is a self-complementary genome, composed of modified AAV2 ITRs, and an eGFP reporter gene under the control of a CBH promoter. The animals were sacrificed 4 weeks post-dosing. The eyes were enucleated, fixed, halved into two pieces (top and bottom) and embedded in paraffin to enable sectioning for histology. (A) Schematic illustrating where the cross-section of the retina was collected. Briefly a 5 μm section from the top surface of the bottom half of the eye was collected and stained for eGFP. (B) Representative image of a whole cross section of the eye collected. eGFP is shown as white dots in the retina. The arrow represents the starting position of the close up retinal image shown in (C). (C) Close up of the retinal images from each end of the eye shown in (B) stitched together linearly. White dots represent eGFP expression delivered from VAR-8. ONL=outer nuclear layer/photoreceptor layer, INL=inner nuclear layer, GCL=ganglion cell layer.

FIG. 9. Retinal distribution of AAV VAR-8-eGFP from a lower dose (8.08e10vg) intravitreal ocular injection in cynomolgus monkey. The genome packaged in the VAR-8 capsid is a self-complementary genome, composed of modified AAV2 ITRs, and an eGFP reporter gene under the control of a CBH promoter. The animals were sacrificed 4 weeks post-dosing. The eyes were enucleated, fixed, halved into two pieces (top and bottom) and embedded in paraffin to enable sectioning for histology. (A) Schematic illustrating where the cross-section of the retina was collected. Briefly a 5 μm section from ˜⅓ into the bottom half of the eye was collected and stained for eGFP. (B) Representative image of a whole cross section of the eye collected. eGFP is shown as white dots in the retina. The arrow represents the starting position of the close up retinal image shown in (C). (C) Close up of the retinal image from each end of the eye shown in (B) stitched together linearly. White dots represent eGFP expression delivered from VAR-8. ONL=outer nuclear layer/photoreceptor layer, INL=inner nuclear layer, GCL=ganglion cell layer.

### ENUMERATED EMBODIMENTS

1. A variant capsid polypeptide comprising a polypeptide that has at least 85, 90, 91, 92, 93, 94, 95, 96, 97, 98, 99%, or 100% identity to a VP1, VP2, or VP3 sequence of SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

2. The variant capsid polypeptide of embodiment 1, wherein the polypeptide comprises: a mutation selected from a mutation associated with any of VAR-1 to VAR-16.

3. The variant capsid polypeptide of embodiment 2, wherein:
the mutation associated with any of VAR-1 to VAR-16 comprises mutations at positions corresponding to residues 550-597 as compared to SEQ ID NO: 1.

4. The variant capsid polypeptide of any of the preceding embodiments, wherein the polypeptide comprises a sequence having at least 85, 90, 91, 92, 93, 94, 95, 96, 97, 98, 99% identity to SEQ ID NO:1 and comprises a mutation selected from a mutation associated with any of VAR-1 to VAR-16.

5. The variant capsid polypeptide of any of the preceding embodiments, wherein the polypeptide comprises a sequence having at least 85, 90, 91, 92, 93, 94, 95, 96, 97, 98, 99% identity to SEQ ID NO:1 and wherein the variant capsid polypeptide comprises a mutation that corresponds to a mutation at position 550, 559, 561, 586, 587, 592, 593, 597, or any combination thereof, an insertion between positions 584 and 585, 586 and 587, or 587 and 588, or any combination thereof according to SEQ ID NO: 1, optionally wherein the mutation comprises an insertion, a deletion or a substitution.

6. The variant capsid polypeptide of any of the preceding embodiments, wherein the capsid polypeptide comprises a mutation that corresponds to an insertion at position between position 587 and 588 as compared to SEQ ID NO: 1.

7. The variant capsid polypeptide of any of the preceding embodiments, wherein the capsid polypeptide comprises a mutation that corresponds to an insertion at position between position 586 and 587 as compared to SEQ ID NO: 1.

8. The variant capsid polypeptide of any of the preceding embodiments, wherein the capsid polypeptide comprises a mutation that corresponds to a mutation at position 587 as compared to SEQ ID NO: 1, and an insertion at position between position 586 and 587 as compared to SEQ ID NO: 1.

9. The variant capsid polypeptide of any of the preceding embodiments, wherein the capsid polypeptide comprises a mutation that corresponds to a mutation at position 587 and 593 as compared to SEQ ID NO: 1, and an insertion between positions 586 and 587 as compared to SEQ ID NO: 1.

10. The variant capsid polypeptide of any of the preceding embodiments, wherein the capsid polypeptide comprises a mutation that corresponds to a mutation at position 586 and 587 as compared to SEQ ID NO: 1, and an insertion between positions 584 and 585 as compared to SEQ ID NO: 1.

11. The variant capsid polypeptide of any of the preceding embodiments, wherein the capsid polypeptide comprises a mutation that corresponds to a mutation at position 592 and 597 as compared to SEQ ID NO: 1, and an insertion between positions 587 and 588 as compared to SEQ ID NO: 1.

12. The variant capsid polypeptide of any of the preceding embodiments, wherein the capsid polypeptide comprises a mutation that corresponds to a mutation at position 561, 587, and 597 as compared to SEQ ID NO: 1, and an insertion between positions 586 and 587 as compared to SEQ ID NO: 1.

13. The variant capsid polypeptide of any of the preceding embodiments, wherein the capsid polypeptide comprises a mutation that corresponds to a mutation at position 550, 586, and 587 as compared to SEQ ID NO: 1, and an insertion between positions 584 and 585 as compared to SEQ ID NO: 1.

14. The variant capsid polypeptide of any of the preceding embodiments, wherein the capsid polypeptide comprises a mutation that corresponds to a mutation at position 559, and 587 as compared to SEQ ID NO: 1, and an insertion between positions 586 and 587 as compared to SEQ ID NO: 1.

15. The variant capsid polypeptide of any of the preceding embodiments, wherein the capsid polypeptide comprises an insertion, e.g., an insertion of 1 or more amino acids, e.g., 1 amino acid, e.g., 1-2 amino acids, that corresponds to an insertion between 584 and 585, 586 and 587, or 587 and 588, as compared to SEQ ID NO: 1.

16. The variant capsid polypeptide of any of the preceding embodiments, wherein the capsid polypeptide comprises an insertion at a position between position 586 and 587 as compared to SEQ ID NO: 1, wherein the insertion comprises a polypeptide of LALGEQTRPA (SEQ ID NO: 44), or a fragment of at least 5, at least 6, at least 7, at least 8, or at least 9 amino acids thereof.

17. The variant capsid polypeptide of any of the preceding embodiments, wherein the capsid polypeptide comprises an insertion at a position between position 587 and 588 as compared to SEQ ID NO: 1, wherein the insertion comprises a polypeptide of LAIEQTRPA (SEQ ID NO: 45), or a fragment of at least 5, at least 6, at least 7, or at least 8 amino acids thereof.

18. The variant capsid polypeptide of any of the preceding embodiments, wherein the capsid polypeptide comprises a mutation of N587A, and an insertion at a position between position 586 and 587 as compared to SEQ ID NO: 1, wherein the insertion comprises a polypeptide of LALAEITRP (SEQ ID NO: 46), or a fragment of at least 5, at least 6, at least 7, or at least 8 amino acids thereof.

19. The variant capsid polypeptide of any of the preceding embodiments, wherein the capsid polypeptide comprises a mutation of N587A, and an insertion at a position between position 586 and 587 as compared to SEQ ID NO: 1, wherein the insertion comprises a polypeptide of LKNAETARP (SEQ ID NO: 47), or a fragment of at least 5, at least 6, at least 7, or at least 8 amino acids thereof.

20. The variant capsid polypeptide of any of the preceding embodiments, wherein the capsid polypeptide comprises a mutation of N587A and A593T, and an insertion at a position between position 586 and 587 as compared to SEQ ID NO: 1, wherein the insertion comprises a polypeptide of LNLAIEQTRP (SEQ ID NO: 48), or a fragment of at least 5, at least 6, at least 7, or at least 8 amino acids thereof.

21. The variant capsid polypeptide of any of the preceding embodiments, wherein the capsid polypeptide comprises a mutation of N587A, and an insertion at a position between position 586 and 587 as compared to SEQ ID NO: 1, wherein the insertion comprises a polypeptide of MLNEQTRP (SEQ ID NO: 49), or a fragment of at least 4, at least 5, at least 6, or at least 7 amino acids thereof.

22. The variant capsid polypeptide of any of the preceding embodiments, wherein the capsid polypeptide comprises a mutation of G586P and N587A, and an insertion at a position between position 584 and 585 as compared to SEQ ID NO: 1, wherein the insertion comprises a polypeptide of RSGNRADSETA (SEQ ID NO: 50), or a fragment of at least 5, at least 6, at least 7, at least 8, at least 9, or at least 10 amino acids thereof.

23. The variant capsid polypeptide of any of the preceding embodiments, wherein the capsid polypeptide comprises a mutation of N587A, and an insertion at a position between position 586 and 587 as compared to SEQ ID NO: 1, wherein the insertion comprises a polypeptide of TGDTRP (SEQ ID NO: 51), or a fragment of at least 3, at least 4, or at least 5 amino acids thereof.

24. The variant capsid polypeptide of any of the preceding embodiments, wherein the capsid polypeptide comprises an insertion at a position between position 587 and 588 as compared to SEQ ID NO: 1, wherein the insertion comprises a polypeptide of LQGETIRPA (SEQ ID NO: 52), or a fragment of at least 5, at least 6, at least 7, at least 8, at least 9, or at least 10 amino acids thereof.

25. The variant capsid polypeptide of any of the preceding embodiments, wherein the capsid polypeptide comprises a mutation of N587A, and an insertion at a position between position 586 and 587 as compared to SEQ ID NO: 1, wherein the insertion comprises a polypeptide of QNLANPETTRP (SEQ ID NO: 53), or a fragment of at least 5, at least 6, at least 7, at least 8, at least 9, or at least 10 amino acids thereof.

26. The variant capsid polypeptide of any of the preceding embodiments, wherein the capsid polypeptide comprises a mutation of T592A and T597W, and an insertion at a position between position 587 and 588 as compared to SEQ ID NO: 1, wherein the insertion comprises a polypeptide of RAPQETTRPA (SEQ ID NO: 54), or a fragment of at least 5, at least 6, at least 7, at least 8, or at least 9 amino acids thereof.

27. The variant capsid polypeptide of any of the preceding embodiments, wherein the capsid polypeptide comprises a mutation of N587A, and an insertion at a position between position 586 and 587 as compared to SEQ ID NO: 1, wherein the insertion comprises a polypeptide of ANLTTTRP (SEQ ID NO: 55), or a fragment of at least 4, at least 5, at least 6, or at least 7 amino acids thereof.

28. The variant capsid polypeptide of any of the preceding embodiments, wherein the capsid polypeptide comprises a mutation of N587A, and an insertion at a position between position 586 and 587 as compared to SEQ ID NO: 1, wherein the insertion comprises a polypeptide of ALLAGEQTRP (SEQ ID NO: 56), or a fragment of at least 5, at least 6, at least 7, at least 8, or at least 9 amino acids thereof.

29. The variant capsid polypeptide of any of the preceding embodiments, wherein the capsid polypeptide comprises a mutation of D561C, N587A, and T597N, and an insertion at a position between position 586 and 587 as compared to SEQ ID NO: 1, wherein the insertion comprises a polypeptide of GLRAEQTRP (SEQ ID NO: 57), or a fragment of at least 5, at least 6, at least 7, or at least 8 amino acids thereof.

30. The variant capsid polypeptide of any of the preceding embodiments, wherein the capsid polypeptide comprises a mutation of T550N, G586P, and N587A, and an insertion at a position between position 584 and 585 as compared to SEQ ID NO: 1, wherein the insertion comprises a polypeptide of RARLDETA (SEQ ID NO: 58), or a fragment of at least 4, at least 5, at least 6, or at least 7 amino acids thereof.

31. The variant capsid polypeptide of any of the preceding embodiments, wherein the capsid polypeptide comprises a mutation of I559L, and N587A, and an insertion at a position between position 586 and 587 as compared to SEQ ID NO: 1, wherein the insertion comprises a polypeptide of TNLARGETARP (SEQ ID NO: 59), or a fragment of at least 5, at least 6, at least 7, at least 8, at least 9, or at least 10 amino acids thereof.

32. A variant capsid polypeptide, comprising (a) a polypeptide of any one of SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27, (b) the VP2 or VP3 sequence of any one of SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27, (c) a polypeptide comprising a sequence having at least 90%, at least 95%, at least 96%, at least 97%, at least 98%, or at least 99% identity thereto, wherein said sequence comprises at least one (e.g., one, two, three or more, e.g., all) of the mutation differences associated with any of SEQ ID NO: 12 through SEQ ID NO: 27, relative to SEQ ID NO: 1; or (d) a polypeptide having at least 1, but no more than 20, no more than 19, no more than 18, no more than 17, no more than 16, no more than 15, no more than 14, no more than 13, no more than 12, no more than 10, no more than 9, no more than 8, no more than 7, no more than 6, no more than 5, no more than 3, or no more than 2 amino acid mutations relative to the polypeptide of (a) or (b), wherein said polypeptide comprises at least one (e.g., one, two, three or more, e.g., all) of the mutation differences associated with any of SEQ ID NO: 12 through SEQ ID NO: 27, relative to SEQ ID NO: 1.

33. The variant capsid polypeptide of any of the preceding embodiments, wherein the variant capsid polypeptide is a VP1 polypeptide, a VP2 polypeptide or a VP3 polypeptide.

34. A variant capsid polypeptide comprising:
an amino acid sequence that has 95% or more amino acid sequence identity to an amino acid sequence of one of SEQ ID NOs: 12-27; and
has at least 80% of the mutations in said amino acid sequence of one of SEQ ID NO: 12-27 as compared to SEQ ID NO: 1.

35. A variant capsid polypeptide comprising:
an amino acid sequence that has less than 95% amino acid sequence identity to an amino acid sequence of one of SEQ ID NOs: 12-27; and
has at least 80% of the mutations in said amino acid sequence of one of SEQ ID NO: 12-27 as compared to SEQ ID NO: 1.

36. A variant capsid polypeptide comprising:
an amino acid sequence that has 95% or more amino acid sequence identity to an amino acid sequence of one of SEQ ID NOs: 12-27; and
has less than 80% of the mutations in said amino acid sequence of one of SEQ ID NO: 12-27 as compared to SEQ ID NO: 1.

37. A nucleic acid molecule comprising a sequence encoding a variant capsid polypeptide of any one of embodiments 1-36.

38. The nucleic acid molecule of embodiment 37, comprising one or more regulatory elements operably linked to the sequence encoding the variant capsid polypeptide.

39. The nucleic acid molecule of any of embodiments 37-38, comprising SEQ ID NO: 28, 29, 30, 31, 32, 33, 34, 35, 36, 37, 38, 39, 40, 41, 42, or 43, or a fragment thereof, or a variant thereof having at least 85, 90, 91, 92, 93, 94, 95, 96, 97, 98, 99, or 100% sequence identity thereto.

40. A virus particle (e.g., adeno-associated virus (“AAV”) particle) comprising the variant capsid polypeptide of any one of embodiments 1-36 or comprising a variant capsid polypeptide encoded by the nucleic acid molecule of any one of embodiments 37-39.

41. The virus particle of embodiment 40, comprising a nucleic acid comprising a heterologous transgene and one or more regulatory elements.

42. A virus particle of any of embodiments 40-41 comprising the variant capsid polypeptide of any one of embodiments 1-36, wherein said virus particle, or a virus particle comprising said variant capsid polypeptide or a virus particle comprising a variant capsid polypeptide encoded by a nucleic acid molecule of any one of embodiments 37-39 exhibits increased ocular transduction, e.g., as measured in a mouse or in NHP, e.g., as described herein, relative to wild-type AAV2 (e.g., a virus particle comprising capsid polypeptides of SEQ ID NO: 1 or encoded by SEQ ID NO: 2).

43. The nucleic acid molecule of any one of embodiments 37-39 or virus particle of any one of embodiments 40-42 wherein the nucleic acid molecule is double-stranded or single-stranded, optionally wherein the nucleic acid molecule is linear or circular, e.g., wherein the nucleic acid molecule is a plasmid.

44. A method of producing a virus particle comprising a variant AAV2 capsid polypeptide, said method comprising introducing a nucleic acid molecule of any one of embodiments 37-39 or 43 into a cell (e.g., a HEK293 cell), and harvesting said virus particle therefrom.

45. A method of delivering a payload (e.g., a nucleic acid) to a cell comprising contacting the cell with a dependoparvovirus particle comprising a variant capsid polypeptide of any one of embodiments 1-36 or the virus particle of any of embodiments 40-42 and a payload.

46. The method of embodiment 45, wherein the cell is an ocular cell.

47. The method of embodiment 46, wherein the ocular cell is in the retina, the macula, or the trabecular meshwork.

48. A method of delivering a payload (e.g., a nucleic acid) to a subject comprising administering to the subject a dependoparvovirus particle comprising a variant capsid polypeptide of any one of embodiments 1-36 and the payload, or administering to the subject the virus particle of any one of embodiments 40-42.

49. The method of embodiment 48, wherein the particle delivers the payload to the eye.

50. The method of embodiment 48, wherein the particle delivers the payload to the retina, the macular, or the trabecular meshwork.

51. The method of any one of embodiments 48-50, wherein the particle delivers the payload to the eye with increased transduction in one or more regions of the eye as compared to a virus particle comprising capsid polypeptides of SEQ ID NO: 1.

52. The method of embodiment 51, wherein the one or more regions of the eye is selected from the retina, the macula, the trabecular meshwork, or any combination thereof.

53. The method of embodiment 51, wherein the retina comprises non-macular retina.

54. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 64-times, 100-times, 128-times, 200-times, 300-times, 400-times, 500-times, or 1000-times as compared to a virus particle comprising capsid polypeptides of SEQ ID NO: 1.

55. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, or 32-times as compared to a virus particle comprising capsid polypeptides of SEQ ID NO: 1, and wherein the increase in transduction is specific to non-macular retina tissue relative to macular tissue.

56. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to macular tissue relative to non-macular retina tissue.

57. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to macular tissue relative to trabecular meshwork tissue.

58. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, or 32-times as compared to a virus particle comprising capsid polypeptides of SEQ ID NO: 1, and wherein the increase in transduction is specific to non-macular retina tissue relative to trabecular meshwork tissue.

59. The variant capsid polypeptide of any of embodiments 1-36, the virus particle of any of embodiments 40-42 or the method of any one of embodiments 44-53, wherein the (e.g., particle comprising the variant capsid polypeptide) particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, or 32-times as compared to a virus particle comprising capsid polypeptides of SEQ ID NO: 1, and wherein the increase in transduction is specific to macular tissue and non-macular retina tissue relative to trabecular meshwork tissue.

60. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue relative to macular tissue and non-macular retina tissue.

61. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue relative to macular tissue.

62. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, or 16-times as compared to a virus particle comprising capsid polypeptides of SEQ ID NO: 1, and wherein the increase in transduction is specific to trabecular meshwork tissue relative to non-macular retina tissue.

63. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue, macular tissue, and non-macular retina tissue.

64. The variant capsid polypeptide of any of embodiments 1-36, the virus particle of any of embodiments 40-42 or the method of any one of embodiments 44-53, wherein the particle (e.g., particle comprising the variant capsid polypeptide) delivers the payload to the eye with increased transduction specificity in one or more regions of the eye as compared to a virus particle comprising capsid polypeptides of SEQ TD NO: 1 without increased biodistribution in one or more regions of the eye as compared to a virus particle comprising capsid polypeptides of SEQ ID NO: 1.

65. The method of any one of embodiments 45-53, wherein the administration to the subject is via an intravitreal injection, or an intracameral injection.

66. A method of treating a disease or condition in a subject, comprising administering to the subject a dependoparvovirus particle in an amount effective to treat the disease or condition, wherein the dependoparvovirus particle is a particle comprising a capsid polypeptide of any one of embodiments 1-36 and 54-64, or encoded by the nucleic acid of any one of embodiments 37-39 or 43, or is a virus particle of any one of embodiments 40-42.

67. A cell, cell-free system, or other translation system, comprising the capsid polypeptide, nucleic acid molecule, or virus particle of any one of embodiments 1-43 or 54-64.

68. A method of making a dependoparvovirus (e.g., an adeno-associated dependoparvovirus (AAV) particle, comprising:
providing a cell, cell-free system, or other translation system, comprising a nucleic acid of any of embodiments 37-39 or 43; and
cultivating the cell, cell-free system, or other translation system, under conditions suitable for the production of the dependoparvovirus particle,
thereby making the dependoparvovirus particle.

69. The method of embodiment 68, wherein the cell, cell-free system, or other translation system comprises a second nucleic acid molecule and said second nucleic acid molecule is packaged in the dependoparvovirus particle.

70. The method of embodiment 68, wherein the second nucleic acid comprises a payload, e.g., a heterologous nucleic acid sequence encoding a therapeutic product.

71. The method of any one of embodiments 68-70, wherein the nucleic acid of any of embodiments 37-39 or 43 mediates the production of a dependoparvovirus particle which does not include said nucleic acid of any of embodiments 37-39 or 43.

72. The method of any one of embodiments 68-71, wherein the nucleic acid of any of embodiments 37-39 or 43 mediates the production of a dependoparvovirus particle at a level at least 10%, at least 20%, at least 50%, at least 100%, at least 200% or greater than the production level mediated by the nucleic acid of SEQ ID NO: 2.

74. A composition, e.g., a pharmaceutical composition, comprising a virus particle of any one of embodiments 40-42 or a virus particle produced by the method of any one of embodiments 43 or 68-72, and a pharmaceutically acceptable carrier.

75. The variant capsid polypeptide of any of embodiments 1-36 and 54-64, the nucleic acid molecule of any of embodiments 37-39 or 43, or the virus particle of any of embodiments 40-42 for use in treating a disease or condition in a subject.

76. The variant capsid polypeptide of any of embodiments 1-36 and 54-64, the nucleic acid molecule of any of embodiments 37-39 or 43, or the virus particle of any of embodiments 40-42 for use in the manufacture of a medicament for use in treating a disease or condition in a subject.

### DETAILED DESCRIPTION

The present disclosure is directed, in part, to capsid polypeptides and dependoparvovirus particles comprising the same. In some embodiments, the dependoparvovirus particles have increased ocular transduction and can be used to deliver a transgene or molecule of interest to an eye with higher transduction efficiency in the eye as compared to a dependoparvovirus particle without the variant capsid polypeptides. Accordingly, provided herein are capsid polypeptides, nucleic acid molecules encoding the same, viral particles comprising the variant capsid polypeptides, and methods of making and using the same.

### Definitions

A, An, The: As used herein, the singular forms “a,” “an” and “the” include plural referents unless the context clearly dictates otherwise.

About, Approximately: As used herein, the terms “about” and “approximately” shall generally mean an acceptable degree of error for the quantity measured given the nature or precision of the measurements. Exemplary degrees of error are within 15 percent (%), typically, within 10%, and more typically, within 5% of a given value or range of values.

Dependoparvovirus capsid: As used herein, the term “dependoparvovirus capsid” refers to an assembled viral capsid comprising dependoparvovirus polypeptides. In some embodiments, a dependoparvovirus capsid is a functional dependoparvovirus capsid, e.g., is fully folded and/or assembled, is competent to infect a target cell, or remains stable (e.g., folded/assembled and/or competent to infect a target cell) for at least a threshold time.

Dependoparvovirus particle: As used herein, the term “dependoparvovirus particle” refers to an assembled viral capsid comprising dependoparvovirus polypeptides and a packaged nucleic acid, e.g., comprising a payload, one or more components of a dependoparvovirus genome (e.g., a whole dependoparvovirus genome), or both. In some embodiments, a dependoparvovirus particle is a functional dependoparvovirus particle, e.g., comprises a desired payload, is fully folded and/or assembled, is competent to infect a target cell, or remains stable (e.g., folded/assembled and/or competent to infect a target cell) for at least a threshold time.

Dependoparvovirus X particle/capsid: As used herein, the term “dependoparvovirus X particle/capsid” refers to a dependoparvovirus particle/capsid comprising at least one polypeptide or polypeptide encoding nucleic acid sequence derived from a naturally occurring dependoparvovirus X species. For example, a dependoparvovirus B particle refers to a dependoparvovirus particle comprising at least one polypeptide or polypeptide encoding nucleic acid sequence derived from a naturally occurring dependoparvovirus B sequence. Derived from, as used in this context, means having at least 80, 85, 90, 91, 92, 93, 94, 95, 96, 97, 98, 99, or 100% identity to the sequence in question. Correspondingly, an AAVX particle/capsid, as used herein, refers to an AAV particle/capsid comprising at least one polypeptide or polypeptide encoding nucleic acid sequence derived from a naturally occurring AAV X serotype. For example, an AAV2 particle refers to an AAV particle comprising at least one polypeptide or polypeptide encoding nucleic acid sequence derived from a naturally occurring AAV2 sequence.

Exogenous: As used herein, the term “exogenous” refers to a feature, sequence, or component present in a circumstance (e.g., in a nucleic acid, polypeptide, or cell) that does not naturally occur in said circumstance. For example, a nucleic acid sequence comprising a mutant capsid polypeptide or a nucleic acid molecule encoding the same may comprise a capsid polypeptide. Use of the term exogenous in this fashion means that the polypeptide or the nucleic acid molecule encoding a polypeptide comprising the mutation in question at this position does not occur naturally, e.g., is not present in AAV2, e.g., is not present in SEQ ID NO: 1.

Functional: As used herein in reference to a polypeptide component of a dependoparvovirus capsid (e.g., Cap (e.g., VP1, VP2, and/or VP3) or Rep), the term “functional” refers to a polypeptide which provides at least 50, 60, 70, 80, 90, or 100% of the activity of a naturally occurring version of that polypeptide component (e.g., when present in a host cell). For example, a functional VP1 polypeptide may stably fold and assemble into a dependoparvovirus capsid (e.g., that is competent for packaging and/or secretion). As used herein in reference to a dependoparvovirus capsid or particle, “functional” refers to a capsid or particle comprising one or more of the following production characteristics: comprises a desired payload, is fully folded and/or assembled, is competent to infect a target cell, or remains stable (e.g., folded/assembled and/or competent to infect a target cell) for at least a threshold time.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid monomer (e.g., a nucleotide and/or nucleoside); in some embodiments, “nucleic acid” refers to an oligonucleotide chain comprising individual nucleic acid monomers or a longer polynucleotide chain comprising many individual nucleic acid monomers. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid is, comprises, or consists of one or more modified, synthetic, or non-naturally occurring nucleotides. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid has a nucleotide sequence that encodes a functional gene product such as an RNA or protein. In some embodiments, a nucleic acid is partly or wholly single stranded; in some embodiments, a nucleic acid is partly or wholly double stranded.

Variant: As used herein, a “variant capsid polypeptide” refers to a polypeptide that differs from a reference sequence (e.g. SEQ ID NO: 1). The variant can, for example, comprise a mutation (e.g. substitution, deletion, or insertion). In some embodiments, the variant is about, or at least, 85%, 86%, 87%, 88%, 89%, 90%, 91%, 92%, 93%, 94%, 95%, 96%, 97%, 98%, or 99% identical to the reference sequence. In some embodiments, the reference sequence is a polypeptide comprising SEQ ID NO: 1.

### Capsid Polypeptides and Nucleic Acids Encoding the Same

The disclosure is directed, in part, to capsid polypeptides comprising a mutation (insertion, deletion, or substitution) as compared to the wild-type sequence, viral particles comprising variant capsid polypeptides, such as those described here, nucleic acid molecules, and nucleic acid molecules encoding capsid polypeptides such as those described herein. In some embodiments, the wild-type sequence is SEQ ID NO: 1. The disclosure is directed, in part, to variant capsid polypeptides comprising SEQ ID NO: 1 with one or more mutations as compared to SEQ ID NO: 1. The mutation can be, for example, an insertion, deletion, or substitution as compared to the wild-type sequence. In some embodiments, the wild-type sequence is SEQ ID NO: 1. The disclosure is directed, in part, to a variant capsid polypeptide comprising any one of SEQ ID NO: 12 to SEQ ID NO: 27. The disclosure is directed, in part, to a variant capsid polypeptide comprising a VP1 sequence of any one of SEQ ID NO: 12 to SEQ ID NO: 27. The disclosure is directed, in part, to a variant capsid polypeptide comprising a VP2 sequence of any one of SEQ ID NO: 12 to SEQ ID NO: 27. The disclosure is directed, in part, to a variant capsid polypeptide comprising a VP3 sequence of any one of SEQ ID NO: 12 to SEQ ID NO: 27.

In some embodiments, the capsid polypeptide comprises a mutation selected from the mutation differences disclosed in any of Tables 1A-1G, e.g., selected from the mutation differences associated with any variant disclosed in any of Tables 1A-1G. In some embodiments, the mutation differences disclosed in any of Tables 1A-1G, e.g., selected from the mutation differences associated with any variant disclosed in any of Tables 1A-1G comprises mutations at positions corresponding to residues 550-597 as compared to SEQ ID NO: 1. In some embodiments, the capsid polypeptide comprises a mutation within the 550-597 amino acid region of SEQ ID NO: 1. In some embodiments, the capsid polypeptide comprises a mutation selected from the mutation differences disclosed in any of Tables 1A-1G, e.g., selected from the mutation differences associated with any variant disclosed in any of Tables 1A-1G. In some embodiments, the mutation selected from the mutation differences disclosed in any of Tables 1A-1G, e.g., selected from the mutation differences associated with any variant disclosed in any of Tables 1A-1G is a substitution, e.g., a substitution of 2 or more residues that correspond to a substitution at positions between 550 and 597 as compared to SEQ ID NO: 1. In some embodiments, the mutation selected from the mutation differences disclosed in any of Tables 1A-1G, e.g., selected from the mutation differences associated with any variant disclosed in any of Tables 1A-1G is a substitution and further comprises at least one other mutation between positions 550 and 597, wherein the mutations are substitutions, insertions, or deletions. In some embodiments, the mutation selected from the mutation differences disclosed in any of Tables 1A-1G, e.g., selected from the mutation differences associated with any variant disclosed in any of Tables 1A-1G is an insertion, e.g., an insertion of 1 or more amino acids, e.g. 1 amino acid, e.g., 1-2 amino acids, that correspond to an insertion between positions 584 and 585, 586 and 587, or 587 and 588 as compared to SEQ ID NO. 1; and a substitution, e.g., a substitution of 2 or more residues that correspond to a substitution at positions between 550 and 597 as compared to SEQ ID NO: 1.

In some embodiments, the capsid polypeptide comprises a mutation that corresponds to a mutation at position 550, 559, 561, 586, 587, 592, 593, 597, or any combination thereof, an insertion between positions 584 and 585, 586 and 587, or 587 and 588, or any combination thereof according to SEQ ID NO: 1, and optionally wherein the mutation comprises an insertion, a deletion or a substitution.

In some embodiments, the capsid polypeptide comprises a mutation that corresponds to a mutation at position 550 as compared to SEQ ID NO: 1. In some embodiments, the capsid polypeptide comprises a mutation that corresponds to a mutation at position 559 as compared to SEQ ID NO: 1. In some embodiments, the capsid polypeptide comprises a mutation that corresponds to a mutation at position 561 as compared to SEQ ID NO: 1. In some embodiments, the capsid polypeptide comprises a mutation that corresponds to a mutation at position 586 as compared to SEQ ID NO: 1. In some embodiments, the capsid polypeptide comprises a mutation that corresponds to a mutation at position 587 as compared to SEQ ID NO: 1. In some embodiments, the capsid polypeptide comprises a mutation that corresponds to a mutation at position 592 as compared to SEQ ID NO: 1. In some embodiments, the capsid polypeptide comprises a mutation that corresponds to a mutation at position 593 as compared to SEQ ID NO: 1. In some embodiments, the capsid polypeptide comprises a mutation that corresponds to a mutation at position 597 as compared to SEQ ID NO: 1.

In some embodiments, the capsid polypeptide comprises a mutation that corresponds to an insertion at position between positions 584 and 585 as compared to SEQ ID NO: 1. In some embodiments, the capsid polypeptide comprises a mutation that corresponds to an insertion at position between positions 586 and 587 as compared to SEQ ID NO: 1. In some embodiments, the capsid polypeptide comprises a mutation that corresponds to an insertion at position between positions 587 and 588 as compared to SEQ ID NO: 1.

In some embodiments, the capsid polypeptide comprises a mutation that corresponds to a mutation at position 550, 559, 561, 586, 587, 592, 593, 597, or any combination thereof, an insertion between positions 584 and 585, 586 and 587, or 587 and 588, or any combination thereof according to SEQ ID NO: 1, and wherein the mutation comprises an insertion, a deletion or a substitution.

In some embodiments, the capsid polypeptide comprises a mutation that corresponds to a mutation at position 587 and 593 as compared to SEQ ID NO: 1.

In some embodiments, the capsid polypeptide comprises a mutation that corresponds to a mutation at position 586 and 587 as compared to SEQ ID NO: 1.

In some embodiments, the capsid polypeptide comprises a mutation that corresponds to a mutation at position 592 and 597 as compared to SEQ ID NO: 1.

In some embodiments, the capsid polypeptide comprises a mutation that corresponds to a mutation at position 587 and 597 as compared to SEQ ID NO: 1.

In some embodiments, the capsid polypeptide comprises a mutation that corresponds to a mutation at position 550, 586, and 587 as compared to SEQ ID NO: 1.

In some embodiments, the capsid polypeptide comprises a mutation that corresponds to a mutation at position 559 and 587 as compared to SEQ ID NO: 1.

In some embodiments, the capsid polypeptide comprises a mutation that corresponds to a mutation at position 587, and an insertion between positions 586 and 587 as compared to SEQ ID NO: 1.

In some embodiments, the capsid polypeptide comprises a mutation that corresponds to a mutation at position 587 and 593, and an insertion between positions 586 and 587 as compared to SEQ ID NO: 1.

In some embodiments, the capsid polypeptide comprises a mutation that corresponds to a mutation at position 586 and 587, and an insertion between positions 584 and 585 as compared to SEQ ID NO: 1.

In some embodiments, the capsid polypeptide comprises a mutation that corresponds to a mutation at position 592 and 597, and an insertion between positions 587 and 588 as compared to SEQ ID NO: 1.

In some embodiments, the capsid polypeptide comprises a mutation that corresponds to a mutation at position 561, 587, and 597, and an insertion between positions 586 and 587 as compared to SEQ ID NO: 1.

In some embodiments, the capsid polypeptide comprises a mutation that corresponds to a mutation at position 550, 586, and 587, and an insertion between positions 584 and 585 as compared to SEQ ID NO: 1.

In some embodiments, the capsid polypeptide comprises a mutation that corresponds to a mutation at position 559, and 587, and an insertion between positions 586 and 587 as compared to SEQ ID NO: 1.

In some embodiments, the mutation that corresponds to position 550 is a substitution as compared to SEQ ID NO: 1. In some embodiments, the substitution is to a naturally occurring amino acid. In some embodiments, the substitution is asparagine (N). In some embodiments, the substitution at position 550 is T550N according to SEQ ID NO: 1. In some embodiments, the substitution at a position corresponding to T550 of SEQ ID NO: 1 is a substitution to asparagine (N) at the position corresponding to T550 of SEQ ID NO: 1 in a reference capsid sequence other than SEQ ID NO: 1, e.g., as described herein. In some embodiments, the capsid polypeptide comprises a mutation that corresponds to a mutation T550N mutation as compared to SEQ ID NO: 1.

In some embodiments, the mutation that corresponds to position 559 is a substitution as compared to SEQ ID NO: 1. In some embodiments, the substitution is to a naturally occurring amino acid. In some embodiments, the substitution is leucine (L). In some embodiments, the substitution at position 559 is I559L according to SEQ ID NO: 1. In some embodiments, the substitution at a position corresponding to I559 of SEQ ID NO: 1 is a substitution to leucine (L) at the position corresponding to I559 of SEQ ID NO: 1 in a reference capsid sequence other than SEQ ID NO: 1, e.g., as described herein. In some embodiments, the capsid polypeptide comprises a mutation that corresponds to a mutation I559L mutation as compared to SEQ ID NO: 1.

In some embodiments, the mutation that corresponds to position 561 is a substitution as compared to SEQ ID NO: 1. In some embodiments, the substitution is to a naturally occurring amino acid. In some embodiments, the substitution is cysteine (C). In some embodiments, the substitution at position 561 is D561 according to SEQ ID NO: 1. In some embodiments, the substitution at a position corresponding to D561 of SEQ ID NO: 1 is a substitution to cysteine (C) at the position corresponding to D561 of SEQ ID NO: 1 in a reference capsid sequence other than SEQ ID NO: 1, e.g., as described herein. In some embodiments, the capsid polypeptide comprises a mutation that corresponds to a mutation D561C mutation as compared to SEQ ID NO: 1.

In some embodiments, the mutation that corresponds to position 586 is a substitution as compared to SEQ ID NO: 1. In some embodiments, the substitution is to a naturally occurring amino acid. In some embodiments, the substitution is proline (P). In some embodiments, the substitution at position 586 is G586P according to SEQ ID NO: 1. In some embodiments, the substitution at a position corresponding to G586 of SEQ ID NO: 1 is a substitution to proline (P) at the position corresponding to G586 of SEQ ID NO: 1 in a reference capsid sequence other than SEQ ID NO: 1, e.g., as described herein. In some embodiments, the capsid polypeptide comprises a mutation that corresponds to a mutation G586P mutation as compared to SEQ ID NO: 1.

In some embodiments, the mutation that corresponds to position 587 is a substitution as compared to SEQ ID NO: 1. In some embodiments, the substitution is to a naturally occurring amino acid. In some embodiments, the substitution is alanine (A). In some embodiments, the substitution at position 587 is N587A according to SEQ ID NO: 1. In some embodiments, the substitution at a position corresponding to N587 of SEQ ID NO: 1 is a substitution to alanine (A) at the position corresponding to N587 of SEQ ID NO: 1 in a reference capsid sequence other than SEQ ID NO: 1, e.g., as described herein. In some embodiments, the capsid polypeptide comprises a mutation that corresponds to a mutation N587A mutation as compared to SEQ ID NO: 1.

In some embodiments, the mutation that corresponds to position 592 is a substitution as compared to SEQ ID NO: 1. In some embodiments, the substitution is to a naturally occurring amino acid. In some embodiments, the substitution is alanine (A). In some embodiments, the substitution at position 592 is T592A according to SEQ ID NO: 1. In some embodiments, the substitution at a position corresponding to T592 of SEQ ID NO: 1 is a substitution to alanine (A) at the position corresponding to T592 of SEQ ID NO: 1 in a reference capsid sequence other than SEQ ID NO: 1, e.g., as described herein. In some embodiments, the capsid polypeptide comprises a mutation that corresponds to a mutation T592A mutation as compared to SEQ ID NO: 1.

In some embodiments, the mutation that corresponds to position 593 is a substitution as compared to SEQ ID NO: 1. In some embodiments, the substitution is to a naturally occurring amino acid. In some embodiments, the substitution is threonine (T). In some embodiments, the substitution at position 593 is A593T according to SEQ ID NO: 1. In some embodiments, the substitution at a position corresponding to A593 of SEQ ID NO: 1 is a substitution to threonine (T) at the position corresponding to A593 of SEQ ID NO: 1 in a reference capsid sequence other than SEQ ID NO: 1, e.g., as described herein. In some embodiments, the capsid polypeptide comprises a mutation that corresponds to a mutation A593T mutation as compared to SEQ ID NO: 1.

In some embodiments, the mutation that corresponds to position 597 is a substitution as compared to SEQ ID NO: 1. In some embodiments, the substitution is to a naturally occurring amino acid. In some embodiments, the substitution is asparagine (N). In some embodiments, the substitution is to a naturally occurring amino acid. In some embodiments, the substitution is tryptophan (W). In some embodiments, the substitution at position 597 is T597N according to SEQ ID NO: 1. In some embodiments, the substitution at position 597 is T597W according to SEQ ID NO: 1. In some embodiments, the substitution at a position corresponding to T597 of SEQ ID NO: 1 is a substitution to asparagine (N) at the position corresponding to T597 of SEQ ID NO: 1 in a reference capsid sequence other than SEQ ID NO: 1, e.g., as described herein. In some embodiments, the substitution at a position corresponding to T597 of SEQ ID NO: 1 is a substitution to tryptophan (W) at the position corresponding to T597 of SEQ ID NO: 1 in a reference capsid sequence other than SEQ ID NO: 1, e.g., as described herein. In some embodiments, the capsid polypeptide comprises a mutation that corresponds to a mutation T597N mutation as compared to SEQ ID NO: 1. In some embodiments, the capsid polypeptide comprises a mutation that corresponds to a mutation T597W mutation as compared to SEQ ID NO: 1.

In some embodiments, the capsid polypeptide comprises a mutation that corresponds to an insertion between residues 587 and 588 as compared to SEQ ID NO: 1, wherein the insertion comprises, e.g., consists of, a polypeptide of LALGEQTRPA (SEQ ID NO: 44). In some embodiments, the insertion comprises a polypeptide that has at least 50%, 60%, 70%, 80%, 90%, or 100% identity to LALGEQTRPA (SEQ ID NO: 44). In some embodiments, the insertion comprises a polypeptide that has at least 1, 2, 3, 4, or 5 mutations as compared to LALGEQTRPA (SEQ ID NO: 44). In some embodiments, the insertion comprises a polypeptide comprising a fragment of at least 5 amino acids of LALGEQTRPA (SEQ ID NO: 44). In some embodiments, the insertion comprises a polypeptide comprising a fragment of at least 6 amino acids of LALGEQTRPA (SEQ ID NO: 44). In some embodiments, the insertion comprises a polypeptide comprising a fragment of at least 7 amino acids of LALGEQTRPA (SEQ ID NO: 44). In some embodiments, the insertion comprises a polypeptide comprising a fragment of at least 8 amino acids of LALGEQTRPA (SEQ ID NO: 44). In some embodiments, the insertion comprises a polypeptide comprising a fragment of at least 9 amino acids of LALGEQTRPA (SEQ ID NO: 44).

In some embodiments, the capsid polypeptide comprises a mutation that corresponds to an insertion between residues 587 and 588 as compared to SEQ ID NO: 1, wherein the insertion comprises, e.g., consists of, a polypeptide of LAIEQTRPA (SEQ ID NO: 45). In some embodiments, the insertion comprises a polypeptide that has at least 44.4%, 55.5%, 66.6%, 77.7%, 88.8%, or 100% identity to LAIEQTRPA (SEQ ID NO: 45). In some embodiments, the insertion comprises a polypeptide that has at least 1, 2, 3, 4, or 5 mutations as compared to LAIEQTRPA (SEQ ID NO: 45). In some embodiments, the insertion comprises a polypeptide comprising a fragment of at least 5 amino acids of LAIEQTRPA (SEQ ID NO: 45). In some embodiments, the insertion comprises a polypeptide comprising a fragment of at least 6 amino acids of LAIEQTRPA (SEQ ID NO: 45). In some embodiments, the insertion comprises a polypeptide comprising a fragment of at least 7 amino acids of LAIEQTRPA (SEQ ID NO: 45). In some embodiments, the insertion comprises a polypeptide comprising a fragment of at least 8 amino acids of LAIEQTRPA (SEQ ID NO: 45).

In some embodiments, the capsid polypeptide comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of LALAEITRP (SEQ ID NO: 46). In some embodiments, the insertion comprises a polypeptide that has at least 44.4%, 55.5%, 66.6%, 77.7%, 88.8%, or 100% identity to LALAEITRP (SEQ ID NO: 46). In some embodiments, the insertion comprises a polypeptide that has at least 1, 2, 3, 4, or 5 mutations as compared to LALAEITRP (SEQ ID NO: 46). In some embodiments, the insertion comprises a polypeptide comprising a fragment of at least 5 amino acids of LALAEITRP (SEQ ID NO: 46). In some embodiments, the insertion comprises a polypeptide comprising a fragment of at least 6 amino acids of LALAEITRP (SEQ ID NO: 46). In some embodiments, the insertion comprises a polypeptide comprising a fragment of at least 7 amino acids of LALAEITRP (SEQ ID NO: 46). In some embodiments, the insertion comprises a polypeptide comprising a fragment of at least 8 amino acids of LALAEITRP (SEQ ID NO: 46).

In some embodiments, the capsid polypeptide comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of LKNAETARP (SEQ ID NO: 47). In some embodiments, the insertion comprises a polypeptide that has at least 44.4%, 55.5%, 66.6%, 77.7%, 88.8%, or 100% identity to LKNAETARP (SEQ ID NO: 47). In some embodiments, the insertion comprises a polypeptide that has at least 1, 2, 3, 4, or 5 mutations as compared to LKNAETARP (SEQ ID NO: 47). In some embodiments, the insertion comprises a polypeptide comprising a fragment of at least 5 amino acids of LKNAETARP (SEQ ID NO: 47). In some embodiments, the insertion comprises a polypeptide comprising a fragment of at least 6 amino acids of LKNAETARP (SEQ ID NO: 47). In some embodiments, the insertion comprises a polypeptide comprising a fragment of at least 7 amino acids of LKNAETARP (SEQ ID NO: 47). In some embodiments, the insertion comprises a polypeptide comprising a fragment of at least 8 amino acids of LKNAETARP (SEQ ID NO: 47).

In some embodiments, the capsid polypeptide comprises a mutation that corresponds a N587A and A593T mutations as compared to SEQ ID NO: 1, and an insertion between residues 586 and 587 as compared to SEQ ID NO: 1, wherein the insertion comprises, e.g., consists of, a polypeptide of LNLAIEQTRP (SEQ ID NO: 48). In some embodiments, the insertion comprises a polypeptide that has at least 50%, 60%, 70%, 80%, 90%, or 100% identity to LNLAIEQTRP (SEQ ID NO: 48). In some embodiments, the insertion comprises a polypeptide that has at least 1, 2, 3, 4, or 5 mutations as compared to LNLAIEQTRP (SEQ ID NO: 48). In some embodiments, the insertion comprises a polypeptide comprising a fragment of at least 5 amino acids of LNLAIEQTRP (SEQ ID NO: 48). In some embodiments, the insertion comprises a polypeptide comprising a fragment of at least 6 amino acids of LNLAIEQTRP (SEQ ID NO: 48). In some embodiments, the insertion comprises a polypeptide comprising a fragment of at least 7 amino acids of LNLAIEQTRP (SEQ ID NO: 48). In some embodiments, the insertion comprises a polypeptide comprising a fragment of at least 8 amino acids of LNLAIEQTRP (SEQ ID NO: 48). In some embodiments, the insertion comprises a polypeptide comprising a fragment of at least 9 amino acids of LNLAIEQTRP (SEQ ID NO: 48).

In some embodiments, the capsid polypeptide comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of MLNEQTRP (SEQ ID NO: 49). In some embodiments, the insertion comprises a polypeptide that has at least 50%, 62.5%, 75%, 87.5%, or 100% identity to MLNEQTRP (SEQ ID NO: 49). In some embodiments, the insertion comprises a polypeptide that has at least 1, 2, 3, or 4 mutations as compared to MLNEQTRP (SEQ ID NO: 49). In some embodiments, the insertion comprises a polypeptide comprising a fragment of at least 4 amino acids of MLNEQTRP (SEQ ID NO: 49). In some embodiments, the insertion comprises a polypeptide comprising a fragment of at least 5 amino acids of MLNEQTRP (SEQ ID NO: 49). In some embodiments, the insertion comprises a polypeptide comprising a fragment of at least 6 amino acids of MLNEQTRP (SEQ ID NO: 49). In some embodiments, the insertion comprises a polypeptide comprising a fragment of at least 7 amino acids of MLNEQTRP (SEQ ID NO: 49).

In some embodiments, the capsid polypeptide comprises a mutation that corresponds to a G586P and N587A mutation as compared to SEQ ID NO: 1, and an insertion between residues 584 and 585 as compared to SEQ ID NO: 1, wherein the insertion comprises, e.g., consists of, a polypeptide of RSGNRADSETA (SEQ ID NO: 50). In some embodiments, the insertion comprises a polypeptide that has at least 45%, 54%, 63%, 72%, 81%, 90%, or 100% identity to RSGNRADSETA (SEQ ID NO: 50). In some embodiments, the insertion comprises a polypeptide that has at least 1, 2, 3, 4, 5, or 6 mutations as compared to RSGNRADSETA (SEQ ID NO: 50). In some embodiments, the insertion comprises a polypeptide comprising a fragment of at least 5 amino acids of RSGNRADSETA (SEQ ID NO: 50). In some embodiments, the insertion comprises a polypeptide comprising a fragment of at least 6 amino acids of RSGNRADSETA (SEQ ID NO: 50). In some embodiments, the insertion comprises a polypeptide comprising a fragment of at least 7 amino acids of RSGNRADSETA (SEQ ID NO: 50). In some embodiments, the insertion comprises a polypeptide comprising a fragment of at least 8 amino acids of RSGNRADSETA (SEQ ID NO: 50). In some embodiments, the insertion comprises a polypeptide comprising a fragment of at least 9 amino acids of RSGNRADSETA (SEQ ID NO: 50). In some embodiments, the insertion comprises a polypeptide comprising a fragment of at least 10 amino acids of RSGNRADSETA (SEQ ID NO: 50).

In some embodiments, the capsid polypeptide comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of TGDTRP (SEQ ID NO: 51). In some embodiments, the insertion comprises a polypeptide that has at least 50.1%, 66.8%, 83.5%, or 100% identity to TGDTRP (SEQ ID NO: 51). In some embodiments, the insertion comprises a polypeptide that has at least 1, 2, or 3 mutations as compared to TGDTRP (SEQ ID NO: 51). In some embodiments, the insertion comprises a polypeptide comprising a fragment of at least 3 amino acids of TGDTRP (SEQ ID NO: 51). In some embodiments, the insertion comprises a polypeptide comprising a fragment of at least 4 amino acids of TGDTRP (SEQ ID NO: 51). In some embodiments, the insertion comprises a polypeptide comprising a fragment of at least 5 amino acids of TGDTRP (SEQ ID NO: 51).

In some embodiments, the capsid polypeptide comprises a mutation that corresponds to an insertion between residues 587 and 588 as compared to SEQ ID NO: 1, wherein the insertion comprises, e.g., consists of, a polypeptide of LQGETIRPA (SEQ ID NO: 52). In some embodiments, the insertion comprises a polypeptide that has at least 44.4%, 55.5%, 66.6%, 77.7%, 88.8%, or 100% identity to LQGETIRPA (SEQ ID NO: 52). In some embodiments, the insertion comprises a polypeptide that has at least 1, 2, 3, 4, or 5 mutations as compared to LQGETIRPA (SEQ ID NO: 52). In some embodiments, the insertion comprises a polypeptide comprising a fragment of at least 5 amino acids of LQGETIRPA (SEQ ID NO: 52). In some embodiments, the insertion comprises a polypeptide comprising a fragment of at least 6 amino acids of LQGETIRPA (SEQ ID NO: 52). In some embodiments, the insertion comprises a polypeptide comprising a fragment of at least 7 amino acids of LQGETIRPA (SEQ TD NO: 52). In some embodiments, the insertion comprises a polypeptide comprising a fragment of at least 8 amino acids of LQGETIRPA (SEQ ID NO: 52).

In some embodiments, the capsid polypeptide comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of QNLANPETTRP (SEQ ID NO: 53). In some embodiments, the insertion comprises a polypeptide that has at least 45%, 54%, 63%, 72%, 81%, 90%, or 100% identity to QNLANPETTRP (SEQ ID NO: 53). In some embodiments, the insertion comprises a polypeptide that has at least 1, 2, 3, 4, 5, or 6 mutations as compared to QNLANPETTRP (SEQ ID NO: 53). In some embodiments, the insertion comprises a polypeptide comprising a fragment of at least 5 amino acids of QNLANPETTRP (SEQ ID NO: 53). In some embodiments, the insertion comprises a polypeptide comprising a fragment of at least 6 amino acids of QNLANPETTRP (SEQ ID NO: 53). In some embodiments, the insertion comprises a polypeptide comprising a fragment of at least 7 amino acids of QNLANPETTRP (SEQ ID NO: 53). In some embodiments, the insertion comprises a polypeptide comprising a fragment of at least 8 amino acids of QNLANPETTRP (SEQ ID NO: 53). In some embodiments, the insertion comprises a polypeptide comprising a fragment of at least 9 amino acids of QNLANPETTRP (SEQ ID NO: 53). In some embodiments, the insertion comprises a polypeptide comprising a fragment of at least 10 amino acids of QNLANPETTRP (SEQ ID NO: 53).

In some embodiments, the capsid polypeptide comprises a mutation that corresponds a T592A and T597W mutations as compared to SEQ ID NO: 1, and an insertion between residues 587 and 588 as compared to SEQ ID NO: 1, wherein the insertion comprises, e.g., consists of, a polypeptide of RAPQETTRPA (SEQ ID NO: 54). In some embodiments, the insertion comprises a polypeptide that has at least 50%, 60%, 70%, 80%, 90%, or 100% identity to RAPQETTRPA (SEQ ID NO: 54). In some embodiments, the insertion comprises a polypeptide that has at least 1, 2, 3, 4, or 5 mutations as compared to RAPQETTRPA (SEQ ID NO: 54). In some embodiments, the insertion comprises a polypeptide comprising a fragment of at least 5 amino acids of RAPQETTRPA (SEQ ID NO: 54). In some embodiments, the insertion comprises a polypeptide comprising a fragment of at least 6 amino acids of RAPQETTRPA (SEQ ID NO: 54). In some embodiments, the insertion comprises a polypeptide comprising a fragment of at least 7 amino acids of RAPQETTRPA (SEQ ID NO: 54). In some embodiments, the insertion comprises a polypeptide comprising a fragment of at least 8 amino acids of RAPQETTRPA (SEQ ID NO: 54). In some embodiments, the insertion comprises a polypeptide comprising a fragment of at least 9 amino acids of RAPQETTRPA (SEQ ID NO: 54).

In some embodiments, the capsid polypeptide comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of ANLTTTRP (SEQ ID NO: 55). In some embodiments, the insertion comprises a polypeptide that has at least 50%, 62.5%, 75%, 87.5%, or 100% identity to ANLTTTRP (SEQ ID NO: 55). In some embodiments, the insertion comprises a polypeptide that has at least 1, 2, 3, or 4 mutations as compared to ANLTTTRP (SEQ ID NO: 55). In some embodiments, the insertion comprises a polypeptide comprising a fragment of at least 4 amino acids of ANLTTTRP (SEQ ID NO: 55). In some embodiments, the insertion comprises a polypeptide comprising a fragment of at least 5 amino acids of ANLTTTRP (SEQ ID NO: 55). In some embodiments, the insertion comprises a polypeptide comprising a fragment of at least 6 amino acids of ANLTTTRP (SEQ ID NO: 55). In some embodiments, the insertion comprises a polypeptide comprising a fragment of at least 7 amino acids of ANLTTTRP (SEQ ID NO: 55).

In some embodiments, the capsid polypeptide comprises a mutation that corresponds a N587A mutation as compared to SEQ ID NO: 1, and an insertion between residues 586 and 587 as compared to SEQ ID NO: 1, wherein the insertion comprises, e.g., consists of, a polypeptide of ALLAGEQTRP (SEQ ID NO: 56). In some embodiments, the insertion comprises a polypeptide that has at least 50%, 60%, 70%, 80%, 90%, or 100% identity to ALLAGEQTRP (SEQ ID NO: 56). In some embodiments, the insertion comprises a polypeptide that has at least 1, 2, 3, 4, or 5 mutations as compared to ALLAGEQTRP (SEQ ID NO: 56). In some embodiments, the insertion comprises a polypeptide comprising a fragment of at least 5 amino acids of ALLAGEQTRP (SEQ ID NO: 56). In some embodiments, the insertion comprises a polypeptide comprising a fragment of at least 6 amino acids of ALLAGEQTRP (SEQ ID NO: 56). In some embodiments, the insertion comprises a polypeptide comprising a fragment of at least 7 amino acids of ALLAGEQTRP (SEQ ID NO: 56). In some embodiments, the insertion comprises a polypeptide comprising a fragment of at least 8 amino acids of ALLAGEQTRP (SEQ ID NO: 56). In some embodiments, the insertion comprises a polypeptide comprising a fragment of at least 9 amino acids of ALLAGEQTRP (SEQ ID NO: 56).

In some embodiments, the capsid polypeptide comprises a mutation that corresponds to a D561C, N587A, and T597N mutation as compared to SEQ ID NO: 1, and an insertion between residues 586 and 587 as compared to SEQ ID NO: 1, wherein the insertion comprises, e.g., consists of, a polypeptide of GLRAEQTRP (SEQ ID NO: 57). In some embodiments, the insertion comprises a polypeptide that has at least 44.4%, 55.5%, 66.6%, 77.7%, 88.8%, or 100% identity to GLRAEQTRP (SEQ ID NO: 57). In some embodiments, the insertion comprises a polypeptide that has at least 1, 2, 3, 4, or 5 mutations as compared to GLRAEQTRP (SEQ ID NO: 57). In some embodiments, the insertion comprises a polypeptide comprising a fragment of at least 5 amino acids of GLRAEQTRP (SEQ ID NO: 57). In some embodiments, the insertion comprises a polypeptide comprising a fragment of at least 6 amino acids of GLRAEQTRP (SEQ ID NO: 57). In some embodiments, the insertion comprises a polypeptide comprising a fragment of at least 7 amino acids of GLRAEQTRP (SEQ ID NO: 57). In some embodiments, the insertion comprises a polypeptide comprising a fragment of at least 8 amino acids of GLRAEQTRP (SEQ ID NO: 57).

In some embodiments, the capsid polypeptide comprises a mutation that corresponds to a T550N, G586P, and N587A mutation as compared to SEQ ID NO: 1, and an insertion between residues 584 and 585 as compared to SEQ ID NO: 1, wherein the insertion comprises, e.g., consists of, a polypeptide of RARLDETA (SEQ ID NO: 58). In some embodiments, the insertion comprises a polypeptide that has at least 50%, 62.5%, 75%, 87.5%, or 100% identity to RARLDETA (SEQ ID NO: 58). In some embodiments, the insertion comprises a polypeptide that has at least 1, 2, 3, or 4 mutations as compared to RARLDETA (SEQ ID NO: 58). In some embodiments, the insertion comprises a polypeptide comprising a fragment of at least 4 amino acids of RARLDETA (SEQ ID NO: 58). In some embodiments, the insertion comprises a polypeptide comprising a fragment of at least 5 amino acids of RARLDETA (SEQ ID NO: 58). In some embodiments, the insertion comprises a polypeptide comprising a fragment of at least 6 amino acids of RARLDETA (SEQ ID NO: 58). In some embodiments, the insertion comprises a polypeptide comprising a fragment of at least 7 amino acids of RARLDETA (SEQ ID NO: 58).

In some embodiments, the capsid polypeptide comprises a mutation that corresponds to a I559L, and N587A mutation as compared to SEQ ID NO: 1, and an insertion between residues 586 and 587 as compared to SEQ ID NO: 1, wherein the insertion comprises, e.g., consists of, a polypeptide of TNLARGETARP (SEQ ID NO: 59). In some embodiments, the insertion comprises a polypeptide that has at least 45%, 54%, 63%, 72%, 81%, 90%, or 100% identity to TNLARGETARP (SEQ ID NO: 59). In some embodiments, the insertion comprises a polypeptide that has at least 1, 2, 3, 4, 5, or 6 mutations as compared to TNLARGETARP (SEQ ID NO: 59). In some embodiments, the insertion comprises a polypeptide comprising a fragment of at least 5 amino acids of TNLARGETARP (SEQ ID NO: 59). In some embodiments, the insertion comprises a polypeptide comprising a fragment of at least 6 amino acids of TNLARGETARP (SEQ ID NO: 59). In some embodiments, the insertion comprises a polypeptide comprising a fragment of at least 7 amino acids of TNLARGETARP (SEQ ID NO: 59). In some embodiments, the insertion comprises a polypeptide comprising a fragment of at least 8 amino acids of TNLARGETARP (SEQ ID NO: 59). In some embodiments, the insertion comprises a polypeptide comprising a fragment of at least 9 amino acids of TNLARGETARP (SEQ ID NO: 59). In some embodiments, the insertion comprises a polypeptide comprising a fragment of at least 10 amino acids of TNLARGETARP (SEQ ID NO: 59).

In some embodiments, a nucleic acid molecule is provided. In some embodiments, the nucleic acid molecule has the sequence selected from Table 2. In some embodiments, the nucleic acid molecule has the sequence of SEQ ID NO: 28-43. In some embodiments, the nucleic acid molecule has the sequence of SEQ ID NO: 28. In some embodiments, the nucleic acid molecule has the sequence of SEQ ID NO: 29. In some embodiments, the nucleic acid molecule has the sequence of SEQ ID NO: 30. In some embodiments, the nucleic acid molecule has the sequence of SEQ ID NO: 31. In some embodiments, the nucleic acid molecule has the sequence of SEQ ID NO: 32. In some embodiments, the nucleic acid molecule has the sequence of SEQ ID NO: 33. In some embodiments, the nucleic acid molecule has the sequence of SEQ ID NO: 34. In some embodiments, the nucleic acid molecule has the sequence of SEQ ID NO: 35. In some embodiments, the nucleic acid molecule has the sequence of SEQ ID NO: 36. In some embodiments, the nucleic acid molecule has the sequence of SEQ ID NO: 37. In some embodiments, the nucleic acid molecule has the sequence of SEQ ID NO: 38. In some embodiments, the nucleic acid molecule has the sequence of SEQ ID NO: 39. In some embodiments, the nucleic acid molecule has the sequence of SEQ ID NO: 40. In some embodiments, the nucleic acid molecule has the sequence of SEQ ID NO: 41. In some embodiments, the nucleic acid molecule has the sequence of SEQ ID NO: 42. In some embodiments, the nucleic acid molecule has the sequence of SEQ ID NO: 43.

In some embodiments, the nucleic acid molecule encodes a capsid polypeptide that comprises a mutation that corresponds to a mutation at position 550, 559, 561, 586, 587, 592, 593, 597, or any combination thereof, an insertion between positions 584 and 585, 586 and 587, or 587 and 588, or any combination thereof according to SEQ ID NO: 1, and optionally wherein the mutation comprises an insertion, a deletion or a substitution.

In some embodiments, the nucleic acid molecule comprises a sequence that encodes a capsid polypeptide that comprises a mutation that corresponds to a mutation at position 550 as compared to SEQ ID NO: 1. In some embodiments, the nucleic acid molecule encodes a capsid polypeptide that comprises a mutation that corresponds to a mutation at position 559 as compared to SEQ ID NO: 1. In some embodiments, the nucleic acid molecule encodes a capsid polypeptide that comprises a mutation that corresponds to a mutation at position 561 as compared to SEQ ID NO: 1. In some embodiments, the nucleic acid molecule encodes a capsid polypeptide that comprises a mutation that corresponds to a mutation at position 586 as compared to SEQ ID NO: 1. In some embodiments, the nucleic acid molecule encodes a capsid polypeptide that comprises a mutation that corresponds to a mutation at position 587 as compared to SEQ ID NO: 1. In some embodiments, the nucleic acid molecule encodes a capsid polypeptide that comprises a mutation that corresponds to a mutation at position 592 as compared to SEQ ID NO: 1. In some embodiments, the nucleic acid molecule encodes a capsid polypeptide that comprises a mutation that corresponds to a mutation at position 593 as compared to SEQ ID NO: 1. In some embodiments, the nucleic acid molecule encodes a capsid polypeptide that comprises a mutation that corresponds to a mutation at position 597 as compared to SEQ ID NO: 1.

In some embodiments, the nucleic acid molecule encodes a capsid polypeptide that comprises a mutation that corresponds to an insertion at position between positions 584 and 585 as compared to SEQ ID NO: 1. In some embodiments, the nucleic acid molecule encodes a capsid polypeptide that comprises a mutation that corresponds to an insertion at position between positions 586 and 587 as compared to SEQ ID NO: 1. In some embodiments, the nucleic acid molecule encodes a capsid polypeptide that comprises a mutation that corresponds to an insertion at position between positions 587 and 588 as compared to SEQ ID NO: 1.

In some embodiments, the nucleic acid molecule encodes a capsid polypeptide that comprises a mutation that corresponds to a mutation at position 550, 559, 561, 586, 587, 592, 593, 597, or any combination thereof, an insertion between positions 584 and 585, 586 and 587, or 587 and 588, or any combination thereof according to SEQ ID NO: 1, and wherein the mutation comprises an insertion, a deletion or a substitution.

In some embodiments, the nucleic acid molecule encodes a capsid polypeptide that comprises a mutation that corresponds to a mutation at position 587 and 593 as compared to SEQ ID NO: 1.

In some embodiments, the nucleic acid molecule encodes a capsid polypeptide that comprises a mutation that corresponds to a mutation at position 586 and 587 as compared to SEQ ID NO: 1.

In some embodiments, the nucleic acid molecule encodes a capsid polypeptide that comprises a mutation that corresponds to a mutation at position 592 and 597 as compared to SEQ ID NO: 1.

In some embodiments, the nucleic acid molecule encodes a capsid polypeptide that comprises a mutation that corresponds to a mutation at position 587 and 597 as compared to SEQ ID NO: 1.

In some embodiments, the nucleic acid molecule encodes a capsid polypeptide that comprises a mutation that corresponds to a mutation at position 550, 586, and 587 as compared to SEQ ID NO: 1.

In some embodiments, the nucleic acid molecule encodes a capsid polypeptide that comprises a mutation that corresponds to a mutation at position 559 and 587 as compared to SEQ ID NO: 1.

In some embodiments, the nucleic acid molecule encodes a capsid polypeptide that comprises a mutation that corresponds to a mutation at position 451, 456, 457, 458, 459, and 461 and an insertion between positions 449 and 450 as compared to SEQ ID NO: 1.

In some embodiments, the nucleic acid molecule encodes a capsid polypeptide that comprises a mutation that corresponds to a mutation at position 587, and an insertion between positions 586 and 587 as compared to SEQ ID NO: 1.

In some embodiments, the nucleic acid molecule encodes a capsid polypeptide that comprises a mutation that corresponds to a mutation at position 587 and 593, and an insertion between positions 586 and 587 as compared to SEQ ID NO: 1.

In some embodiments, the nucleic acid molecule encodes a capsid polypeptide that comprises a mutation that corresponds to a mutation at position 586 and 587, and an insertion between positions 584 and 585 as compared to SEQ ID NO: 1.

In some embodiments, the nucleic acid molecule encodes a capsid polypeptide that comprises a mutation that corresponds to a mutation at position 592 and 597, and an insertion between positions 587 and 588 as compared to SEQ ID NO: 1.

In some embodiments, the nucleic acid molecule encodes a capsid polypeptide that comprises a mutation that corresponds to a mutation at position 561, 587, and 597, and an insertion between positions 586 and 587 as compared to SEQ ID NO: 1.

In some embodiments, the nucleic acid molecule encodes a capsid polypeptide that comprises a mutation that corresponds to a mutation at position 550, 586, and 587, and an insertion between positions 584 and 585 as compared to SEQ ID NO: 1.

In some embodiments, the nucleic acid molecule encodes a capsid polypeptide that comprises a mutation that corresponds to a mutation at position 559, and 587, and an insertion between positions 586 and 587 as compared to SEQ ID NO: 1.

In some embodiments, the nucleic acid molecule encodes a capsid polypeptide that comprises a mutation that corresponds to a mutation T550N mutation as compared to SEQ ID NO: 1.

In some embodiments, the nucleic acid molecule encodes a capsid polypeptide that comprises a mutation that corresponds to a mutation I559L mutation as compared to SEQ ID NO: 1.

In some embodiments, the nucleic acid molecule encodes a capsid polypeptide that comprises a mutation that corresponds to a mutation D561C mutation as compared to SEQ ID NO: 1.

In some embodiments, the nucleic acid molecule encodes a capsid polypeptide that comprises a mutation that corresponds to a mutation G586P mutation as compared to SEQ ID NO: 1.

In some embodiments, the nucleic acid molecule encodes a capsid polypeptide that comprises a mutation that corresponds to a mutation N587A mutation as compared to SEQ ID NO: 1.

In some embodiments, the nucleic acid molecule encodes a capsid polypeptide that comprises a mutation that corresponds to a mutation T592A mutation as compared to SEQ ID NO: 1.

In some embodiments, the nucleic acid molecule encodes a capsid polypeptide that comprises a mutation that corresponds to a mutation A593T mutation as compared to SEQ ID NO: 1.

In some embodiments, the nucleic acid molecule encodes a capsid polypeptide that comprises a mutation that corresponds to a mutation T597N mutation as compared to SEQ ID NO: 1.

In some embodiments, the nucleic acid molecule encodes a capsid polypeptide that comprises a mutation that corresponds to a mutation T597W mutation as compared to SEQ ID NO: 1.

In some embodiments, the nucleic acid molecule encodes a capsid polypeptide that comprises a mutation that corresponds to an insertion between residues 587 and 588 as compared to SEQ ID NO: 1, wherein the insertion comprises, e.g., consists of, a polypeptide of LALGEQTRPA (SEQ ID NO: 44), and wherein the nucleic acid has a sequence of SEQ ID NO: 28. In some embodiments, the insertion comprises a polypeptide that has at least 50%, 60%, 70%, 80%, 90%, or 100% identity to LALGEQTRPA (SEQ ID NO: 44). In some embodiments, the insertion comprises a polypeptide that has at least 1, 2, 3, 4, or 5 mutations as compared to LALGEQTRPA (SEQ ID NO: 44). In some embodiments, the insertion comprises a polypeptide comprising a fragment of at least 5 amino acids of LALGEQTRPA (SEQ ID NO: 44). In some embodiments, the insertion comprises a polypeptide comprising a fragment of at least 6 amino acids of LALGEQTRPA (SEQ ID NO: 44). In some embodiments, the insertion comprises a polypeptide comprising a fragment of at least 7 amino acids of LALGEQTRPA (SEQ ID NO: 44). In some embodiments, the insertion comprises a polypeptide comprising a fragment of at least 8 amino acids of LALGEQTRPA (SEQ ID NO: 44). In some embodiments, the insertion comprises a polypeptide comprising a fragment of at least 9 amino acids of LALGEQTRPA (SEQ ID NO: 44).

In some embodiments, the nucleic acid molecule encodes a capsid polypeptide that comprises a mutation that corresponds to an insertion between residues 587 and 588 as compared to SEQ ID NO: 1, wherein the insertion comprises, e.g., consists of, a polypeptide of LAIEQTRPA (SEQ ID NO: 45), and wherein the nucleic acid has a sequence of SEQ ID NO: 29. In some embodiments, the insertion comprises a polypeptide that has at least 44.4%, 55.5%, 66.6%, 77.7%, 88.8%, or 100% identity to LAIEQTRPA (SEQ ID NO: 45). In some embodiments, the insertion comprises a polypeptide that has at least 1, 2, 3, 4, or 5 mutations as compared to LAIEQTRPA (SEQ ID NO: 45). In some embodiments, the insertion comprises a polypeptide comprising a fragment of at least 5 amino acids of LAIEQTRPA (SEQ ID NO: 45). In some embodiments, the insertion comprises a polypeptide comprising a fragment of at least 6 amino acids of LAIEQTRPA (SEQ ID NO: 45). In some embodiments, the insertion comprises a polypeptide comprising a fragment of at least 7 amino acids of LAIEQTRPA (SEQ ID NO: 45). In some embodiments, the insertion comprises a polypeptide comprising a fragment of at least 8 amino acids of LAIEQTRPA (SEQ ID NO: 45).

In some embodiments, the nucleic acid molecule encodes a capsid polypeptide that comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of LALAEITRP (SEQ ID NO: 46), and wherein the nucleic acid has a sequence of SEQ ID NO: 30. In some embodiments, the insertion comprises a polypeptide that has at least 44.4%, 55.5%, 66.6%, 77.7%, 88.8%, or 100% identity to LALAEITRP (SEQ ID NO: 46). In some embodiments, the insertion comprises a polypeptide that has at least 1, 2, 3, 4, or 5 mutations as compared to LALAEITRP (SEQ ID NO: 46). In some embodiments, the insertion comprises a polypeptide comprising a fragment of at least 5 amino acids of LALAEITRP (SEQ ID NO: 46). In some embodiments, the insertion comprises a polypeptide comprising a fragment of at least 6 amino acids of LALAEITRP (SEQ ID NO: 46). In some embodiments, the insertion comprises a polypeptide comprising a fragment of at least 7 amino acids of LALAEITRP (SEQ ID NO: 46). In some embodiments, the insertion comprises a polypeptide comprising a fragment of at least 8 amino acids of LALAEITRP (SEQ ID NO: 46).

In some embodiments, the nucleic acid molecule encodes a capsid polypeptide that comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of LKNAETARP (SEQ ID NO: 47), and wherein the nucleic acid has a sequence of SEQ ID NO: 31. In some embodiments, the insertion comprises a polypeptide that has at least 44.4%, 55.5%, 66.6%, 77.7%, 88.8%, or 100% identity to LKNAETARP (SEQ ID NO: 47). In some embodiments, the insertion comprises a polypeptide that has at least 1, 2, 3, 4, or 5 mutations as compared to LKNAETARP (SEQ ID NO: 47). In some embodiments, the insertion comprises a polypeptide comprising a fragment of at least 5 amino acids of LKNAETARP (SEQ ID NO: 47). In some embodiments, the insertion comprises a polypeptide comprising a fragment of at least 6 amino acids of LKNAETARP (SEQ ID NO: 47). In some embodiments, the insertion comprises a polypeptide comprising a fragment of at least 7 amino acids of LKNAETARP (SEQ ID NO: 47). In some embodiments, the insertion comprises a polypeptide comprising a fragment of at least 8 amino acids of LKNAETARP (SEQ ID NO: 47).

In some embodiments, the nucleic acid molecule encodes a capsid polypeptide that comprises a mutation that corresponds a N587A and A593T mutations as compared to SEQ ID NO: 1, and an insertion between residues 586 and 587 as compared to SEQ ID NO: 1, wherein the insertion comprises, e.g., consists of, a polypeptide of LNLAIEQTRP (SEQ ID NO: 48), and wherein the nucleic acid has a sequence of SEQ ID NO: 32. In some embodiments, the insertion comprises a polypeptide that has at least 50%, 60%, 70%, 80%, 90%, or 100% identity to LNLAIEQTRP (SEQ ID NO: 48). In some embodiments, the insertion comprises a polypeptide that has at least 1, 2, 3, 4, or 5 mutations as compared to LNLAIEQTRP (SEQ ID NO: 48). In some embodiments, the insertion comprises a polypeptide comprising a fragment of at least 5 amino acids of LNLAIEQTRP (SEQ ID NO: 48). In some embodiments, the insertion comprises a polypeptide comprising a fragment of at least 6 amino acids of LNLAIEQTRP (SEQ ID NO: 48). In some embodiments, the insertion comprises a polypeptide comprising a fragment of at least 7 amino acids of LNLAIEQTRP (SEQ ID NO: 48). In some embodiments, the insertion comprises a polypeptide comprising a fragment of at least 8 amino acids of LNLAIEQTRP (SEQ ID NO: 48). In some embodiments, the insertion comprises a polypeptide comprising a fragment of at least 9 amino acids of LNLAIEQTRP (SEQ ID NO: 48).

In some embodiments, the nucleic acid molecule encodes a capsid polypeptide that comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of MLNEQTRP (SEQ ID NO: 49), and wherein the nucleic acid has a sequence of SEQ ID NO: 33. In some embodiments, the insertion comprises a polypeptide that has at least 50%, 62.5%, 75%, 87.5%, or 100% identity to MLNEQTRP (SEQ ID NO: 49). In some embodiments, the insertion comprises a polypeptide that has at least 1, 2, 3, or 4 mutations as compared to MLNEQTRP (SEQ ID NO: 49). In some embodiments, the insertion comprises a polypeptide comprising a fragment of at least 4 amino acids of MLNEQTRP (SEQ ID NO: 49). In some embodiments, the insertion comprises a polypeptide comprising a fragment of at least 5 amino acids of MLNEQTRP (SEQ ID NO: 49). In some embodiments, the insertion comprises a polypeptide comprising a fragment of at least 6 amino acids of MLNEQTRP (SEQ ID NO: 49). In some embodiments, the insertion comprises a polypeptide comprising a fragment of at least 7 amino acids of MLNEQTRP (SEQ ID NO: 49).

In some embodiments, the nucleic acid molecule encodes a capsid polypeptide that comprises a mutation that corresponds to a G586P and N587A mutation as compared to SEQ ID NO: 1, and an insertion between residues 584 and 585 as compared to SEQ ID NO: 1, wherein the insertion comprises, e.g., consists of, a polypeptide of RSGNRADSETA (SEQ ID NO: 50), and wherein the nucleic acid has a sequence of SEQ ID NO: 34. In some embodiments, the insertion comprises a polypeptide that has at least 45%, 54%, 63%, 72%, 81%, 90%, or 100% identity to RSGNRADSETA (SEQ ID NO: 50). In some embodiments, the insertion comprises a polypeptide that has at least 1, 2, 3, 4, 5, or 6 mutations as compared to RSGNRADSETA (SEQ ID NO: 50). In some embodiments, the insertion comprises a polypeptide comprising a fragment of at least 5 amino acids of RSGNRADSETA (SEQ ID NO: 50). In some embodiments, the insertion comprises a polypeptide comprising a fragment of at least 6 amino acids of RSGNRADSETA (SEQ ID NO: 50). In some embodiments, the insertion comprises a polypeptide comprising a fragment of at least 7 amino acids of RSGNRADSETA (SEQ ID NO: 50). In some embodiments, the insertion comprises a polypeptide comprising a fragment of at least 8 amino acids of RSGNRADSETA (SEQ ID NO: 50). In some embodiments, the insertion comprises a polypeptide comprising a fragment of at least 9 amino acids of RSGNRADSETA (SEQ ID NO: 50). In some embodiments, the insertion comprises a polypeptide comprising a fragment of at least 10 amino acids of RSGNRADSETA (SEQ ID NO: 50).

In some embodiments, the nucleic acid molecule encodes a capsid polypeptide that comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of TGDTRP (SEQ ID NO: 51), and wherein the nucleic acid has a sequence of SEQ ID NO: 35. In some embodiments, the insertion comprises a polypeptide that has at least 50.1%, 66.8%, 83.5%, or 100% identity to TGDTRP (SEQ ID NO: 51). In some embodiments, the insertion comprises a polypeptide that has at least 1, 2, or 3 mutations as compared to TGDTRP (SEQ ID NO: 51). In some embodiments, the insertion comprises a polypeptide comprising a fragment of at least 3 amino acids of TGDTRP (SEQ ID NO: 51). In some embodiments, the insertion comprises a polypeptide comprising a fragment of at least 4 amino acids of TGDTRP (SEQ ID NO: 51). In some embodiments, the insertion comprises a polypeptide comprising a fragment of at least 5 amino acids of TGDTRP (SEQ ID NO: 51).

In some embodiments, the nucleic acid molecule encodes a capsid polypeptide that comprises a mutation that corresponds to an insertion between residues 587 and 588 as compared to SEQ ID NO: 1, wherein the insertion comprises, e.g., consists of, a polypeptide of LQGETIRPA (SEQ ID NO: 52), and wherein the nucleic acid has a sequence of SEQ ID NO: 36. In some embodiments, the insertion comprises a polypeptide that has at least 44.4%, 55.5%, 66.6%, 77.7%, 88.8%, or 100% identity to LQGETIRPA (SEQ ID NO: 52). In some embodiments, the insertion comprises a polypeptide that has at least 1, 2, 3, 4, or 5 mutations as compared to LQGETIRPA (SEQ ID NO: 52). In some embodiments, the insertion comprises a polypeptide comprising a fragment of at least 5 amino acids of LQGETIRPA (SEQ ID NO: 52). In some embodiments, the insertion comprises a polypeptide comprising a fragment of at least 6 amino acids of LQGETIRPA (SEQ ID NO: 52). In some embodiments, the insertion comprises a polypeptide comprising a fragment of at least 7 amino acids of LQGETIRPA (SEQ ID NO: 52). In some embodiments, the insertion comprises a polypeptide comprising a fragment of at least 8 amino acids of LQGETIRPA (SEQ ID NO: 52).

In some embodiments, the nucleic acid molecule encodes a capsid polypeptide that comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of QNLANPETTRP (SEQ ID NO: 53), and wherein the nucleic acid has a sequence of SEQ ID NO: 37. In some embodiments, the insertion comprises a polypeptide that has at least 45%, 54%, 63%, 72%, 81%, 90%, or 100% identity to QNLANPETTRP (SEQ ID NO: 53). In some embodiments, the insertion comprises a polypeptide that has at least 1, 2, 3, 4, 5, or 6 mutations as compared to QNLANPETTRP (SEQ ID NO: 53). In some embodiments, the insertion comprises a polypeptide comprising a fragment of at least 5 amino acids of QNLANPETTRP (SEQ ID NO: 53). In some embodiments, the insertion comprises a polypeptide comprising a fragment of at least 6 amino acids of QNLANPETTRP (SEQ ID NO: 53). In some embodiments, the insertion comprises a polypeptide comprising a fragment of at least 7 amino acids of QNLANPETTRP (SEQ ID NO: 53). In some embodiments, the insertion comprises a polypeptide comprising a fragment of at least 8 amino acids of QNLANPETTRP (SEQ ID NO: 53). In some embodiments, the insertion comprises a polypeptide comprising a fragment of at least 9 amino acids of QNLANPETTRP (SEQ ID NO: 53). In some embodiments, the insertion comprises a polypeptide comprising a fragment of at least 10 amino acids of QNLANPETTRP (SEQ ID NO: 53).

In some embodiments, the nucleic acid molecule encodes a capsid polypeptide that comprises a mutation that corresponds a T592A and T597W mutations as compared to SEQ ID NO: 1, and an insertion between residues 587 and 588 as compared to SEQ ID NO: 1, wherein the insertion comprises, e.g., consists of, a polypeptide of RAPQETTRPA (SEQ ID NO: 54), and wherein the nucleic acid has a sequence of SEQ ID NO: 38. In some embodiments, the insertion comprises a polypeptide that has at least 50%, 60%, 70%, 80%, 90%, or 100% identity to RAPQETTRPA (SEQ ID NO: 54). In some embodiments, the insertion comprises a polypeptide that has at least 1, 2, 3, 4, or 5 mutations as compared to RAPQETTRPA (SEQ ID NO: 54). In some embodiments, the insertion comprises a polypeptide comprising a fragment of at least 5 amino acids of RAPQETTRPA (SEQ ID NO: 54). In some embodiments, the insertion comprises a polypeptide comprising a fragment of at least 6 amino acids of RAPQETTRPA (SEQ ID NO: 54). In some embodiments, the insertion comprises a polypeptide comprising a fragment of at least 7 amino acids of RAPQETTRPA (SEQ ID NO: 54). In some embodiments, the insertion comprises a polypeptide comprising a fragment of at least 8 amino acids of RAPQETTRPA (SEQ ID NO: 54). In some embodiments, the insertion comprises a polypeptide comprising a fragment of at least 9 amino acids of RAPQETTRPA (SEQ ID NO: 54).

In some embodiments, the nucleic acid molecule encodes a capsid polypeptide that comprises a mutation that corresponds to a N587A mutation as compared to SEQ ID NO: 1, and an insertion between residues 586 and 587 as compared to SEQ ID NO: 1, wherein the insertion comprises, e.g., consists of, a polypeptide of ANLTTTRP (SEQ ID NO: 55), and wherein the nucleic acid has a sequence of SEQ ID NO: 39. In some embodiments, the insertion comprises a polypeptide that has at least 50%, 62.5%, 75%, 87.5%, or 100% identity to ANLTTTRP (SEQ ID NO: 55). In some embodiments, the insertion comprises a polypeptide that has at least 1, 2, 3, or 4 mutations as compared to ANLTTTRP (SEQ ID NO: 55). In some embodiments, the insertion comprises a polypeptide comprising a fragment of at least 4 amino acids of ANLTTTRP (SEQ ID NO: 55). In some embodiments, the insertion comprises a polypeptide comprising a fragment of at least 5 amino acids of ANLTTTRP (SEQ ID NO: 55). In some embodiments, the insertion comprises a polypeptide comprising a fragment of at least 6 amino acids of ANLTTTRP (SEQ ID NO: 55). In some embodiments, the insertion comprises a polypeptide comprising a fragment of at least 7 amino acids of ANLTTTRP (SEQ ID NO: 55).

In some embodiments, the nucleic acid molecule encodes a capsid polypeptide that comprises a mutation that corresponds a N587A mutation as compared to SEQ ID NO: 1, and an insertion between residues 586 and 587 as compared to SEQ ID NO: 1, wherein the insertion comprises, e.g., consists of, a polypeptide of ALLAGEQTRP (SEQ ID NO: 56), and wherein the nucleic acid has a sequence of SEQ ID NO: 40. In some embodiments, the insertion comprises a polypeptide that has at least 50%, 60%, 70%, 80%, 90%, or 100% identity to ALLAGEQTRP (SEQ ID NO: 56). In some embodiments, the insertion comprises a polypeptide that has at least 1, 2, 3, 4, or 5 mutations as compared to ALLAGEQTRP (SEQ ID NO: 56). In some embodiments, the insertion comprises a polypeptide comprising a fragment of at least 5 amino acids of ALLAGEQTRP (SEQ ID NO: 56). In some embodiments, the insertion comprises a polypeptide comprising a fragment of at least 6 amino acids of ALLAGEQTRP (SEQ ID NO: 56). In some embodiments, the insertion comprises a polypeptide comprising a fragment of at least 7 amino acids of ALLAGEQTRP (SEQ ID NO: 56). In some embodiments, the insertion comprises a polypeptide comprising a fragment of at least 8 amino acids of ALLAGEQTRP (SEQ ID NO: 56). In some embodiments, the insertion comprises a polypeptide comprising a fragment of at least 9 amino acids of ALLAGEQTRP (SEQ ID NO: 56).

In some embodiments, the nucleic acid molecule encodes a capsid polypeptide that comprises a mutation that corresponds to a D561C, N587A, and T597N mutation as compared to SEQ ID NO: 1, and an insertion between residues 586 and 587 as compared to SEQ ID NO: 1, wherein the insertion comprises, e.g., consists of, a polypeptide of GLRAEQTRP (SEQ ID NO: 57), and wherein the nucleic acid has a sequence of SEQ ID NO: 41. In some embodiments, the insertion comprises a polypeptide that has at least 44.4%, 55.5%, 66.6%, 77.7%, 88.8%, or 100% identity to GLRAEQTRP (SEQ ID NO: 57). In some embodiments, the insertion comprises a polypeptide that has at least 1, 2, 3, 4, or 5 mutations as compared to GLRAEQTRP (SEQ ID NO: 57). In some embodiments, the insertion comprises a polypeptide comprising a fragment of at least 5 amino acids of GLRAEQTRP (SEQ ID NO: 57). In some embodiments, the insertion comprises a polypeptide comprising a fragment of at least 6 amino acids of GLRAEQTRP (SEQ ID NO: 57). In some embodiments, the insertion comprises a polypeptide comprising a fragment of at least 7 amino acids of GLRAEQTRP (SEQ ID NO: 57). In some embodiments, the insertion comprises a polypeptide comprising a fragment of at least 8 amino acids of GLRAEQTRP (SEQ ID NO: 57).

In some embodiments, the nucleic acid molecule encodes a capsid polypeptide that comprises a mutation that corresponds to a T550N, G586P, and N587A mutation as compared to SEQ ID NO: 1, and an insertion between residues 584 and 585 as compared to SEQ ID NO: 1, wherein the insertion comprises, e.g., consists of, a polypeptide of RARLDETA (SEQ ID NO: 58), and wherein the nucleic acid has a sequence of SEQ ID NO: 42. In some embodiments, the insertion comprises a polypeptide that has at least 50%, 62.5%, 75%, 87.5%, or 100% identity to RARLDETA (SEQ ID NO: 58). In some embodiments, the insertion comprises a polypeptide that has at least 1, 2, 3, or 4 mutations as compared to RARLDETA (SEQ ID NO: 58). In some embodiments, the insertion comprises a polypeptide comprising a fragment of at least 4 amino acids of RARLDETA (SEQ ID NO: 58). In some embodiments, the insertion comprises a polypeptide comprising a fragment of at least 5 amino acids of RARLDETA (SEQ ID NO: 58). In some embodiments, the insertion comprises a polypeptide comprising a fragment of at least 6 amino acids of RARLDETA (SEQ ID NO: 58). In some embodiments, the insertion comprises a polypeptide comprising a fragment of at least 7 amino acids of RARLDETA (SEQ ID NO: 58).

In some embodiments, the nucleic acid molecule encodes a capsid polypeptide that comprises a mutation that corresponds to a I559L, and N587A mutation as compared to SEQ ID NO: 1, and an insertion between residues 586 and 587 as compared to SEQ ID NO: 1, wherein the insertion comprises, e.g., consists of, a polypeptide of TNLARGETARP (SEQ ID NO: 59), and wherein the nucleic acid has a sequence of SEQ ID NO: 43. In some embodiments, the insertion comprises a polypeptide that has at least 45%, 54%, 63%, 72%, 81%, 90%, or 100% identity to TNLARGETARP (SEQ ID NO: 59). In some embodiments, the insertion comprises a polypeptide that has at least 1, 2, 3, 4, 5, or 6 mutations as compared to TNLARGETARP (SEQ ID NO: 59). In some embodiments, the insertion comprises a polypeptide comprising a fragment of at least 5 amino acids of TNLARGETARP (SEQ ID NO: 59). In some embodiments, the insertion comprises a polypeptide comprising a fragment of at least 6 amino acids of TNLARGETARP (SEQ ID NO: 59). In some embodiments, the insertion comprises a polypeptide comprising a fragment of at least 7 amino acids of TNLARGETARP (SEQ ID NO: 59). In some embodiments, the insertion comprises a polypeptide comprising a fragment of at least 8 amino acids of TNLARGETARP (SEQ ID NO: 59). In some embodiments, the insertion comprises a polypeptide comprising a fragment of at least 9 amino acids of TNLARGETARP (SEQ ID NO: 59). In some embodiments, the insertion comprises a polypeptide comprising a fragment of at least 10 amino acids of TNLARGETARP (SEQ ID NO: 59).

In some embodiments, including in the embodiments described above, a capsid polypeptide is provided that comprises a sequence that is at least 70%, 75%, 80%, 85%, 90%, 91%, 92%, 93%, 94%, 95%, 96%, 97%, 98%, 99%, or 100% identical to a sequence of a capsid polypeptide provided herein.

In some embodiments, the nucleic acid molecule encodes a capsid polypeptide as provided herein. In some embodiments, the nucleic acid molecule encodes a capsid polypeptide that is at least 70%, 75%, 80%, 85%, 90%, 91%, 92%, 93%, 94%, 95%, 96%, 97%, 98%, 99%, or 100% identical to a capsid polypeptide as provided herein.

In some embodiments, including in the embodiments described above, a capsid polypeptide is provided that comprises a sequence that is at least 70%, 75%, 80%, 85%, 90%, 91%, 92%, 93%, 94%, 95%, 96%, 97%, 98%, or 99 identical to SEQ ID NO: 1.

In some embodiments, including in the embodiments described above, a capsid polypeptide is provided that comprises a sequence that is at least 70%, 75%, 80%, 85%, 90%, 91%, 92%, 93%, 94%, 95%, 96%, 97%, 98%, or 99 identical to any one of SEQ ID NO: 3, 5, 7, 9 or 10.

In some embodiments, the reference polypeptide for purposes of % identity, comprises a sequence of SEQ ID NOs: 1, 3, 5, 7, 9, or 10.

In some embodiments, the reference nucleic acid for purposes of % identity, comprises a sequence of SEQ ID NOs: 2, 4, 6, 8, or 11.

In some embodiments, the capsid polypeptide, or the reference polypeptide for purposes of % identity, comprises a sequence of SEQ ID NOs: 12, 13, 14, 15, 16, 17, 18, 19, 20, 21, 22, 23, 24, 25, 26, or 27.

In some embodiments, the nucleic acid molecules encoding the capsid polypeptide, or the reference polypeptide for purposes of % identity, comprises a sequence of SEQ ID NOs: 28, 29, 30, 31, 32, 33, 34, 35, 36, 37, 38, 39, 40, 41, 42, or 43. In some embodiments, the nucleic acid molecules encoding the capsid polypeptide, or the reference polypeptide for purposes of % identity, comprises a sequence of SEQ ID NO: 28. In some embodiments, the nucleic acid molecules encoding the capsid polypeptide, or the reference polypeptide for purposes of % identity, comprises a sequence of SEQ ID NO: 29. In some embodiments, the nucleic acid molecules encoding the capsid polypeptide, or the reference polypeptide for purposes of % identity, comprises a sequence of SEQ ID NO: 30. In some embodiments, the nucleic acid molecules encoding the capsid polypeptide, or the reference polypeptide for purposes of % identity, comprises a sequence of SEQ ID NO: 31. In some embodiments, the nucleic acid molecules encoding the capsid polypeptide, or the reference polypeptide for purposes of % identity, comprises a sequence of SEQ ID NO: 32. In some embodiments, the nucleic acid molecules encoding the capsid polypeptide, or the reference polypeptide for purposes of % identity, comprises a sequence of SEQ ID NO: 33. In some embodiments, the nucleic acid molecules encoding the capsid polypeptide, or the reference polypeptide for purposes of % identity, comprises a sequence of SEQ ID NO: 34. In some embodiments, the nucleic acid molecules encoding the capsid polypeptide, or the reference polypeptide for purposes of % identity, comprises a sequence of SEQ ID NO: 35. In some embodiments, the nucleic acid molecules encoding the capsid polypeptide, or the reference polypeptide for purposes of % identity, comprises a sequence of SEQ ID NO: 36. In some embodiments, the nucleic acid molecules encoding the capsid polypeptide, or the reference polypeptide for purposes of % identity, comprises a sequence of SEQ ID NO: 37. In some embodiments, the nucleic acid molecules encoding the capsid polypeptide, or the reference polypeptide for purposes of % identity, comprises a sequence of SEQ ID NO: 38. In some embodiments, the nucleic acid molecules encoding the capsid polypeptide, or the reference polypeptide for purposes of % identity, comprises a sequence of SEQ ID NO: 39. In some embodiments, the nucleic acid molecules encoding the capsid polypeptide, or the reference polypeptide for purposes of % identity, comprises a sequence of SEQ ID NO: 40. In some embodiments, the nucleic acid molecules encoding the capsid polypeptide, or the reference polypeptide for purposes of % identity, comprises a sequence of SEQ ID NO: 41. In some embodiments, the nucleic acid molecules encoding the capsid polypeptide, or the reference polypeptide for purposes of % identity, comprises a sequence of SEQ ID NO: 42. In some embodiments, the nucleic acid molecules encoding the capsid polypeptide, or the reference polypeptide for purposes of % identity, comprises a sequence of SEQ ID NO: 43.

In some embodiments, the nucleic acid molecules encoding the capsid polypeptide, or the reference polypeptide for purposes of % identity, comprises a sequence of SEQ ID NOs: 28, 29, 30, 31, 32, 33, 34, 35, 36, 37, 38, 39, 40, 41, 42, or 43 that encodes a sequence of SEQ ID NOs: 12, 13, 14, 15, 16, 17, 18, 19, 20, 21, 22, 23, 24, 25, 26, or 27, respectively. In some embodiments, the nucleic acid molecules encoding the capsid polypeptide, or the reference polypeptide for purposes of % identity, comprises a sequence of SEQ ID NO: 28 that encodes a sequence of SEQ ID NO: 12. In some embodiments, the nucleic acid molecules encoding the capsid polypeptide, or the reference polypeptide for purposes of % identity, comprises a sequence of SEQ ID NO: 29 that encodes a sequence of SEQ ID NO: 13. In some embodiments, the nucleic acid molecules encoding the capsid polypeptide, or the reference polypeptide for purposes of % identity, comprises a sequence of SEQ ID NO: 30 that encodes a sequence of SEQ ID NO: 14. In some embodiments, the nucleic acid molecules encoding the capsid polypeptide, or the reference polypeptide for purposes of % identity, comprises a sequence of SEQ ID NO: 31 that encodes a sequence of SEQ ID NO: 15. In some embodiments, the nucleic acid molecules encoding the capsid polypeptide, or the reference polypeptide for purposes of % identity, comprises a sequence of SEQ ID NO: 32 that encodes a sequence of SEQ ID NO: 16. In some embodiments, the nucleic acid molecules encoding the capsid polypeptide, or the reference polypeptide for purposes of % identity, comprises a sequence of SEQ ID NO: 33 that encodes a sequence of SEQ ID NO: 17. In some embodiments, the nucleic acid molecules encoding the capsid polypeptide, or the reference polypeptide for purposes of % identity, comprises a sequence of SEQ ID NO: 34 that encodes a sequence of SEQ ID NO: 18. In some embodiments, the nucleic acid molecules encoding the capsid polypeptide, or the reference polypeptide for purposes of % identity, comprises a sequence of SEQ ID NO: 35 that encodes a sequence of SEQ ID NO: 19. In some embodiments, the nucleic acid molecules encoding the capsid polypeptide, or the reference polypeptide for purposes of % identity, comprises a sequence of SEQ ID NO: 36 that encodes a sequence of SEQ ID NO: 20. In some embodiments, the nucleic acid molecules encoding the capsid polypeptide, or the reference polypeptide for purposes of % identity, comprises a sequence of SEQ ID NO: 37 that encodes a sequence of SEQ ID NO: 21. In some embodiments, the nucleic acid molecules encoding the capsid polypeptide, or the reference polypeptide for purposes of % identity, comprises a sequence of SEQ ID NO: 38 that encodes a sequence of SEQ ID NO: 22. In some embodiments, the nucleic acid molecules encoding the capsid polypeptide, or the reference polypeptide for purposes of % identity, comprises a sequence of SEQ ID NO: 39 that encodes a sequence of SEQ ID NO: 23. In some embodiments, the nucleic acid molecules encoding the capsid polypeptide, or the reference polypeptide for purposes of % identity, comprises a sequence of SEQ ID NO: 40 that encodes a sequence of SEQ ID NO: 24. In some embodiments, the nucleic acid molecules encoding the capsid polypeptide, or the reference polypeptide for purposes of % identity, comprises a sequence of SEQ ID NO: 41 that encodes a sequence of SEQ ID NO: 25. In some embodiments, the nucleic acid molecules encoding the capsid polypeptide, or the reference polypeptide for purposes of % identity, comprises a sequence of SEQ ID NO: 42 that encodes a sequence of SEQ ID NO: 26. In some embodiments, the nucleic acid molecules encoding the capsid polypeptide, or the reference polypeptide for purposes of % identity, comprises a sequence of SEQ ID NO: 43 that encodes a sequence of SEQ ID NO: 27.

In some embodiments, the capsid polypeptide, or the reference polypeptide for purposes of % identity, comprises a sequence of SEQ ID NOs: 12, 13, 14, 15, 16, 17, 18, 19, 20, 21, 22, 23, 24, 25, 26, or 27, that is encoded by a nucleotide sequence of SEQ ID NOs: 28, 29, 30, 31, 32, 33, 34, 35, 36, 37, 38, 39, 40, 41, 42, or 43, respectively. In some embodiments, the capsid polypeptide, or the reference polypeptide for purposes of % identity, comprises a sequence of SEQ ID NO: 12 that is encoded by a nucleotide sequence of SEQ ID NO: 28. In some embodiments, the capsid polypeptide, or the reference polypeptide for purposes of % identity, comprises a sequence of SEQ ID NO: 13 that is encoded by a nucleotide sequence of SEQ ID NO: 29. In some embodiments, the capsid polypeptide, or the reference polypeptide for purposes of % identity, comprises a sequence of SEQ ID NO: 14 that is encoded by a nucleotide sequence of SEQ ID NO: 30. In some embodiments, the capsid polypeptide, or the reference polypeptide for purposes of % identity, comprises a sequence of SEQ ID NO: 15 that is encoded by a nucleotide sequence of SEQ ID NO: 31. In some embodiments, the capsid polypeptide, or the reference polypeptide for purposes of % identity, comprises a sequence of SEQ ID NO: 16 that is encoded by a nucleotide sequence of SEQ ID NO: 32. In some embodiments, the capsid polypeptide, or the reference polypeptide for purposes of % identity, comprises a sequence of SEQ ID NO: 17 that is encoded by a nucleotide sequence of SEQ ID NO: 33. In some embodiments, the capsid polypeptide, or the reference polypeptide for purposes of % identity, comprises a sequence of SEQ ID NO: 18 that is encoded by a nucleotide sequence of SEQ ID NO: 34. In some embodiments, the capsid polypeptide, or the reference polypeptide for purposes of % identity, comprises a sequence of SEQ ID NO: 19 that is encoded by a nucleotide sequence of SEQ ID NO: 35. In some embodiments, the capsid polypeptide, or the reference polypeptide for purposes of % identity, comprises a sequence of SEQ ID NO: 20 that is encoded by a nucleotide sequence of SEQ ID NO: 36. In some embodiments, the capsid polypeptide, or the reference polypeptide for purposes of % identity, comprises a sequence of SEQ ID NO: 21 that is encoded by a nucleotide sequence of SEQ ID NO: 37. In some embodiments, the capsid polypeptide, or the reference polypeptide for purposes of % identity, comprises a sequence of SEQ ID NO: 22 that is encoded by a nucleotide sequence of SEQ ID NO: 38. In some embodiments, the capsid polypeptide, or the reference polypeptide for purposes of % identity, comprises a sequence of SEQ ID NO: 23 that is encoded by a nucleotide sequence of SEQ ID NO: 39. In some embodiments, the capsid polypeptide, or the reference polypeptide for purposes of % identity, comprises a sequence of SEQ ID NO: 24 that is encoded by a nucleotide sequence of SEQ ID NO: 40. In some embodiments, the capsid polypeptide, or the reference polypeptide for purposes of % identity, comprises a sequence of SEQ ID NO: 25 that is encoded by a nucleotide sequence of SEQ ID NO: 41. In some embodiments, the capsid polypeptide, or the reference polypeptide for purposes of % identity, comprises a sequence of SEQ ID NO: 26 that is encoded by a nucleotide sequence of SEQ ID NO: 42. In some embodiments, the capsid polypeptide, or the reference polypeptide for purposes of % identity, comprises a sequence of SEQ ID NO: 27 that is encoded by a nucleotide sequence of SEQ ID NO: 43.

In some embodiments, the capsid polypeptide comprises a sequence that includes all of the mutation differences associated with any one of VAR-1 through VAR-16 (e.g., as indicated in Tables 1A, 1B, 1C, 1D, 1E, 1F, and 1G), and further includes no more than 30, no more than 20, no more than 10, no more than 9, no more than 8, no more than 7, no more than 6, no more than 5, no more than 4, no more than 3, no more than 2 or no more than 1 additional mutations relative to SEQ ID NO: 1.

In some embodiments, the capsid polypeptide is a VP1 capsid polypeptide. In some embodiments, the capsid polypeptide is a VP2 capsid polypeptide. In some embodiments, the capsid polypeptide is a VP3 capsid polypeptide. With respect to reference sequence SEQ ID NO: 1, a VP1 capsid polypeptide comprises amino acids 1-724 of SEQ ID NO: 1. With respect to reference sequence SEQ ID NO: 1, a VP2 capsid polypeptide comprises amino acids 138-724 of SEQ ID NO: 1. With respect to reference sequence SEQ ID NO: 1, a VP3 capsid polypeptide comprises amino acids 203-724 of SEQ ID NO: 1.

Table 1A, Table 1B, Table 1C, Table 1D, Table 1E, Table 1F, and Table 1G list information regarding exemplary variant dependoparvovirus particles comprising the variant capsids, and describing the ocular transduction properties and production characteristics of said non-limiting exemplary variants. Exemplary sequences of capsid polypeptides and nucleic acid molecules encoding the same are provided in Table 2. Table 2 illustrates the VP1, VP2 and VP3 polypeptide starting amino acid sequences of each of SEQ ID NO: 12 to SEQ ID NO: 27. The exemplary nucleic acid sequences provided in Table 2 include a stop codon at the 3′-end of the sequence (e.g., the TAA stop codon). It will be understood by a skilled artisan that in some embodiments, the TAA stop codon is removed or replaced with a different stop codon (e.g., TGA or TAG).

Table 1A, Table 1B, and Table 1C represent data produced in first (Table 1A) high throughput experiment (Library Experiment 1) and second (Table 1B-1C) high throughput experiment (Library Experiment 2). Tables 1D, 1E, 1F, and 1G represent data produced in a medium throughput experiment (Library Experiment 3). Transduction and virus production of exemplary variant dependoparvovirus (e.g., AVV) particles comprising variant capsid polypeptides. Injection rout is as indicated in the column headings. Substitutions are notated as n ###N where “N” is the final amino acid, “n” is the reference amino acid and “###” is the reference amino acid position of SEQ ID NO:1; deletions are notated as n ###- where “-” indicates the deletion of “n” at position “###” of the reference sequence SEQ ID NO: 1; insertions are notated as ###_Naa_###_(n)y, where “###” are the amino acid positions in the reference sequence SEQ ID NO: 1 between which the insertion occurs, “Naa” refers to the length of the insertion (having “N” amino acids) and “(n)y” providing the sequence of the insertion). Each individual Mutation Difference (e.g., within a row, each mutation in quotations (‘ ’) in column 8 of Table 1A, column 7 of Table 1B, column 7 of Table 1C, column 6 of Table 1D, column 6 of Table 1E, column 7 of Table 1F, column 6 of Table 1G) and combinations of such individual mutation differences is sometimes referred to herein as a “mutation associated with VAR-X”, where VAR-X is the variant identifier listed in the “Name column.” Macular Transduction refers to transduction of the neural retina layer of tissues in the macula. In Tables 1A and 1C, Non-Macular Transduction refers to transduction of the neural retina layer of the retina excluding the macula. Retinal Transduction refers to the aggregated measurements from Macular Transduction and Non-Macular Transduction. For purposes of Table 1A, Average Retinal Transduction is calculated as the average of Macular Transduction and Non-Macular Retina Transduction. Trabecular Transduction refers to transduction of tissue samples collected from the trabecular meshwork and/or Schlemm's canal. For purposes of Tables 1D and 1E, Neural Retina Transduction refers to transduction of tissue samples collected from the neural retina layer, including the macular region. “Not Measured” indicates the variant was not detected in the indicated sample. Unless otherwise indicated, measurements are made relative to wild-type AAV2 (SEQ ID NO: 1). Trabecular transduction measurements shown in Tables 1F and 1G are shown relative to an AAV2 variant identified from the ocular literature having a capsid with polypeptide of SEQ ID NO: 60, encoded by exemplary nucleic acid SEQ ID NO: 61. Data shown in Tables 1A, 1B, and 1C is on a log 2 scale.

TABLE 1A

| | | | Average | | Non-Macular | | |
|---|---|---|---|---|---|---|---|
| | | | Retinal | Trabecular | Retina | | |
| | | Macular | Trans- | Trans- | Trans- | | |
| | | Transduction | duction | duction | duction | | |
| | | (Intravitreal | (Intravitreal | (Intracameral | (Intravitreal | Virus | |
| | SEQ ID | administration) | administration) | administration) | administration) | Production as | |
| | NO: of | as compared | as compared | as compared | as compared | compared to | Mutation |
| | VP1 | to wild-type | to wild-type | to wild-type | to wild-type | wild-type | Differences |
| | capsid | SEQ ID | SEQ ID | SEQ ID | SEQ ID | SEQ ID | as compared |
| | poly- | NO: 1 | NO: 1 | NO: 1 | NO: 1 | NO: 1 | to SEQ ID |
| Name | peptide | (Log2) | (Log2) | (Log2) | (Log2) | (Log2) | NO: 1 |
| VAR-1 | 12 | 7.45 | 6.26 | 2.51 | 5.07 | 1.37 | 587_10aa_588_LALGEQTRPA |
| VAR-2 | 13 | 7.18 | 6.43 | 1.85 | 5.68 | 2.92 | 587_9aa_588_LAIEQTRPA |
| VAR-3 | 14 | 7.15 | 6.08 | 2.55 | 5.01 | 2.07 | 586_9aa_587_LALAEITRP, |
| | | | | | | | N587A |

TABLE 1B

| | | Trabecular | Trabecular | Virus | Virus | |
|---|---|---|---|---|---|---|
| | | Transduction | Transduction | Production | Production | |
| | | (intracameral) | (intravitreal) | (intracameral | (intravitreal | |
| | SEQ ID | administration | administration | library) as | library) as | |
| | NO: of | as compared | as compared | compared to | compared to | |
| | VP1 | to wild-type | to wild-type | wild-type | wild-type | Mutation |
| | capsid | SEQ ID | SEQ ID | SEQ ID | SEQ ID | Differences as |
| | poly- | NO: 1 | NO: 1 | NO: 1 | NO: 1 | compared to SEQ |
| Name | peptide | (Log2) | (Log2) | (Log2) | (Log2) | ID NO. 1 |
| VAR-1 | 12 | Not Measured | −0.64 | 0.87 | 2.83 | 587_10aa_588_LALGEQTRPA |
| VAR-2 | 13 | −1.14 | −0.97 | −3.05 | 1.05 | 587_9aa_588_LAIEQTRPA |
| VAR-3 | 14 | 3.54 | −0.06 | −1.74 | 0.90 | 586_9aa_587_LALAEITRP, |
| | | | | | | N587A |
| VAR-4 | 15 | 1.35 | 0.37 | −2.58 | 1.34 | 586_9aa_587_LKNAETARP, |
| | | | | | | N587A |
| VAR-5 | 16 | Not Measured | −2.77 | −7.85 | 0.93 | 586_10aa_587_LNLAIEQTRP, |
| | | | | | | N587A, A593T |
| VAR-6 | 17 | Not Measured | −0.53 | −4.69 | 1.50 | 586_8aa_587_MLNEQTRP, |
| | | | | | | N587A |
| VAR-7 | 18 | Not Measured | −1.15 | 0.47 | 2.09 | 584_11aa_585_RSGNRADSETA, |
| | | | | | | G586P, N587A |
| VAR-8 | 19 | Not Measured | −0.61 | −4.22 | 1.75 | 586_6aa_587_TGDTRP, |
| | | | | | | N587A |
| VAR-9 | 20 | −1.53 | 0.08 | −2.71 | 0.45 | 587_9aa_588_LQGETIRPA |
| VAR- | 21 | Not Measured | −0.52 | −2.30 | 1.41 | 586_11aa_587_QNLANPETTRP, |
| 10 | | | | | | N587A |
| VAR- | 22 | Not Measured | 2.45 | Not Measured | 1.29 | 587_10aa_588_RAPQETTRPA, |
| 11 | | | | | | T592A, T597W |
| VAR- | 23 | −1.51 | −0.92 | −2.82 | −0.06 | 586_8aa_587_ANLTTTRP, |
| 12 | | | | | | N587A |
| VAR- | 24 | 3.71 | 2.43 | −0.78 | 2.96 | 586_10aa_587_ALLAGEQTRP, |
| 13 | | | | | | N587A |
| VAR- | 25 | 2.76 | 3.79 | −6.99 | −0.01 | D561C, |
| 14 | | | | | | 586_9aa_587_GLRAEQTRP, |
| | | | | | | N587A, T597N |
| VAR- | 26 | −1.53 | 0.08 | −2.71 | 0.45 | T550N, |
| 15 | | | | | | 584_8aa_585_RARLDETA, |
| | | | | | | G586P, N587A |
| VAR- | 27 | 2.45 | 3.01 | −3.34 | 1.54 | I559L, |
| 16 | | | | | | 586_11aa_587_TNLARGETARP, |
| | | | | | | N587A |

TABLE 1C

| | | | Non-Macular | Macular | | |
|---|---|---|---|---|---|---|
| | | Choroidal | Retina | Retina | Retinal | |
| | | Transduction | Transduction | Transduction | Transduction | |
| | | (intravitreal | (intravitreal | (intravitreal | (intravitreal | |
| | SEQ ID | administration) | administration) | administration) | administration) | |
| | NO: of | as compared | as compared | as compared | as compared | |
| | VP1 | to wild-type | to wild-type | to wild-type | to wild type | Mutation |
| | capsid | SEQ ID | SEQ ID | SEQ ID | SEQ ID | Differences as |
| | poly- | NO: 1 | NO: 1 | NO: 1 | NO: 1 | compared to SEQ |
| Name | peptide | (Log2) | (Log2) | (Log2) | (Log2) | ID NO: 1 |
| VAR-1 | 12 | 0.84 | 0.33 | 0.57 | 0.37 | 587_10aa_588_LALGEQTRPA |
| VAR-2 | 13 | 0.13 | −0.01 | 0.10 | 0.01 | 587_9aa_588_LAIEQTRPA |
| VAR-3 | 14 | −1.06 | −0.38 | −0.14 | −0.34 | 586_9aa_587_LALAEITRP, |
| | | | | | | N587A |
| VAR-4 | 15 | 1.20 | 0.84 | 1.83 | 1.04 | 586_9aa_587_LKNAETARP, |
| | | | | | | N587A |
| VAR-5 | 16 | 0.61 | 1.34 | 2.25 | 1.52 | 586_10aa_587_LNLAIEQTRP, |
| | | | | | | N587A, A593T |
| VAR-6 | 17 | 0.82 | 1.23 | 1.80 | 1.34 | 586_8aa_587_MLNEQTRP, |
| | | | | | | N587A |
| VAR-7 | 18 | 0.38 | 1.28 | 2.01 | 1.42 | 584_11aa_585_RSGNRADSETA, |
| | | | | | | G586P, N587A |
| VAR-8 | 19 | 0.54 | 1.14 | 1.89 | 1.29 | 586_6aa_587_TGDTRP, |
| | | | | | | N587A |
| VAR-9 | 20 | 1.01 | 0.87 | 1.36 | 0.96 | 587_9aa_588_LQGETIRPA |
| VAR- | 21 | 0.85 | 1.21 | 1.53 | 1.27 | 586_11aa_587_QNLANPETTRP, |
| 10 | | | | | | N587A |
| VAR- | 22 | −0.11 | 1.30 | 2.20 | 1.48 | 587_10aa_588_RAPQETTRPA, |
| 11 | | | | | | T592A, T597W |
| VAR- | 23 | 0.60 | 0.88 | 1.53 | 1.00 | 586_8aa_587_ANLTTTRP, |
| 12 | | | | | | N587A |
| VAR- | 24 | −0.46 | −0.71 | −0.33 | −0.64 | 586_10aa_587_ALLAGEQTRP, |
| 13 | | | | | | N587A |
| VAR- | 25 | −1.69 | 0.32 | 1.10 | 0.47 | D561C, |
| 14 | | | | | | 586_9aa_587_GLRAEQTRP, |
| | | | | | | N587A, T597N |
| VAR- | 26 | 1.01 | 0.87 | 1.36 | 0.96 | T550N, |
| 15 | | | | | | 584_8aa_585_RARLDETA, |
| | | | | | | G586P, N587A |
| VAR- | 27 | −1.34 | −0.04 | 0.95 | 0.16 | I559L, |
| 16 | | | | | | 586_11aa_587_TNLARGETARP, |
| | | | | | | N587A |

TABLE 1D

| | | | Neural Retina | | |
|---|---|---|---|---|---|
| | | | Transduction | | |
| | | Virus | (intravitreal | | |
| | | Production | administration) as | | |
| | | (intravitreal | compared to wild-type | | |
| | | administration) | SEQ ID NO: 1 | | |
| | SEQ ID NO: | as compared | | Standard | Mutation |
| | of VP1 | to wild- | | Error of | Differences as |
| | capsid | type SEQ | | the Mean | compared to |
| Name | polypeptide | ID NO: 1 | Mean | (SEM) | SEQ ID NO: 1 |
| VAR-1 | 12 | 3.07 | 48 | 1.33 | [‘587_10aa_588_LALGEQTRPA’] |
| VAR-2 | 13 | 2.68 | 15.42 | 1.42 | [‘587_9aa_588_LAIEQTRPA’] |
| VAR-4 | 15 | 1.47 | 6.78 | 1.19 | [‘586_9aa 587_LKNAETARP’, |
| | | | | | ‘N587A’] |
| VAR-5 | 16 | 1.65 | 28.48 | 1.54 | [‘586_10aa_587_LNLAIEQTRP’, |
| | | | | | ‘N587A’, ‘A593T’] |
| VAR-6 | 17 | 3.43 | 71.75 | 1.4 | [‘586_8aa_587_MLNEQTRP’, |
| | | | | | ‘N587A’] |
| VAR-7 | 18 | 2.97 | 47.17 | 1.22 | [‘584_11aa_585_RSGNRADSETA’, |
| | | | | | ‘G586P’, ‘N587A’] |
| VAR-8 | 19 | 2.66 | 80.82 | 1.3 | [‘586_6aa_587_ TGDTRP’, |
| | | | | | ‘N587A’ ] |
| VAR-11 | 22 | 1.21 | 3.81 | 1.24 | [‘587_10aa_588_RAPQETTRPA’, |
| | | | | | ‘T592A’, ‘T597W’] |

TABLE 1E

| | | | Neural Retina | | |
|---|---|---|---|---|---|
| | | | Transduction | | |
| | | Virus | (intravitreal | | |
| | | Production | administration) as | | |
| | | (intravitreal | compared to wild-type | | |
| | SEQ ID | administration) | SEQ ID NO: 1 | | |
| | NO: of | as compared | | Standard | Mutation |
| | VP1 | to wild- | | Error of | Differences as |
| | capsid | type SEQ | | the Mean | compared to |
| Name | polypeptide | ID NO: 1 | Mean | (SEM) | SEQ ID NO: 1 |
| VAR-1 | 12 | 3.07 | 51.69 | 1.17 | [‘587_10aa_588_LALGEQTRPA’] |
| VAR-2 | 13 | 2.68 | 13.82 | 1.23 | [‘587_9aa_588_LAIEQTRPA’] |
| VAR-4 | 15 | 1.47 | 9.3 | 1.16 | [‘586_9aa_587_LKNAETARP’, |
| | | | | | ‘N587A’] |
| VAR-5 | 16 | 1.65 | 36.96 | 1.22 | [‘586_10aa_587_LNLAIEQTRP’, |
| | | | | | ‘N587A’, ‘A593T‘] |
| VAR-6 | 17 | 3.43 | 70.1 | 1.21 | [‘586_8aa_587_MLNEQTRP’, |
| | | | | | ‘N587A’] |
| VAR-7 | 18 | 2.97 | 53.11 | 1.17 | [‘584_11aa_585_ RSGNRADSETA’, |
| | | | | | ‘G586P’, ‘N587A’] |
| VAR-8 | 19 | 2.66 | 105.72 | 1.16 | [‘586_6aa_587_ TGDTRP’, |
| | | | | | ‘N587A’] |
| VAR-11 | 22 | 1.21 | 4.77 | 1.2 | [‘587_10aa_588_RAPQETTRPA’, |
| | | | | | ‘T592A’, |
| | | | | | ‘T597W’] |

TABLE 1F

| | | | | Trabecular Transduction | | |
|---|---|---|---|---|---|---|
| | | Macular | | (intravitreal | | |
| | | Transduction | | administration) as | | |
| | | (intravitreal | | compared to virus | | |
| | | administration) as | | particle with capsid | | |
| | | compared to wild- | | polypeptides of | | |
| | SEQ ID | type SEQ ID NO: 1 | | SEQ ID NO: 60 | | Mutation |
| | NO: of | | Standard | | Standard | Differences |
| | VP1 | | Error of | | Error of | as compared |
| | capsid | | the Mean | | the Mean | to SEQ |
| Name | polypeptide | Mean | (SEM) | Mean | (SEM) | ID NO: 1 |
| VAR-1 | 12 | 332.46 | 1.21 | 1.53 | 1.15 | [‘587_10aa_588_LALGEQTRPA’] |
| VAR-2 | 13 | 116.94 | 1.32 | 1.58 | 1.15 | [‘587_9aa_588_LAIEQTRPA’] |
| VAR-4 | 15 | 61.71 | 1.36 | 0.02 | 1.2 | [‘586_9aa_587_LKNAETARP’, |
| | | | | | | ‘N587A’] |
| VAR-5 | 16 | 239.59 | 1.22 | 0.36 | 1.38 | [‘586_10aa_587_LNLAIEQTRP’, |
| | | | | | | ‘N587A’, ‘A593T’] |
| VAR-6 | 17 | 523.4 | 1.3 | 2.75 | 1.11 | [‘586_8aa_587_MLNEQTRP’, |
| | | | | | | ‘N587A’] |
| VAR-7 | 18 | 466.67 | 1.13 | 0.06 | 1.49 | [‘584_11aa_585_RSGNRADSETA’, |
| | | | | | | ‘G586P’, ‘N587A’] |
| VAR-8 | 19 | 1036.93 | 1.16 | 1.45 | 1.12 | [‘586_6aa_587_TGDTRP’, |
| | | | | | | ‘N587A’] |
| VAR-11 | 22 | 28.09 | 1.33 | 0.02 | 1.28 | [‘587_10aa_588_RAPQETTRPA’, |
| | | | | | | ‘T592A’, ‘T597W’] |

TABLE 1G

| | | | Trabecular Transduction | | |
|---|---|---|---|---|---|
| | | | (intracameral | | |
| | | | administration) as | | |
| | | Virus | compared to virus | | |
| | | Production | particle with capsid | | |
| | | (intracameral | polypeptides of | | |
| | SEQ ID | administration) | SEQ ID NO: 60 | | |
| | NO: of | as compared | | Standard | Mutation |
| | VP1 | to wild- | | Error of | Differences as |
| | capsid | type SEQ | | the Mean | compared to |
| Name | polypeptide | ID NO: 1 | Mean | (SEM) | SEQ ID NO: 1 |
| VAR-2 | 13 | 0.55 | 0.08 | 1.64 | [‘587_9aa_588_LAIEQTRPA’] |
| VAR-3 | 14 | 1.79 | 0.55 | 1.34 | [‘586_9aa_587_LALAEITRP’, |
| | | | | | ‘N587A’] |
| VAR-13 | 24 | 6.31 | 0.28 | 1.22 | [‘586_10aa_ 587_ ALLAGEQTRP’, |
| | | | | | ‘N587A’] |
| VAR-14 | 25 | 1.3 | 0.31 | 1.62 | [‘D561C’, |
| | | | | | ‘586_9aa_587_GLRAEQTRP’, |
| | | | | | ‘N587A’, |
| | | | | | ‘T597N’] |

TABLE 2

| | Amino Acid Sequence of VP1 capsid | |
|---|---|---|
| | polypeptide (SEQ ID NO; starting | |
| | amino acid of VP2 is underlined; | |
| Capsid | starting amino acid of VP3 is in | Exemplary Nucleic Acid Molecule |
| Variant | bold. | Sequence (SEQ ID NO) |
| VAR-1 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGNLALGEQTRPARQAATADVNTQGVLP | TGGAGACGCAGACTCAGTACCTGACCCCCAG |
| | GMVWQDRDVYLQGPIWAKIPHTDGHFHPSPLMGG | CCTCTCGGACAGCCACCAGCAGCCCCCTCTG |
| | FGLKHPPPQILIKNTPVPANPSTTFSAAKFASFI | GTCTGGGAACTAATACGATGGCTACAGGCAG |
| | TQYSTGQVSVEIEWELQKENSKRWNPEIQYTSNY | TGGCGCACCAATGGCAGACAATAACGAGGGC |
| | NKSVNVDFTVDTNGVYSEPRPIGTRYLTRNL | GCCGACGGAGTGGGTAATTCCTCGGGAAATT |
| | (SEQ ID NO: 12) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCAACTTGGCT |
| | | TTAGGGGAGCAAACAAGACCTGCAAGACAAG |
| | | CAGCTACCGCAGATGTCAACACACAAGGCGT |
| | | TCTTCCAGGCATGGTCTGGCAGGACAGAGAT |
| | | GTGTACCTTCAGGGGCCCATCTGGGCAAAGA |
| | | TTCCACACACGGACGGACATTTTCACCCCTC |
| | | TCCCCTCATGGGTGGATTCGGACTTAAACAC |
| | | CCTCCTCCACAGATTCTCATCAAGAACACCC |
| | | CGGTACCTGCGAATCCTTCGACCACCTTCAG |
| | | TGCGGCAAAGTTTGCTTCCTTCATCACACAG |
| | | TACTCCACGGGACAGGTCAGCGTGGAGATCG |
| | | AGTGGGAGCTGCAGAAGGAAAACAGCAAACG |
| | | CTGGAATCCCGAAATTCAGTACACTTCCAAC |
| | | TACAACAAGTCTGTTAATGTGGACTTTACTG |
| | | TGGACACTAATGGCGTGTATTCAGAGCCTCG |
| | | CCCCATTGGCACCAGATACCTGACTCGTAAT |
| | | CTGTAA (SEQ ID NO: 28) |
| VAR-2 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGNLAIEQTRPARQAATADVNTQGVLPG | TGGAGACGCAGACTCAGTACCTGACCCCCAG |
| | MVWQDRDVYLQGPIWAKIPHTDGHFHPSPLMGGF | CCTCTCGGACAGCCACCAGCAGCCCCCTCTG |
| | GLKHPPPQILIKNTPVPANPSTTFSAAKFASFIT | GTCTGGGAACTAATACGATGGCTACAGGCAG |
| | QYSTGQVSVEIEWELQKENSKRWNPEIQYTSNYN | TGGCGCACCAATGGCAGACAATAACGAGGGC |
| | KSVNVDFTVDTNGVYSEPRPIGTRYLTRNL | GCCGACGGAGTGGGTAATTCCTCGGGAAATT |
| | (SEQ ID NO: 13) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCAACCTTGCT |
| | | ATAGAACAAACACGCCCCGCCAGACAAGCAG |
| | | CTACCGCAGATGTCAACACACAAGGCGTTCT |
| | | TCCAGGCATGGTCTGGCAGGACAGAGATGTG |
| | | TACCTTCAGGGGCCCATCTGGGCAAAGATTC |
| | | CACACACGGACGGACATTTTCACCCCTCTCC |
| | | CCTCATGGGTGGATTCGGACTTAAACACCCT |
| | | CCTCCACAGATTCTCATCAAGAACACCCCGG |
| | | TACCTGCGAATCCTTCGACCACCTTCAGTGC |
| | | GGCAAAGTTTGCTTCCTTCATCACACAGTAC |
| | | TCCACGGGACAGGTCAGCGTGGAGATCGAGT |
| | | GGGAGCTGCAGAAGGAAAACAGCAAACGCTG |
| | | GAATCCCGAAATTCAGTACACTTCCAACTAC |
| | | AACAAGTCTGTTAATGTGGACTTTACTGTGG |
| | | ACACTAATGGCGTGTATTCAGAGCCTCGCCC |
| | | CATTGGCACCAGATACCTGACTCGTAATCTG |
| | | TAA (SEQ ID NO: 29) |
| VAR-3 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGLALAEITRPARQAATADVNTQGVLPG | TGGAGACGCAGACTCAGTACCTGACCCCCAG |
| | MVWQDRDVYLQGPIWAKIPHTDGHFHPSPLMGGF | CCTCTCGGACAGCCACCAGCAGCCCCCTCTG |
| | GLKHPPPQILIKNTPVPANPSTTFSAAKFASFIT | GTCTGGGAACTAATACGATGGCTACAGGCAG |
| | QYSTGQVSVEIEWELQKENSKRWNPEIQYTSNYN | TGGCGCACCAATGGCAGACAATAACGAGGGC |
| | KSVNVDFTVDTNGVYSEPRPIGTRYLTRNL | GCCGACGGAGTGGGTAATTCCTCGGGAAATT |
| | (SEQ ID NO: 14) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCCTCGCGTTA |
| | | GCAGAGATAACTCGACCCGCGAGACAAGCAG |
| | | CTACCGCAGATGTCAACACACAAGGCGTTCT |
| | | TCCAGGCATGGTCTGGCAGGACAGAGATGTG |
| | | TACCTTCAGGGGCCCATCTGGGCAAAGATTC |
| | | CACACACGGACGGACATTTTCACCCCTCTCC |
| | | CCTCATGGGTGGATTCGGACTTAAACACCCT |
| | | CCTCCACAGATTCTCATCAAGAACACCCCGG |
| | | TACCTGCGAATCCTTCGACCACCTTCAGTGC |
| | | GGCAAAGTTTGCTTCCTTCATCACACAGTAC |
| | | TCCACGGGACAGGTCAGCGTGGAGATCGAGT |
| | | GGGAGCTGCAGAAGGAAAACAGCAAACGCTG |
| | | GAATCCCGAAATTCAGTACACTTCCAACTAC |
| | | AACAAGTCTGTTAATGTGGACTTTACTGTGG |
| | | ACACTAATGGCGTGTATTCAGAGCCTCGCCC |
| | | CATTGGCACCAGATACCTGACTCGTAATCTG |
| | | TAA (SEQ ID NO: 30) |
| VAR-4 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGLKNAETARPARQAATADVNTQGVLPG | TGGAGACGCAGACTCAGTACCTGACCCCCAG |
| | MVWQDRDVYLQGPIWAKIPHTDGHFHPSPLMGGF | CCTCTCGGACAGCCACCAGCAGCCCCCTCTG |
| | GLKHPPPQILIKNTPVPANPSTTFSAAKFASFIT | GTCTGGGAACTAATACGATGGCTACAGGCAG |
| | QYSTGQVSVEIEWELQKENSKRWNPEIQYTSNYN | TGGCGCACCAATGGCAGACAATAACGAGGGC |
| | KSVNVDFTVDTNGVYSEPRPIGTRYLTRNL | GCCGACGGAGTGGGTAATTCCTCGGGAAATT |
| | (SEQ ID NO: 15) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCCTGAAGAAC |
| | | GCGGAAACAGCACGTCCCGCAAGACAAGCAG |
| | | CTACCGCAGATGTCAACACACAAGGCGTTCT |
| | | TCCAGGCATGGTCTGGCAGGACAGAGATGTG |
| | | TACCTTCAGGGGCCCATCTGGGCAAAGATTC |
| | | CACACACGGACGGACATTTTCACCCCTCTCC |
| | | CCTCATGGGTGGATTCGGACTTAAACACCCT |
| | | CCTCCACAGATTCTCATCAAGAACACCCCGG |
| | | TACCTGCGAATCCTTCGACCACCTTCAGTGC |
| | | GGCAAAGTTTGCTTCCTTCATCACACAGTAC |
| | | TCCACGGGACAGGTCAGCGTGGAGATCGAGT |
| | | GGGAGCTGCAGAAGGAAAACAGCAAACGCTG |
| | | GAATCCCGAAATTCAGTACACTTCCAACTAC |
| | | AACAAGTCTGTTAATGTGGACTTTACTGTGG |
| | | ACACTAATGGCGTGTATTCAGAGCCTCGCCC |
| | | CATTGGCACCAGATACCTGACTCGTAATCTG |
| | | TAA (SEQ ID NO: 31) |
| VAR-5 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHESPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGLNLAIEQTRPARQAATTDVNTQGVLP | TGGAGACGCAGACTCAGTACCTGACCCCCAG |
| | GMVWQDRDVYLQGPIWAKIPHTDGHFHPSPLMGG | CCTCTCGGACAGCCACCAGCAGCCCCCTCTG |
| | FGLKHPPPQILIKNTPVPANPSTTFSAAKFASFI | GTCTGGGAACTAATACGATGGCTACAGGCAG |
| | TQYSTGQVSVEIEWELQKENSKRWNPEIQYTSNY | TGGCGCACCAATGGCAGACAATAACGAGGGC |
| | NKSVNVDFTVDTNGVYSEPRPIGTRYLTRNL | GCCGACGGAGTGGGTAATTCCTCGGGAAATT |
| | (SEQ ID NO: 16) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCCTCAACCTT |
| | | GCGATAGAGCAAACCAGGCCCGCGAGACAAG |
| | | CAGCTACCACCGATGTCAACACACAAGGCGT |
| | | TCTTCCAGGCATGGTCTGGCAGGACAGAGAT |
| | | GTGTACCTTCAGGGGCCCATCTGGGCAAAGA |
| | | TTCCACACACGGACGGACATTTTCACCCCTC |
| | | TCCCCTCATGGGTGGATTCGGACTTAAACAC |
| | | CCTCCTCCACAGATTCTCATCAAGAACACCC |
| | | CGGTACCTGCGAATCCTTCGACCACCTTCAG |
| | | TGCGGCAAAGTTTGCTTCCTTCATCACACAG |
| | | TACTCCACGGGACAGGTCAGCGTGGAGATCG |
| | | AGTGGGAGCTGCAGAAGGAAAACAGCAAACG |
| | | CTGGAATCCCGAAATTCAGTACACTTCCAAC |
| | | TACAACAAGTCTGTTAATGTGGACTTTACTG |
| | | TGGACACTAATGGCGTGTATTCAGAGCCTCG |
| | | CCCCATTGGCACCAGATACCTGACTCGTAAT |
| | | CTGTAA (SEQ ID NO: 32) |
| VAR-6 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGMLNEQTRPARQAATADVNTQGVLPGM | TGGAGACGCAGACTCAGTACCTGACCCCCAG |
| | VWQDRDVYLQGPIWAKIPHTDGHFHPSPLMGGFG | CCTCTCGGACAGCCACCAGCAGCCCCCTCTG |
| | LKHPPPQILIKNTPVPANPSTTFSAAKFASFITQ | GTCTGGGAACTAATACGATGGCTACAGGCAG |
| | YSTGQVSVEIEWELQKENSKRWNPEIQYTSNYNK | TGGCGCACCAATGGCAGACAATAACGAGGGC |
| | SVNVDFTVDTNGVYSEPRPIGTRYLTRNL | GCCGACGGAGTGGGTAATTCCTCGGGAAATT |
| | (SEQ ID NO: 17) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCATGTTAAAT |
| | | GAGCAAACTCGGCCAGCCAGACAAGCAGCTA |
| | | CCGCAGATGTCAACACACAAGGCGTTCTTCC |
| | | AGGCATGGTCTGGCAGGACAGAGATGTGTAC |
| | | CTTCAGGGGCCCATCTGGGCAAAGATTCCAC |
| | | ACACGGACGGACATTTTCACCCCTCTCCCCT |
| | | CATGGGTGGATTCGGACTTAAACACCCTCCT |
| | | CCACAGATTCTCATCAAGAACACCCCGGTAC |
| | | CTGCGAATCCTTCGACCACCTTCAGTGCGGC |
| | | AAAGTTTGCTTCCTTCATCACACAGTACTCC |
| | | ACGGGACAGGTCAGCGTGGAGATCGAGTGGG |
| | | AGCTGCAGAAGGAAAACAGCAAACGCTGGAA |
| | | TCCCGAAATTCAGTACACTTCCAACTACAAC |
| | | AAGTCTGTTAATGTGGACTTTACTGTGGACA |
| | | CTAATGGCGTGTATTCAGAGCCTCGCCCCAT |
| | | TGGCACCAGATACCTGACTCGTAATCTGTAA |
| | | (SEQ ID NO: 33) |
| VAR-7 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHESPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRSGNRADSETARPARQAATADVNTQGVL | TGGAGACGCAGACTCAGTACCTGACCCCCAG |
| | PGMVWQDRDVYLQGPIWAKIPHTDGHFHPSPLMG | CCTCTCGGACAGCCACCAGCAGCCCCCTCTG |
| | GFGLKHPPPQILIKNTPVPANPSTTFSAAKFASE | GTCTGGGAACTAATACGATGGCTACAGGCAG |
| | ITQYSTGQVSVEIEWELQKENSKRWNPEIQYTSN | TGGCGCACCAATGGCAGACAATAACGAGGGC |
| | YNKSVNVDFTVDTNGVYSEPRPIGTRYLTRNL | GCCGACGGAGTGGGTAATTCCTCGGGAAATT |
| | (SEQ ID NO: 18) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGCGGAGCGGCAACCGA |
| | | GCAGATAGCGAGACTGCAAGACCCGCTAGAC |
| | | AAGCAGCTACCGCAGATGTCAACACACAAGG |
| | | CGTTCTTCCAGGCATGGTCTGGCAGGACAGA |
| | | GATGTGTACCTTCAGGGGCCCATCTGGGCAA |
| | | AGATTCCACACACGGACGGACATTTTCACCC |
| | | CTCTCCCCTCATGGGTGGATTCGGACTTAAA |
| | | CACCCTCCTCCACAGATTCTCATCAAGAACA |
| | | CCCCGGTACCTGCGAATCCTTCGACCACCTT |
| | | CAGTGCGGCAAAGTTTGCTTCCTTCATCACA |
| | | CAGTACTCCACGGGACAGGTCAGCGTGGAGA |
| | | TCGAGTGGGAGCTGCAGAAGGAAAACAGCAA |
| | | ACGCTGGAATCCCGAAATTCAGTACACTTCC |
| | | AACTACAACAAGTCTGTTAATGTGGACTTTA |
| | | CTGTGGACACTAATGGCGTGTATTCAGAGCC |
| | | TCGCCCCATTGGCACCAGATACCTGACTCGT |
| | | AATCTGTAA (SEQ ID NO: 34) |
| VAR-8 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGTGDTRPARQAATADVNTQGVLPGMVW | TGGAGACGCAGACTCAGTACCTGACCCCCAG |
| | QDRDVYLQGPIWAKIPHTDGHFHPSPLMGGFGLK | CCTCTCGGACAGCCACCAGCAGCCCCCTCTG |
| | HPPPQILIKNTPVPANPSTTFSAAKFASFITQYS | GTCTGGGAACTAATACGATGGCTACAGGCAG |
| | TGQVSVEIEWELQKENSKRWNPEIQYTSNYNKSV | TGGCGCACCAATGGCAGACAATAACGAGGGC |
| | NVDFTVDTNGVYSEPRPIGTRYLTRNL (SEQ | GCCGACGGAGTGGGTAATTCCTCGGGAAATT |
| | ID NO: 19) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCACCGGTGAT |
| | | ACCCGGCCTGCTAGACAAGCAGCTACCGCAG |
| | | ATGTCAACACACAAGGCGTTCTTCCAGGCAT |
| | | GGTCTGGCAGGACAGAGATGTGTACCTTCAG |
| | | GGGCCCATCTGGGCAAAGATTCCACACACGG |
| | | ACGGACATTTTCACCCCTCTCCCCTCATGGG |
| | | TGGATTCGGACTTAAACACCCTCCTCCACAG |
| | | ATTCTCATCAAGAACACCCCGGTACCTGCGA |
| | | ATCCTTCGACCACCTTCAGTGCGGCAAAGTT |
| | | TGCTTCCTTCATCACACAGTACTCCACGGGA |
| | | CAGGTCAGCGTGGAGATCGAGTGGGAGCTGC |
| | | AGAAGGAAAACAGCAAACGCTGGAATCCCGA |
| | | AATTCAGTACACTTCCAACTACAACAAGTCT |
| | | GTTAATGTGGACTTTACTGTGGACACTAATG |
| | | GCGTGTATTCAGAGCCTCGCCCCATTGGCAC |
| | | CAGATACCTGACTCGTAATCTGTAA (SEQ |
| | | ID NO: 35) |
| VAR-9 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGNLQGETIRPARQAATADVNTQGVLPG | TGGAGACGCAGACTCAGTACCTGACCCCCAG |
| | MVWQDRDVYLQGPIWAKIPHTDGHFHPSPLMGGF | CCTCTCGGACAGCCACCAGCAGCCCCCTCTG |
| | GLKHPPPQILIKNTPVPANPSTTFSAAKFASFIT | GTCTGGGAACTAATACGATGGCTACAGGCAG |
| | QYSTGQVSVEIEWELQKENSKRWNPEIQYTSNYN | TGGCGCACCAATGGCAGACAATAACGAGGGC |
| | KSVNVDFTVDTNGVYSEPRPIGTRYLTRNL | GCCGACGGAGTGGGTAATTCCTCGGGAAATT |
| | (SEQ ID NO: 20) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCAACCTACAA |
| | | GGTGAGACAATTAGACCCGCGAGACAAGCAG |
| | | CTACCGCAGATGTCAACACACAAGGCGTTCT |
| | | TCCAGGCATGGTCTGGCAGGACAGAGATGTG |
| | | TACCTTCAGGGGCCCATCTGGGCAAAGATTC |
| | | CACACACGGACGGACATTTTCACCCCTCTCC |
| | | CCTCATGGGTGGATTCGGACTTAAACACCCT |
| | | CCTCCACAGATTCTCATCAAGAACACCCCGG |
| | | TACCTGCGAATCCTTCGACCACCTTCAGTGC |
| | | GGCAAAGTTTGCTTCCTTCATCACACAGTAC |
| | | TCCACGGGACAGGTCAGCGTGGAGATCGAGT |
| | | GGGAGCTGCAGAAGGAAAACAGCAAACGCTG |
| | | GAATCCCGAAATTCAGTACACTTCCAACTAC |
| | | AACAAGTCTGTTAATGTGGACTTTACTGTGG |
| | | ACACTAATGGCGTGTATTCAGAGCCTCGCCC |
| | | CATTGGCACCAGATACCTGACTCGTAATCTG |
| | | TAA (SEQ ID NO: 36) |
| VAR-10 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGQNLANPETTRPARQAATADVNTQGVL | TGGAGACGCAGACTCAGTACCTGACCCCCAG |
| | PGMVWQDRDVYLQGPIWAKIPHTDGHFHPSPLMG | CCTCTCGGACAGCCACCAGCAGCCCCCTCTG |
| | GFGLKHPPPQILIKNTPVPANPSTTFSAAKFASF | GTCTGGGAACTAATACGATGGCTACAGGCAG |
| | ITQYSTGQVSVEIEWELQKENSKRWNPEIQYTSN | TGGCGCACCAATGGCAGACAATAACGAGGGC |
| | YNKSVNVDFTVDTNGVYSEPRPIGTRYLTRNL | GCCGACGGAGTGGGTAATTCCTCGGGAAATT |
| | (SEQ ID NO: 21) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCCAAAACCTC |
| | | GCGAACCCAGAGACAACACGTCCTGCGAGAC |
| | | AAGCAGCTACCGCAGATGTCAACACACAAGG |
| | | CGTTCTTCCAGGCATGGTCTGGCAGGACAGA |
| | | GATGTGTACCTTCAGGGGCCCATCTGGGCAA |
| | | AGATTCCACACACGGACGGACATTTTCACCC |
| | | CTCTCCCCTCATGGGTGGATTCGGACTTAAA |
| | | CACCCTCCTCCACAGATTCTCATCAAGAACA |
| | | CCCCGGTACCTGCGAATCCTTCGACCACCTT |
| | | CAGTGCGGCAAAGTTTGCTTCCTTCATCACA |
| | | CAGTACTCCACGGGACAGGTCAGCGTGGAGA |
| | | TCGAGTGGGAGCTGCAGAAGGAAAACAGCAA |
| | | ACGCTGGAATCCCGAAATTCAGTACACTTCC |
| | | AACTACAACAAGTCTGTTAATGTGGACTTTA |
| | | CTGTGGACACTAATGGCGTGTATTCAGAGCC |
| | | TCGCCCCATTGGCACCAGATACCTGACTCGT |
| | | AATCTGTAA (SEQ ID NO: 37) |
| VAR-11 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGNRAPQETTRPARQAAAADVNWQGVLP | TGGAGACGCAGACTCAGTACCTGACCCCCAG |
| | GMVWQDRDVYLQGPIWAKIPHTDGHFHPSPLMGG | CCTCTCGGACAGCCACCAGCAGCCCCCTCTG |
| | FGLKHPPPQILIKNTPVPANPSTTFSAAKFASFI | GTCTGGGAACTAATACGATGGCTACAGGCAG |
| | TQYSTGQVSVEIEWELQKENSKRWNPEIQYTSNY | TGGCGCACCAATGGCAGACAATAACGAGGGC |
| | NKSVNVDFTVDTNGVYSEPRPIGTRYLTRNL | GCCGACGGAGTGGGTAATTCCTCGGGAAATT |
| | (SEQ ID NO: 22) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCAACCGTGCA |
| | | CCGCAGGAGACAACCCGGCCCGCCAGACAAG |
| | | CAGCTGCAGCAGATGTCAACTGGCAAGGCGT |
| | | TCTTCCAGGCATGGTCTGGCAGGACAGAGAT |
| | | GTGTACCTTCAGGGGCCCATCTGGGCAAAGA |
| | | TTCCACACACGGACGGACATTTTCACCCCTC |
| | | TCCCCTCATGGGTGGATTCGGACTTAAACAC |
| | | CCTCCTCCACAGATTCTCATCAAGAACACCC |
| | | CGGTACCTGCGAATCCTTCGACCACCTTCAG |
| | | TGCGGCAAAGTTTGCTTCCTTCATCACACAG |
| | | TACTCCACGGGACAGGTCAGCGTGGAGATCG |
| | | AGTGGGAGCTGCAGAAGGAAAACAGCAAACG |
| | | CTGGAATCCCGAAATTCAGTACACTTCCAAC |
| | | TACAACAAGTCTGTTAATGTGGACTTTACTG |
| | | TGGACACTAATGGCGTGTATTCAGAGCCTCG |
| | | CCCCATTGGCACCAGATACCTGACTCGTAAT |
| | | CTGTAA (SEQ ID NO: 38) |
| VAR-12 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGANLTTTRPARQAATADVNTQGVLPGM | TGGAGACGCAGACTCAGTACCTGACCCCCAG |
| | VWQDRDVYLQGPIWAKIPHTDGHFHPSPLMGGFG | CCTCTCGGACAGCCACCAGCAGCCCCCTCTG |
| | LKHPPPQILIKNTPVPANPSTTFSAAKFASFITQ | GTCTGGGAACTAATACGATGGCTACAGGCAG |
| | YSTGQVSVEIEWELQKENSKRWNPEIQYTSNYNK | TGGCGCACCAATGGCAGACAATAACGAGGGC |
| | SVNVDFTVDTNGVYSEPRPIGTRYLTRNL (SEQ | GCCGACGGAGTGGGTAATTCCTCGGGAAATT |
| | ID NO: 23) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCGCCAACTTA |
| | | ACTACGACTCGTCCCGCGAGACAAGCAGCTA |
| | | CCGCAGATGTCAACACACAAGGCGTTCTTCC |
| | | AGGCATGGTCTGGCAGGACAGAGATGTGTAC |
| | | CTTCAGGGGCCCATCTGGGCAAAGATTCCAC |
| | | ACACGGACGGACATTTTCACCCCTCTCCCCT |
| | | CATGGGTGGATTCGGACTTAAACACCCTCCT |
| | | CCACAGATTCTCATCAAGAACACCCCGGTAC |
| | | CTGCGAATCCTTCGACCACCTTCAGTGCGGC |
| | | AAAGTTTGCTTCCTTCATCACACAGTACTCC |
| | | ACGGGACAGGTCAGCGTGGAGATCGAGTGGG |
| | | AGCTGCAGAAGGAAAACAGCAAACGCTGGAA |
| | | TCCCGAAATTCAGTACACTTCCAACTACAAC |
| | | AAGTCTGTTAATGTGGACTTTACTGTGGACA |
| | | CTAATGGCGTGTATTCAGAGCCTCGCCCCAT |
| | | TGGCACCAGATACCTGACTCGTAATCTGTAA |
| | | (SEQ ID NO: 39) |
| VAR-13 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITDEEEIRTTNPVATEQYGS | CTGCAAGAAAAAGATTGAATTTTGGTCAGAC |
| | VSTNLQRGALLAGEQTRPARQAATADVNTQGVLP | TGGAGACGCAGACTCAGTACCTGACCCCCAG |
| | GMVWQDRDVYLQGPIWAKIPHTDGHFHPSPLMGG | CCTCTCGGACAGCCACCAGCAGCCCCCTCTG |
| | FGLKHPPPQILIKNTPVPANPSTTFSAAKFASFI | GTCTGGGAACTAATACGATGGCTACAGGCAG |
| | TQYSTGQVSVEIEWELQKENSKRWNPEIQYTSNY | TGGCGCACCAATGGCAGACAATAACGAGGGC |
| | NKSVNVDFTVDTNGVYSEPRPIGTRYLTRNL | GCCGACGGAGTGGGTAATTCCTCGGGAAATT |
| | (SEQ ID NO: 24) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGAGGCGCTCTGCTG |
| | | GCGGGCGAGCAGACAAGACCGGCAAGACAAG |
| | | CAGCTACCGCAGATGTCAACACACAAGGCGT |
| | | TCTTCCAGGCATGGTCTGGCAGGACAGAGAT |
| | | GTGTACCTTCAGGGGCCCATCTGGGCAAAGA |
| | | TTCCACACACGGACGGACATTTTCACCCCTC |
| | | TCCCCTCATGGGTGGATTCGGACTTAAACAC |
| | | CCTCCTCCACAGATTCTCATCAAGAACACCC |
| | | CGGTACCTGCGAATCCTTCGACCACCTTCAG |
| | | TGCGGCAAAGTTTGCTTCCTTCATCACACAG |
| | | TACTCCACGGGACAGGTCAGCGTGGAGATCG |
| | | AGTGGGAGCTGCAGAAGGAAAACAGCAAACG |
| | | CTGGAATCCCGAAATTCAGTACACTTCCAAC |
| | | TACAACAAGTCTGTTAATGTGGACTTTACTG |
| | | TGGACACTAATGGCGTGTATTCAGAGCCTCG |
| | | CCCCATTGGCACCAGATACCTGACTCGTAAT |
| | | CTGTAA (SEQ ID NO: 40) |
| VAR-14 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMITCEEEIRTTNPVATEQYGS | CTGCAAGAAAAAGATTGAATTTTGGTCAGAC |
| | VSTNLQRGGLRAEQTRPARQAATADVNNQGVLPG | TGGAGACGCAGACTCAGTACCTGACCCCCAG |
| | MVWQDRDVYLQGPIWAKIPHTDGHFHPSPLMGGF | CCTCTCGGACAGCCACCAGCAGCCCCCTCTG |
| | GLKHPPPQILIKNTPVPANPSTTESAAKFASFIT | GTCTGGGAACTAATACGATGGCTACAGGCAG |
| | QYSTGQVSVEIEWELQKENSKRWNPEIQYTSNYN | TGGCGCACCAATGGCAGACAATAACGAGGGC |
| | KSVNVDFTVDTNGVYSEPRPIGTRYLTRNL | GCCGACGGAGTGGGTAATTCCTCGGGAAATT |
| | (SEQ ID NO: 25) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | ATTACATGTGAAGAGGAAATCAGGACAACCA |
| | | ATCCCGTGGCTACGGAGCAGTATGGTTCTGT |
| | | ATCTACCAACCTCCAGAGAGGCGGTTTGAGA |
| | | GCTGAGCAAACCCGCCCAGCCAGACAAGCAG |
| | | CTACCGCAGATGTCAACAATCAAGGCGTTCT |
| | | TCCAGGCATGGTCTGGCAGGACAGAGATGTG |
| | | TACCTTCAGGGGCCCATCTGGGCAAAGATTC |
| | | CACACACGGACGGACATTTTCACCCCTCTCC |
| | | CCTCATGGGTGGATTCGGACTTAAACACCCT |
| | | CCTCCACAGATTCTCATCAAGAACACCCCGG |
| | | TACCTGCGAATCCTTCGACCACCTTCAGTGC |
| | | GGCAAAGTTTGCTTCCTTCATCACACAGTAC |
| | | TCCACGGGACAGGTCAGCGTGGAGATCGAGT |
| | | GGGAGCTGCAGAAGGAAAACAGCAAACGCTG |
| | | GAATCCCGAAATTCAGTACACTTCCAACTAC |
| | | AACAAGTCTGTTAATGTGGACTTTACTGTGG |
| | | ACACTAATGGCGTGTATTCAGAGCCTCGCCC |
| | | CATTGGCACCAGATACCTGACTCGTAATCTG |
| | | TAA (SEQ ID NO: 41) |
| VAR-15 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHFSPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKNNVDIEKVMITDEEEIRTTNPVATEQYGS | CTGCAAGAAAAAGATTGAATTTTGGTCAGAC |
| | VSTNLQRARLDETARPARQAATADVNTQGVLPGM | TGGAGACGCAGACTCAGTACCTGACCCCCAG |
| | VWQDRDVYLQGPIWAKIPHTDGHFHPSPLMGGFG | CCTCTCGGACAGCCACCAGCAGCCCCCTCTG |
| | LKHPPPQILIKNTPVPANPSTTFSAAKFASFITQ | GTCTGGGAACTAATACGATGGCTACAGGCAG |
| | YSTGQVSVEIEWELQKENSKRWNPEIQYTSNYNK | TGGCGCACCAATGGCAGACAATAACGAGGGC |
| | SVNVDFTVDTNGVYSEPRPIGTRYLTRNL (SEQ | GCCGACGGAGTGGGTAATTCCTCGGGAAATT |
| | ID NO: 26) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAAACAATGTGGACATTGAAAAGGTCATG |
| | | ATTACAGACGAAGAGGAAATCAGGACAACCA |
| | | ATCCCGTGGCTACGGAGCAGTATGGTTCTGT |
| | | ATCTACCAACCTCCAGAGGGCGCGTCTTGAC |
| | | GAAACTGCCAGACCTGCGAGACAAGCAGCTA |
| | | CCGCAGATGTCAACACACAAGGCGTTCTTCC |
| | | AGGCATGGTCTGGCAGGACAGAGATGTGTAC |
| | | CTTCAGGGGCCCATCTGGGCAAAGATTCCAC |
| | | ACACGGACGGACATTTTCACCCCTCTCCCCT |
| | | CATGGGTGGATTCGGACTTAAACACCCTCCT |
| | | CCACAGATTCTCATCAAGAACACCCCGGTAC |
| | | CTGCGAATCCTTCGACCACCTTCAGTGCGGC |
| | | AAAGTTTGCTTCCTTCATCACACAGTACTCC |
| | | ACGGGACAGGTCAGCGTGGAGATCGAGTGGG |
| | | AGCTGCAGAAGGAAAACAGCAAACGCTGGAA |
| | | TCCCGAAATTCAGTACACTTCCAACTACAAC |
| | | AAGTCTGTTAATGTGGACTTTACTGTGGACA |
| | | CTAATGGCGTGTATTCAGAGCCTCGCCCCAT |
| | | TGGCACCAGATACCTGACTCGTAATCTGTAA |
| | | (SEQ ID NO: 42) |
| VAR-16 | MAADGYLPDWLEDTLSEGIRQWWKLKPGPPPPKP | ATGGCTGCCGATGGTTATCTTCCAGATTGGC |
| | AERHKDDSRGLVLPGYKYLGPFNGLDKGEPVNEA | TCGAGGACACTCTCTCTGAAGGAATAAGACA |
| | DAAALEHDKAYDRQLDSGDNPYLKYNHADAEFQE | GTGGTGGAAGCTCAAACCTGGCCCACCACCA |
| | RLKEDTSFGGNLGRAVFQAKKRVLEPLGLVEEPV | CCAAAGCCCGCAGAGCGGCATAAGGACGACA |
| | KTAPGKKRPVEHSPVEPDSSSGTGKAGQQPARKR | GCAGGGGTCTTGTGCTTCCTGGGTACAAGTA |
| | LNFGQTGDADSVPDPQPLGQPPAAPSGLGTNTMA | CCTCGGACCCTTCAACGGACTCGACAAGGGA |
| | TGSGAPMADNNEGADGVGNSSGNWHCDSTWMGDR | GAGCCGGTCAACGAGGCAGACGCCGCGGCCC |
| | VITTSTRTWALPTYNNHLYKQISSQSGASNDNHY | TCGAGCACGACAAAGCCTACGACCGGCAGCT |
| | FGYSTPWGYFDFNRFHCHESPRDWQRLINNNWGF | CGACAGCGGAGACAACCCGTACCTCAAGTAC |
| | RPKRLNFKLFNIQVKEVTQNDGTTTIANNLTSTV | AACCACGCCGACGCGGAGTTTCAGGAGCGCC |
| | QVFTDSEYQLPYVLGSAHQGCLPPFPADVFMVPQ | TTAAAGAAGATACGTCTTTTGGGGGCAACCT |
| | YGYLTLNNGSQAVGRSSFYCLEYFPSQMLRTGNN | CGGACGAGCAGTCTTCCAGGCGAAAAAGAGG |
| | FTFSYTFEDVPFHSSYAHSQSLDRLMNPLIDQYL | GTTCTTGAACCTCTGGGCCTGGTTGAGGAAC |
| | YYLSRTNTPSGTTTQSRLQFSQAGASDIRDQSRN | CTGTTAAGACGGCTCCGGGAAAAAAGAGGCC |
| | WLPGPCYRQQRVSKTSADNNNSEYSWTGATKYHL | GGTAGAGCACTCTCCTGTGGAGCCAGACTCC |
| | NGRDSLVNPGPAMASHKDDEEKFFPQSGVLIFGK | TCCTCGGGAACCGGAAAGGCGGGCCAGCAGC |
| | QGSEKTNVDIEKVMLTDEEEIRTTNPVATEQYGS | CTGCAAGAAAAAGATTGAATTTTGGTCAGAC |
| | VSTNLQRGTNLARGETARPARQAATADVNTQGVL | TGGAGACGCAGACTCAGTACCTGACCCCCAG |
| | PGMVWQDRDVYLQGPIWAKIPHTDGHFHPSPLMG | CCTCTCGGACAGCCACCAGCAGCCCCCTCTG |
| | GFGLKHPPPQILIKNTPVPANPSTTFSAAKFASF | GTCTGGGAACTAATACGATGGCTACAGGCAG |
| | ITQYSTGQVSVEIEWELQKENSKRWNPEIQYTSN | TGGCGCACCAATGGCAGACAATAACGAGGGC |
| | YNKSVNVDFTVDTNGVYSEPRPIGTRYLTRNL | GCCGACGGAGTGGGTAATTCCTCGGGAAATT |
| | (SEQ ID NO: 27) | GGCATTGCGATTCCACATGGATGGGCGACAG |
| | | AGTCATCACCACCAGCACCCGAACCTGGGCC |
| | | CTGCCCACCTACAACAACCACCTCTACAAAC |
| | | AAATTTCCAGCCAATCAGGAGCCTCGAACGA |
| | | CAATCACTACTTTGGCTACAGCACCCCTTGG |
| | | GGGTATTTTGACTTCAACAGATTCCACTGCC |
| | | ACTTTTCACCACGTGACTGGCAAAGACTCAT |
| | | CAACAACAACTGGGGATTCCGACCCAAGAGA |
| | | CTCAACTTCAAGCTCTTTAACATTCAAGTCA |
| | | AAGAGGTCACGCAGAATGACGGTACGACGAC |
| | | GATTGCCAATAACCTTACCAGCACGGTTCAG |
| | | GTGTTTACTGACTCGGAGTACCAGCTCCCGT |
| | | ACGTCCTCGGCTCGGCGCATCAAGGATGCCT |
| | | CCCGCCGTTCCCAGCAGACGTCTTCATGGTG |
| | | CCACAGTATGGATACCTCACCCTGAACAACG |
| | | GGAGTCAGGCAGTAGGACGCTCTTCATTTTA |
| | | CTGCCTGGAGTACTTTCCTTCTCAGATGCTG |
| | | CGTACCGGAAACAACTTTACCTTCAGCTACA |
| | | CTTTTGAGGACGTTCCTTTCCACAGCAGCTA |
| | | CGCTCACAGCCAGAGTCTGGACCGTCTCATG |
| | | AATCCTCTCATCGACCAGTACCTGTATTACT |
| | | TGAGCAGAACAAACACTCCAAGTGGAACCAC |
| | | CACGCAGTCAAGGCTTCAGTTTTCTCAGGCC |
| | | GGAGCGAGTGACATTCGGGACCAGTCTAGGA |
| | | ACTGGCTTCCTGGACCCTGTTACCGCCAGCA |
| | | GCGAGTATCAAAGACATCTGCGGATAACAAC |
| | | AACAGTGAATACTCGTGGACTGGAGCTACCA |
| | | AGTACCACCTCAATGGCAGAGACTCTCTGGT |
| | | GAATCCGGGCCCGGCCATGGCAAGCCACAAG |
| | | GACGATGAAGAAAAGTTTTTTCCTCAGAGCG |
| | | GGGTTCTCATCTTTGGGAAGCAAGGCTCAGA |
| | | GAAAACAAATGTGGACATTGAAAAGGTCATG |
| | | TTAACAGACGAAGAGGAAATCAGGACAACCA |
| | | ATCCCGTGGCTACGGAGCAGTATGGTTCTGT |
| | | ATCTACCAACCTCCAGAGAGGCACAAACCTA |
| | | GCGCGGGGTGAAACCGCTAGACCAGCGAGAC |
| | | AAGCAGCTACCGCAGATGTCAACACACAAGG |
| | | CGTTCTTCCAGGCATGGTCTGGCAGGACAGA |
| | | GATGTGTACCTTCAGGGGCCCATCTGGGCAA |
| | | AGATTCCACACACGGACGGACATTTTCACCC |
| | | CTCTCCCCTCATGGGTGGATTCGGACTTAAA |
| | | CACCCTCCTCCACAGATTCTCATCAAGAACA |
| | | CCCCGGTACCTGCGAATCCTTCGACCACCTT |
| | | CAGTGCGGCAAAGTTTGCTTCCTTCATCACA |
| | | CAGTACTCCACGGGACAGGTCAGCGTGGAGA |
| | | TCGAGTGGGAGCTGCAGAAGGAAAACAGCAA |
| | | ACGCTGGAATCCCGAAATTCAGTACACTTCC |
| | | AACTACAACAAGTCTGTTAATGTGGACTTTA |
| | | CTGTGGACACTAATGGCGTGTATTCAGAGCC |
| | | TCGCCCCATTGGCACCAGATACCTGACTCGT |
| | | AATCTGTAA (SEQ ID NO: 43) |

In some embodiments, the capsid polypeptide has at least 85, 90, 91, 92, 93, 94, 95, 96, 97, 98, 99%, or 100% identity to a VP1, VP2, or VP3 sequence as provided in Table 2. In some embodiments, the capsid polypeptide has at least 85, 90, 91, 92, 93, 94, 95, 96, 97, 98, 99%, or 100% identity to a VP1, VP2, or VP3 sequence of SEQ ID NO: 12. In some embodiments, the capsid polypeptide has at least 85, 90, 91, 92, 93, 94, 95, 96, 97, 98, 99%, or 100% identity to a VP1, VP2, or VP3 sequence of SEQ ID NO: 13. In some embodiments, the capsid polypeptide has at least 85, 90, 91, 92, 93, 94, 95, 96, 97, 98, 99%, or 100% identity to a VP1, VP2, or VP3 sequence of SEQ ID NO: 14. In some embodiments, the capsid polypeptide has at least 85, 90, 91, 92, 93, 94, 95, 96, 97, 98, 99%, or 100% identity to a VP1, VP2, or VP3 sequence of SEQ ID NO: 15. In some embodiments, the capsid polypeptide has at least 85, 90, 91, 92, 93, 94, 95, 96, 97, 98, 99%, or 100% identity to a VP1, VP2, or VP3 sequence of SEQ ID NO: 16. In some embodiments, the capsid polypeptide has at least 85, 90, 91, 92, 93, 94, 95, 96, 97, 98, 99%, or 100% identity to a VP1, VP2, or VP3 sequence of SEQ ID NO: 17. In some embodiments, the capsid polypeptide has at least 85, 90, 91, 92, 93, 94, 95, 96, 97, 98, 99%, or 100% identity to a VP1, VP2, or VP3 sequence of SEQ ID NO: 18. In some embodiments, the capsid polypeptide has at least 85, 90, 91, 92, 93, 94, 95, 96, 97, 98, 99%, or 100% identity to a VP1, VP2, or VP3 sequence of SEQ ID NO: 19. In some embodiments, the capsid polypeptide has at least 85, 90, 91, 92, 93, 94, 95, 96, 97, 98, 99%, or 100% identity to a VP1, VP2, or VP3 sequence of SEQ ID NO: 20. In some embodiments, the capsid polypeptide has at least 85, 90, 91, 92, 93, 94, 95, 96, 97, 98, 99%, or 100% identity to a VP1, VP2, or VP3 sequence of SEQ ID NO: 21. In some embodiments, the capsid polypeptide has at least 85, 90, 91, 92, 93, 94, 95, 96, 97, 98, 99%, or 100% identity to a VP1, VP2, or VP3 sequence of SEQ ID NO: 22. In some embodiments, the capsid polypeptide has at least 85, 90, 91, 92, 93, 94, 95, 96, 97, 98, 99%, or 100% identity to a VP1, VP2, or VP3 sequence of SEQ ID NO: 23. In some embodiments, the capsid polypeptide has at least 85, 90, 91, 92, 93, 94, 95, 96, 97, 98, 99%, or 100% identity to a VP1, VP2, or VP3 sequence of SEQ ID NO: 24. In some embodiments, the capsid polypeptide has at least 85, 90, 91, 92, 93, 94, 95, 96, 97, 98, 99%, or 100% identity to a VP1, VP2, or VP3 sequence of SEQ ID NO: 25. In some embodiments, the capsid polypeptide has at least 85, 90, 91, 92, 93, 94, 95, 96, 97, 98, 99%, or 100% identity to a VP1, VP2, or VP3 sequence of SEQ ID NO: 26. In some embodiments, the capsid polypeptide has at least 85, 90, 91, 92, 93, 94, 95, 96, 97, 98, 99%, or 100% identity to a VP1, VP2, or VP3 sequence of SEQ ID NO: 27.

In some embodiments, the capsid polypeptide has a sequence of SEQ ID NO: 12-27. In some embodiments, the capsid polypeptide has a sequence of SEQ ID NO: 12. In some embodiments, the capsid polypeptide has a sequence of SEQ ID NO: 13. In some embodiments, the capsid polypeptide has a sequence of SEQ ID NO: 14. In some embodiments, the capsid polypeptide has a sequence of SEQ ID NO: 15. In some embodiments, the capsid polypeptide has a sequence of SEQ ID NO: 16. In some embodiments, the capsid polypeptide has a sequence of SEQ ID NO: 17. In some embodiments, the capsid polypeptide has a sequence of SEQ ID NO: 18. In some embodiments, the capsid polypeptide has a sequence of SEQ ID NO: 19. In some embodiments, the capsid polypeptide has a sequence of SEQ ID NO: 20. In some embodiments, the capsid polypeptide has a sequence of SEQ ID NO: 21. In some embodiments, the capsid polypeptide has a sequence of SEQ ID NO: 22. In some embodiments, the capsid polypeptide has a sequence of SEQ ID NO: 23. In some embodiments, the capsid polypeptide has a sequence of SEQ ID NO: 24. In some embodiments, the capsid polypeptide has a sequence of SEQ ID NO: 25. In some embodiments, the capsid polypeptide has a sequence of SEQ ID NO: 26. In some embodiments, the capsid polypeptide has a sequence of SEQ ID NO: 27.

In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence as provided in Table 2. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 12. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 13. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 14. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 15. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 16. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 17. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 18. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 19. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 20. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 21. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 22. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 23. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 24. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 25. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 26. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 27.

In some embodiments, the capsid polypeptide comprises a reference capsid sequence, such as SEQ ID NO: 1, and at least, or about, 80%, 85%, 90%, or 95%, or 100% of the mutations (insertions, deletions, or substitutions) as shown in the Mutation Differences column of Table 1A, Table 1B, Table 1C, Table 1D, Table 1E, Table 1F, and Table 1G of VAR-1, VAR-2, VAR-3, VAR-4, VAR-5, VAR-6, VAR-7, VAR-8, VAR-9, VAR-10, VAR-11, VAR-12, VAR-13, VAR-14, VAR-15, or VAR-16. In some embodiments, the reference capsid sequence comprises at least, about, or exactly, 80% of the mutations (insertions, deletions, or substitutions). In some embodiments, the reference capsid sequence comprises at least, about, or exactly, 85% of the mutations (insertions, deletions, or substitutions). In some embodiments, the reference capsid sequence comprises at least, about, or exactly, 90% of the mutations (insertions, deletions, or substitutions). In some embodiments, the reference capsid sequence comprises at least, about, or exactly, 95% of the mutations (insertions, deletions, or substitutions). In some embodiments, the reference capsid sequence comprises 100% of the mutations (insertions, deletions, or substitutions).

In some embodiments, the capsid polypeptide comprises a reference capsid sequence, such as SEQ ID NO: 1, and at least, or about, or exactly, 80%, 85%, 90%, or 95%, or 100% of one of the following groups of mutations (the terminology for these groups of mutations is provided for in the legend of Table 1A, Table 1B, Table 1C, Table 1D, Table 1E, Table 1F, and Table 1G above):
[587_10aa_588_LALGEQTRPA];
[587_9aa_588_LAIEQTRPA];
[586_9aa_587_LALAEITRP, N587A];
[586_9aa_587_LKNAETARP, N587A];
[586_10aa_587_LNLAIEQTRP, N587A, A593T];
[586_8aa_587_MLNEQTRP, N587A];
[584_11aa_585_RSGNRADSETA, G586P, N587A];
[586_6aa_587_TGDTRP, N587A];
[587_9aa_588_LQGETIRPA];
[586_11aa_587_QNLANPETTRP, N587A];
[587_10aa_588_RAPQETTRPA, T592A, T597W];
[586_8aa_587_ANLTTTRP, N587A];
[586_10aa_587_ALLAGEQTRP, N587A];
[D561C, 586_9aa_587_GLRAEQTRP, N587A, T597N];
[T550N, 584_8aa_585_RARLDETA, G586P, N587A]; and
[I559L, 586_11aa_587_TNLARGETARP, N587A].

In some embodiments, the capsid polypeptide comprises a reference capsid sequence, such as SEQ ID NO: 1, and at least, or about, or exactly, 80%, 85%, 90%, or 95%, or 100% of [587_10aa_588_LALGEQTRPA]. In some embodiments, the capsid polypeptide comprises at least 8 or all of the amino acid residues of the ten amino acid insertion.

In some embodiments, the capsid polypeptide comprises a reference capsid sequence, such as SEQ ID NO: 1, and at least, or about, or exactly, 80%, 85%, 90%, or 95%, or 100% of [587_9aa_588_LAIEQTRPA]. In some embodiments, the capsid polypeptide comprises at least 7 or all of the amino acid residues of the nine amino acid insertion.

In some embodiments, the capsid polypeptide comprises a reference capsid sequence, such as SEQ ID NO: 1, and at least, or about, or exactly, 80%, 85%, 90%, or 95%, or 100% of [586_9aa_587_LALAEITRP, N587A]. In some embodiments, the capsid polypeptide comprises at least 7 or all of the amino acid residues of the nine amino acid insertion. In some embodiments, the capsid polypeptide comprises at least 8 or all of the amino acid residues of the nine amino acid insertion and the N587A mutation.

In some embodiments, the capsid polypeptide comprises a reference capsid sequence, such as SEQ ID NO: 1, and at least, or about, or exactly, 80%, 85%, 90%, or 95%, or 100% of [586_9aa_587_LKNAETARP, N587A]. In some embodiments, the capsid polypeptide comprises at least 7 or all of the amino acid residues of the nine amino acid insertion. In some embodiments, the capsid polypeptide comprises at least 8 or all of the amino acid residues of the nine amino acid insertion and the N587A mutation.

In some embodiments, the capsid polypeptide comprises a reference capsid sequence, such as SEQ ID NO: 1, and at least, or about, or exactly, 80%, 85%, 90%, or 95%, or 100% of [586_10aa_587_LNLAIEQTRP, N587A, A593T]. In some embodiments, the capsid polypeptide comprises at least 8 or all of the amino acid residues of the ten amino acid insertion. In some embodiments, the capsid polypeptide comprises at least 9 or all of the amino acid residues of the ten amino acid insertion and the N587A and A593T mutation.

In some embodiments, the capsid polypeptide comprises a reference capsid sequence, such as SEQ ID NO: 1, and at least, or about, or exactly, 80%, 85%, 90%, or 95%, or 100% of [586_8aa_587_MLNEQTRP, N587A]. In some embodiments, the capsid polypeptide comprises at least 6 or all of the amino acid residues of the eight amino acid insertion. In some embodiments, the capsid polypeptide comprises at least 7 or all of the amino acid residues of the eight amino acid insertion and the N587A mutation.

In some embodiments, the capsid polypeptide comprises a reference capsid sequence, such as SEQ ID NO: 1, and at least, or about, or exactly, 80%, 85%, 90%, or 95%, or 100% of [584_11aa_585_RSGNRADSETA, G586P, N587A]. In some embodiments, the capsid polypeptide comprises at least 9 or all of the amino acid residues of the eleven amino acid insertion. In some embodiments, the capsid polypeptide comprises at least 10 or all of the amino acid residues of the eleven amino acid insertion and the G586P, and N587A mutation.

In some embodiments, the capsid polypeptide comprises a reference capsid sequence, such as SEQ ID NO: 1, and at least, or about, or exactly, 80%, 85%, 90%, or 95%, or 100% of [586_6aa_587_TGDTRP, N587A]. In some embodiments, the capsid polypeptide comprises at least 5 or all of the amino acid residues of the six amino acid insertion. In some embodiments, the capsid polypeptide comprises at least 6 or all of the amino acid residues of the six amino acid insertion and the N587A mutation.

In some embodiments, the capsid polypeptide comprises a reference capsid sequence, such as SEQ ID NO: 1, and at least, or about, or exactly, 80%, 85%, 90%, or 95%, or 100% of [587_9aa_588_LQGETIRPA]. In some embodiments, the capsid polypeptide comprises at least 7 or all of the amino acid residues of the nine amino acid insertion.

In some embodiments, the capsid polypeptide comprises a reference capsid sequence, such as SEQ ID NO: 1, and at least, or about, or exactly, 80%, 85%, 90%, or 95%, or 100% of [586_11aa_587_QNLANPETTRP, N587A]. In some embodiments, the capsid polypeptide comprises at least 9 or all of the amino acid residues of the eleven amino acid insertion. In some embodiments, the capsid polypeptide comprises at least 10 or all of the amino acid residues of the eleven amino acid insertion and the N587A mutation.

In some embodiments, the capsid polypeptide comprises a reference capsid sequence, such as SEQ ID NO: 1, and at least, or about, or exactly, 80%, 85%, 90%, or 95%, or 100% of [587_10aa_588_RAPQETTRPA, T592A, T597W]. In some embodiments, the capsid polypeptide comprises at least 8 or all of the amino acid residues of the ten amino acid insertion. In some embodiments, the capsid polypeptide comprises at least 9 or all of the amino acid residues of the ten amino acid insertion and the T592A, and T597W mutation.

In some embodiments, the capsid polypeptide comprises a reference capsid sequence, such as SEQ ID NO: 1, and at least, or about, or exactly, 80%, 85%, 90%, or 95%, or 100% of [586_8aa 587 ANLTTTRP, N587A]. In some embodiments, the capsid polypeptide comprises at least 6 or all of the amino acid residues of the eight amino acid insertion. In some embodiments, the capsid polypeptide comprises at least 7 or all of the amino acid residues of the eight amino acid insertion and the N587A mutation.

In some embodiments, the capsid polypeptide comprises a reference capsid sequence, such as SEQ ID NO: 1, and at least, or about, or exactly, 80%, 85%, 90%, or 95%, or 100% of [586_10aa_587_ALLAGEQTRP, N587A]. In some embodiments, the capsid polypeptide comprises at least 8 or all of the amino acid residues of the ten amino acid insertion. In some embodiments, the capsid polypeptide comprises at least 8 or all of the amino acid residues of the ten amino acid insertion and the N587A mutation.

In some embodiments, the capsid polypeptide comprises a reference capsid sequence, such as SEQ ID NO: 1, and at least, or about, or exactly, 80%, 85%, 90%, or 95%, or 100% of [D561C, 586_9aa_587_GLRAEQTRP, N587A, T597N]. In some embodiments, the capsid polypeptide comprises at least 7 or all of the amino acid residues of the nine amino acid insertion. In some embodiments, the capsid polypeptide comprises at least 9 or all of the amino acid residues of the nine amino acid insertion and the D561C, N587A, and T597N mutation.

In some embodiments, the capsid polypeptide comprises a reference capsid sequence, such as SEQ ID NO: 1, and at least, or about, or exactly, 80%, 85%, 90%, or 95%, or 100% of [T550N, 584_8aa_585_RARLDETA, G586P, N587A]. In some embodiments, the capsid polypeptide comprises at least 6 or all of the amino acid residues of the eight amino acid insertion. In some embodiments, the capsid polypeptide comprises at least 8 or all of the amino acid residues of the eight amino acid insertion and the T550N, G586P, and N587A mutation.

In some embodiments, the capsid polypeptide comprises a reference capsid sequence, such as SEQ ID NO: 1, and at least, or about, or exactly, 80%, 85%, 90%, or 95%, or 100% of [I559L, 586_11aa_587_TNLARGETARP, N587A]. In some embodiments, the capsid polypeptide comprises at least 8 or all of the amino acid residues of the eleven amino acid insertion. In some embodiments, the capsid polypeptide comprises at least 10 or all of the amino acid residues of the eleven amino acid insertion and the I559L, and N587A mutation.

In some embodiments, a variant capsid polypeptide is provided that comprises an amino acid sequence that has 95% or more amino acid sequence identity with an amino acid sequence of SEQ ID NO: 12; and has at least 80% of the mutations in SEQ ID NO: 12 as compared to SEQ ID NO: 1. In some embodiments, a variant capsid polypeptide is provided that comprises an amino acid sequence that has less than 95% amino acid sequence identity to an amino acid sequence of SEQ ID NO: 12; and has at least 80% of the mutations in SEQ ID NO: 12 as compared to SEQ ID NO: 1. In some embodiments, a variant capsid polypeptide is provided that comprises an amino acid sequence that has 95% or more amino acid sequence identity with SEQ ID NO: 12; and has less than 80% of the mutations in SEQ ID NO: 12 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, the increase is at least 2-fold, at least 4-fold, or at least 5-fold over the transduction of a virus particle comprising capsid polypeptides of a reference sequence, e.g., SEQ ID NO: 1. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, at least 128-fold, or at least 300-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 13; and has at least 80% of the mutations in SEQ ID NO: 13 as compared to SEQ ID NO: 1. In some embodiments, a variant capsid polypeptide is provided that comprises an amino acid sequence that has less than 95% amino acid sequence identity to an amino acid sequence of SEQ ID NO: 13; and has at least 80% of the mutations in SEQ ID NO: 13 as compared to SEQ ID NO: 1. In some embodiments, a variant capsid polypeptide is provided that comprises an amino acid sequence that has 95% or more amino acid sequence identity with SEQ ID NO: 13; and has less than 80% of the mutations in SEQ ID NO: 13 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, the increase is at least 2-fold or at least 3-fold over the transduction of a virus particle comprising capsid polypeptides of a reference sequence, e.g., SEQ ID NO: 1. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, at least 110-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 14; and has at least 80% of the mutations in SEQ ID NO: 14 as compared to SEQ ID NO: 1. In some embodiments, a variant capsid polypeptide is provided that comprises an amino acid sequence that has less than 95% amino acid sequence identity to an amino acid sequence of SEQ ID NO: 14; and has at least 80% of the mutations in SEQ ID NO: 14 as compared to SEQ ID NO: 1. In some embodiments, a variant capsid polypeptide is provided that comprises an amino acid sequence that has 95% or more amino acid sequence identity with SEQ ID NO: 14; and has less than 80% of the mutations in SEQ ID NO:14 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, the increase is at least 2-fold, at least 4-fold, or at least 8-fold over the transduction of a virus particle comprising capsid polypeptides of a reference sequence, e.g., SEQ ID NO: 1. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 15; and has at least 80% of the mutations in SEQ ID NO: 15 as compared to SEQ ID NO: 1. In some embodiments, a variant capsid polypeptide is provided that comprises an amino acid sequence that has less than 95% amino acid sequence identity to an amino acid sequence of SEQ ID NO: 15; and has at least 80% of the mutations in SEQ ID NO: 15 as compared to SEQ ID NO: 1. In some embodiments, a variant capsid polypeptide is provided that comprises an amino acid sequence that has 95% or more amino acid sequence identity with SEQ ID NO: 15; and has less than 80% of the mutations in SEQ ID NO: 15 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, the increase is at least 2-fold over the transduction of a virus particle comprising capsid polypeptides of a reference sequence, e.g., SEQ ID NO: 1. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increase is at least 60-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 16; and has at least 80% of the mutations in SEQ ID NO: 16 as compared to SEQ ID NO: 1. In some embodiments, a variant capsid polypeptide is provided that comprises an amino acid sequence that has less than 95% amino acid sequence identity to an amino acid sequence of SEQ ID NO: 16; and has at least 80% of the mutations in SEQ ID NO: 16 as compared to SEQ ID NO: 1. In some embodiments, a variant capsid polypeptide is provided that comprises an amino acid sequence that has 95% or more amino acid sequence identity with SEQ ID NO: 16; and has less than 80% of the mutations in SEQ ID NO: 16 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, at least 128-fold, or at least 230-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 17; and has at least 80% of the mutations in SEQ ID NO: 17 as compared to SEQ ID NO: 1. In some embodiments, a variant capsid polypeptide is provided that comprises an amino acid sequence that has less than 95% amino acid sequence identity to an amino acid sequence of SEQ ID NO: 17; and has at least 80% of the mutations in SEQ ID NO: 17 as compared to SEQ ID NO: 1. In some embodiments, a variant capsid polypeptide is provided that comprises an amino acid sequence that has 95% or more amino acid sequence identity with SEQ ID NO: 17; and has less than 80% of the mutations in SEQ ID NO: 17 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, at least 70-fold, at least 128-fold, or at least 520-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 18; and has at least 80% of the mutations in SEQ ID NO: 18 as compared to SEQ ID NO: 1. In some embodiments, a variant capsid polypeptide is provided that comprises an amino acid sequence that has less than 95% amino acid sequence identity to an amino acid sequence of SEQ ID NO: 18; and has at least 80% of the mutations in SEQ ID NO: 18 as compared to SEQ ID NO: 1. In some embodiments, a variant capsid polypeptide is provided that comprises an amino acid sequence that has 95% or more amino acid sequence identity with SEQ ID NO: 18; and has less than 80% of the mutations in SEQ ID NO: 18 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 50-fold, at least 64-fold, at least 128-fold, or at least 460-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 19; and has at least 80% of the mutations in SEQ ID NO: 19 as compared to SEQ ID NO: 1. In some embodiments, a variant capsid polypeptide is provided that comprises an amino acid sequence that has less than 95% amino acid sequence identity to an amino acid sequence of SEQ ID NO: 19; and has at least 80% of the mutations in SEQ ID NO: 19 as compared to SEQ ID NO: 1. In some embodiments, a variant capsid polypeptide is provided that comprises an amino acid sequence that has 95% or more amino acid sequence identity with SEQ ID NO: 19; and has less than 80% of the mutations in SEQ ID NO: 19 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, at least 100-fold, at least 128-fold, or at least 1000-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 20; and has at least 80% of the mutations in SEQ ID NO: 20 as compared to SEQ ID NO: 1. In some embodiments, a variant capsid polypeptide is provided that comprises an amino acid sequence that has less than 95% amino acid sequence identity to an amino acid sequence of SEQ ID NO: 20; and has at least 80% of the mutations in SEQ ID NO: 20 as compared to SEQ ID NO: 1. In some embodiments, a variant capsid polypeptide is provided that comprises an amino acid sequence that has 95% or more amino acid sequence identity with SEQ ID NO: 20; and has less than 80% of the mutations in SEQ ID NO: 20 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 21; and has at least 80% of the mutations in SEQ ID NO: 21 as compared to SEQ ID NO: 1. In some embodiments, a variant capsid polypeptide is provided that comprises an amino acid sequence that has less than 95% amino acid sequence identity to an amino acid sequence of SEQ ID NO: 21; and has at least 80% of the mutations in SEQ ID NO: 21 as compared to SEQ ID NO: 1. In some embodiments, a variant capsid polypeptide is provided that comprises an amino acid sequence that has 95% or more amino acid sequence identity with SEQ ID NO: 21; and has less than 80% of the mutations in SEQ ID NO: 21 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 22; and has at least 80% of the mutations in SEQ ID NO: 22 as compared to SEQ ID NO: 1. In some embodiments, a variant capsid polypeptide is provided that comprises an amino acid sequence that has less than 95% amino acid sequence identity to an amino acid sequence of SEQ ID NO: 22; and has at least 80% of the mutations in SEQ ID NO: 22 as compared to SEQ ID NO: 1. In some embodiments, a variant capsid polypeptide is provided that comprises an amino acid sequence that has 95% or more amino acid sequence identity with SEQ ID NO: 22; and has less than 80% of the mutations in SEQ ID NO: 22 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, the increase is at least 2-fold, at least 4-fold, or at least 5-fold over the transduction of a virus particle comprising capsid polypeptides of a reference sequence, e.g., SEQ ID NO: 1. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 28-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 23; and has at least 80% of the mutations in SEQ ID NO: 23 as compared to SEQ ID NO: 1. In some embodiments, a variant capsid polypeptide is provided that comprises an amino acid sequence that has less than 95% amino acid sequence identity to an amino acid sequence of SEQ ID NO: 23; and has at least 80% of the mutations in SEQ ID NO: 23 as compared to SEQ ID NO: 1. In some embodiments, a variant capsid polypeptide is provided that comprises an amino acid sequence that has 95% or more amino acid sequence identity with SEQ ID NO: 23; and has less than 80% of the mutations in SEQ ID NO: 23 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 24; and has at least 80% of the mutations in SEQ ID NO: 24 as compared to SEQ ID NO: 1. In some embodiments, a variant capsid polypeptide is provided that comprises an amino acid sequence that has less than 95% amino acid sequence identity to an amino acid sequence of SEQ ID NO: 24; and has at least 80% of the mutations in SEQ ID NO: 24 as compared to SEQ ID NO: 1. In some embodiments, a variant capsid polypeptide is provided that comprises an amino acid sequence that has 95% or more amino acid sequence identity with SEQ ID NO: 24; and has less than 80% of the mutations in SEQ ID NO: 24 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, the increase is at least 2-fold, at least 4-fold, or at least 5-fold over the transduction of a virus particle comprising capsid polypeptides of a reference sequence, e.g., SEQ ID NO: 1. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 25; and has at least 80% of the mutations in SEQ ID NO: 25 as compared to SEQ ID NO: 1. In some embodiments, a variant capsid polypeptide is provided that comprises an amino acid sequence that has less than 95% amino acid sequence identity to an amino acid sequence of SEQ ID NO: 25; and has at least 80% of the mutations in SEQ ID NO: 25 as compared to SEQ ID NO: 1. In some embodiments, a variant capsid polypeptide is provided that comprises an amino acid sequence that has 95% or more amino acid sequence identity with SEQ ID NO: 25; and has less than 80% of the mutations in SEQ ID NO: 25 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, the increase is at least 2-fold, at least 4-fold, at least 8-fold, or at least 12-fold over the transduction of a virus particle comprising capsid polypeptides of a reference sequence, e.g., SEQ ID NO: 1. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 26; and has at least 80% of the mutations in SEQ ID NO: 26 as compared to SEQ ID NO: 1. In some embodiments, a variant capsid polypeptide is provided that comprises an amino acid sequence that has less than 95% amino acid sequence identity to an amino acid sequence of SEQ ID NO: 26; and has at least 80% of the mutations in SEQ ID NO: 26 as compared to SEQ ID NO: 1. In some embodiments, a variant capsid polypeptide is provided that comprises an amino acid sequence that has 95% or more amino acid sequence identity with SEQ ID NO: 26; and has less than 80% of the mutations in SEQ ID NO: 26 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

In some embodiments, a variant capsid polypeptide is provided that comprises an amino acid sequence that has 95% or more amino acid sequence identity with an amino acid sequence of SEQ ID NO: 27; and has at least 80% of the mutations in SEQ ID NO: 27 as compared to SEQ ID NO: 1. In some embodiments, a variant capsid polypeptide is provided that comprises an amino acid sequence that has less than 95% amino acid sequence identity to an amino acid sequence of SEQ ID NO: 27; and has at least 80% of the mutations in SEQ ID NO: 27 as compared to SEQ ID NO: 1. In some embodiments, a variant capsid polypeptide is provided that comprises an amino acid sequence that has 95% or more amino acid sequence identity with SEQ ID NO: 27; and has less than 80% of the mutations in SEQ ID NO: 27 as compared to SEQ ID NO: 1. In some embodiments, a virus particle comprising the variant capsid polypeptide has increased ocular transduction of a transgene in the front third of the eye, which includes the structures in front of the vitreous humor, such as the structures described herein, including, but not limited to the cornea, iris, ciliary body, lens, trabecular meshwork, or Schlemm's canal, or any combination thereof. In some embodiments, the increase is at least 2-fold, at least 4-fold, or at least 8-fold over the transduction of a virus particle comprising capsid polypeptides of a reference sequence, e.g., SEQ ID NO: 1. In some embodiments, a virus particle comprising the variant capsid polypeptide has increased ocular transduction of a transgene posterior to the lens, such as in the anterior hyaloid membrane and all of the optical structures behind it, such as the vitreous humor, retina, choroid or optic nerve, or any combination thereof. In some embodiments, the increase is at least 2-fold, at least 4-fold, at least 8-fold, at least 16-fold, at least 32-fold, at least 64-fold, or at least 128-fold over the transduction of a virus particle comprising capsid polypeptides of a reference sequence, e.g., SEQ ID NO: 1. In some embodiments, the increased transduction is measured as described in the Examples, e.g., by NGS sequencing of viral RNA in cells of the target tissue. In some embodiments, the transduction is as measured after intravitreal administration. In some embodiments, the transduction is as measured after intracameral injection.

As used herein, the phrase “80% of the mutations” in reference to a variant capsid sequence means that the variant has at least 80% of the mutations present in the variant capsid sequence, wherein the total number of mutations are based on a comparison to a reference sequence, such as a wild-type sequence. If a variant capsid polypeptide is a mixture of an insertion and substitution or deletion, then each amino acid residue of the insert is counted in the total number of mutations. For example, if the variant capsid polypeptide has a mutation that is a mixture of substitutions of ‘T550N, ‘G586P’, and ‘N587A’, and an insertion of a polypeptide comprising the sequence of RARLDETA (SEQ ID NO: 58) then then the total number of mutations is 11, which is the 8 amino acid insertion and the three amino acid substitutions, and the variant capsid having “80% of the mutations” will comprise at least 9, 10, or all of the mutations.

### Variant Capsids (Corresponding Positions)

The mutations to capsid polypeptide sequences described herein are described in relation to a position and/or amino acid at a position within a reference sequence, e.g., SEQ ID NO: 1. Thus, in some embodiments, the capsid polypeptides described herein are variant capsid polypeptides of the reference sequence, e.g., SEQ ID NO: 1, e.g., include capsid polypeptides comprising at least 80%, at least 85%, at least 90%, at least 91%, at least 92%, at least 93%, at least 94%, at least 95%, at least 96%, at least 97%, at least 98%, or at least 99% identity to the reference capsid polypeptide sequence (e.g., reference capsid polypeptide VP1, VP2 and/or VP3 sequence), e.g., SEQ ID NO: 1 (or VP2 or VP3 sequence comprised therein) and further including one or more mutations described herein.

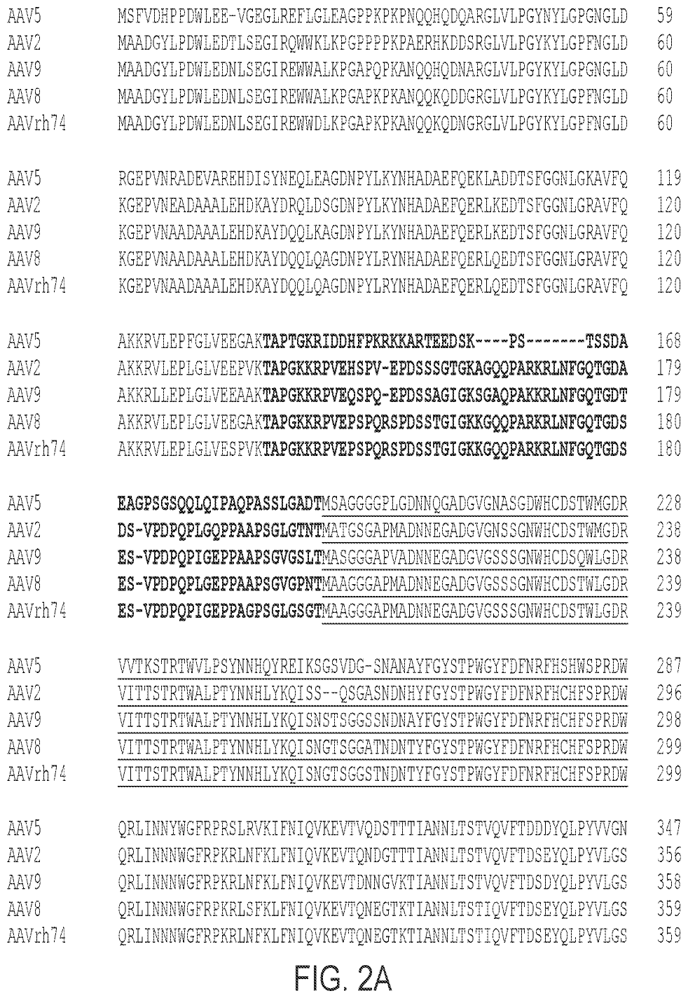

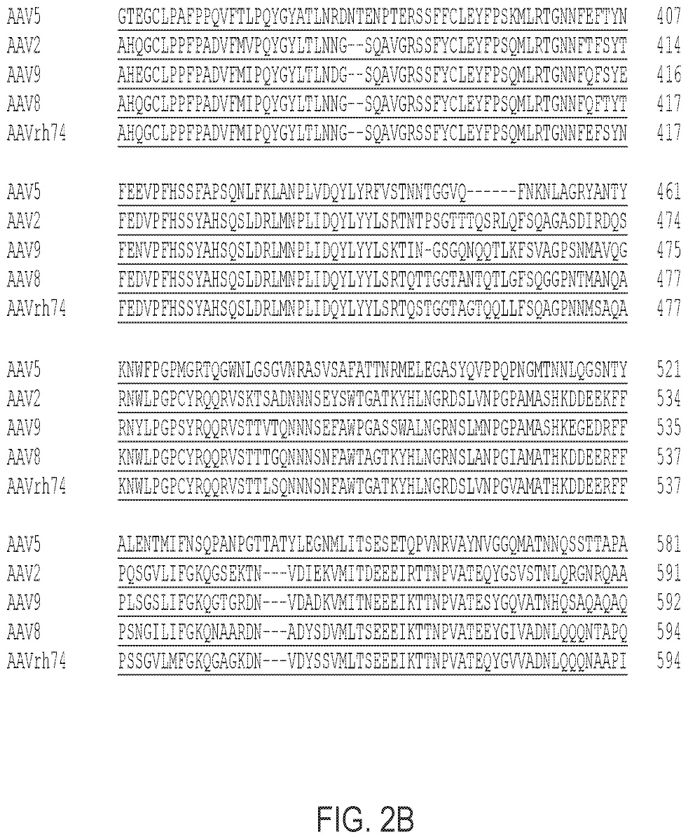

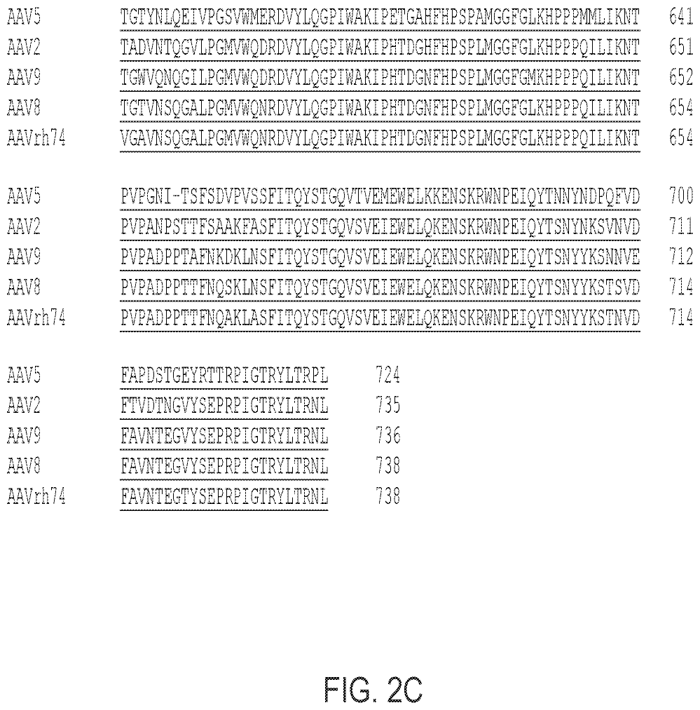

It will be understood by the skilled artisan, and without being bound by theory, that each amino acid position within a reference sequence corresponds to a position within the sequence of other capsid polypeptides such as capsid polypeptides derived from dependoparvoviruses with different serotypes. Such corresponding positions are identified using sequence alignment tools known in the art. A particularly preferred sequence alignment tool is Clustal Omega (Sievers F., et al., Mol. Syst. Biol. 7:359, 2011, DOI: 10.1038/msb.2011.75, which is incorporated herein by reference in its entirety). An alignment of exemplary reference capsid polypeptides is shown in FIGS. 2A-2C. Thus, in some embodiments, the variant capsid polypeptides of the invention include variants of reference capsid polypeptides that include one or more mutations described herein in such reference capsid polypeptides at positions corresponding to the position of the mutation described herein in relation to a different reference capsid polypeptide. Thus, for example, a mutation described as XnnnY relative to SEQ ID NO: 1 (where X is the amino acid present at position nnn in SEQ ID NO: 1 and Y is the amino acid mutation at that position, e.g., described herein), the disclosure provides variant capsid polypeptides comprising at least 80%, at least 85%, at least 90%, at least 91%, at least 92%, at least 93%, at least 94%, at least 95%, at least 96%, at least 97%, at least 98%, or at least 99 identity to the reference capsid polypeptide sequence (e.g., reference capsid polypeptide VP1, VP2 and/or VP3 sequence) other than SEQ ID NO: 1 (or VP2 or VP3 sequence comprised therein) and further comprising the disclosed mutation at a position corresponding to position nnn of SEQ ID NO: 1 (e.g., comprising Y at the position in the new variant capsid polypeptide sequence that corresponds to position nnn of SEQ ID NO: 1). As described above, such corresponding position is determined using a sequence alignment tool, such as, for example, the clustal omega tool described above. Examples of corresponding amino acid positions of exemplary known AAV serotypes is provided in FIGS. 2A-2C. In some embodiments, the variant is a variant of the AAV2 capsid polypeptide, which can be referred to as a “variant AAV2 capsid polypeptide.”

Thus, in some embodiments, the disclosure provides capsid polypeptide sequences that are variants of a reference sequence other than SEQ ID NO: 1, e.g., a reference sequence other than SEQ ID NO: 1 as described herein, which include one or more mutation corresponding to the mutations described herein. In some embodiments, such variants include mutations corresponding to all of the mutations associated with any one of VAR-1 through VAR-16 according to Table 1A, Table 1B, Table 1C, Table 1D, Table 1E, Table 1F, and Table 1G.

As used herein, the term “corresponds to” as used in reference to a position in a sequence, such as an amino acid or nucleic acid sequence, can be used in reference to an entire capsid polypeptide or polynucleotide sequence, such as the full length sequence of the capsid polypeptide that comprises a VP1, VP2, and VP3 polypeptide, or a nucleic acid molecule encoding the same. In some embodiments, the term “corresponds to” can be used in reference to a region or domain of the capsid polypeptide. For example, a position that corresponds to a position in the VP1 section of the reference capsid polypeptide can correspond to the VP1 portion of the polypeptide of the variant capsid polypeptide. Thus, when aligning the two sequences to determine whether a position corresponds to another position the full length polypeptide can be used or domains (regions) can be used to determine whether a position corresponds to a specific position. In some embodiments, the region is the VP1 polypeptide. In some embodiments, the region is the VP2 polypeptide. In some embodiments, the region is the VP3 polypeptide. In some embodiments, when the reference polypeptide is the wild-type sequence (e.g., full length or region) of a certain serotype of AAV, the variant polypeptide can be of the same serotype with a mutation made at such corresponding position as compared to the reference sequence (e.g., full length or region). In some embodiments, the variant capsid polypeptide is a different serotype as compared to the reference sequence.

The variant capsid polypeptides described herein are optionally variants of reference capsids serotypes known in the art. Non-limiting examples of such reference AAV serotypes include AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu 11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof.

In some embodiments, the reference AAV capsid sequence comprises an AAV2 sequence. In some embodiments, the reference AAV capsid sequence comprises an AAV5 sequence. In some embodiments, the reference AAV capsid sequence comprises an AAV8 sequence. In some embodiments, the reference AAV capsid sequence comprises an AAV9 sequence. In some embodiments, the reference AAV capsid sequence comprises an AAVrh74 sequence. While not wishing to be bound by theory, it is understood that a reference AAV capsid sequence comprises a VP1 region. In certain embodiments, a reference AAV capsid sequence comprises a VP1, VP2 and/or VP3 region, or any combination thereof. A reference VP1 sequence may be considered synonymous with a reference AAV capsid sequence.

An exemplary reference sequence of SEQ ID NO: 1 (wild-type AAV2) is as follows:

(SEQ ID NO: 1)
MAADGYLPDWLEDTLSEGIRQWWKLKPGPPPPKPAERHKDDSRGLVLPGYKYLGPFNGLDKGEPVNEADAAALEHDK
AYDRQLDSGDNPYLKYNHADAEFQERLKEDTSFGGNLGRAVFQAKKRVLEPLGLVEEPVKTAPGKKRPVEHSPVEPD
SSSGTGKAGQQPARKRLNFGQTGDADSVPDPQPLGQPPAAPSGLGTNTMATGSGAPMADNNEGADGVGNSSGNWHCD
STWMGDRVITTSTRTWALPTYNNHLYKQISSQSGASNDNHYFGYSTPWGYFDFNRFHCHFSPRDWQRLINNNWGFRP
KRLNFKLFNIQVKEVTQNDGTTTIANNLTSTVQVFTDSEYQLPYVLGSAHQGCLPPFPADVFMVPQYGYLTLNNGSQ
AVGRSSFYCLEYFPSQMLRTGNNFTFSYTFEDVPFHSSYAHSQSLDRLMNPLIDQYLYYLSRTNTPSGTTTQSRLQF
SQAGASDIRDQSRNWLPGPCYRQQRVSKTSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNRQAATADVNTQGVLPGMVWQDRDVYLQGP
IWAKIPHTDGHFHPSPLMGGFGLKHPPPQILIKNTPVPANPSTTFSAAKFASFITQYSTGQVSVEIEWELQKENSKR
WNPEIQYTSNYNKSVNVDFTVDINGVYSEPRPIGTRYLTRNL.

Unless otherwise noted, SEQ ID NO: 1 is the reference sequence. In the sequence above, the sequence found in VP1, VP2 and VP3 is underlined (e.g., a VP3 capsid polypeptide includes, e.g., consists of, amino acids corresponding to amino acids 203-735 of SEQ ID NO: 1), the sequence found in both VP1 and VP2 is in bold (e.g., a VP2 capsid polypeptide includes, e.g., consists of, the sequence corresponding to amino acids 138-735 of SEQ ID NO: 1) and the sequence that is not underlined or bold is found only in VP1 (e.g., a VP1 capsid polypeptide includes, e.g., consists of, amino acids corresponding to amino acids 1-735 of SEQ ID NO: 1).

An example nucleic acid sequence encoding SEQ ID NO: 1 is SEQ ID NO: 2:

(SEQ ID NO: 2)
ATGGCTGCCGATGGTTATCTTCCAGATTGGCTCGAGGACACTCTCTCTGAAGGAATAAGACAGTGGT
GGAAGCTCAAACCTGGCCCACCACCACCAAAGCCCGCAGAGCGGCATAAGGACGACAGCAGGGGT
CTTGTGCTTCCTGGGTACAAGTACCTCGGACCCTTCAACGGACTCGACAAGGGAGAGCCGGTCAAC
GAGGCAGACGCCGCGGCCCTCGAGCACGACAAAGCCTACGACCGGCAGCTCGACAGCGGAGACAA
CCCGTACCTCAAGTACAACCACGCCGACGCGGAGTTTCAGGAGCGCCTTAAAGAAGATACGTCTTTT
GGGGGCAACCTCGGACGAGCAGTCTTCCAGGCGAAAAAGAGGGTTCTTGAACCTCTGGGCCTGGTT
GAGGAACCTGTTAAGACGGCTCCGGGAAAAAAGAGGCCGGTAGAGCACTCTCCTGTGGAGCCAGA
CTCCTCCTCGGGAACCGGAAAGGCGGGCCAGCAGCCTGCAAGAAAAAGATTGAATTTTGGTCAGAC
TGGAGACGCAGACTCAGTACCTGACCCCCAGCCTCTCGGACAGCCACCAGCAGCCCCCTCTGGTCT
GGGAACTAATACGATGGCTACAGGCAGTGGCGCACCAATGGCAGACAATAACGAGGGCGCCGACG
GAGTGGGTAATTCCTCGGGAAATTGGCATTGCGATTCCACATGGATGGGCGACAGAGTCATCACCA
CCAGCACCCGAACCTGGGCCCTGCCCACCTACAACAACCACCTCTACAAACAAATTTCCAGCCAATC
AGGAGCCTCGAACGACAATCACTACTTTGGCTACAGCACCCCTTGGGGGTATTTTGACTTCAACAGA
TTCCACTGCCACTTTTCACCACGTGACTGGCAAAGACTCATCAACAACAACTGGGGATTCCGACCCA
AGAGACTCAACTTCAAGCTCTTTAACATTCAAGTCAAAGAGGTCACGCAGAATGACGGTACGACGAC
GATTGCCAATAACCTTACCAGCACGGTTCAGGTGTTTACTGACTCGGAGTACCAGCTCCCGTACGTC
CTCGGCTCGGCGCATCAAGGATGCCTCCCGCCGTTCCCAGCAGACGTCTTCATGGTGCCACAGTAT
GGATACCTCACCCTGAACAACGGGAGTCAGGCAGTAGGACGCTCTTCATTTTACTGCCTGGAGTACT
TTCCTTCTCAGATGCTGCGTACCGGAAACAACTTTACCTTCAGCTACACTTTTGAGGACGTTCCTTTC
CACAGCAGCTACGCTCACAGCCAGAGTCTGGACCGTCTCATGAATCCTCTCATCGACCAGTACCTGT
ATTACTTGAGCAGAACAAACACTCCAAGTGGAACCACCACGCAGTCAAGGCTTCAGTTTTCTCAGGC
CGGAGCGAGTGACATTCGGGACCAGTCTAGGAACTGGCTTCCTGGACCCTGTTACCGCCAGCAGCG
AGTATCAAAGACATCTGCGGATAACAACAACAGTGAATACTCGTGGACTGGAGCTACCAAGTACCAC
CTCAATGGCAGAGACTCTCTGGTGAATCCGGGCCCGGCCATGGCAAGCCACAAGGACGATGAAGAA
AAGTTTTTTCCTCAGAGCGGGGTTCTCATCTTTGGGAAGCAAGGCTCAGAGAAAACAAATGTGGACA
TTGAAAAGGTCATGATTACAGACGAAGAGGAAATCAGGACAACCAATCCCGTGGCTACGGAGCAGTA
TGGTTCTGTATCTACCAACCTCCAGAGAGGCAACAGACAAGCAGCTACCGCAGATGTCAACACACAA
GGCGTTCTTCCAGGCATGGTCTGGCAGGACAGAGATGTGTACCTTCAGGGGCCCATCTGGGCAAAG
ATTCCACACACGGACGGACATTTTCACCCCTCTCCCCTCATGGGTGGATTCGGACTTAAACACCCTC
CTCCACAGATTCTCATCAAGAACACCCCGGTACCTGCGAATCCTTCGACCACCTTCAGTGCGGCAAA
GTTTGCTTCCTTCATCACACAGTACTCCACGGGACAGGTCAGCGTGGAGATCGAGTGGGAGCTGCA
GAAGGAAAACAGCAAACGCTGGAATCCCGAAATTCAGTACACTTCCAACTACAACAAGTCTGTTAAT
GTGGACTTTACTGTGGACACTAATGGCGTGTATTCAGAGCCTCGCCCCATTGGCACCAGATACCTGA
CTCGTAATCTGTAA

An exemplary reference sequence of wild type AAV5, SEQ ID NO: 3 (wild-type AAV5), is as follows:

(SEQ ID NO: 3)
MSFVDHPPDWLEEVGEGLREFLGLEAGPPKPKPNQQHQDQARGLVLPGYNYLGPGNGLDRGEPVNRADEVAREHDIS
YNEQLEAGDNPYLKYNHADAEFQEKLADDTSFGGNLGKAVFQAKKRVLEPFGLVEEGAKTAPTGKRIDDHFPKRKKA
RTEEDSKPSTSSDAEAGPSGSQQLQIPAQPASSLGADTMSAGGGGPLGDNNQGADGVGNASGDWHCDSTWMGDRVVT
KSTRTWVLPSYNNHQYREIKSGSVDGSNANAYFGYSTPWGYFDFNRFHSHWSPRDWQRLINNYWGFRPRSLRVKIFN
IQVKEVTVQDSTTTIANNLTSTVQVFTDDDYQLPYVVGNGTEGCLPAFPPQVFTLPQYGYATLNRDNTENPTERSSF
FCLEYFPSKMLRTGNNFEFTYNFEEVPFHSSFAPSQNLFKLANPLVDQYLYRFVSTNNTGGVQFNKNLAGRYANTYK
NWFPGPMGRTQGWNLGSGVNRASVSAFATTNRMELEGASYQVPPQPNGMTNNLQGSNTYALFNTMIFNSQPANPGTT
ATYLEGNMLITSESETQPVNRVAYNVGGQMATNNQSSTTAPATGTYNLQEIVPGSVWMERDVYLQGPIWAKIPETGA
HFHPSPAMGGFGLKHPPPMMLIKNTPVPGNITSFSDVPVSSFITQYSTGQVTVEMEWELKKENSKRWNPEIQYTNNY
NDPQFVDFAPDSTGEYRTTRPIGTRYLTRPL

In the sequence above, the sequence found in VP1, VP2 and VP3 is underlined (e.g., a VP3 capsid polypeptide includes, e.g., consists of, amino acids corresponding to amino acids 193-725 of SEQ ID NO: 3), the sequence found in both VP1 and VP2 is in bold (e.g., a VP2 capsid polypeptide includes, e.g., consists of, the sequence corresponding to amino acids 137-725 of SEQ ID NO: 3) and the sequence that is not underlined or bold is found only in VP1 (e.g., a VP1 capsid polypeptide includes, e.g., consists of, amino acids corresponding to amino acids 1-725 of SEQ ID NO: 3).

An example nucleic acid sequence encoding SEQ ID NO: 3 is SEQ ID NO: 4:

(SEQ ID NO: 4)
ATGTCTTTTGTTGATCACCCTCCAGATTGGTTGGAAGAAGTTGGTGAAGGTCTTCGCGAGTTTTTGG
GCCTTGAAGCGGGCCCACCGAAACCAAAACCCAATCAGCAGCATCAAGATCAAGCCCGTGGTCTTG
TGCTGCCTGGTTATAACTATCTCGGACCCGGAAACGGGCTCGATCGAGGAGAGCCTGTCAACAGGG
CAGACGAGGTCGCGCGAGAGCACGACATCTCGTACAACGAGCAGCTTGAGGCGGGAGACAACCCC
TACCTCAAGTACAACCACGCGGACGCCGAGTTTCAGGAGAAGCTCGCCGACGACACATCCTTCGGG
GGAAACCTCGGAAAGGCAGTCTTTCAGGCCAAGAAAAGGGTTCTCGAACCTTTTGGCCTGGTTGAA
GAGGGTGCTAAGACGGCCCCTACCGGAAAGCGGATAGACGACCACTTTCCAAAAAGAAAGAAGGCT
CGGACCGAAGAGGACTCCAAGCCTTCCACCTCGTCAGACGCCGAAGCTGGACCCAGCGGATCCCA
GCAGCTGCAAATCCCAGCCCAACCAGCCTCAAGTTTGGGAGCTGATACAATGTCTGCGGGAGGTGG
CGGCCCATTGGGCGACAATAACCAAGGTGCCGATGGAGTGGGCAATGCCTCGGGAGATTGGCATT
GCGATTCCACGTGGATGGGGGACAGAGTCGTCACCAAGTCCACCCGAACCTGGGTGCTGCCCAGC
TACAACAACCACCAGTACCGAGAGATCAAAAGCGGCTCCGTCGACGGAAGCAACGCCAACGCCTAC
TTTGGATACAGCACCCCCTGGGGGTACTTTGACTTTAACCGCTTCCACAGCCACTGGAGCCCCCGA
GACTGGCAAAGACTCATCAACAACTACTGGGGCTTCAGACCCCGGTCCCTCAGAGTCAAAATCTTCA
ACATTCAAGTCAAAGAGGTCACGGTGCAGGACTCCACCACCACCATCGCCAACAACCTCACCTCCAC
CGTCCAAGTGTTTACGGACGACGACTACCAGCTGCCCTACGTCGTCGGCAACGGGACCGAGGGAT
GCCTGCCGGCCTTCCCTCCGCAGGTCTTTACGCTGCCGCAGTACGGTTACGCGACGCTGAACCGC
GACAACACAGAAAATCCCACCGAGAGGAGCAGCTTCTTCTGCCTAGAGTACTTTCCCAGCAAGATGC
TGAGAACGGGCAACAACTTTGAGTTTACCTACAACTTTGAGGAGGTGCCCTTCCACTCCAGCTTCGC
TCCCAGTCAGAACCTGTTCAAGCTGGCCAACCCGCTGGTGGACCAGTACTTGTACCGCTTCGTGAG
CACAAATAACACTGGCGGAGTCCAGTTCAACAAGAACCTGGCCGGGAGATACGCCAACACCTACAA
AAACTGGTTCCCGGGGCCCATGGGCCGAACCCAGGGCTGGAACCTGGGCTCCGGGGTCAACCGCG
CCAGTGTCAGCGCCTTCGCCACGACCAATAGGATGGAGCTCGAGGGCGCGAGTTACCAGGTGCCC
CCGCAGCCGAACGGCATGACCAACAACCTCCAGGGCAGCAACACCTATGCCCTGGAGAACACTATG
ATCTTCAACAGCCAGCCGGCGAACCCGGGCACCACCGCCACGTACCTCGAGGGCAACATGCTCATC
ACCAGCGAGAGCGAGACGCAGCCGGTGAACCGCGTGGCGTACAACGTCGGCGGGCAGATGGCCA
CCAACAACCAGAGCTCCACCACTGCCCCCGCGACCGGCACGTACAACCTCCAGGAAATCGTGCCCG
GCAGCGTGTGGATGGAGAGGGACGTGTACCTCCAAGGACCCATCTGGGCCAAGATCCCAGAGACG
GGGGCGCACTTTCACCCCTCTCCGGCCATGGGCGGATTCGGACTCAAACACCCACCGCCCATGATG
CTCATCAAGAACACGCCTGTGCCCGGAAATATCACCAGCTTCTCGGACGTGCCCGTCAGCAGCTTC
ATCACCCAGTACAGCACCGGGCAGGTCACCGTGGAGATGGAGTGGGAGCTCAAGAAGGAAAACTC
CAAGAGGTGGAACCCAGAGATCCAGTACACAAACAACTACAACGACCCCCAGTTTGTGGACTTTGCC
CCGGACAGCACCGGGGAATACAGAACCACCAGACCTATCGGAACCCGATACCTTACCCGACCCCTT
TAA

An exemplary reference sequence of wild-type AAV8, SEQ ID NO: 5 (wild-type AAV8), is as follows:

(SEQ ID NO: 5)
MAADGYLPDWLEDNLSEGIREWWALKPGAPKPKANQQKQDDGRGLVLPGYKYLGPFNGLDKGEPVNAADAAALEHDK
AYDQQLQAGDNPYLRYNHADAEFQERLQEDTSFGGNLGRAVFQAKKRVLEPLGLVEEGAKTAPGKKRPVEPSPQRSP
DSSTGIGKKGQQPARKRLNFGQTGDSESVPDPQPLGEPPAAPSGVGPNTMAAGGGAPMADNNEGADGVGSSSGNWHC
DSTWLGDRVITTSTRTWALPTYNNHLYKQISNGTSGGATNDNTYFGYSTPWGYFDFNRFHCHFSPRDWQRLINNNWG
FRPKRLSFKLFNIQVKEVTQNEGTKTIANNLTSTIQVFTDSEYQLPYVLGSAHQGCLPPFPADVFMIPQYGYLTLNN
GSQAVGRSSFYCLEYFPSQMLRTGNNFQFTYTFEDVPFHSSYAHSQSLDRLMNPLIDQYLYYLSRTQTTGGTANTQT
LGFSQGGPNTMANQAKNWLPGPCYRQQRVSTTTGQNNNSNFAWTAGTKYHLNGRNSLANPGIAMATHKDDEERFFPS
NGILIFGKQNAARDNADYSDVMLTSEEEIKTTNPVATEEYGIVADNLQQQNTAPQIGTVNSQGALPGMVWQNRDVYL
QGPIWAKIPHTDGNFHPSPLMGGFGLKHPPPQILIKNTPVPADPPTTFNQSKINSFITQYSTGQVSVEIEWELQKEN
SKRWNPEIQYTSNYYKSTSVDFAVNTEGVYSEPRPIGTRYLTRNL

In the sequence above, the sequence found in VP1, VP2 and VP3 is underlined (e.g., a VP3 capsid polypeptide includes, e.g., consists of, amino acids corresponding to amino acids 204-739 of SEQ ID NO: 5), the sequence found in both VP1 and VP2 is in bold (e.g., a VP2 capsid polypeptide includes, e.g., consists of, the sequence corresponding to amino acids 138-735 of SEQ ID NO: 5) and the sequence that is not underlined or bold is found only in VP1 (e.g., a VP1 capsid polypeptide includes, e.g., consists of, amino acids corresponding to amino acids 1-739 of SEQ ID NO: 5).

An example nucleic acid sequence encoding SEQ ID NO: 5 is SEQ ID NO: 6:

(SEQ ID NO: 6)
ATGGCTGCCGATGGTTATCTTCCAGATTGGCTCGAGGACAACCTCTCTGAGGGCATTCGCGAGTGG
TGGGCGCTGAAACCTGGAGCCCCGAAGCCCAAAGCCAACCAGCAAAAGCAGGACGACGGCCGGGG
TCTGGTGCTTCCTGGCTACAAGTACCTCGGACCCTTCAACGGACTCGACAAGGGGGAGCCCGTCAA
CGCGGCGGACGCAGCGGCCCTCGAGCACGACAAGGCCTACGACCAGCAGCTGCAGGCGGGTGAC
AATCCGTACCTGCGGTATAACCACGCCGACGCCGAGTTTCAGGAGCGTCTGCAAGAAGATACGTCT
TTTGGGGGCAACCTCGGGCGAGCAGTCTTCCAGGCCAAGAAGCGGGTTCTCGAACCTCTCGGTCTG
GTTGAGGAAGGCGCTAAGACGGCTCCTGGAAAGAAGAGACCGGTAGAGCCATCACCCCAGCGTTCT
CCAGACTCCTCTACGGGCATCGGCAAGAAAGGCCAACAGCCCGCCAGAAAAAGACTCAATTTTGGT
CAGACTGGCGACTCAGAGTCAGTTCCAGACCCTCAACCTCTCGGAGAACCTCCAGCAGCGCCCTCT
GGTGTGGGACCTAATACAATGGCTGCAGGCGGTGGCGCACCAATGGCAGACAATAACGAAGGCGC
CGACGGAGTGGGTAGTTCCTCGGGAAATTGGCATTGCGATTCCACATGGCTGGGCGACAGAGTCAT
CACCACCAGCACCCGAACCTGGGCCCTGCCCACCTACAACAACCACCTCTACAAGCAAATCTCCAA
CGGGACATCGGGAGGAGCCACCAACGACAACACCTACTTCGGCTACAGCACCCCCTGGGGGTATTT
TGACTTTAACAGATTCCACTGCCACTTTTCACCACGTGACTGGCAGCGACTCATCAACAACAACTGG
GGATTCCGGCCCAAGAGACTCAGCTTCAAGCTCTTCAACATCCAGGTCAAGGAGGTCACGCAGAAT
GAAGGCACCAAGACCATCGCCAATAACCTCACCAGCACCATCCAGGTGTTTACGGACTCGGAGTAC
CAGCTGCCGTACGTTCTCGGCTCTGCCCACCAGGGCTGCCTGCCTCCGTTCCCGGCGGACGTGTTC
ATGATTCCCCAGTACGGCTACCTAACACTCAACAACGGTAGTCAGGCCGTGGGACGCTCCTCCTTCT
ACTGCCTGGAATACTTTCCTTCGCAGATGCTGAGAACCGGCAACAACTTCCAGTTTACTTACACCTTC
GAGGACGTGCCTTTCCACAGCAGCTACGCCCACAGCCAGAGCTTGGACCGGCTGATGAATCCTCTG
ATTGACCAGTACCTGTACTACTTGTCTCGGACTCAAACAACAGGAGGCACGGCAAATACGCAGACTC
TGGGCTTCAGCCAAGGTGGGCCTAATACAATGGCCAATCAGGCAAAGAACTGGCTGCCAGGACCCT
GTTACCGCCAACAACGCGTCTCAACGACAACCGGGCAAAACAACAATAGCAACTTTGCCTGGACTGC
TGGGACCAAATACCATCTGAATGGAAGAAATTCATTGGCTAATCCTGGCATCGCTATGGCAACACAC
AAAGACGACGAGGAGCGTTTTTTTCCCAGTAACGGGATCCTGATTTTTGGCAAACAAAATGCTGCCA
GAGACAATGCGGATTACAGCGATGTCATGCTCACCAGCGAGGAAGAAATCAAAACCACTAACCCTGT
GGCTACAGAGGAATACGGTATCGTGGCAGATAACTTGCAGCAGCAAAACACGGCTCCTCAAATTGG
AACTGTCAACAGCCAGGGGGCCTTACCCGGTATGGTCTGGCAGAACCGGGACGTGTACCTGCAGG
GTCCCATCTGGGCCAAGATTCCTCACACGGACGGCAACTTCCACCCGTCTCCGCTGATGGGGGGCT
TTGGCCTGAAACATCCTCCGCCTCAGATCCTGATCAAGAACACGCCTGTACCTGCGGATCCTCCGAC
CACCTTCAACCAGTCAAAGCTGAACTCTTTCATCACGCAATACAGCACCGGACAGGTCAGCGTGGAA
ATTGAATGGGAGCTGCAGAAGGAAAACAGCAAGCGCTGGAACCCCGAGATCCAGTACACCTCCAAC
TACTACAAATCTACAAGTGTGGACTTTGCTGTTAATACAGAAGGCGTGTACTCTGAACCCCGCCCCAT
TGGCACCCGTTACCTCACCCGTAATCTGTAA

An exemplary reference sequence of wild-type AAV9, SEQ ID NO: 7 (wild-type AAV9), is as follows:

(SEQ ID NO: 7)
MAADGYLPDWLEDNLSEGIREWWALKPGAPQPKANQQHQDNARGLVLPGYKYLGPGNGLDKGEPVNAADAAALEHDK
AYDQQLKAGDNPYLKYNHADAEFQERLKEDTSFGGNLGRAVFQAKKRLLEPLGLVEEAAKTAPGKKRPVEQSPQEPD
SSAGIGKSGAQPAKKRLNFGQTGDTESVPDPQPIGEPPAAPSGVGSLTMASGGGAPVADNNEGADGVGSSSGNWHCD
SQWLGDRVITTSTRTWALPTYNNHLYKQISNSTSGGSSNDNAYFGYSTPWGYFDFNRFHCHFSPRDWQRLINNNWGF
RPKRLNFKLFNIQVKEVTDNNGVKTIANNLTSTVQVFTDSDYQLPYVLGSAHEGCLPPFPADVFMIPQYGYLTLNDG
SQAVGRSSFYCLEYFPSQMLRTGNNFQFSYEFENVPFHSSYAHSQSLDRLMNPLIDQYLYYLSKTINGSGQNQQTLK
FSVAGPSNMAVQGRNYIPGPSYRQQRVSTTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQAQAQTGWVQNQGILPGMVWQDRDVYLQG
PIWAKIPHTDGNFHPSPLMGGFGMKHPPPQILIKNTPVPADPPTAFNKDKLNSFITQYSTGQVSVEIEWELQKENSK
RWNPEIQYTSNYYKSNNVEFAVNTEGVYSEPRPIGTRYLTRNL

In the sequence above, the sequence found in VP1, VP2 and VP3 is underlined (e.g., a VP3 capsid polypeptide includes, e.g., consists of, amino acids corresponding to amino acids 203-737 of SEQ ID NO: 7), the sequence found in both VP1 and VP2 is in bold (e.g., a VP2 capsid polypeptide includes, e.g., consists of, the sequence corresponding to amino acids 138-737 of SEQ ID NO: 7) and the sequence that is not underlined or bold is found only in VP1 (e.g., a VP1 capsid polypeptide includes, e.g., consists of, amino acids corresponding to amino acids 1-737 of SEQ ID NO: 7).

An example nucleic acid sequence encoding SEQ ID NO: 7 is SEQ ID NO: 8:

(SEQ ID NO: 8)
ATGGCTGCCGATGGTTATCTTCCAGATTGGCTCGAGGACAACCTTAGTGAAGGTATTCGCGAGTGGT
GGGCTTTGAAACCTGGAGCCCCTCAACCCAAGGCAAATCAACAACATCAAGACAACGCTCGAGGTC
TTGTGCTTCCGGGTTACAAATACCTTGGACCCGGCAACGGACTCGACAAGGGGGAGCCGGTCAACG
CAGCAGACGCGGCGGCCCTCGAGCACGACAAGGCCTACGACCAGCAGCTCAAGGCCGGAGACAAC
CCGTACCTCAAGTACAACCACGCCGACGCCGAGTTCCAGGAGCGGCTCAAAGAAGATACGTCTTTT
GGGGGCAACCTCGGGCGAGCAGTCTTCCAGGCCAAAAAGAGGCTTCTTGAACCTCTTGGTCTGGTT
GAGGAAGCGGCTAAGACGGCTCCTGGAAAGAAGAGGCCTGTAGAGCAGTCTCCTCAGGAACCGGA
CTCCTCCGCGGGTATTGGCAAATCGGGTGCACAGCCCGCTAAAAAGAGACTCAATTTCGGTCAGAC
TGGCGACACAGAGTCAGTCCCAGACCCTCAACCAATCGGAGAACCTCCCGCAGCCCCCTCAGGTGT
GGGATCTCTTACAATGGCTTCAGGTGGTGGCGCACCAGTGGCAGACAATAACGAAGGTGCCGATGG
AGTGGGTAGTTCCTCGGGAAATTGGCATTGCGATTCCCAATGGCTGGGGGACAGAGTCATCACCAC
CAGCACCCGAACCTGGGCCCTGCCCACCTACAACAATCACCTCTACAAGCAAATCTCCAACAGCACA
TCTGGAGGATCTTCAAATGACAACGCCTACTTCGGCTACAGCACCCCCTGGGGGTATTTTGACTTCA
ACAGATTCCACTGCCACTTCTCACCACGTGACTGGCAGCGACTCATCAACAACAACTGGGGATTCCG
GCCTAAGCGACTCAACTTCAAGCTCTTCAACATTCAGGTCAAAGAGGTTACGGACAACAATGGAGTC
AAGACCATCGCCAATAACCTTACCAGCACGGTCCAGGTCTTCACGGACTCAGACTATCAGCTCCCGT
ACGTGCTCGGGTCGGCTCACGAGGGCTGCCTCCCGCCGTTCCCAGCGGACGTTTTCATGATTCCTC
AGTACGGGTATCTGACGCTTAATGATGGAAGCCAGGCCGTGGGTCGTTCGTCCTTTTACTGCCTGGA
ATATTTCCCGTCGCAAATGCTAAGAACGGGTAACAACTTCCAGTTCAGCTACGAGTTTGAGAACGTA
CCTTTCCATAGCAGCTACGCTCACAGCCAAAGCCTGGACCGACTAATGAATCCACTCATCGACCAAT
ACTTGTACTATCTCTCAAAGACTATTAACGGTTCTGGACAGAATCAACAAACGCTAAAATTCAGTGTG
GCCGGACCCAGCAACATGGCTGTCCAGGGAAGAAACTACATACCTGGACCCAGCTACCGACAACAA
CGTGTCTCAACCACTGTGACTCAAAACAACAACAGCGAATTTGCTTGGCCTGGAGCTTCTTCTTGGG
CTCTCAATGGACGTAATAGCTTGATGAATCCTGGACCTGCTATGGCCAGCCACAAAGAAGGAGAGGA
CCGTTTCTTTCCTTTGTCTGGATCTTTAATTTTTGGCAAACAAGGAACTGGAAGAGACAACGTGGATG
CGGACAAAGTCATGATAACCAACGAAGAAGAAATTAAAACTACTAACCCGGTAGCAACGGAGTCCTA
TGGACAAGTGGCCACAAACCACCAGAGTGCCCAAGCACAGGCGCAGACCGGCTGGGTTCAAAACC
AAGGAATACTTCCGGGTATGGTTTGGCAGGACAGAGATGTGTACCTGCAAGGACCCATTTGGGCCA
AAATTCCTCACACGGACGGCAACTTTCACCCTTCTCCGCTGATGGGAGGGTTTGGAATGAAGCACCC
GCCTCCTCAGATCCTCATCAAAAACACACCTGTACCTGCGGATCCTCCAACGGCCTTCAACAAGGAC
AAGCTGAACTCTTTCATCACCCAGTATTCTACTGGCCAAGTCAGCGTGGAGATCGAGTGGGAGCTGC
AGAAGGAAAACAGCAAGCGCTGGAACCCGGAGATCCAGTACACTTCCAACTATTACAAGTCTAATAA
TGTTGAATTTGCTGTTAATACTGAAGGTGTATATAGTGAACCCCGCCCCATTGGCACCAGATACCTGA
CTCGTAATCTGTAA

An exemplary reference sequence of wild-type AAVrh74, SEQ ID NO: 9 (wild-type AAVrh74), is as follows:

(SEQ ID NO: 9)
MAADGYLPDWLEDNLSEGIREWWDLKPGAPKPKANQQKQDNGRGLVLPGYKYLGPFNGLDKGEPVNAADAAALEHDK
AYDQQLQAGDNPYLRYNHADAEFQERLQEDTSFGGNLGRAVFQAKKRVLEPLGLVESPVKTAPGKKRPVEPSPQRSP
DSSTGIGKKGQQPAKKRLNFGQTGDSESVPDPQPIGEPPAGPSGLGSGTMAAGGGAPMADNNEGADGVGSSSGNWHC
DSTWLGDRVITTSTRTWALPTYNNHLYKQISNGTSGGSTNDNTYFGYSTPWGYFDFNRFHCHFSPRDWQRLINNNWG
FRPKRLNFKLFNIQVKEVTQNEGTKTIANNLTSTIQVFTDSEYQLPYVLGSAHQGCLPPFPADVFMIPQYGYLTLNN
GSQAVGRSSFYCLEYFPSQMLRTGNNFEFSYNFEDVPFHSSYAHSQSLDRLMNPLIDQYLYYLSRTQSTGGTAGTQQ
LLFSQAGPNNMSAQAKNWLPGPCYRQQRVSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQQNAAPIVGAVNSQGALPGMVWQNRDVYL
QGPIWAKIPHTDGNFHPSPLMGGFGLKHPPPQILIKNTPVPADPPTTFNQAKLASFITQYSTGQVSVEIEWELQKEN
SKRWNPEIQYTSNYYKSTNVDFAVNTEGTYSEPRPIGTRYLTRNL

An alternative exemplary reference sequence of SEQ ID NO: 10 (alternate wild-type AAVrh74 is as follows:

(SEQ ID NO: 10)
MAADGYLPDWLEDNLSEGIREWWDLKPGAPKPKANQQKQDNGRGLVLPGYKYLGPFNGLDKGEPVNAADAAALEHDK
AYDQQLQAGDNPYLRYNHADAEFQERLQEDTSFGGNLGRAVFQAKKRVLEPLGLVESPVKTAPGKKRPVEPSPQRSP
DSSTGIGKKGQQPAKKRLNFGQTGDSESVPDPQPIGEPPAGPSGLGSGTMAAGGGAPMADNNEGADGVGSSSGNWHC
DSTWLGDRVITTSTRTWALPTYNNHLYKQISNGTSGGSTNDNTYFGYSTPWGYFDFNRFHCHFSPRDWQRLINNNWG
FRPKRLNFKLFNIQVKEVTQNEGTKTIANNLTSTIQVFTDSEYQLPYVLGSAHQGCLPPFPADVFMIPQYGYLTLNN
GSQAVGRSSFYCLEYFPSQMLRTGNNFEFSYNFEDVPFHSSYAHSQSLDRLMNPLIDQYLYYLSRTQSTGGTAGTQQ
LLFSQAGPNNMSAQAKNWLPGPCYRQQRVSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQQNAAPIVGAVNSQGALPGMVWQNRDVYL
QGPIWAKIPHTDGNFHPSPLMGGFGLKHPPPQILIKNTPVPADPPTTFTKAKLASFITQYSTGQVSVEIEWELQKEN
SKRWNPEIQYTSNYYKSTNVDFAVNTEGTYSEPRPIGTRYLTRNL

In the sequences above (SEQ ID NO: 9 or SEQ ID NO: 10), the sequence found in VP1, VP2 and VP3 is underlined (e.g., a VP3 capsid polypeptide includes, e.g., consists of, amino acids corresponding to amino acids 204-739 of SEQ ID NO: 9), the sequence found in both VP1 and VP2 is in bold (e.g., a VP2 capsid polypeptide includes, e.g., consists of, the sequence corresponding to amino acids 137-739 of SEQ ID NO: 9) and the sequence that is not underlined or bold is found only in VP1 (e.g., a VP1 capsid polypeptide includes, e.g., consists of, amino acids corresponding to amino acids 1-739 of SEQ ID NO: 9).

An example nucleic acid sequence encoding SEQ ID NO: 9 is SEQ ID NO: 11.

(SEQ ID NO: 11)
ATGGCTGCCGATGGTTATCTTCCAGATTGGCTCGAGGACAACCTCTCTGAGGGCATTCGCGAGTGG
TGGGACCTGAAACCTGGAGCCCCGAAACCCAAAGCCAACCAGCAAAAGCAGGACAACGGCCGGGG
TCTGGTGCTTCCTGGCTACAAGTACCTCGGACCCTTCAACGGACTCGACAAGGGGGAGCCCGTCAA
CGCGGCGGACGCAGCGGCCCTCGAGCACGACAAGGCCTACGACCAGCAGCTCCAAGCGGGTGAC
AATCCGTACCTGCGGTATAATCACGCCGACGCCGAGTTTCAGGAGCGTCTGCAAGAAGATACGTCTT
TTGGGGGCAACCTCGGGCGCGCAGTCTTCCAGGCCAAAAAGCGGGTTCTCGAACCTCTGGGCCTG
GTTGAATCGCCGGTTAAGACGGCTCCTGGAAAGAAGAGGCCGGTAGAGCCATCACCCCAGCGCTCT
CCAGACTCCTCTACGGGCATCGGCAAGAAAGGCCAGCAGCCCGCAAAAAAGAGACTCAATTTTGGG
CAGACTGGCGACTCAGAGTCAGTCCCCGACCCTCAACCAATCGGAGAACCACCAGCAGGCCCCTCT
GGTCTGGGATCTGGTACAATGGCTGCAGGCGGTGGCGCTCCAATGGCAGACAATAACGAAGGCGC
CGACGGAGTGGGTAGTTCCTCAGGAAATTGGCATTGCGATTCCACATGGCTGGGCGACAGAGTCAT
CACCACCAGCACCCGCACCTGGGCCCTGCCCACCTACAACAACCACCTCTACAAGCAAATCTCCAA
CGGGACCTCGGGAGGAAGCACCAACGACAACACCTACTTCGGCTACAGCACCCCCTGGGGGTATTT
TGACTTCAACAGATTCCACTGCCACTTTTCACCACGTGACTGGCAGCGACTCATCAACAACAACTGG
GGATTCCGGCCCAAGAGGCTCAACTTCAAGCTCTTCAACATCCAAGTCAAGGAGGTCACGCAGAAT
GAAGGCACCAAGACCATCGCCAATAACCTTACCAGCACGATTCAGGTCTTTACGGACTCGGAATACC
AGCTCCCGTACGTGCTCGGCTCGGCGCACCAGGGCTGCCTGCCTCCGTTCCCGGCGGACGTCTTC
ATGATTCCTCAGTACGGGTACCTGACTCTGAACAATGGCAGTCAGGCTGTGGGCCGGTCGTCCTTCT
ACTGCCTGGAGTACTTTCCTTCTCAAATGCTGAGAACGGGCAACAACTTTGAATTCAGCTACAACTTC
GAGGACGTGCCCTTCCACAGCAGCTACGCGCACAGCCAGAGCCTGGACCGGCTGATGAACCCTCT
CATCGACCAGTACTTGTACTACCTGTCCCGGACTCAAAGCACGGGCGGTACTGCAGGAACTCAGCA
GTTGCTATTTTCTCAGGCCGGGCCTAACAACATGTCGGCTCAGGCCAAGAACTGGCTACCCGGTCC
CTGCTACCGGCAGCAACGTGTCTCCACGACACTGTCGCAGAACAACAACAGCAACTTTGCCTGGAC
GGGTGCCACCAAGTATCATCTGAATGGCAGAGACTCTCTGGTGAATCCTGGCGTTGCCATGGCTAC
CCACAAGGACGACGAAGAGCGATTTTTTCCATCCAGCGGAGTCTTAATGTTTGGGAAACAGGGAGCT
GGAAAAGACAACGTGGACTATAGCAGCGTGATGCTAACCAGCGAGGAAGAAATAAAGACCACCAAC
CCAGTGGCCACAGAACAGTACGGCGTGGTGGCCGATAACCTGCAACAGCAAAACGCCGCTCCTATT
GTAGGGGCCGTCAATAGTCAAGGAGCCTTACCTGGCATGGTGTGGCAGAACCGGGACGTGTACCTG
CAGGGTCCCATCTGGGCCAAGATTCCTCATACGGACGGCAACTTTCATCCCTCGCCGCTGATGGGA
GGCTTTGGACTGAAGCATCCGCCTCCTCAGATCCTGATTAAAAACACACCTGTTCCCGCGGATCCTC
CGACCACCTTCAATCAGGCCAAGCTGGCTTCTTTCATCACGCAGTACAGTACCGGCCAGGTCAGCG
TGGAGATCGAGTGGGAGCTGCAGAAGGAGAACAGCAAACGCTGGAACCCAGAGATTCAGTACACTT
CCAACTACTACAAATCTACAAATGTGGACTTTGCTGTCAATACTGAGGGTACTTATTCCGAGCCTCGC
CCCATTGGCACCCGTTACCTCACCCGTAATCTGTAA

An exemplary reference sequence of an AAV2 variant identified from the ocular literature, SEQ ID NO: 60, is as follows:

(SEQ ID NO: 60)
MAADGYLPDWLEDTLSEGIRQWWKLKPGPPPPKPAERHKDDSRGLVLPGYKYLGPFNGLDKGEPVNEADAAALEHDK
AYDRQLDSGDNPYLKYNHADAEFQERLKEDTSFGGNLGRAVFQAKKRVLEPLGLVEEPVKTAPGKKRPVEHSPVEPD
SSSGIGKAGQQPARKRLNFGQTGDADSVPDPQPLGQPPAAPSGLGTNTMATGSGAPMADNNEGADGVGNSSGNWHCD
STWMGDRVITTSTRTWALPTYNNHLYKQISSQSGASNDNHYFGYSTPWGYFDFNRFHCHFSPRDWQRLINNNWGFRP
KRLNFKLFNIQVKEVTQNDGITTIANNLTSTVQVETDSEYQLPYVLGSAHQGCLPPFPADVFMVPQYGYLTLNNGSQ
AVGRSSFYCLEYFPSQMLRTGNNFTFSYTFEDVPFHSSYAHSQSLDRLMNPLIDQYLYYLSRTNTPSGTTTQSRLQF
SQAGASDIRDQSRNWLPGPCYRQQRVSKTSADNNNSEYSWTGATKYHLNGRDSLVNPGPAMASHKDDEEKFFPQSGV
LIFGKQGSEKTNVDIEKVMITDEEEIRTINPVATEQYGSVSTNLQRGNLALGETTRPARQAATADVNTQGVLPGMVW
QDRDVYLQGPIWAKIPHTDGHFHPSPLMGGFGLKHPPPQILIKNTPVPANPSTTFSAAKFASFITQYSTGQVSVEIE
WELQKENSKRWNPEIQYTSNYNKSVNVDFTVDINGVYSEPRPIGTRYLTRNL%%

An example nucleic acid sequence encoding SEQ ID NO: 60 is SEQ ID NO: 61.

(SEQ ID NO: 61)
ATGGCTGCCGATGGTTATCTTCCAGATTGGCTCGAGGACACTCTCTCTGAAGGAATAAGACAGTGGTGGAAGCT
CAAACCTGGCCCACCACCACCAAAGCCCGCAGAGCGGCATAAGGACGACAGCAGGGGTCTTGTGCTTCCTGGG
TACAAGTACCTCGGACCCTTCAACGGACTCGACAAGGGAGAGCCGGTCAACGAGGCAGACGCCGCGGCCCTC
GAGCACGACAAAGCCTACGACCGGCAGCTCGACAGCGGAGACAACCCGTACCTCAAGTACAACCACGCCGACG
CGGAGTTTCAGGAGCGCCTTAAAGAAGATACGTCTTTTGGGGGCAACCTCGGACGAGCAGTCTTCCAGGCGAA
AAAGAGGGTTCTTGAACCTCTGGGCCTGGTTGAGGAACCTGTTAAGACGGCTCCGGGAAAAAAGAGGCCGGTA
GAGCACTCTCCTGTGGAGCCAGACTCCTCCTCGGGAACCGGAAAGGCGGGCCAGCAGCCTGCAAGAAAAAGAT
TGAATTTTGGTCAGACTGGAGACGCAGACTCAGTACCTGACCCCCAGCCTOTOGGACAGCCACCAGCAGCCCC
CTCTGGTCTGGGAACTAATACGATGGCTACAGGCAGTGGCGCACCAATGGCAGACAATAACGAGGGCGCCGAC
GGAGTGGGTAATTCCTCGGGAAATTGGCATTGCGATTCCACATGGATGGGCGACAGAGTCATCACCACCAGCAC
CCGAACCTGGGCCCTGCCCACCTACAACAACCACCTCTACAAACAAATTTCCAGCCAATCAGGAGCCTOGAACG
ACAATCACTACTTTGGCTACAGCACCCCTTGGGGGTATTTTGACTTCAACAGATTCCACTGCCACTTTTCACCAC
GTGACTGGCAAAGACTCATCAACAACAACTGGGGATTCCGACCCAAGAGACTCAACTTCAAGCTCTTTAACATTC
AAGTCAAAGAGGTCACGCAGAATGACGGTACGACGACGATTGCCAATAACCTTACCAGCACGGTTCAGGTGTTT
ACTGACTOGGAGTACCAGCTCCCGTACGTCCTCGGCTCGGCGCATCAAGGATGCCTCCCGCCGTTOCCAGCAG
ACGTCTTCATGGTGCCACAGTATGGATACCTCACCCTGAACAACGGGAGTCAGGCAGTAGGACGCTCTTCATTT
TACTGCCTGGAGTACTTTCCTTCTCAGATGCTGCGTACCGGAAACAACTTTACCTTCAGCTACACTTTTGAGGAC
GTTCCTTTCCACAGCAGCTACGCTCACAGCCAGAGTCTGGACCGTCTCATGAATCCTOTCATCGACCAGTACCT
GTATTACTTGAGCAGAACAAACACTCCAAGTGGAACCACCACGCAGTCAAGGCTTCAGTTTTCTCAGGCCGGAG
CGAGTGACATTCGGGACCAGTCTAGGAACTGGCTTCCTGGACCCTGTTACCGCCAGCAGCGAGTATCAAAGACA
TCTGCGGATAACAACAACAGTGAATACTCGTGGACTGGAGCTACCAAGTACCACCTCAATGGCAGAGACTCTOT
GGTGAATCCGGGCCCGGCCATGGCAAGCCACAAGGACGATGAAGAAAAGTTTTTTCCTCAGAGGGGGGTTCTC
ATCTTTGGGAAGCAAGGCTCAGAGAAAACAAATGTGGACATTGAAAAGGTCATGATTACAGACGAAGAGGAAAT
CAGGACAACCAATCCCGTGGCTACGGAGCAGTATGGTTCTGTATCTACCAACCTCCAGAGAGGCAACTTGGCTC
TTGGGGAAACTACGCGTCCTGCAAGACAAGCAGCTACCGCAGATGTCAACACACAAGGCGTTCTACCAGGCATG
GTCTGGCAGGACAGAGATGTGTACCTTCAGGGGCCCATCTGGGCAAAGATTCCACACACGGACGGACATTTTCA
CCCCTCTCCCCTCATGGGTGGATTOGGACTTAAACACCCTOCTOCACAGATTCTCATCAAGAACACCCCGGTAC
CTGCGAATCCTTCGACCACCTTCAGTGCGGCAAAGTTTGCTTCCTTCATCACACAGTACTCCACGGGACAGGTC
AGCGTGGAGATCGAGTGGGAGCTGCAGAAGGAAAACAGCAAACGCTGGAATCCCGAAATTCAGTACACTTCCAA
CTACAACAAGTCTGTTAATGTGGACTTTACTGTGGACACTAATGGCGTGTATTCAGAGCCTCGCCCCATTGGCAC
CAGATACCTGACTCGTAATCTGTAA%%

In some embodiments, described herein are capsid polypeptides, e.g., as described in Table 2, that when included in a virus particle comprising a payload, provide increased delivery of such payload to one or more tissues or cell types of the eye (such as, for example, the neural retina, the macula, and/or the choroid/RPE), e.g., after intravitreal administration, relative to an otherwise identical virus particle comprising the capsid polypeptides of SEQ ID NO: 60. In some embodiments, described herein are capsid polypeptides, e.g., as described in Table 2, that when included in a virus particle comprising a payload, provide increased delivery of such payload to one or more tissues or cell types of the eye (such as, for example, tissues or cells of the trabecular meshwork and Schlemm's canal), e.g., after intravitreal or intracameral administration, relative to an otherwise identical virus particle comprising the capsid polypeptides of SEQ ID NO: 60.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the polypeptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins. Where the Met/AA-clipping is incomplete, a mixture of one or more (one, two or three) VP capsid proteins comprising the viral capsid can be produced, some of which include a Met1/AA1 amino acid (Met+/AA+) and some of which lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods.2017 Oct.28(5):255-267; Hwang, et al. N-Terminal Acetylation of Cellular Proteins Creates Specific Degradation Signals. Science. 2010 February 19.327(5968): 973-977; the contents of which are each incorporated herein by reference in its entirety. According to the present disclosure, references to capsid polypeptides is not limited to either clipped (Met−/AA−) or unclipped (Met+/AA+) and, in context, also refer to independent capsid polypeptides, viral capsids comprised of a mixture of capsid proteins, and/or polynucleotide sequences (or fragments thereof) which encode, describe, produce or result in capsid polypeptides of the present disclosure. A direct reference to a “capsid polypeptide” (such as VP1, VP2 or VP3) also comprise VP capsid proteins which include a Met1/AA1 amino acid (Met+/AA+) as well as corresponding VP capsid polypeptide which lack the Met1/AA1 amino acid e.g. as a result of Met/AA-clipping (Met−/AA−). Further according to the present disclosure, a reference to a specific SEQ ID NO: (whether a protein or nucleic acid) which comprises or encodes, respectively, one or more capsid polypeptides which include a Met1/AA1 amino acid (Met+/AA+) should be understood to teach the VP capsid polypeptides which lack the Met1/AA1 amino acid as upon review of the sequence, it is readily apparent that the first listed amino acid (whether or not Met1/AA1) may be absent. As a non-limiting example, reference to a VP1 polypeptide sequence which is 736 amino acids in length and which includes a “Met1” amino acid (Met+) encoded by the AUG/ATG start codon is also understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon can also be understood to teach a VP1 polypeptide sequence which is 735 amino acids in length and which does not include the “AA1” amino acid (AA1−) of the 736 amino acid AA1+ sequence. References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid e.g. as a result of Met/AA1-clipping (Met−/AA1−), and combinations thereof (Met+/AA1+ and Met−/AA1−). As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In some embodiments, the reference AAV capsid sequence comprises an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%9, 92%, 93%, 94%, 95%, 96%, 97%, 98%, 99%, or 100% identity to any of the those described above.

In some embodiments, the reference AAV capsid sequence is encoded by a nucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described above. In certain embodiments, the reference sequence is not an AAV capsid sequence and is instead a different vector (e.g., lentivirus, plasmid, etc.).

In some embodiments, a nucleic acid of the disclosure (e.g., encoding an AAV2 variant capsid protein) comprises conventional control elements or sequences which are operably linked to the nucleic acid molecule in a manner which permits transcription, translation and/or expression in a cell transfected with the nucleic acid (e.g., a plasmid vector comprising said nucleic acid) or infected with a virus comprising said nucleic acid. As used herein, “operably linked” sequences include both expression control sequences that are contiguous with the gene of interest and expression control sequences that act in trans or at a distance to control the gene of interest.

Expression control sequences include efficient RNA processing signals such as splicing and polyadenylation (polyA) signals; appropriate transcription initiation, termination, promoter and enhancer sequences; sequences that stabilize cytoplasmic mRNA; sequences that enhance protein stability; sequences that enhance translation efficiency (e.g., Kozak consensus sequence); and in some embodiments, sequences that enhance secretion of the encoded transgene product. Expression control sequences, including promoters which are native, constitutive, inducible and/or tissue-specific, are known in the art and may be utilized with the compositions and methods disclosed herein.

In some embodiments, the native promoter for the transgene may be used. Without wishing to be bound by theory, the native promoter may mimic native expression of the transgene, or provide temporal, developmental, or tissue-specific expression, or expression in response to specific transcriptional stimuli. In some embodiment, the transgene may be operably linked to other native expression control elements, such as enhancer elements, polyadenylation sites or Kozak consensus sequences, e.g., to mimic the native expression.

In some embodiments, the transgene is operably linked to a tissue-specific promoter.

In some embodiments, a vector, e.g., a plasmid, carrying a transgene may also include a selectable marker or a reporter gene. Such selectable reporters or marker genes can be used to signal the presence of the vector, e.g., plasmid, in bacterial cells. Other components of the vector, e.g., plasmid, may include an origin of replication. Selection of these and other promoters and vector elements are conventional and many such sequences are available (see, e.g., Sambrook et al, and references cited therein).

In some embodiments, the capsid polypeptide present in a viral particle increases transduction in the eye as compared to a viral particle with a reference capsid polypeptide, for example, with the wild-type capsid polypeptide (SEQ ID NO: 1). In some embodiments, the capsid polypeptide present in a viral particle increases transduction in the retina as compared to a viral particle with a reference capsid polypeptide, for example, with the wild-type capsid polypeptide (SEQ ID NO: 1). In some embodiments, the capsid polypeptide present in a viral particle increases transduction in the non-macular retina as compared to a viral particle with a reference capsid polypeptide, for example, with the wild-type capsid polypeptide (SEQ ID NO: 1). In some embodiments, the capsid polypeptide present in a viral particle increases transduction in the macula as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to retina as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to non-macular retina as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to macula as compared to a viral particle with a reference capsid polypeptide, for example, with the wild-type capsid polypeptide (SEQ ID NO: 1). In some embodiments, the capsid polypeptide present in a viral particle increases transduction in the macula relative to retina as compared to a viral particle with a reference capsid polypeptide, for example, with the wild-type capsid polypeptide (SEQ ID NO: 1). In some embodiments, the capsid polypeptide present in a viral particle increases transduction in the macula relative to non-macular retina as compared to a viral particle with a reference capsid polypeptide, for example, with the wild-type capsid polypeptide (SEQ ID NO: 1). In some embodiments, the capsid polypeptide present in a viral particle increases transduction in the macula relative to trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the macula relative to non-macular retina and trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the macula relative to retina and trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the retina relative to macula and trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the non-macular retina relative to macula and trabecular meshwork as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to macula and retina as compared to a viral particle with a reference capsid polypeptide, for example, with the wild-type capsid polypeptide (SEQ ID NO: 1). In some embodiments, the capsid polypeptide present in a viral particle increases transduction in the trabecular meshwork relative to macula and non-macular retina as compared to a viral particle with a reference capsid polypeptide, for example, with the wild-type capsid polypeptide (SEQ ID NO: 1).

In some embodiments, the capsid polypeptide present in a viral particle increases ocular transduction at least 1-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction at least 2-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 4-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 6-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 8-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 10-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 15-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 16-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 32-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 50-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 70-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 100-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 200-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 300-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 400-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 500-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 1000-fold, e.g., as compared to a viral particle with a reference capsid polypeptide, for example, with the wild-type capsid polypeptide (SEQ ID NO: 1). In some embodiments, increased ocular transduction is measured by comparing the level of mRNA in the target tissue (e.g., in a cell or population of cells of the target tissue) produced from a nucleic acid packaged in the variant viral particle with the level of mRNA in the target tissue (e.g., in a cell or population of cells of the target tissue) produced from a nucleic acid packaged in a reference viral particle (e.g., packaged in a capsid comprising capsid polypeptides of SEQ ID NO: 1).

In some embodiments, the capsid polypeptide present in a viral particle increases transduction in the eye as compared to a viral particle with a reference capsid polypeptide, for example, with a reference capsid polypeptide of SEQ ID NO: 60. In some embodiments, the capsid polypeptide present in a viral particle increases transduction in the trabecular meshwork as compared to a viral particle with a reference capsid polypeptide, for example, a reference capsid polypeptide of SEQ ID NO: 60. In some embodiments, the capsid polypeptide present in a viral particle increases ocular transduction at least 1-fold, e.g., as compared to a viral particle with a reference capsid polypeptide, for example, a reference capsid polypeptide of SEQ ID NO: 60. In some embodiments, the capsid polypeptide present in a viral particle increases ocular transduction at least 1.5-fold, e.g., as compared to a viral particle with a reference capsid polypeptide, for example, a reference capsid polypeptide of SEQ ID NO: 60. In some embodiments, the capsid polypeptide present in a viral particle increases ocular transduction at least 2-fold, e.g., as compared to a viral particle with a reference capsid polypeptide, for example, a reference capsid polypeptide of SEQ ID NO: 60. In some embodiments, the capsid polypeptide present in a viral particle increases ocular transduction at least 2.5-fold, e.g., as compared to a viral particle with a reference capsid polypeptide, for example, a reference capsid polypeptide of SEQ ID NO: 60. In some embodiments, the increased ocular transduction is measured in the trabecular meshwork, e.g., as described in Example 3. In some embodiments, the capsid polypeptide is an isolated or purified polypeptide (e.g., isolated or purified from a cell, other biological component, or contaminant). In some embodiments, the variant polypeptide is present in a dependoparvovirus particle, e.g., described herein. In some embodiments, the variant capsid polypeptide is present in a cell, cell-free system, or translation system, e.g., described herein.

In some embodiments, the cell is a non-human cell. In other embodiments, the cell is not a human pluripotent stem cell, e.g. it is not a human embryonic stem cell.

In some embodiments, the capsid polypeptide is present in a dependoparvovirus B (e.g., AAV2) particle. In some embodiments, the capsid particle has increased ocular transduction.

In some embodiments, a dependoparvovirus particle comprises an amino acid sequence that has at least 80, 85, 90, 91, 92, 93, 94, 95, 96, 97, 98, 99, or 100% identity to the amino acid sequences provided for herein (e.g., SEQ ID NO: 12-27). In some embodiments, the variant capsid polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from the amino acid sequence of a variant capsid polypeptide provided for herein.

In some embodiments, the additional alteration improves a production characteristic of a viral particle, such as but not limited to, a dependoparvovirus particle or method of making the same. In some embodiments, the additional alteration improves or alters another characteristic of a viral particle, such as but not limited to, a dependoparvovirus particle, e.g., tropism.

In embodiments, the improved transduction is as measured by quantification of viral RNA from the target tissue. In some embodiments, the improved biodistribution is as measured by quantification of viral DNA from the target tissue. In some embodiments, the improved transduction is as measured following production from HEK293 cells, for example as described in the Examples.

### VP1 Nucleic Acids and Polypeptides

The disclosure is further directed, in part, to a nucleic acid comprising a sequence encoding a capsid polypeptide, such as but not limited to, a dependoparvovirus (e.g., dependoparvovirus B, e.g., an AAV2) capsid polypeptide as provided for herein, as well as to a VP1 polypeptide encoded by the same. In some embodiments, the polypeptide comprises a sequence of SEQ ID NOs: 12, 13, 14, 15, 16, 17, 18, 19, 20, 21, 22, 23, 24, 25, 26, or 27.

### Viral Particles

The disclosure is also directed, in part, to a viral particle, such as but not limited to, a dependoparvovirus particle (e.g., a functional dependoparvovirus particle) comprising a nucleic acid or polypeptide described herein or produced by a method described herein.

Dependoparvovirus is a single-stranded DNA parvovirus that grows only in cells in which certain functions are provided, e.g., by a co-infecting helper virus. Several species of dependoparvovirus are known, including dependoparvovirus A and dependoparvovirus B, which include serotypes known in the art as adeno-associated viruses (AAV). At least thirteen serotypes of AAV that have been characterized. General information and reviews of AAV can be found in, for example, Carter, Handbook of Parvoviruses, Vol. 1, pp. 169-228 (1989), and Berns, Virology, pp. 1743-1764, Raven Press, (New York, 1990). AAV serotypes, and to a degree, dependoparvovirus species, are significantly interrelated structurally and functionally. (See, for example, Blacklowe, pp. 165-174 of Parvoviruses and Human Disease, J. R. Pattison, ed. (1988); and Rose, Comprehensive Virology 3:1-61 (1974)). For example, all AAV serotypes apparently exhibit very similar replication properties mediated by homologous rep genes; and all bear three related capsid proteins. In addition, heteroduplex analysis reveals extensive cross-hybridization between serotypes along the length of the genome, further suggesting interrelatedness. Dependoparvoviruses genomes also comprise self-annealing segments at the termini that correspond to “inverted terminal repeat sequences” (ITRs).

The genomic organization of naturally occurring dependoparvoviruses, e.g., AAV serotypes, is very similar. For example, the genome of AAV is a linear, single-stranded DNA molecule that is approximately 5,000 nucleotides (nt) in length or less. Inverted terminal repeats (ITRs) flank the unique coding nucleotide sequences for the non-structural replication (Rep) proteins and the structural capsid (Cap) proteins. Three different viral particle (VP) proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins: Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In some embodiments, a dependoparvovirus particle of the disclosure comprises a nucleic acid comprising a capsid polypeptide provided for herein. In some embodiments, the particle comprises a polypeptide as provided for herein.

In some embodiments, the dependoparvovirus particle of the disclosure may be an AAV2 particle or a variant thereof. In some embodiments, the AAV2 particle comprises a capsid polypeptide as provided for herein or a nucleic acid molecule encoding the same.

In some embodiments the dependoparvovirus particle comprises a capsid comprising a variant capsid polypeptide described herein. In some embodiments, the dependoparvovirus particle comprises variant capsid polypeptide described herein and a nucleic acid molecule. In some embodiments, the dependoparvovirus particle comprises variant capsid polypeptide described herein and a nucleic acid molecule comprising one or more inverted terminal repeat sequences (ITRs), for example, ITRs derived from an AAV2 dependoparvovirus, one or more regulatory elements (for example, a promoter), and a payload (e.g., as described herein). In some embodiments, at least one of the ITRs is modified. In some embodiments, the nucleic acid molecule is single-stranded. In some embodiments, the nucleic acid molecule is self-complementary.

In some embodiments, the viral particle comprises a variant capsid polypeptide such as those provided herein. In some embodiments, the viral particle comprises a variant capsid polypeptide having at least 85, 90, 91, 92, 93, 94, 95, 96, 97, 98, 99%, or 100% identity to a VP1, VP2, or VP3 sequence as provided in Table 2. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 12. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 13. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 14. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 15. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 16. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 17. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 18. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 19. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 20. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 21. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 22. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 23. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 24. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 25. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 26. In some embodiments, the viral particle comprises a variant capsid polypeptide having at least 85, 90, 91, 92, 93, 94, 95, 96, 97, 98, 99%, or 100% identity to a VP1, VP2, or VP3 sequence of SEQ ID NO: 27.

In some embodiments, the viral particle comprises a variant capsid polypeptide having a sequence of SEQ ID NO: 12. In some embodiments, the viral particle comprises a variant capsid polypeptide having a sequence of SEQ ID NO: 13. In some embodiments, the viral particle comprises a variant capsid polypeptide having a sequence of SEQ ID NO: 14. In some embodiments, the viral particle comprises a variant capsid polypeptide having a sequence of SEQ ID NO: 15. In some embodiments, the viral particle comprises a variant capsid polypeptide having a sequence of SEQ ID NO: 16. In some embodiments, the viral particle comprises a variant capsid polypeptide having a sequence of SEQ ID NO: 17. In some embodiments, the viral particle comprises a variant capsid polypeptide having a sequence of SEQ ID NO: 18. In some embodiments, the viral particle comprises a variant capsid polypeptide having a sequence of SEQ ID NO: 19. In some embodiments, the viral particle comprises a variant capsid polypeptide having a sequence of SEQ ID NO: 20. In some embodiments, the viral particle comprises a variant capsid polypeptide having a sequence of SEQ ID NO: 21. In some embodiments, the viral particle comprises a variant capsid polypeptide having a sequence of SEQ ID NO: 22. In some embodiments, the viral particle comprises a variant capsid polypeptide having a sequence of SEQ ID NO: 23. In some embodiments, the viral particle comprises a variant capsid polypeptide having a sequence of SEQ ID NO: 24. In some embodiments, the viral particle comprises a variant capsid polypeptide having a sequence of SEQ ID NO: 25. In some embodiments, the viral particle comprises a variant capsid polypeptide having a sequence of SEQ ID NO: 26. In some embodiments, the viral particle comprises a variant capsid polypeptide having a sequence of SEQ ID NO: 27.

### Increased Ocular Transduction Characteristics

The disclosure is directed, in part, to nucleic acids, polypeptides, cells, cell free systems, translation systems, viral particles, and methods associated with making the same to produce virus particles that have increased ocular transduction as compared to a virus particle having capsid polypeptides of a reference sequence, e.g., with a wild-type sequence of SEQ ID NO: 1. In some embodiments, a use of a viral particle comprising the variant capsid polypeptide leads to increased ocular transduction of a transgene in the eye, and, therefore, expression of the transgene in the eye. In some embodiments, a use of a viral particle comprising the variant capsid polypeptide leads to increased ocular transduction of a transgene in the retina, and, therefore, expression of the transgene in the retina. In some embodiments, a use of a viral particle comprising the variant capsid polypeptide leads to increased ocular transduction of a transgene in the non-macular retina, and, therefore, expression of the transgene in the non-macular retina. In some embodiments, a use of a viral particle comprising the variant capsid polypeptide leads to increased ocular transduction of a transgene in the macula, and, therefore, expression of the transgene in the macula. In some embodiments, a use of a viral particle comprising the variant capsid polypeptide leads to increased ocular transduction of a transgene in the trabecular meshwork, and, therefore, expression of the transgene in the trabecular meshwork. In some embodiments, a use of a viral particle comprising the variant capsid polypeptide leads to increased ocular transduction of a transgene in the front third of the eye, which includes the structures in front of the vitreous humor. Examples of structures in front of the vitreous humor, include the cornea, iris, ciliary body, lens, trabecular meshwork, and Schlemm's canal. Accordingly, in some embodiments, use of a viral particle comprising the variant capsid polypeptide leads to increased ocular transduction of a transgene in the cornea, iris, ciliary body, lens, trabecular meshwork, or Schlemm's canal, or any combination thereof. In some embodiments, a use of a viral particle comprising the variant capsid polypeptide leads to increased ocular transduction of a transgene posterior to the lens, such as in the anterior hyaloid membrane and all of the optical structures behind it, such as the vitreous humor, retina, choroid or optic nerve, or any combination thereof. Accordingly, in some embodiments, use of a viral particle comprising the variant capsid polypeptide leads to increased ocular transduction of a transgene in the anterior hyaloid membrane and all of the optical structures behind it, such as the vitreous humor, retina, choroid or optic nerve, or any combination thereof. In some embodiments, a use of a viral particle comprising the variant capsid polypeptide leads to increased ocular transduction of a transgene in the front third of the eye and posterior to the lens.

In some embodiments, the increase in ocular transduction is, on a log 2 scale, about 1-7 times better (e.g., about 2-5 times better, e.g., about 3-5 times better) than a virus particle having a reference sequence capsid polypeptide, e.g., having the wild-type capsid polypeptide SEQ ID NO: 1.

In some embodiments, the capsid polypeptide present in a viral particle increases transduction without increasing the biodistribution of the variant capsid polypeptide in the eye relative to SEQ ID NO: 1. In some embodiments, the capsid polypeptide present in a viral particle increases transduction without increasing the biodistribution of the variant capsid polypeptide in the retina relative to SEQ ID NO: 1. In some embodiments, the capsid polypeptide present in a viral particle increases transduction without increasing the biodistribution of the variant capsid polypeptide in the trabecular meshwork relative to SEQ ID NO: 1.

Tables 3, 4A, 4B, 4C, and 4D list information regarding biodistribution of variant dependoparvovirus particles comprising capsid polypeptides of the indicated variant capsid in the different layers, structures, and/or parts of the eye. In Table 3, biodistribution in retina is as measured following IVT injection and biodistribution in trabecular meshwork is as measured following IC injection, in all cases in the Library Experiment 1. In Table 4A, biodistribution in retina is as measured following IVT injection, biodistribution in trabecular meshwork is as measured following IC or IVT injection (as indicated in the table), and biodistribution in the choroid is as measured following IVT injection, in all cases in the Library Experiment 2. In Table 4B, biodistribution in neural retina tissue is as measured following IVT injection, in all cases in the Library Experiment 3. In Table 4C, biodistribution in neural retina tissue, biodistribution in macula tissue, and biodistribution in trabecular meshwork is as measured following IVT injection, in all cases in the Library Experiment 3. In Table 4D, biodistribution in trabecular meshwork is as measured following IC injection, in all cases in the Library Experiment 3. Unless otherwise indicated, measurements are made relative to wild-type AAV2 (SEQ ID NO: 1). Trabecular biodistribution measurements shown in Tables 4C and 4D are shown relative to an AAV2 variant identified from the ocular literature having a capsid polypeptide SEQ ID NO: 60 and encoded by nucleic acid SEQ ID NO: 61. Data shown in Tables 3 and 4A is on a log 2 scale.

TABLE 3

| | SEQ ID | Biodistribution in | |
|---|---|---|---|
| | NO: of | Nonmacular Retina | Biodistribution in |
| | VP1 | as compared to | Trabecular Meshwork |
| | capsid | wild-type | as compared to wild- |
| | poly- | SEQ ID NO: 1 | type SEQ ID NO: 1 |
| Name | peptide | (Log2) | (Log2) |
| VAR-1 | 12 | 2.62 | 0.94 |
| VAR-2 | 13 | 1.70 | 0.19 |
| VAR-3 | 14 | 1.62 | 1.05 |

TABLE 4A

| | | Biodistribution | Biodistribution | Biodistribution | |
|---|---|---|---|---|---|
| | | in Retina | in Trabecular | in Trabecular | Biodistribution |
| | SEQ ID | (intravitreal | Meshwork | Meshwork | in Choroid as |
| | NO: of | administration) | (intracameral | as compared to | compared to |
| | VP1 | as compared to | administration) | (intravitreal | wild-type |
| | capsid | wild-type SEQ | as compared to | administration) | SEQ ID |
| | poly- | ID NO: 1 | wild-type SEQ | wild-type SEQ | NO: 1 |
| Name | peptide | (Log2) | ID NO: 1 (Log2) | ID NO: 1 (Log2) | (Log2) |
| VAR-1 | 12 | 1.32 | −0.27 | 0.58 | 1.13 |
| VAR-2 | 13 | −1.02 | −0.06 | −0.41 | −0.89 |
| VAR-3 | 14 | −1.13 | 0.18 | −0.90 | −0.84 |
| VAR-4 | 15 | 2.32 | −2.21 | 0.46 | 1.87 |
| VAR-5 | 16 | 1.17 | 3.92 | −0.02 | 0.96 |
| VAR-6 | 17 | 0.38 | −0.38 | 0.13 | 0.45 |
| VAR-7 | 18 | 1.61 | −1.42 | 0.13 | 1.27 |
| VAR-8 | 19 | 2.40 | 0.49 | 0.67 | 1.99 |
| VAR-9 | 20 | 1.71 | 0.82 | 0.37 | 1.38 |
| VAR-10 | 21 | 1.32 | 1.74 | 0.28 | 1.00 |
| VAR-11 | 22 | 2.55 | Not Measured | 0.49 | 1.84 |
| VAR-12 | 23 | 2.27 | −0.99 | 0.31 | 1.65 |
| VAR-13 | 24 | 1.43 | −0.52 | 0.86 | 1.12 |
| VAR-14 | 25 | 2.47 | 0.28 | 0.50 | 2.02 |
| VAR-15 | 26 | 1.71 | 0.82 | 0.37 | 1.38 |
| VAR-16 | 27 | 2.16 | Not Measured | 0.55 | 1.72 |

TABLE 4B

| | | Neural Retina Biodistribution | |
|---|---|---|---|
| | | (intravitreal administration) as | |
| | | compared to wild-type SEQ ID NO: 1 | |
| | SEQ ID NO: | | Standard |
| | of VP1 | | Error of |
| | capsid | | the Mean |
| Name | polypeptide | Mean | (SEM) |
| VAR-1 | 12 | 21.57 | 1.49 |
| VAR-2 | 13 | 4.59 | 1.27 |
| VAR-4 | 15 | 8.08 | 2.22 |
| VAR-5 | 16 | 7.53 | 1.66 |
| VAR-6 | 17 | 18.02 | 1.4 |
| VAR-7 | 18 | 17.82 | 1.65 |
| VAR-8 | 19 | 26.2 | 1.47 |
| VAR-11 | 22 | 3.95 | 1.58 |

TABLE 4C

| | | | | | | Trabecular | |
|---|---|---|---|---|---|---|---|
| | | Neural Retina | | Macular | | Biodistribution | |
| | | Biodistribution | | Biodistribution | | (intravitreal | |
| | | (intravitreal | | (intravitreal | | administration) as | |
| | | administration) | | administration) | | compared to virus | |
| | | as compared to | | as compared to | | particle with capsid | |
| | | wild-type SEQ | | wild-type SEQ | | polypeptides of | |
| | SEQ ID | ID NO: 1 | | ID NO: 1 | | SEQ ID NO: 60 | |
| | NO: of | | Standard | | Standard | | Standard |
| | VP1 | | Error of | | Error of | | Error of |
| | capsid | | the Mean | | the Mean | | the Mean |
| Name | polypeptide | Mean | (SEM) | Mean | (SEM) | Mean | (SEM) |
| VAR-1 | 12 | 13.48 | 1.51 | 30.9 | 1.95 | 2.34 | 1.01 |
| VAR-2 | 13 | 2.08 | 1.6 | 3.93 | 2.22 | 3.07 | 1.03 |
| VAR-4 | 15 | 7.64 | 1.5 | 180.51 | 2.11 | 0.22 | 1.02 |
| VAR-5 | 16 | 5.28 | 1.51 | 14.62 | 2.28 | 0.71 | 1.02 |
| VAR-6 | 17 | 9.76 | 1.54 | 19.4 | 1.93 | 2.14 | 1.02 |
| VAR-7 | 18 | 11.14 | 1.43 | 115.23 | 1.93 | 0.38 | 1.01 |
| VAR-8 | 19 | 20.62 | 1.42 | 83.85 | 1.56 | 1.56 | 1.02 |
| VAR-11 | 22 | 3.76 | 1.31 | 62.71 | 1.66 | 0.24 | 1.03 |

TABLE 4D

| | | Trabecular Biodistribution | |
|---|---|---|---|
| | | (intracameral administration) as | |
| | | compared to virus particle with capsid | |
| | | polypeptides of SEQ ID NO: 60 | |
| | SEQ ID NO: | | Standard |
| | of VP1 | | Error of |
| | capsid | | the Mean |
| Name | polypeptide | Mean | (SEM) |
| VAR-2 | 13 | 0.28 | 1.02 |
| VAR-3 | 14 | 0.26 | 1.02 |
| VAR-13 | 24 | 0.63 | 1.01 |
| VAR-14 | 25 | 0.14 | 1.04 |

According to some embodiments, variant capsid polypeptides described herein comprise a N587 substitution mutation in combination with a N-terminally juxtaposed insertion peptide (e.g., an insertion peptide originating after glycine (G) at position 586 (according to WT AAV2 VP1 numbering; SEQ ID NO: 1)). In some embodiments, the peptide insertion is 6 or fewer amino acids. In some embodiments, the peptide insertion consists of 6 amino acids. In some embodiments, the peptide insertion consists of 7 amino acids. In some embodiments, the peptide insertion is 7 or fewer amino acids. In some embodiments, the peptide insertion is 7 or more amino acids, e.g., 7, 8, 9, 10, or 11 amino acids. In some embodiments, the peptide insertion is between 6 and 11 amino acids. In some embodiments, the peptide insertion consists of 11 amino acids. In some embodiments, the insertion peptide comprises a threonine-arginine-proline (“TRP”) sequence at its C-terminal end (e.g., as in VAR-3, VAR-5, VAR-6, VAR-8, VAR-11, VAR-13, and VAR-14). In some embodiments, a viral particle comprising the variant capsid polypeptide comprising the N587 substitution mutation (e.g., a N587A mutation) in combination with the N-terminally juxtaposed insertion peptide exhibits increased ocular transduction and/or biodistribution relative to a virus particle comprising a variant capsid polypeptide having a reference sequence, e.g., having a wild-type capsid protein, e.g., having capsid polypeptides of SEQ ID NO:1.

According to some embodiments, variant capsid polypeptides described herein comprise a threonine-arginine-proline-alanine (“TRPA”) sequence. In some embodiments, the variant capsid polypeptide comprises a peptide insertion (relative to SEQ ID NO: 1) comprising at least some of the TRPA sequence (e.g., 3 or 4 amino acids forming the TRPA sequence). For example, VAR-1, VAR-2, VAR-3, VAR-5, VAR-6, VAR-8, VAR-11, VAR-13, and VAR-14 each comprise a TRPA sequence. In some embodiments, the insertion site is after glycine (G) at position 586 (according to WT AAV2 VP1 numbering; SEQ ID NO: 1). In some embodiments, the insertion site is after asparagine (N) at position 587 (according to WT AAV2 VP1 numbering; SEQ ID NO: 1). In some embodiments, the TRPA sequence is formed by a peptide insertion comprising a C-terminal TRPA sequence. For example, VAR-1 and VAR-2 both comprise a peptide insertion comprising a TRPA sequence at its C-terminal, optionally wherein the peptide insertion originates after asparagine (N) at position 587 (according to WT AAV2 VP1 numbering; SEQ ID NO: 1). In some embodiments, the TRPA sequence is formed by a peptide insertion comprising a threonine-arginine-proline (“TRP”) sequence at its C-terminal end followed by an alanine (A) substitution (e.g., N587A substitution). In such embodiments, the alanine (A) substitution mutation in combination with a N-terminally juxtaposed insertion peptide comprising a TRP sequence forms the “TRPA” sequence (e.g., as in VAR-3, VAR-5, VAR-6, VAR-8, VAR-11, VAR-13, and VAR-14). In some embodiments, the peptide insertion is after glycine (G) at position 586 and the alanine substitution is at position 587 (according to WT AAV2 VP1 numbering; SEQ ID NO: 1). In some embodiments, the peptide insertion is 6 or fewer amino acids. In some embodiments, the peptide insertion consists of 6 amino acids. In some embodiments, the peptide insertion is 7 or fewer amino acids. In some embodiments, the peptide insertion consists of 7 amino acids. In some embodiments, the peptide insertion is 7 or more amino acids, e.g., 7, 8, 9, 10, or 11 amino acids. In some embodiments, the peptide insertion is between 6 and 11 amino acids. In some embodiments, the peptide insertion consists of 7 amino acids. In some embodiments, a viral particle comprising the variant capsid polypeptide comprising the TRPA sequence exhibits increased ocular transduction and/or biodistribution relative to a virus particle comprising a variant capsid polypeptide having a reference sequence, e.g., having a wild-type capsid protein, e.g., having capsid polypeptides of SEQ ID NO:1.

According to some embodiments, variant capsid polypeptides described herein comprise a threonine-alanine-arginine-proline-alanine (“TARPA”) sequence. In some embodiments, the variant capsid polypeptide comprises a peptide insertion comprising at least some of the TARPA sequence (e.g., 2, 3 or 4 amino acids forming the TARPA sequence). In some embodiments, the variant capsid polypeptide comprises a peptide insertion (e.g., relative to SEQ ID NO: 1) comprising all of the TARPA sequence. In some embodiments, the insertion site is after glycine (G) at position 586 (according to WT AAV2 VP1 numbering; SEQ ID NO: 1). In some embodiments, the insertion site is after glutamine (Q) at position 584 (according to WT AAV2 VP1 numbering; SEQ ID NO: 1). In some embodiments, the TARPA sequence is formed as a combination of the peptide insertion and a substitution mutation (e.g., a G586P mutation, a N587A mutation) following the peptide insertion, e.g., a peptide insertion comprising TAR at its C-terminal end (according to WT AAV2 VP1 numbering; SEQ ID NO: 1). In some embodiments, the TARPA sequence is formed as a combination of the peptide insertion, a substitution mutation (e.g., a G586P mutation, a N587A mutation) following the peptide insertion (according to WT AAV2 VP1 numbering; SEQ ID NO: 1) and an amino acid occurring in WT AAV2 VP1 (SEQ ID NO: 1), e.g., arginine (R) at position 585. In some embodiments, the variant capsid polypeptide comprises a peptide insertion comprising a threonine-alanine-arginine-proline (“TARP”) sequence at its C-terminal end and inserted after glycine (G) at position 586 followed by an alanine substitution is at position 587 (according to WT AAV2 VP1 numbering; SEQ ID NO: 1), e.g., as in VAR-4 and VAR-16. In some embodiments, the variant capsid polypeptide comprises a peptide insertion comprising a threonine-alanine (“TA”) sequence at its C-terminal end and inserted after glutamine (Q) at position 584 followed by a proline (P) substitution at position 586 and an alanine (A) substitution at position 587 (according to WT AAV2 VP1 numbering; SEQ ID NO: 1), e.g., as in VAR-7, where the arginine (R) at 585 forms the TARPA sequence. In some embodiments, the peptide insertion is 9 or fewer amino acids. In some embodiments, the peptide insertion is 9 or more amino acids, e.g., 10, or 11 amino acids. In some embodiments, the peptide insertion is between 9 and 11 amino acids. In some embodiments, the peptide insertion consists of 9 amino acids. In some embodiments, the peptide insertion consists of 11 amino acids. In some embodiments, a viral particle comprising the variant capsid polypeptide comprising the TARPA sequence exhibits increased ocular transduction and/or biodistribution relative to a virus particle comprising a variant capsid polypeptide having a reference sequence, e.g., having a wild-type capsid protein, e.g., having capsid polypeptides of SEQ ID NO:1.

Category A (Neural Retina Transduction): In some aspects of the disclosure, a viral particle comprising the variant capsid polypeptide, e.g., the variant capsid polypeptide described herein, exhibits increased neural retina transduction as compared to a viral particle with the wild-type capsid polypeptide (SEQ ID NO: 1). In some embodiments, the increased neural retina transduction is as defined as any one of embodiments A-1 through A-8.

Embodiment A-1: In an embodiment of Category A, the transduction is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment A-2: In an embodiment of Category A, the transduction is about (or at least about) 4 times better relative to a virus particle comprising a variant capsid polypeptide having a reference sequence, e.g., having the wild-type capsid protein, e.g., having capsid polypeptides of SEQ ID NO:1.

Embodiment A-3: In an embodiment of Category A, the transduction is about (or at least about) 6 times better relative to a virus particle comprising a variant capsid polypeptide having a reference sequence, e.g., having the wild-type capsid protein, e.g., having capsid polypeptides of SEQ ID NO:1.

Embodiment A-4: In an embodiment of Category A, the transduction is about (or at least about) 10 times better relative to a virus particle comprising a variant capsid polypeptide having a reference sequence, e.g., having the wild-type capsid protein, e.g., having capsid polypeptides of SEQ ID NO:1.

Embodiment A-5: In an embodiment of Category A, the transduction is about (or at least about) 30 times better relative to a virus particle comprising a variant capsid polypeptide having a reference sequence, e.g., having the wild-type capsid protein, e.g., having capsid polypeptides of SEQ ID NO:1.

Embodiment A-6: In an embodiment of Category A, the transduction is about (or at least about) 50 times better relative to a virus particle comprising a variant capsid polypeptide having a reference sequence, e.g., having the wild-type capsid protein, e.g., having capsid polypeptides of SEQ ID NO:1.

Embodiment A-7: In an embodiment of Category A, the transduction is about (or at least about) 70 times better relative to a virus particle comprising a variant capsid polypeptide having a reference sequence, e.g., having the wild-type capsid protein, e.g., having capsid polypeptides of SEQ ID NO:1.

Embodiment A-8: In an embodiment of Category A, the transduction is about (or at least about) 100 times better relative to a virus particle comprising a variant capsid polypeptide having a reference sequence, e.g., having the wild-type capsid protein, e.g., having capsid polypeptides of SEQ ID NO:1.

In some embodiments, the improved transduction is in a range bounded by any two values set forth in embodiments A-1 to A-8. Exemplary ranges are set forth in embodiments A-9 to A-14 below.

Embodiment A-9: In an embodiment of Category A, the transduction ranges are between about 2 and about 100 times better relative to a virus particle comprising a variant capsid polypeptide having a reference sequence, e.g., having the wild-type capsid protein, e.g., having capsid polypeptides of SEQ ID NO:1.

Embodiment A-10: In an embodiment of Category A, the transduction ranges are between about 4 and about 100 times better relative to a virus particle comprising a variant capsid polypeptide having a reference sequence, e.g., having the wild-type capsid protein, e.g., having capsid polypeptides of SEQ ID NO:1.

Embodiment A-11: In an embodiment of Category A, the transduction ranges are between about 6 and about 100 times better relative to a virus particle comprising a variant capsid polypeptide having a reference sequence, e.g., having the wild-type capsid protein, e.g., having capsid polypeptides of SEQ ID NO:1.

Embodiment A-12: In an embodiment of Category A, the transduction ranges are between about 10 and about 100 times better relative to a virus particle comprising a variant capsid polypeptide having a reference sequence, e.g., having the wild-type capsid protein, e.g., having capsid polypeptides of SEQ ID NO:1.

Embodiment A-13: In an embodiment of Category A, the transduction ranges are between about 30 and about 100 times better relative to a virus particle comprising a variant capsid polypeptide having a reference sequence, e.g., having the wild-type capsid protein, e.g., having capsid polypeptides of SEQ ID NO:1.

Embodiment A-14: In an embodiment of Category A, the transduction ranges are between about 50 and about 100 times better relative to a virus particle comprising a variant capsid polypeptide having a reference sequence, e.g., having the wild-type capsid protein, e.g., having capsid polypeptides of SEQ ID NO:1.

Category B (Macular Transduction): In some aspects of the disclosure, a viral particle comprising the variant capsid polypeptide, e.g., the variant capsid polypeptide described herein, exhibits increased macular transduction as compared to a viral particle with the wild-type capsid polypeptide (SEQ ID NO: 1). In some embodiments, the increased macular transduction is as defined as any one of embodiments B-1 through B-10.

Embodiment B-1: In an embodiment of Category B, the transduction is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment B-2: In an embodiment of Category B, the transduction is about (or at least about) 4 times better relative to a virus particle comprising a variant capsid polypeptide having a reference sequence, e.g., having the wild-type capsid protein, e.g., having capsid polypeptides of SEQ ID NO:1.

Embodiment B-3: In an embodiment of Category B, the transduction is about (or at least about) 20 times better relative to a virus particle comprising a variant capsid polypeptide having a reference sequence, e.g., having the wild-type capsid protein, e.g., having capsid polypeptides of SEQ ID NO:1.

Embodiment B-4: In an embodiment of Category B, the transduction is about (or at least about) 50 times better relative to a virus particle comprising a variant capsid polypeptide having a reference sequence, e.g., having the wild-type capsid protein, e.g., having capsid polypeptides of SEQ ID NO: 1.

Embodiment B-5: In an embodiment of Category B, the transduction is about (or at least about) 100 times better relative to a virus particle comprising a variant capsid polypeptide having a reference sequence, e.g., having the wild-type capsid protein, e.g., having capsid polypeptides of SEQ ID NO:1.

Embodiment B-6: In an embodiment of Category B, the transduction is about (or at least about) 200 times better relative to a virus particle comprising a variant capsid polypeptide having a reference sequence, e.g., having the wild-type capsid protein, e.g., having capsid polypeptides of SEQ ID NO:1.

Embodiment B-7: In an embodiment of Category B, the transduction is about (or at least about) 300 times better relative to a virus particle comprising a variant capsid polypeptide having a reference sequence, e.g., having the wild-type capsid protein, e.g., having capsid polypeptides of SEQ ID NO:1.

Embodiment B-8: In an embodiment of Category B, the transduction is about (or at least about) 400 times better relative to a virus particle comprising a variant capsid polypeptide having a reference sequence, e.g., having the wild-type capsid protein, e.g., having capsid polypeptides of SEQ ID NO:1.

Embodiment B-9: In an embodiment of Category B, the transduction is about (or at least about) 500 times better relative to a virus particle comprising a variant capsid polypeptide having a reference sequence, e.g., having the wild-type capsid protein, e.g., having capsid polypeptides of SEQ ID NO: 1.

Embodiment B-10: In an embodiment of Category B, the transduction is about (or at least about) 1000 times better relative to a virus particle comprising a variant capsid polypeptide having a reference sequence, e.g., having the wild-type capsid protein, e.g., having capsid polypeptides of SEQ ID NO:1.

In some embodiments, the improved transduction is in a range bounded by any two values set forth in embodiments B-1 to B-10. Exemplary ranges are set forth in embodiments B-11 to B-17 below.

Embodiment B-11: In an embodiment of Category B, the transduction ranges are between about 2 and about 1000 times better relative to a virus particle comprising a variant capsid polypeptide having a reference sequence, e.g., having the wild-type capsid protein, e.g., having capsid polypeptides of SEQ ID NO: 1.

Embodiment B-12: In an embodiment of Category B, the transduction ranges are between about 4 and about 1000 times better relative to a virus particle comprising a variant capsid polypeptide having a reference sequence, e.g., having the wild-type capsid protein, e.g., having capsid polypeptides of SEQ ID NO:1.

Embodiment B-13: In an embodiment of Category B, the transduction ranges are between about 20 and about 1000 times better relative to a virus particle comprising a variant capsid polypeptide having a reference sequence, e.g., having the wild-type capsid protein, e.g., having capsid polypeptides of SEQ ID NO:1.

Embodiment B-14: In an embodiment of Category B, the transduction ranges are between about 50 and about 1000 times better relative to a virus particle comprising a variant capsid polypeptide having a reference sequence, e.g., having the wild-type capsid protein, e.g., having capsid polypeptides of SEQ ID NO:1.

Embodiment B-15: In an embodiment of Category B, the transduction ranges are between about 100 and about 1000 times better relative to a virus particle comprising a variant capsid polypeptide having a reference sequence, e.g., having the wild-type capsid protein, e.g., having capsid polypeptides of SEQ ID NO:1.

Embodiment B-16: In an embodiment of Category B, the transduction ranges are between about 200 and about 1000 times better relative to a virus particle comprising a variant capsid polypeptide having a reference sequence, e.g., having the wild-type capsid protein, e.g., having capsid polypeptides of SEQ ID NO: 1.

Embodiment B-17: In an embodiment of Category B, the transduction ranges are between about 300 and about 1000 times better relative to a virus particle comprising a variant capsid polypeptide having a reference sequence, e.g., having the wild-type capsid protein, e.g., having capsid polypeptides of SEQ ID NO:1.

Category C (Trabecular Transduction): In some aspects of the disclosure, a viral particle comprising the variant capsid polypeptide, e.g., the variant capsid polypeptide described herein, exhibits increased trabecular transduction as compared to a viral particle with the wild-type capsid polypeptide (SEQ ID NO: 1). In some embodiments, the increased trabecular transduction is as defined as any one of embodiments C-1 through C-4.

Embodiment C-1: In an embodiment of Category C, the transduction is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment C-2: In an embodiment of Category C, the transduction is about (or at least about) 4 times better relative to a virus particle comprising a variant capsid polypeptide having a reference sequence, e.g., having the wild-type capsid protein, e.g., having capsid polypeptides of SEQ ID NO:1.

Embodiment C-3: In an embodiment of Category C, the transduction is about (or at least about) 8 times better relative to a virus particle comprising a variant capsid polypeptide having a reference sequence, e.g., having the wild-type capsid protein, e.g., having capsid polypeptides of SEQ ID NO:1.

Embodiment C-4: In an embodiment of Category C, the transduction is about (or at least about) 12 times better relative to a virus particle comprising a variant capsid polypeptide having a reference sequence, e.g., having the wild-type capsid protein, e.g., having capsid polypeptides of SEQ ID NO:1.

In some embodiments, the improved transduction is in a range bounded by any two values set forth in embodiments C-1 to C-4. Exemplary ranges are set forth in embodiments C-5 to C-7 below.

Embodiment C-5: In an embodiment of Category C, the transduction ranges are between about 2 and about 12 times better relative to a virus particle comprising a variant capsid polypeptide having a reference sequence, e.g., having the wild-type capsid protein, e.g., having capsid polypeptides of SEQ ID NO:1.

Embodiment C-6: In an embodiment of Category C, the transduction ranges are between about 4 and about 12 times better relative to a virus particle comprising a variant capsid polypeptide having a reference sequence, e.g., having the wild-type capsid protein, e.g., having capsid polypeptides of SEQ ID NO:1.

Embodiment C-7: In an embodiment of Category C, the transduction ranges are between about 8 and about 12 times better relative to a virus particle comprising a variant capsid polypeptide having a reference sequence, e.g., having the wild-type capsid protein, e.g., having capsid polypeptides of SEQ ID NO:1.

Category D (Trabecular Transduction): In some aspects of the disclosure, a viral particle comprising the variant capsid polypeptide, e.g., the variant capsid polypeptide described herein, exhibits increased trabecular transduction as compared to a viral particle with capsid polypeptides of SEQ ID NO: 60. In some embodiments, the increased trabecular transduction is as defined as any one of embodiments D-1 through D-4.

Embodiment D-1: In an embodiment of Category D, the transduction is about (or at least about) 1.4 times better relative to a virus particle comprising a variant capsid polypeptide having a reference sequence, e.g., having capsid polypeptides of SEQ ID NO: 60.

Embodiment D-2: In an embodiment of Category D, the transduction is about (or at least about) 1.5 times better relative to a virus particle comprising a variant capsid polypeptide having a reference sequence, e.g., having capsid polypeptides of SEQ ID NO: 60.

Embodiment D-3: In an embodiment of Category D, the transduction is about (or at least about) 2 times better relative to a virus particle comprising a variant capsid polypeptide having a reference sequence, e.g., having capsid polypeptides of SEQ ID NO: 60.

Embodiment D-4: In an embodiment of Category D, the transduction is about (or at least about) 2.7 times better relative to a virus particle comprising a variant capsid polypeptide having a reference sequence, e.g., having capsid polypeptides of SEQ ID NO: 60.

In some embodiments, the improved transduction is in a range bounded by any two values set forth in embodiments D-1 to D-4. Exemplary ranges are set forth in embodiments D-5 and D-6 below.

Embodiment D-5: In an embodiment of Category D, the transduction ranges are between about 1.4 and about 2.7 times better relative to a virus particle comprising a variant capsid polypeptide having a reference sequence, e.g., having capsid polypeptides of SEQ ID NO: 60.

Embodiment D-6: In an embodiment of Category D, the transduction ranges are between about 1.5 and about 2.7 times better relative to a virus particle comprising a variant capsid polypeptide having a reference sequence, e.g., having capsid polypeptides of SEQ ID NO: 60.

Category E (Choroidal Transduction): In some aspects of the disclosure, a viral particle comprising the variant capsid polypeptide, e.g., the variant capsid polypeptide described herein, exhibits increased choroidal transduction as compared to a viral particle with the wild-type capsid polypeptide (SEQ ID NO: 1). In some embodiments, the increased choroidal transduction is as defined as any one of embodiments E-1 through E-3.

Embodiment E-1: In an embodiment of Category E, the transduction is about (or at least about) 1.5 times better relative to a virus particle comprising a variant capsid polypeptide having a reference sequence, e.g., having the wild-type capsid protein, e.g., having capsid polypeptides of SEQ ID NO:1.

Embodiment E-2: In an embodiment of Category E, the transduction is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment E-3: In an embodiment of Category E, the improved transduction is in a range between about 1.5 and about 2 times better relative to a virus particle comprising a variant capsid polypeptide having a reference sequence, e.g., having the wild-type capsid protein, e.g., having capsid polypeptides of SEQ ID NO:1.

Category F (Non-Macular Transduction): In some aspects of the disclosure, a viral particle comprising the variant capsid polypeptide, e.g., the variant capsid polypeptide described herein, exhibits increased non-macular transduction as compared to a viral particle with the wild-type capsid polypeptide (SEQ ID NO: 1). In some embodiments, the increased non-macular transduction is as defined as any one of embodiments F-1 through F-6.

Embodiment F-1: In an embodiment of Category F, the transduction is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment F-2: In an embodiment of Category F, the transduction is about (or at least about) 4 times better relative to a virus particle comprising a variant capsid polypeptide having a reference sequence, e.g., having the wild-type capsid protein, e.g., having capsid polypeptides of SEQ ID NO:1.

Embodiment F-3: In an embodiment of Category F, the transduction is about (or at least about) 8 times better relative to a virus particle comprising a variant capsid polypeptide having a reference sequence, e.g., having the wild-type capsid protein, e.g., having capsid polypeptides of SEQ ID NO:1.

Embodiment F-4: In an embodiment of Category F, the transduction is about (or at least about) 15 times better relative to a virus particle comprising a variant capsid polypeptide having a reference sequence, e.g., having the wild-type capsid protein, e.g., having capsid polypeptides of SEQ ID NO: 1.

Embodiment F-5: In an embodiment of Category F, the transduction is about (or at least about) 30 times better relative to a virus particle comprising a variant capsid polypeptide having a reference sequence, e.g., having the wild-type capsid protein, e.g., having capsid polypeptides of SEQ ID NO:1.

Embodiment F-6: In an embodiment of Category F, the transduction is about (or at least about) 50 times better relative to a virus particle comprising a variant capsid polypeptide having a reference sequence, e.g., having the wild-type capsid protein, e.g., having capsid polypeptides of SEQ ID NO:1.

In some embodiments, the improved transduction is in a range bounded by any two values set forth in embodiments F-1 to F-6. Exemplary ranges are set forth in embodiments F-7 to F-9 below.

Embodiment F-7: In an embodiment of Category F, the transduction ranges are between about 2 and about 50 times better relative to a virus particle comprising a variant capsid polypeptide having a reference sequence, e.g., having the wild-type capsid protein, e.g., having capsid polypeptides of SEQ ID NO:1.

Embodiment F-8: In an embodiment of Category F, the transduction ranges are between about 4 and about 50 times better relative to a virus particle comprising a variant capsid polypeptide having a reference sequence, e.g., having the wild-type capsid protein, e.g., having capsid polypeptides of SEQ ID NO:1.

Embodiment F-9: In an embodiment of Category F, the transduction ranges are between about 15 and about 50 times better relative to a virus particle comprising a variant capsid polypeptide having a reference sequence, e.g., having the wild-type capsid protein, e.g., having capsid polypeptides of SEQ ID NO:1.

Category G (Virus Production): In some aspects of the disclosure, a viral particle comprising the variant capsid polypeptide, e.g., the variant capsid polypeptide described herein, exhibits increased virus production as compared to a viral particle with the wild-type capsid polypeptide (SEQ ID NO: 1). In some embodiments, the increased virus production is as defined as any one of embodiments G-1 through G-8.

Embodiment G-1: In an embodiment of Category G, the virus production is about (or at least about) 1.2 times better relative to a virus particle comprising a variant capsid polypeptide having a reference sequence, e.g., having the wild-type capsid protein, e.g., having capsid polypeptides of SEQ ID NO:1.

Embodiment G-2: In an embodiment of Category G, the virus production is about (or at least about) 1.4 times better relative to a virus particle comprising a variant capsid polypeptide having a reference sequence, e.g., having the wild-type capsid protein, e.g., having capsid polypeptides of SEQ ID NO:1.

Embodiment G-3: In an embodiment of Category G, the virus production is about (or at least about) 1.6 times better relative to a virus particle comprising a variant capsid polypeptide having a reference sequence, e.g., having the wild-type capsid protein, e.g., having capsid polypeptides of SEQ ID NO:1.

Embodiment G-4: In an embodiment of Category G, the virus production is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment G-5: In an embodiment of Category G, the virus production is about (or at least about) 2.5 times better relative to a virus particle comprising a variant capsid polypeptide having a reference sequence, e.g., having the wild-type capsid protein, e.g., having capsid polypeptides of SEQ ID NO:1.

Embodiment G-6: In an embodiment of Category G, the virus production is about (or at least about) 3 times better relative to a virus particle comprising a variant capsid polypeptide having a reference sequence, e.g., having the wild-type capsid protein, e.g., having capsid polypeptides of SEQ ID NO:1.

Embodiment G-7: In an embodiment of Category G, the virus production is about (or at least about) 4 times better relative to a virus particle comprising a variant capsid polypeptide having a reference sequence, e.g., having the wild-type capsid protein, e.g., having capsid polypeptides of SEQ ID NO:1.

Embodiment G-8: In an embodiment of Category G, the virus production is about (or at least about) 6 times better relative to a virus particle comprising a variant capsid polypeptide having a reference sequence, e.g., having the wild-type capsid protein, e.g., having capsid polypeptides of SEQ ID NO:1.

In some embodiments, the improved virus production is in a range bounded by any two values set forth in embodiments G-1 to G-8. Exemplary ranges are set forth in embodiments G-9 to G-13 below.

Embodiment G-9: In an embodiment of Category G, the virus production ranges are between about 1.2 and about 6 times better relative to a virus particle comprising a variant capsid polypeptide having a reference sequence, e.g., having the wild-type capsid protein, e.g., having capsid polypeptides of SEQ ID NO:1.

Embodiment G-10: In an embodiment of Category G, the virus production ranges are between about 1.2 and about 4 times better relative to a virus particle comprising a variant capsid polypeptide having a reference sequence, e.g., having the wild-type capsid protein, e.g., having capsid polypeptides of SEQ ID NO:1.

Embodiment G-11: In an embodiment of Category G, the virus production ranges are between about 1.4 and about 4 times better relative to a virus particle comprising a variant capsid polypeptide having a reference sequence, e.g., having the wild-type capsid protein, e.g., having capsid polypeptides of SEQ ID NO:1.

Embodiment G-12: In an embodiment of Category G, the virus production ranges are between about 1.6 and about 4 times better relative to a virus particle comprising a variant capsid polypeptide having a reference sequence, e.g., having the wild-type capsid protein, e.g., having capsid polypeptides of SEQ ID NO:1.

Embodiment G-13: In an embodiment of Category G, the virus production ranges are between about 2 and about 4 times better relative to a virus particle comprising a variant capsid polypeptide having a reference sequence, e.g., having the wild-type capsid protein, e.g., having capsid polypeptides of SEQ ID NO:1.

Category H (Non-Macular Biodistribution): In some aspects of the disclosure, a viral particle comprising the variant capsid polypeptide, e.g., the variant capsid polypeptide described herein, exhibits increased non-macular biodistribution as compared to a viral particle with the wild-type capsid polypeptide (SEQ ID NO: 1). In some embodiments, the increased non-macular biodistribution is as defined as any one of embodiments H-1 through H-3.

Embodiment H-1: In an embodiment of Category H, the biodistribution is about (or at least about) 2 times better relative to a virus particle comprising a variant capsid polypeptide having a reference sequence, e.g., having the wild-type capsid protein, e.g., having capsid polypeptides of SEQ ID NO: 1.

Embodiment H-2: In an embodiment of Category H, the biodistribution is about (or at least about) 4 times better relative to a virus particle comprising a variant capsid polypeptide having a reference sequence, e.g., having the wild-type capsid protein, e.g., having capsid polypeptides of SEQ ID NO:1.

Embodiment H-3: In an embodiment of Category H, the biodistribution is about (or at least about) 6 times better relative to a virus particle comprising a variant capsid polypeptide having a reference sequence, e.g., having the wild-type capsid protein, e.g., having capsid polypeptides of SEQ ID NO:1.

In some embodiments, the improved biodistribution is in a range bounded by any two values set forth in embodiments H-1 to H-3. Exemplary ranges are set forth in embodiments H-4 and H-5 below.

Embodiment H-4: In an embodiment of Category H, the improved biodistribution is in a range between about 2 and about 6 times better relative to a virus particle comprising a variant capsid polypeptide having a reference sequence, e.g., having the wild-type capsid protein, e.g., having capsid polypeptides of SEQ ID NO:1.

Embodiment H-5: In an embodiment of Category H, the improved biodistribution is in a range between about 2 and about 4 times better relative to a virus particle comprising a variant capsid polypeptide having a reference sequence, e.g., having the wild-type capsid protein, e.g., having capsid polypeptides of SEQ ID NO:1.

Category I (Trabecular Meshwork Biodistribution): In some aspects of the disclosure, a viral particle comprising the variant capsid polypeptide, e.g., the variant capsid polypeptide described herein, exhibits increased trabecular meshwork biodistribution as compared to a viral particle with the wild-type capsid polypeptide (SEQ ID NO: 1). In some embodiments, the increased trabecular meshwork biodistribution is as defined as any one of embodiments I-1 through 1-3.

Embodiment I-1: In an embodiment of Category I, the biodistribution is about (or at least about) 1.1 times better relative to a virus particle comprising a variant capsid polypeptide having a reference sequence, e.g., having the wild-type capsid protein, e.g., having capsid polypeptides of SEQ ID NO:1.

Embodiment I-2: In an embodiment of Category H, the biodistribution is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment I-3: In an embodiment of Category H, the improved biodistribution is in a range between about 1.1 and about 2 times better relative to a virus particle comprising a variant capsid polypeptide having a reference sequence, e.g., having the wild-type capsid protein, e.g., having capsid polypeptides of SEQ ID NO:1.

Category J (Neural Retina Biodistribution): In some aspects of the disclosure, a viral particle comprising the variant capsid polypeptide, e.g., the variant capsid polypeptide described herein, exhibits increased neural retina biodistribution as compared to a viral particle with the wild-type capsid polypeptide (SEQ ID NO: 1). In some embodiments, the increased neural retina biodistribution is as defined as any one of embodiments J-1 through J-4.

Embodiment J-1: In an embodiment of Category J, the biodistribution is about (or at least about) 2 times better relative to a virus particle comprising a variant capsid polypeptide having a reference sequence, e.g., having the wild-type capsid protein, e.g., having capsid polypeptides of SEQ ID NO:1.

Embodiment J-2: In an embodiment of Category J, the biodistribution is about (or at least about) 5 times better relative to a virus particle comprising a variant capsid polypeptide having a reference sequence, e.g., having the wild-type capsid protein, e.g., having capsid polypeptides of SEQ ID NO:1.

Embodiment J-3: In an embodiment of Category J, the biodistribution is about (or at least about) 10 times better relative to a virus particle comprising a variant capsid polypeptide having a reference sequence, e.g., having the wild-type capsid protein, e.g., having capsid polypeptides of SEQ ID NO:1.

Embodiment J-4: In an embodiment of Category J, the biodistribution is about (or at least about) 20 times better relative to a virus particle comprising a variant capsid polypeptide having a reference sequence, e.g., having the wild-type capsid protein, e.g., having capsid polypeptides of SEQ ID NO:1.

In some embodiments, the improved biodistribution is in a range bounded by any two values set forth in embodiments J-1 to J-4. Exemplary ranges are set forth in embodiments J-5 and J-6 below.

Embodiment J-5: In an embodiment of Category J, the improved biodistribution is in a range between about 2 and about 20 times better relative to a virus particle comprising a variant capsid polypeptide having a reference sequence, e.g., having the wild-type capsid protein, e.g., having capsid polypeptides of SEQ ID NO:1.

Embodiment J-6: In an embodiment of Category J, the improved biodistribution is in a range between about 5 and about 20 times better relative to a virus particle comprising a variant capsid polypeptide having a reference sequence, e.g., having the wild-type capsid protein, e.g., having capsid polypeptides of SEQ ID NO: 1.

Category K (Macular Biodistribution): In some aspects of the disclosure, a viral particle comprising the variant capsid polypeptide, e.g., the variant capsid polypeptide described herein, exhibits increased macular biodistribution as compared to a viral particle with the wild-type capsid polypeptide (SEQ ID NO: 1). In some embodiments, the increased macular biodistribution is as defined as any one of embodiments K-1 through K-7.

Embodiment K-1: In an embodiment of Category K, the biodistribution is about (or at least about) 3 times better relative to a virus particle comprising a variant capsid polypeptide having a reference sequence, e.g., having the wild-type capsid protein, e.g., having capsid polypeptides of SEQ ID NO:1.

Embodiment K-2: In an embodiment of Category K, the biodistribution is about (or at least about) 10 times better relative to a virus particle comprising a variant capsid polypeptide having a reference sequence, e.g., having the wild-type capsid protein, e.g., having capsid polypeptides of SEQ ID NO:1.

Embodiment K-3: In an embodiment of Category K, the biodistribution is about (or at least about) 30 times better relative to a virus particle comprising a variant capsid polypeptide having a reference sequence, e.g., having the wild-type capsid protein, e.g., having capsid polypeptides of SEQ ID NO:1.

Embodiment K-4: In an embodiment of Category K, the biodistribution is about (or at least about) 60 times better relative to a virus particle comprising a variant capsid polypeptide having a reference sequence, e.g., having the wild-type capsid protein, e.g., having capsid polypeptides of SEQ ID NO:1.

Embodiment K-5: In an embodiment of Category K, the biodistribution is about (or at least about) 80 times better relative to a virus particle comprising a variant capsid polypeptide having a reference sequence, e.g., having the wild-type capsid protein, e.g., having capsid polypeptides of SEQ ID NO:1.

Embodiment K-6: In an embodiment of Category K, the biodistribution is about (or at least about) 100 times better relative to a virus particle comprising a variant capsid polypeptide having a reference sequence, e.g., having the wild-type capsid protein, e.g., having capsid polypeptides of SEQ ID NO:1.

Embodiment K-7: In an embodiment of Category K, the biodistribution is about (or at least about) 180 times better relative to a virus particle comprising a variant capsid polypeptide having a reference sequence, e.g., having the wild-type capsid protein, e.g., having capsid polypeptides of SEQ ID NO:1.

In some embodiments, the improved biodistribution is in a range bounded by any two values set forth in embodiments K-1 to K-7. Exemplary ranges are set forth in embodiments K-8 to K-12 below.

Embodiment K-8: In an embodiment of Category K, the biodistribution ranges are between about 3 and about 180 times better relative to a virus particle comprising a variant capsid polypeptide having a reference sequence, e.g., having the wild-type capsid protein, e.g., having capsid polypeptides of SEQ ID NO:1.

Embodiment K-9: In an embodiment of Category K, the biodistribution ranges are between about 10 and about 180 times better relative to a virus particle comprising a variant capsid polypeptide having a reference sequence, e.g., having the wild-type capsid protein, e.g., having capsid polypeptides of SEQ ID NO:1.

Embodiment K-10: In an embodiment of Category K, the biodistribution ranges are between about 30 and about 180 times better relative to a virus particle comprising a variant capsid polypeptide having a reference sequence, e.g., having the wild-type capsid protein, e.g., having capsid polypeptides of SEQ ID NO:1.

Embodiment K-11: In an embodiment of Category K, the biodistribution ranges are between about 60 and about 180 times better relative to a virus particle comprising a variant capsid polypeptide having a reference sequence, e.g., having the wild-type capsid protein, e.g., having capsid polypeptides of SEQ ID NO:1.

Embodiment K-12: In an embodiment of Category K, the biodistribution ranges are between about 80 and about 180 times better relative to a virus particle comprising a variant capsid polypeptide having a reference sequence, e.g., having the wild-type capsid protein, e.g., having capsid polypeptides of SEQ ID NO:1.

Category L (Trabecular Biodistribution): In some aspects of the disclosure, a viral particle comprising the variant capsid polypeptide, e.g., the variant capsid polypeptide described herein, exhibits increased trabecular biodistribution as compared to a viral particle with AAV2 capsid polypeptides of SEQ ID NO: 60. In some embodiments, the increased trabecular biodistribution is as defined as any one of embodiments L-1 through L-4.

Embodiment L-1: In an embodiment of Category L, the transduction is about (or at least about) 1.5 times better relative to a virus particle comprising a variant capsid polypeptide having a reference sequence, e.g., having capsid polypeptides of SEQ ID NO: 60.

Embodiment L-2: In an embodiment of Category L, the transduction is about (or at least about) 2 times better relative to a virus particle comprising a variant capsid polypeptide having a reference sequence, e.g., having capsid polypeptides of SEQ ID NO: 60.

Embodiment L-3: In an embodiment of Category L, the transduction is about (or at least about) 2.3 times better relative to a virus particle comprising a variant capsid polypeptide having a reference sequence, e.g., having capsid polypeptides of SEQ ID NO: 60.

Embodiment L-4: In an embodiment of Category L, the transduction is about (or at least about) 3 times better relative to a virus particle comprising a variant capsid polypeptide having a reference sequence, e.g., having capsid polypeptides of SEQ ID NO: 60.

In some embodiments, the improved transduction is in a range bounded by any two values set forth in embodiments L-1 to L-4. Exemplary ranges are set forth in embodiments L-5 to L-6 below.

Embodiment L-5: In an embodiment of Category L, the transduction ranges are between about 1.5 and about 3 times better relative to a virus particle comprising a variant capsid polypeptide having a reference sequence, e.g., having capsid polypeptides of SEQ ID NO: 60.

Embodiment L-6: In an embodiment of Category L, the transduction ranges are between about 2 and about 3 times better relative to a virus particle comprising a variant capsid polypeptide having a reference sequence, e.g., having capsid polypeptides of SEQ ID NO: 60.

According to some embodiments, a viral particle comprising the variant capsid polypeptide, e.g., the variant capsid polypeptide described herein, exhibits increased transduction of one or more cell types as compared to a viral particle with the wild-type capsid polypeptide (SEQ ID NO: 1). In some embodiments, the cell type(s) is an ocular cell type found in the retinal tissue. In some embodiments, the cell type(s) is an ocular cell type found in the trabecula meshwork tissue. Examples of cell types include amacrine cells, biopolar cells, cones, horizontal cells, microglia, Müller glia, retinal ganglion cells, rods, corneal epithelium, ciliary muscle, melanocytes, Schwann cells, beam cells, juxtacanalicular tissue (JCT), fibroblasts, and pericytes.

In some embodiments, a viral particle comprising the variant capsid polypeptide, e.g., the variant capsid polypeptide described herein, exhibits increased transduction of retinal ganglion cells as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of amacrine cells as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of bipolar cells as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of cones as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of Müller glia as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of rods as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of beam cells as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of ciliary muscle as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of corneal epithelium as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of juxtacanalicular tissue (JCT) as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of melanocytes as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of Schwann cells as compared to a viral particle with the wild-type capsid polypeptide (SEQ ID NO: 1). In some embodiments, a viral particle comprising the variant capsid polypeptide, e.g., the variant capsid polypeptide described herein, exhibits decreased transduction of ciliary muscle as compared to a viral particle with the wild-type capsid polypeptide (SEQ ID NO: 1).

In some embodiments, a viral particle comprising the variant capsid polypeptide, e.g., the variant capsid polypeptide described herein, exhibits increased transduction of one cell type over another cell type as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of retinal ganglion cells over amacrine cells, biopolar cells, cones, Müller glia, or rods as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of amacrine cells over biopolar cells, cones, Müller glia, or rods as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of cones over biopolar cells, amacrine cells, Müller glia, or rods as compared to a viral particle with the wild-type capsid polypeptide (SEQ ID NO: 1). In some embodiments, a viral particle comprising the variant capsid polypeptide, e.g., the variant capsid polypeptide described herein, exhibits increased transduction of juxtacanalicular tissue (JCT) over beam cells, ciliary muscle, corneal epithelium, melanocytes, or Schwann cells as compared to a viral particle with the wild-type capsid polypeptide (SEQ ID NO: 1).

### Methods of Making Compositions Described Herein

The disclosure is directed, in part, to a method of making a capsid polypeptide described herein or a virus particle, such as but not limited to a dependoparvovirus particle, e.g., a dependoparvovirus particle described herein. In some embodiments, a method of making dependoparvovirus particle comprises providing a cell, cell-free system, or other translation system, comprising a nucleic acid described herein encoding a variant capsid polypeptide provided for herein, or a polypeptide provided for herein (e.g., a variant capsid polypeptide); and cultivating the cell, cell-free system, or other translation system under conditions suitable for the production of the dependoparvovirus particle, thereby making the dependoparvovirus particle.

In some embodiments, providing a cell comprising a nucleic acid described herein comprises introducing the nucleic acid to the cell, e.g., transfecting or transforming the cell with the nucleic acid. The nucleic acids of the disclosure may be situated as a part of any genetic element (vector) which may be delivered to a host cell, e.g., naked DNA, a plasmid, phage, transposon, cosmid, episome, a protein in a non-viral delivery vehicle (e.g., a lipid-based carrier), virus, etc. which transfer the sequences carried thereon. Such a vector may be delivered by any suitable method, including transfection, liposome delivery, electroporation, membrane fusion techniques, viral infection, high velocity DNA-coated pellets, and protoplast fusion. A person of skill in the art possesses the knowledge and skill in nucleic acid manipulation to construct any embodiment of this invention and said skills include genetic engineering, recombinant engineering, and synthetic techniques. See, e.g., Sambrook et al, Molecular Cloning: A Laboratory Manual, Cold Spring Harbor Press, Cold Spring Harbor, NY.

In some embodiments, a vector of the disclosure comprises sequences encoding a viral particle, such as but not limited to, a dependoparvovirus variant capsid polypeptide as provided for herein or a fragment thereof. In some embodiments, vectors of the disclosure comprises sequences encoding a viral particle, such as but not limited to, a dependoparvovirus rep protein or a fragment thereof. In some embodiments, such vectors may contain sequence encoding both dependoparvovirus cap (e.g., a variant capsid polypeptide described herein) and rep proteins. In vectors in which both AAV rep and cap are provided, the dependoparvovirus rep and dependoparvovirus cap sequences may both be of the same dependoparvovirus species or serotype origin, such as AAV2. Alternatively, the present disclosure also provides vectors in which the rep sequences are from a dependoparvovirus species or serotype which differs from that from which the cap sequences are derived. In some embodiments, the rep and cap sequences are expressed from separate sources (e.g., separate vectors, or a host cell genome and a vector). In some embodiments, the rep sequences are fused in frame to cap sequences of a different dependoparvovirus species or serotype to form a chimeric dependoparvovirus vector. In some embodiments, the vectors of the invention further contain a payload, e.g., a minigene comprising a selected transgene (e.g., a payload as described herein), e.g., flanked by dependoparvovirus 5′ ITR and dependoparvovirus 3′ ITR.

The vectors described herein, e.g., a plasmid, are useful for a variety of purposes, but are particularly well suited for use in production of recombinant viral particles, such as but not limited to, dependoparvovirus particles comprising dependoparvovirus sequences or a fragment thereof, and in some embodiments, a payload.

In some embodiments, the disclosure provides a method of making a viral particle, such as but not limited to, a dependoparvovirus particle (e.g., a dependoparvovirus B particle, e.g., an AAV2 particle or particle comprising a variant capsid polypeptide as described herein), or a portion thereof. In some embodiments, the method comprises culturing a host cell which contains a nucleic acid sequence encoding a dependoparvovirus variant capsid polypeptide as provided for herein, or fragment thereof; a functional rep gene; a payload (e.g., as described herein), e.g., a minigene comprising dependoparvovirus inverted terminal repeats (ITRs) and a transgene, optionally under the control of a regulatory element such as a promoter; and sufficient helper functions to promote packaging of the payload, e.g., minigene, into the dependoparvovirus capsid. The components necessary to be cultured in the host cell to package a payload, e.g., minigene, in a dependoparvovirus capsid may be provided to the host cell in trans. In some embodiments, any one or more of the required components (e.g., payload (e.g., minigene), rep sequences, cap sequences, and/or helper functions) may be provided by a host cell which has been engineered to stably comprise one or more of the required components using methods known to those of skill in the art. In some embodiments, a host cell which has been engineered to stably comprise the required component(s) comprises it under the control of an inducible promoter. In some embodiments, the required component may be under the control of a constitutive promoter. Examples of suitable inducible and constitutive promoters are provided herein and further examples are known to those of skill in the art. In some embodiments, a selected host cell which has been engineered to stably comprise one or more components may comprise a component under the control of a constitutive promoter and another component under the control of one or more inducible promoters. For example, a host cell which has been engineered to stably comprise the required components may be generated from 293 cells (e.g., which comprise helper functions under the control of a constitutive promoter), which comprises the rep and/or cap proteins under the control of one or more inducible promoters.

The payload (e.g., minigene), rep sequences, cap sequences, and helper functions required for producing a viral particle, such as but not limited to, a dependoparvovirus particle of the disclosure may be delivered to the packaging host cell in the form of any genetic element which transfers the sequences carried thereon (e.g., in a vector or combination of vectors). The genetic element may be delivered by any suitable method, including those described herein. Methods used to construct genetic elements, vectors, and other nucleic acids of the disclosure are known to those with skill and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, J. Virol, 70:520-532 (1993) and U.S. Pat. No. 5,478,745. Unless otherwise specified, the dependoparvovirus ITRs, and other selected dependoparvovirus components described herein, may be readily selected from among any dependoparvovirus species and serotypes, e.g., AAV1, AAV2, AAV3, AAV4, AAV5, AAV6, AAV7, AAV9. ITRs or other dependoparvovirus components may be readily isolated using techniques available to those of skill in the art from a dependoparvovirus species or serotype. Dependoparvovirus species and serotypes may be isolated or obtained from academic, commercial, or public sources (e.g., the American Type Culture Collection, Manassas, VA). In some embodiments, the dependoparvovirus sequences may be obtained through synthetic or other suitable means by reference to published sequences such as are available in the literature or in databases such as, e.g., GenBank or PubMed.

The viral particles, such as but not limited to, dependoparvovirus particles (e.g., including a variant capsid polypeptide and, for example, a payload) of the disclosure may be produced using any invertebrate cell type which allows for production of dependoparvovirus or biologic products and which can be maintained in culture. In some embodiments, an insect cell may be used in production of the compositions described herein or in the methods of making a dependoparvovirus particle described herein. For example, an insect cell line used can be from Spodoptera frugiperda, such as Sf9, SF21, SF900+, drosophila cell lines, mosquito cell lines, e.g., Aedes albopictus derived cell lines, domestic silkworm cell lines, e.g. Bombyxmori cell lines, Trichoplusia ni cell lines such as High Five cells or Lepidoptera cell lines such as Ascalapha odorata cell lines. In some embodiments, the insect cells are susceptible to baculovirus infection, including High Five, Sf9, Se301, SeIZD2109, SeUCR1, SP900+, Sf21, BTI-TN-5B1-4, MG-1, Tn368, HzAml, BM-N, Ha2302, Hz2E5 and Ao38.

In some embodiments, the methods of the disclosure can be carried out with any mammalian cell type which allows for replication of dependoparvovirus or production of biologic products, and which can be maintained in culture. In some embodiments, the mammalian cells used can be HEK293, HEK293T, HeLa, CHO, NS0, SP2/0, PER.C6, Vero, RD, BHK, HT 1080, A549, Cos-7, ARPE-19 or MRC-5 cells. In some embodiments the culture is an adherent cell culture. In some embodiments, the culture is a suspension cell culture.

Methods of expressing proteins (e.g., recombinant or heterologous proteins, e.g., viral polypeptides, such as but not limited to, dependoparvovirus polypeptides) in insect cells are well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J Vir. 66:6922-30 (1992); Kirnbauer et al., Vir. 219:37-44 (1996); Zhao et al., Vir. 272:382-93 (2000); and Samulski et al., U.S. Pat. No. 6,204,059. In some embodiments, a nucleic acid construct encoding dependoparvovirus polypeptides (e.g., a dependoparvovirus genome) in insect cells is an insect cell-compatible vector. An “insect cell-compatible vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cell's genome or remain present extra-chromosomally. The vector may be present permanently or transiently, e.g., as an episomal vector. Vectors may be introduced by any means known in the art. Such means include but are not limited to chemical treatment of the cells, electroporation, or infection. In some embodiments, the vector is a baculovirus, a viral vector, or a plasmid.

In some embodiments, a nucleic acid sequence encoding a viral polypeptide, such as but not limited to, a dependoparvovirus polypeptide is operably linked to regulatory expression control sequences for expression in a specific cell type, such as Sf9 or HEK cells. Techniques known to one skilled in the art for expressing foreign genes in insect host cells or mammalian host cells can be used with the compositions and methods of the disclosure. Methods for molecular engineering and expression of polypeptides in insect cells is described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. Promoters suitable for transcription of a nucleotide sequence encoding a dependoparvovirus polypeptide include the polyhedron, p10, p35 or IE-1 promoters and further promoters described in the above references are also contemplated.

In some embodiments, providing a cell comprising a nucleic acid described herein comprises acquiring a cell comprising the nucleic acid.

Methods of cultivating cells, cell-free systems, and other translation systems are known to those of skill in the art. In some embodiments, cultivating a cell comprises providing the cell with suitable media and incubating the cell and media for a time suitable to achieve viral particle production.

In some embodiments, a method of making a viral particle, such as but not limited to a dependoparvovirus particle, further comprises a purification step comprising isolating the dependoparvovirus particle from one or more other components (e.g., from a cell or media component).

In some embodiments, production of the viral particle, such as but not limited to, the dependoparvovirus particle comprises one or more (e.g., all) of: expression of dependoparvovirus polypeptides, assembly of a dependoparvovirus capsid (e.g., a capsid comprising a variant capsid polypeptide provided for herein), expression (e.g., duplication) of a dependoparvovirus genome, and packaging of the dependoparvovirus genome into the dependoparvovirus capsid to produce a dependoparvovirus particle. In some embodiments, production of the dependoparvovirus particle further comprises secretion of the dependoparvovirus particle.

In some embodiments, and as described elsewhere herein, the nucleic acid molecule encoding the variant capsid polypeptide is disposed in a dependoparvovirus genome. In some embodiments, and as described elsewhere herein, the nucleic acid molecule encoding the variant capsid polypeptide is packaged into a dependoparvovirus particle along with the dependoparvovirus genome as part of a method of making a dependoparvovirus particle described herein. In other embodiments, the nucleic acid molecule encoding the variant capsid polypeptide is not packaged into a dependoparvovirus particle made by a method described herein.

In some embodiments, a method of making a viral particle, such as but not limited to, a dependoparvovirus particle described herein produces a dependoparvovirus particle comprising a payload (e.g., a payload described herein) and the variant capsid polypeptide. In some embodiments, the payload comprises a second nucleic acid (e.g., in addition to the dependoparvovirus genome), and production of the dependoparvovirus particle comprises packaging the second nucleic acid into the dependoparvovirus particle. In some embodiments, a cell, cell-free system, or other translation system for use in a method of making a dependoparvovirus particle comprises the second nucleic acid. In some embodiments, the second nucleic acid comprises an exogenous sequence (e.g., exogenous to the dependoparvovirus, the cell, or to a target cell or subject who will be administered the dependoparvovirus particle). In some embodiments, the exogenous sequence encodes an exogenous polypeptide. In some embodiments, the exogenous sequence encodes a therapeutic product.

In some embodiments, the composition of the invention is a pharmaceutical composition

In some embodiments, a nucleic acid or polypeptide described herein is produced by a method known to one of skill in the art. The nucleic acids, polypeptides, and fragments thereof of the disclosure may be produced by any suitable means, including recombinant production, chemical synthesis, or other synthetic means. Such production methods are within the knowledge of those of skill in the art and are not a limitation of the present invention.

### Applications

The disclosure is directed, in part, to compositions comprising a nucleic acid, polypeptide, or particles described herein. The disclosure is further directed, in part, to methods of utilizing a composition, nucleic acid, polypeptide, or particles described herein. As will be apparent based on the disclosure, nucleic acids, polypeptides, particles, and methods disclosed herein have a variety of utilities.

The disclosure is directed, in part, to a vector comprising a nucleic acid described herein, e.g., a nucleic acid encoding a variant capsid polypeptide. Many types of vectors are known to those of skill in the art. In some embodiments, a vector comprises a plasmid. In some embodiments, the vector is an isolated vector, e.g., removed from a cell or other biological components.

The disclosure is directed, in part to a cell, cell-free system, or other translation system, comprising a nucleic acid or vector described herein, e.g., a nucleic acid or vector comprising a nucleic acid molecule encoding a variant capsid polypeptide. In some embodiments, the cell, cell-free system, or other translation system is capable of producing dependoparvovirus particles comprising the variant capsid polypeptides. In some embodiments, the cell, cell-free system, or other translation system comprises a nucleic acid comprising a dependoparvovirus genome or components of a dependoparvovirus genome sufficient to promote production of dependoparvovirus particles comprising the variant capsid polypeptides.

In some embodiments, the cell, cell-free system, or other translation system further comprises one or more non-dependoparvovirus nucleic acid sequences that promote dependoparvovirus particle production and/or secretion. Said sequences are referred to herein as helper sequences. In some embodiments, a helper sequence comprises one or more genes from another virus, e.g., an adenovirus or herpes virus. In some embodiments, the presence of a helper sequence is necessary for production and/or secretion of a dependoparvovirus particle. In some embodiments, a cell, cell-free system, or other translation system comprises a vector, e.g., plasmid, comprising one or more helper sequences.

In some embodiments, a cell, cell-free system, or other translation system comprises a first nucleic acid and a second nucleic acid, wherein the first nucleic acid comprises a sequences encoding one or more dependoparvovirus genes (e.g., a Cap gene, a Rep gene, or a complete dependoparvovirus genome) and a helper sequence, and wherein the second nucleic acid comprises a payload. In some embodiments, a cell, cell-free system, or other translation system comprises a first nucleic acid and a second nucleic acid, wherein the first nucleic acid comprises a sequences encoding one or more dependoparvovirus genes (e.g., a Cap gene, a Rep gene, or a complete dependoparvovirus genome) and a payload, and wherein the second nucleic acid comprises a helper sequence. In some embodiments, a cell, cell-free system, or other translation system comprises a first nucleic acid and a second nucleic acid, wherein the first nucleic acid comprises a helper sequence and a payload, and wherein the second nucleic acid comprises a sequences encoding one or more dependoparvovirus genes (e.g., a Cap gene, a Rep gene, or a complete dependoparvovirus genome). In some embodiments, a cell, cell-free system, or other translation system comprises a first nucleic acid, a second nucleic acid, and a third nucleic acid, wherein the first nucleic acid comprises a sequences encoding one or more dependoparvovirus genes (e.g., a Cap gene, a Rep gene, or a complete dependoparvovirus genome), the second nucleic acid comprises a helper sequence, and the third nucleic acid comprises a payload.

In some embodiments, the first nucleic acid, second nucleic acid, and optionally third nucleic acid are situated in separate molecules, e.g., separate vectors or a vector and genomic DNA. In some embodiments, one, two, or all of the first nucleic acid, second nucleic acid, and optionally third nucleic acid are integrated (e.g., stably integrated) into the genome of a cell.

A cell of the disclosure may be generated by transfecting a suitable cell with a nucleic acid described herein. In some embodiments, a method of making a dependoparvovirus particle comprising a variant capsid polypeptide as provided for herein or improving a method of making a dependoparvovirus particle comprises providing a cell described herein. In some embodiments, providing a cell comprises transfecting a suitable cell with one or more nucleic acids described herein.

In some embodiments, the virus particle comprising the variant capsid is produced at a level at least 10%, at least 20%, at least 50%, at least 100%, at least 200% or greater than the production level of wt AAV2 from the same producer cell type, e.g., from HEK293 cells, e.g., from adherent culture of HEK293 cells.

Many types and kinds of cells suitable for use with the nucleic acids and vectors described herein are known in the art. In some embodiments, the cell is a human cell. In some embodiments, the cell is an immortalized cell or a cell from a cell line known in the art. In some embodiments, the cell is an HEK293 cell.

### Methods of Delivering a Payload

The disclosure is directed, in part, to a method of delivering a payload to a cell, e.g., a cell in a subject or in a sample. In some embodiments, a method of delivering a payload to a cell comprises contacting the cell with a dependoparvovirus particle comprising a variant capsid polypeptide (e.g., described herein) comprising the payload. In some embodiments, the dependoparvovirus particle is a dependoparvovirus particle described herein and comprises a payload described herein. In some embodiments, the cell is an ocular cell. In some embodiments, the ocular cell is in the retina, macula, or trabecular meshwork. In some embodiments, the ocular cell is in the retina. In some embodiments, the ocular cell is in the macula. In some embodiments, the ocular cell is in the trabecular meshwork.

In some embodiments, the ocular cell is in the front third of the eye, which includes the structures in front of the vitreous humor. Examples of structures in front of the vitreous humor, include the cornea, iris, ciliary body, lens, trabecular meshwork, and Schlemm's canal. Accordingly, in some embodiments, the cell is in the cornea, iris, ciliary body, lens, trabecular meshwork, or Schlemm's canal, or any combination thereof.

In some embodiments, the ocular cell is posterior to the lens, such as in the anterior hyaloid membrane and all of the optical structures behind it, such as the vitreous humor, retina, choroid or optic nerve, or any combination thereof. Accordingly, in some embodiments, the cell is in the anterior hyaloid membrane and all of the optical structures behind it, such as the vitreous humor, retina, choroid or optic nerve, or any combination thereof.

In some embodiments, the cell type is an ocular cell such as, for example, a neural retinal cell, a photoreceptive retinal ganglion cell, a bipolar cell, a horizontal cell, an amacrine cell, a photoreceptor (e.g., a rod or a cone cell), an endothelial cell (e.g., a retinal pigmented epithelial cell), and endothelial-like cell, and the like.

The disclosure is further directed in part to a virus particle comprising a capsid polypeptide described herein. In some embodiments, the virus particle comprises a capsid polypeptide described herein and a nucleic acid expression construct. In some embodiments, the nucleic acid expression construct of the virus particle comprises a payload.

In some embodiments, the payload comprises a transgene. In some embodiments, the transgene is a nucleic acid sequence heterologous to the vector sequences flanking the transgene which encodes a polypeptide, RNA (e.g., a miRNA or siRNA) or other product of interest. The nucleic acid of the transgene may be operatively linked to a regulatory component in a manner sufficient to promote transgene transcription, translation, and/or expression in a host cell.

A transgene may be any polypeptide or RNA encoding sequence and the transgene selected will depend upon the use envisioned. In some embodiments, a transgene comprises a reporter sequence, which upon expression produces a detectable signal. Such reporter sequences include, without limitation, DNA sequences encoding colorimetric reporters (e.g., β-lactamase, β-galactosidase (LacZ), alkaline phosphatase), cell division reporters (e.g., thymidine kinase), fluorescent or luminescence reporters (e.g., green fluorescent protein (GFP) or luciferase), resistance conveying sequences (e.g., chloramphenicol acetyltransferase (CAT)), or membrane bound proteins including to which high affinity antibodies directed thereto exist or can be produced by conventional means, e.g., comprising an antigen tag, e.g., hemagglutinin or Myc.

In some embodiments, a reporter sequence operably linked with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry.

In some embodiments, the transgene encodes a product which is useful in biology and medicine, such as RNA, proteins, peptides, enzymes, dominant negative mutants. In some embodiments, the RNA comprises a tRNA, ribosomal RNA, dsRNA, catalytic RNAs, small hairpin RNA, siRNA, trans-splicing RNA, and antisense RNAs. In some embodiments, the RNA inhibits or abolishes expression of a targeted nucleic acid sequence in a treated subject (e.g., a human or animal subject).

In some embodiments, the transgene may be used to correct or ameliorate gene deficiencies. In some embodiments, gene deficiencies include deficiencies in which normal genes are expressed at less than normal levels or deficiencies in which the functional gene product is not expressed. In some embodiments, the transgene encodes a therapeutic protein or polypeptide which is expressed in a host cell. In some embodiments, a dependoparvovirus particle may comprise or deliver multiple transgenes, e.g., to correct or ameliorate a gene defect caused by a multi-subunit protein. In some embodiments, a different transgene (e.g., each situated/delivered in a different dependoparvovirus particle, or in a single dependoparvovirus particle) may be used to encode each subunit of a protein, or to encode different peptides or proteins, e.g., when the size of the DNA encoding the protein subunit is large, e.g., for immunoglobulin, platelet-derived growth factor, or dystrophin protein. In some embodiments, different subunits of a protein may be encoded by the same transgene, e.g., a single transgene encoding each of the subunits with the DNA for each subunit separated by an internal ribozyme entry site (IRES). In some embodiments, the DNA may be separated by sequences encoding a 2A peptide, which self-cleaves in a post-translational event. See, e.g., Donnelly et al, J Gen. Virol., 78(Pt 1):13-21 (January 1997); Furler, et al, Gene Ther., 8(11):864-873 (June 2001); Klump et al., Gene Ther 8(10):811-817 (May 2001).

In some embodiments, virus particles comprising a genome are provided, wherein the genome includes a nucleic acid expression construct. The nucleic acid expression construct can include a heterologous transgene and one or more regulatory elements. In some embodiments, the particle delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 50-times, 64-times, 70-times, 100-times, 128-times, 200-times, 300-times, 400-times, 500-times, or 1000-times as compared to a virus particle comprising capsid polypeptides of SEQ ID NO: 1. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to non-macular retina tissue relative to macular tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to macular tissue relative to non-macular retina tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to macular tissue relative to trabecular meshwork tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, or 64-times as compared to a virus particle comprising capsid polypeptides of SEQ ID NO: 1, and wherein the increase in transduction is specific to non-macular retina tissue relative to trabecular meshwork tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to macular tissue and non-macular retina tissue relative to trabecular meshwork tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue relative to macular tissue and non-macular retina tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue relative to macular tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue relative to non-macular retina tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue, macular tissue, and non-macular retina tissue. In some embodiments, the particle delivers the payload to the eye with increased transduction specificity in one or more regions of the eye as compared to a virus particle comprising capsid polypeptides of SEQ ID NO: 1 without increased biodistribution in one or more regions of the eye as compared to a virus particle comprising capsid polypeptides of SEQ ID NO: 1. In some embodiments, the regulatory elements include a promotor. In some embodiments, the promoter is a ubiquitous or constitutive promoter active in a mammalian cell, for example a human cell, for example, in a human cell type of interest.

Examples of ubiquitous promoters include, but are not limited, to a CAG promoter (hybrid from a cytomegalovirus early enhancer element, a chicken-beta actin promoter, e.g., the first exon and the first intron of the chicken beta actin gene, and optionally the splice acceptor of the rabbit beta globin gene), chicken-beta actin promoter, CBA promoter, CMV promoter, human PGK promoter, ubiquitin promoter, human EF1-alpha promoter and fragments thereof. In some embodiments, the promoter is a tissue-specific promoter, for example, a promoter specific in ocular tissue or cells of the eye. Examples of ocular tissue-specific promoters include but are not limited to TBG promoters, hAAT promoters, CK8 promoters and SPc5-12 promoters, rho promoters, which are active in rods, or opsin promoters, which are active in cones. In some embodiments, the regulatory element includes a photoreceptor cell-specific regulatory element (e.g., promoter) such as, e.g., a rhodopsin promoter; a rhodopsin kinase promoter; a beta phosphodiesterase gene promoter; a retinitis pigmentosa gene promoter; an interphotoreceptor retinoid-binding protein (IRBP) gene enhancer; an IRBP gene promoter, an opsin gene promoter, a retinoschisin gene promoter, a CRX homeodomain protein gene promoter, a guanine nucleotide binding protein alpha transducing activity polypeptide 1 (GNAT1) gene promoter, a neural retina-specific leucine zipper protein (NRL) gene promoter, human cone arrestin (hCAR) promoter, and the PR2.1, PR1.7, PR1.5, and PR1.1 promoters. In some embodiments, the regulatory element includes, a retinal pigment epithelia (RPE) cell-specific regulatory element (e.g., a RPE-specific promoter), e.g., a regulatory element that confers selective expression of the operably linked gene in a RPE cell, such as, e.g., an RPE65 gene promoter, a cellular retinaldehyde-binding protein (CRALBP) gene promoter, a pigment epithelium-derived factor (PEDF aka serpin F1) gene promoter, and a vitelliform macular dystrophy (VMD2) promoter. In some embodiments, the regulatory element includes a promoter specific to a glial cell, e.g., a regulatory element that confers selective expression of the operably linked payload in a retinal glial cell, such as, e.g., a glial fibrillary acidic protein (GFAP) promoter. In some instances, the regulatory element includes a promoter that is specific to a bipolar cell (e.g., a bipolar-specific promoter), e.g., a regulatory element that confers selective expression of the operably linked payload in a bipolar cell, such as, e.g., a GRM6 promoter. In some embodiments, the promoter sequence is between 100 and 1000 nucleotides in length. In some embodiments, the promoter sequence is about 100, about 200, about 300, about 400, about 500, about 600, about 700, about 800, about 900 or about 1000 nucleotides in length. As used in the preceding sentence, “about” refers to a value within 50 nucleotides of the recited length. Suitable regulatory elements, e.g., promoters, may be readily selected by persons of skill in the art, such as those, but not limited to, those described herein.

In some embodiments, the nucleic acid expression construct comprises an intron. The intron may be disposed between the promoter and the heterologous transgene. In some aspects, the intron is disposed 5′ to the heterologous transgene on the expression construct, for example immediately 5′ to the heterologous transgene or 100 nucleotides or less 5′ to the heterologous transgene. In some aspects, the intron is a chimeric intron derived from human b-globin and Ig heavy chain (also known as b-globin splice donor/immunoglobulin heavy chain splice acceptor intron, or b-globin/IgG chimeric intron; Reed, R., et al. Genes and Development, 1989, incorporated herein by reference in its entirety). In other aspects, the intron is a VH4 intron or a SV40 intron.

As provided herein, in some embodiments, virus particles comprising a payload, wherein the payload includes a nucleic acid that includes a heterologous transgene are provided. In some embodiments, the heterologous transgene encodes an RNA interference agent, for example a siRNA, shRNA or other interfering nucleic acid.

In some embodiments, the payload includes a heterologous transgene that encodes a therapeutic polypeptide. In some aspects, the heterologous transgene is a human gene or fragment thereof. In some aspects, the therapeutic polypeptide is a human protein. In some embodiments, the heterologous transgene of the virus particle encodes a molecule useful in treating a disease, and the virus particle is administered to a patient in need thereof to treat said disease. Examples of diseases (and heterologous transgenes or molecules encoded by said heterologous transgenes) according to the present disclosure include: MPSI (alpha-L-iduronidase (IDUA)); MPS II—Hunter syndrome (iduronate-2-sulfatase (IDS)); Ceroid lipofuscinosis-Batten disease (CLN1, CLN2, CLN10, CLN13, CLN5, CLN11, CLN4, CNL14, CLN3, CLN6, CLN7, CLN8, CLN12); MPS Illa—Sanfilippo Type A syndrome (heparin sulfate sulfatase (also called N-sulfoglucosamine sulfohydrolase (SGSH)); MPS IIIB—Sanfilippo Type b syndrome (N-acetyl-alpha-D-glucosaminidase (NAGLU)); MPS VI—Maroteaux-Lamy syndrome (arylsulfatase B); MPS IV A—Morquio syndrome type A (GALNS); MPS IV B—Morquio syndrome type B (GLB1); Osteogenesis Imperfecgta Type I, II, III or IV (COL1A1 and/or COL1A2); hereditary angioedema (SERPING1, C1NH); Osteogenesis Imperfecta Type V (IFITM5); Osteogenesis Imperfecta Type VI (SERPINF1); Osteogenesis Imperfecta Type VII (CRTAP); Osteogenesis Imperfecta Type VIII (LEPRE1 and/or P3H1); Osteogenesis Imperfecta Type IX (PPIB); Gaucher disease type I, II and III (Glucocerebrosidase; GBA1); Parkinson's Disease (Glucocerebrosidase; GBA1 and/or dopamine decarboxylase); Pompe (acid maltase; GAA; hGAA); Metachromatic leukodystrophy (Aryl sulfatase A); MPS VII—Sly syndrome (beta-glucuronidase); MPS VIII (glucosamine-6-sulfate sulfatase); MPS IX (Hyaluronidase); maple syrup urine disease (BCKDHA, BCKDHB, and/or DBT); Niemann-Pick disease (Sphingomyelinase); Parkinson's disease (anti-alpha synuclein RNAi); Alzheimer's disease (anit-mutant APP RNAi); Niemann-Pick disease without sphingomyelinase deficiency (NPC1 or NPC gene encoding a cholesterol metabolizing enzyme); Tay-Sachs disease (alpha subunit of beta-hexosaminidase); Sandhoff disease (both alpha and beta subunit of beta-hexosaminidase); Fabry Disease (alpha-galactosidase); Fucosidosis (fucosidase (FUCA1)); Alpha-mannosidosis (alpha-mannosidase); Beta-mannosidosis (beta-mannosidase); Wolman disease (cholesterol ester hydrolase); Dravet syndrome (SCN1A, SCN1B, SCN2A, GABRG2); Parkinson's disease (Neurturin); Parkinson's disease (glial derived growth factor (GDGF)); Parkinson's disease (tyrosine hydroxylase); Parkinson's disease (glutamic acid decarboxylase; FGF-2; BDGF); Spinal Muscular Atrophy (SMN, including SMN1 or SMN2); Friedreich's ataxia (Frataxin); Amyotrophic lateral sclerosis (ALS) (SOD1 inhibitor, e.g., anti-SOD1 RNAi); Glycogen Storage Disease la (Glucose-6-phosphatase); XLMTM (MTM1); Crigler Najjar (UGTlA1); CPVT (CASQ2); spinocerebellar ataxia (ATXN2; ATXN3 or other ATXN gene; anti-mutant Machado-Joseph disease/SCA3 allele RNAi); Rett syndrome (MECP2 or fragment thereof); Achromatopsia (CNGB3, CNGA3, GNAT2, PDE6C); Choroidermia (CDM); Danon Disease (LAMP2); Cystic Fibrosis (CFTR or fragment thereof); Duchenne Muscular Dystrophy (Mini-/Micro-Dystrophin Gene); SARS-Cov-2 infection (anti-SARS-Cov-2 RNAi, SARS-Cov-2 genome fragments or S protein (including variants)); Limb Girdle Muscular Dystrophy Type 2C—Gamma-sarcoglycanopathy (human-alpha-sarcoglycan); Advanced Heart Failure (SERCA2a); Rheumatoid Arthritis (TNFR:Fc Fusion; anti-TNF antibody or fragment thereof); Leber Congenital Amaurosis (GAA); X-linked adrenoleukodystrophy (ABCD1); Limb Girdle Muscular Dystrophy Type 2C—Gamma-sarcoglycanopathy (gamma-sarcoglycan); Angelman syndrome (UBE3A); Retinitis Pigmentosa (hMERTK); Age-Related Macular Degeneration (sFLT01); Phelan-McDermid syndrome (SHANK3; 22q13.3 replacement); Becker Muscular Dystrophy and Sporadic Inclusion Body Myositis (huFollistatin344); Parkinson's Disease (GDNF); Metachromatic Leukodystrophy—MLD (cuARSA); Hepatitis C (anti-HCV RNAi); Limb Girdle Muscular Dystrophy Type 2D (hSGCA); Human Immunodeficiency Virus Infections; (PG9DP); Acute Intermittant Porphyria (PBGD); Leber's Hereditary Optical Neuropathy (PIND4v2); Alpha-1 Antitrypsin Deficiency (alphaIAT); X-linked Retinoschisis (RS1); Choroideremia (hCHM); Giant Axonal Neuropathy (GAN); Hemophilia B (Factor IX); Homozygous FH (hLDLR); Dysferlinopathies (DYSF); Achromatopsia (CNGA3 or CNGB3); Progressive supranuclear palsy (MAPT; anti-Tau; anti-MAPT RNAi); Omithine Transcarbamylase deficiency (OTC); Hemophilia A (Factor VIII); Age-related macular degeneration (AMD), including wetAMD (anti-VEGF antibody or RNAi); X-Linked Retinitis Pigmentosa (RPGR); Myotonic dystrophy Type 1 (DMPK; anti-DMPK RNAi, including anti-CTG trinucleotide repeat RNAi); Myotonic dystrophy Type 2 (CNBP); Facioscapulohumeral muscular dystrophy (D4Z4 DNA); oculopharynggeal muscular dystrophy (PABPN1; mutated PABPN1 inhibitor (e.g., RNAi)); Mucopolysaccharidosis Type VI (hARSB); Leber Hereditary Optic Neuropathy (ND4); X-Linked myotubular Myopathy (MTMl); Crigler-Najjar Syndrome (UGTlAl); Retinitis Pigmentosa (hPDE6B); Mucopolysaccharidosis Type 3B (hNAGLU); Duchenne Muscular Dystrophy (GALGT2); Alzheimer's Disease (NGF; ApoE4; ApoE2; ApoE3; Anti-ApoE RNAi); Familial Lipoprotein Lipase Deficiency (LPL); Alpha-1 Antitrypsin Deficiency (hAAT); Leber Congenital Amaurosis 2 (hRPE65v2); Batten Disease; Late Infantile Neuronal Lipofuscinosis (CLN2); Huntington's disease (HTT; anti-HTT RNAi); Fragile X syndrome (FMR1); Leber's Hereditary Optical Neuropathy (PIND4v2); Aromatic Amino Acid Decarboxylase Deficiency (hAADC); Retinitis Pigmentosa (hMERKTK); and Retinitis Pigmentosa (RLBP1).

In some aspects, the heterologous transgene encodes an antibody or fragment thereof (for example an antibody light chain, an antibody heavy chain, a Fab or an scFv). Examples of antibodies or fragments thereof that are encoded by the heterologous transgene include but are not limited to: an anti-Ab antibody (e.g. solanezumab, GSK933776, and lecanemab), anti-sortilin (e.g. AL-001), anti-Tau (e.g. ABBV-8E12, UCB-0107, and NI-105), anti-SEMA4D (e.g. VX15/2503), anti-alpha synuclein (e.g. prasinezumab, NI-202, and MED-1341), anti-SOD1 (e.g. NI-204), anti-CGRP receptor (e.g. eptinezumab, fremanezumab, or galcanezumab), anti-VEGF (e.g., sevacizumab, ranibizumab, bevacizumab, and brolucizumab), anti-EpoR (e.g., LKA-651,), anti-ALKl (e.g., ascrinvacumab), anti-C5 (e.g., tesidolumab, ravulizumab, and eculizumab), anti-CD105 (e.g., carotuximab), anti-CClQ (e.g., ANX-007), anti-TNFa (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g. siltuximab, clazakizumab, sirukumab, olokizumab, and gerilimzumab), anti-IL4R (e.g., dupilumab), anti-IL17A (e.g., ixekizumab and secukinumab), anti-IL5R (e.g. reslizumab), anti-IL-5 (e.g., benralizumab and mepolizumab), anti-IL13 (e.g. tralokinumab), anti-IL12/IL23 (e.g., ustekinumab), anti-CD 19 (e.g., inebilizumab), anti-IL31RA (e.g. nemolizumab), anti-ITGF7 mAb (e.g., etrolizumab), anti-SOST mAb (e.g., romosozumab), anti-IgE (e.g. omalizumab), anti-TSLP (e.g. nemolizumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab), optionally wherein the heavy chain (Fab and Fc region) and the light chain are separated by a self-cleaving furin (F)/F2A or furin (F)/T2A, IRES site, or flexible linker, for example, ensuring expression of equal amounts of the heavy and the light chain polypeptides.

In some embodiments, the payload comprises a nucleic acid encoding a gene product linked to a disorder of the eye, or a fragment thereof. Exemplary gene products linked to a disorder of the eye include, for example, ADP-ribosylation factor-like 6 (ARL6); BBSome interacting protein 1 (BBIP1); BBSome protein 1 (BBS1); BBSome protein 2 (BBS2); BBSome protein 4 (BBS4); BBSome protein 5 (BBS5); BBSome protein 7 (BBS7); BBSome protein 9 (BBS9); BBSome protein 10 (BBS10); BBSome protein 12 (BBS12); centrosomal protein 290 kDa (CEP290); intraflagellar transport protein 172 (IFT172); intraflagellar transport protein 27 (IFT27); inositol polyphosphate-5-phosphatase E (I3% NPP5E); inwardly-rectifying potassium channel subfamily J member 13 (KCNJ13); leucine zipper transcription factor like-1 (LZTFL1); McKusick-Kaufman syndrome protein (MKKS); Meckel syndrome type 1 protein (MKS1); nephronophthisis 3 protein (NPHP1); serologically-defined colon cancer antigen 8 (SDCCAG8); tripartite motif-containing protein 32 (TRIM32); tetratricopeptide repeat domain 8 (TTC8); Batten disease protein (CLN3); cytochrome P450 4V2 (CYP4V2); Rab escort protein 1 (CHM); PR (positive regulatory) domain-containing 13 protein (PRDM13); RPE-retinal G protein-coupled receptor (RGR); TEA domain family member 1 (TEAD1); arylhydrocarbon-interacting receptor protein-like 1 (AIPL1); cone-rod otx-like photoreceptor homeobox transcription factor (CRX); guanylate cyclase activating protein 1A (GUCAlA); retinal-specific guanylate cyclase (GUCY2D); phosphatidylinositol transfer membrane-associated family member 3 (PITPNM3); prominin 1 (PROM1); peripherin (PRPH); peripherin 2 (PRPH2); regulating synaptic membrane exocytosis protein 1 (RIMS1); semaphorin 4A (SEMA4A); human homolog of C. elegans unc119 protein (UNC119); ATP-binding cassette transporter-retinal (ABCA4); ADAM metallopeptidase domain 9 (ADAM9); activating transcription factor 6 (ATF6); chromosome 21 open reading frame 2 (C21orf2); chromosome 8 open reading frame 37 (C8orf37); calcium channel; voltage-dependent; alpha 2/delta subunit 4 (CACNA2D4); cadherin-related family member 1 (protocadherin 21) (CDHR1); ceramide kinase-like protein (CERKL); cone photoreceptor cGMP-gated cation channel alpha subunit (CNGA3); cone cyclic nucleotide-gated cation channel beta 3 subunit (CNGB3); cyclin M4 (CNNM4); guanine nucleotide binding protein (G protein); alpha transducing activity polypeptide 2 (GNAT2); potassium channel subfamily V member 2 (KCNV2); Phosphodiesterase 6C (PDE6C); Phosphodiesterase 6H (PDE6H); proteome of centriole 1 centriolar protein B (POC1B); RAB28 member of RAS oncogene family (RAB28); retina and anterior neural fold homeobox 2 transcription factor (RAX2); 11-cis retinol dehydrogenase 5 (RDH5); RP GTPase regulator-interacting protein 1 (RPGRIP1); tubulin tyrosine ligase-like family member 5 (TTLL5); L-type voltage-gated calcium channel alpha-1 subunit (CACNA1F); retinitis pigmentosa GTPase regulator (RPGR); rod transducin alpha subunit (GNA T1); rod cGMP phosphodiesterase beta subunit (PDE6B); rhodopsin (RHO); calcium binding protein 4 (CABP4); G protein-coupled receptor 179 (GPR179); rhodopsin kinase (GRK1); metabotropic glutamate receptor 6 (GRM6); leucine-rich repeat immunoglobulin-like transmembrane domains protein 3 (LRIT3); arrestin (s-antigen) (SAG); solute carrier family 24 (SLC24A1); transient receptor potential cation channel, subfamily M, member 1 (TRPM1); nyctalopin (NYX); green cone opsin (OPN1LW); red cone opsin (OPN1MW); blue cone opsin (OPN1SW); frataxin (FXN); inosine monophosphate dehydrogenase 1 (IMPDH1); orthodenticle homeobox 2 protein (OTX2); crumbs homolog 1 (CRB1); death domain containing protein 1 (DTHD1); growth differentiation factor 6 (GDF6); intraflagellar transport 140 Chlamydomonas homolog protein (IFT140); IQ motif containing B protein (IQCB1); lebercilin (LCA5); lecithin retinol acyltransferase (LRAT); nicotinamide nucleotide adenylyltransferase 1 (NMNAT1); RD3 protein (RD3); retinol dehydrogenase 12 (RDH12); retinal pigment epithelium-specific 65 kD protein (RPE65); spermatogenesis associated protein 7 (SPATA7); tubby-like protein 1 (TULP1); mitochondrial genes (KSS, LHON, MT-ATP6, MT-TH, MT-TL1, MT-TP, MT-TS2, mitochondrially encoded NADH dehydrogenases [MT-ND]); bestrophin 1 (BEST1); C1q and tumor necrosis-related protein 5 collagen (C1QTNF5); EGF-containing fibrillin-like extracellular matrix protein 1 (EFEMP1); elongation of very long fatty acids protein (ELOVL4); retinal fascin homolog 2, actin bundling protein (FSCN2); guanylate cyclase activating protein 1B (GUCAB); hemicentin 1 (HMCN1); interphotoreceptor matrix proteoglycan 1 (IMPG1); retinitis pigmentosa 1-like protein 1 (RP1L1); tissue inhibitor of metalloproteinases-3 (TTVIP3); complement factor H (CFH); complement factor D (CFD); complement component 2 (C2); complement component 3(C3); complement factor B (CFB); DNA-damage regulated autophagy modulator 2 (DRAM2); chondroitin sulfate proteoglycan 2 (VCAN); mitofusin 2 (MFN2); nuclear receptor subfamily 2 group F member 1 (NR2F1); optic atrophy 1 (OPA1); transmembrane protein 126A (TMEM126A); inner mitochondrial membrane translocase 8 homolog A (TIMM8A); carbonic anhydrase IV (CA4); hexokinase 1 (HK1); kelch-like 7 protein (KLHL7); nuclear receptor subfamily 2 group E3 (NR2E3); neural retina lucine zipper (NRL); olfactory receptor family 2 subfamily W member 3 (OR2W3); pre-mRNA processing factor 3 (PRPF3); pre-mRNA processing factor 4 (PRPF4); pre-mRNA processing factor 6 (PRPF6); pre-mRNA processing factor 8 (PRPF8); pre-mRNA processing factor 31 (PRPF31); retinal outer segment membrane protein 1 (ROM1); retinitis pigmentosa protein 1 (RP1); PIM-kinase associated protein 1 (RP9); small nuclear ribonucleoprotein 200 kDa (SNRNP200); secreted phosphoprotein 2 (SPP2); topoisomerase I binding arginine/serine rich protein (TOPORS); ADP-ribosylation factor-like 2 binding protein (ARL2BP); chromosome 2 open reading frame 71 (C2orf71); clarin-1 (CLRN1); rod cGMP-gated channel alpha subunit (CNGA1); rod cGMP-gated channel beta subunit (CNGB1); cytochrome P450 4V2 (CYP4V2); dehydrodolichyl diphosphate synthetase (DHDDS); DEAH box polypeptide 38 (DHX38); ER membrane protein complex subunit 1 (EMC1); eyes shut/spacemaker homolog (EYS); family with sequence similarity 161 member A (FAM161A); G protein-coupled receptor 125 (GPR125); heparan-alpha-glucosaminide N-acetyltransferase (HGSNAT); NAD(+)-specific isocitrate dehydrogenase 3 beta (IDH3B); interphotoreceptor matrix proteoglycan 2 (IMPG2); KIAA1549 protein (KIAA1549); kizuna centrosomal protein (KIZ); male germ-cell associated kinase (MAK); c-mer protooncogene receptor tyrosine kinase (MERTK); mevalonate kinase (MVK); NIMA (never in mitosis gene A)-related kinase 2 (NEK2); neuronal differentiation protein 1 (NEUROD1); cGMP phosphodiesterase alpha subunit (PDE6A); phosphodiesterase 6G cGMP-specific rod gamma (PDE6G); progressive rod-cone degeneration protein (PRCD); retinol binding protein 3 (RBP3); retinaldehyde-binding protein 1 (RLBP1); solute carrier family 7 member 14 (SLC7A14); usherin (USH2A); zinc finger protein 408 (ZNF408); zinc finger protein 513 (ZNF513); oral-facial-digital syndrome 1 protein (OFD1); retinitis pigmentosa 2 (RP2); retinoschisin (RS1); abhydrolase domain containing protein 12 (ABHD12); cadherin-like gene 23 (CDH23); centrosomal protein 250 kDa (CEP250); calcium and integrin binding family member 2 (CIB2); whirlin (DFNB31); monogenic audiogenic seizure susceptibility 1 homolog (GPR98); histidyl-tRNA synthetase (HARS); myosin VIIA (MYO7A); protocadherin 15 (PCDH15); harmonin (USH1C); human homolog of mouse scaffold protein containing ankyrin repeats and SAM domain (USHIG); dystrophin (DMD); norrin (NDP); phosphoglycerate kinase (PGK1); calpain 5 (CAPN5); frizzled-4 Wnt receptor homolog (FZD4); integral membrane protein 2B (ITM2B); low density lipoprotein receptor-related protein 5 (LRP5); micro RNA 204 (MIR204); retinoblastoma protein 1 (RB1); tetraspanin 12 (TSPAN12); chromosome 12 open reading frame 65 (C12orf65); cadherin 3 (CDH3); membrane-type frizzled-related protein (MFRP); ornithine aminotransferase (OAT); phospholipase A2 group V (PLA2G5); retinol-binding protein 4 (RBP4); regulator of G-protein signaling 9 (RGS9); regulator of G-protein signaling 9-binding protein (RGS9BP); ARMS2; excision repair cross-complementing rodent repair deficiency complementation group 6 protein (ERCC6); fibulin 5 (FBLN5); HtrA serine peptidase 1 (HTRA1); toll-like receptor 3 (TLR3); and toll-like receptor 4 (TLR4), opsin; rhodopsin; channel rhodopsin; halo rhodopsin, and the like.

In some embodiments, the virus particle comprises a heterologous transgene encoding a genome editing system. Examples include a CRISPR genome editing system (e.g., one or more components of a CRISPR genome editing system such as, for example, a guide RNA molecule and/or a RNA-guided nuclease such as a Cas enzyme such as Cas9, Cpf1 and the like), a zinc finger nuclease genome editing system, a TALEN genome editing system or a meganuclease genome editing system. In some embodiments, the genome editing system targets a mammalian, e.g., human, genomic target sequence. In some embodiments, the virus particle includes a heterologous transgene encoding a targetable transcription regulator. Examples include a CRISPR-based transcription regulator (for example, one or more components of a CRISPR-based transcription regulator, for example, a guide RNA molecule and/or a enzymatically-inactive RNA-guided nuclease/transcription factor (“TF”) fusion protein such as a dCas9-TF fusion, dCpf1-TF fusion and the like), a zinc finger transcription factor fusion protein, a TALEN transcription regulator or a meganuclease transcription regulator.

In some embodiments, components of a therapeutic molecule or system are delivered by more than one unique virus particle (e.g., a population that includes more than one unique virus particles). In other embodiments, the therapeutic molecule or components of a therapeutic molecule or system are delivered by a single unique virus particle (e.g., a population that includes a single unique virus particle).

The transgene may also encode any biologically active product or other product, e.g., a product desirable for study. Suitable transgenes may be readily selected by persons of skill in the art, such as those, but not limited to, those described herein.

Other examples of proteins encoded by the transgene include, but are not limited to, colony stimulating factors (CSF); blood factors, such as β-globin, hemoglobin, tissue plasminogen activator, and coagulation factors; interleukins; soluble receptors, such as soluble TNF-α. receptors, soluble VEGF receptors, soluble interleukin receptors (e.g., soluble IL-1 receptors and soluble type II IL-1 receptors), or ligand-binding fragments of a soluble receptor; growth factors, such as keratinocyte growth factor (KGF), stem cell factor (SCF), or fibroblast growth factor (FGF, such as basic FGF and acidic FGF); enzymes; chemokines; enzyme activators, such as tissue plasminogen activator; angiogenic agents, such as vascular endothelial growth factors, glioma-derived growth factor, angiogenin, or angiogenin-2; anti-angiogenic agents, such as a soluble VEGF receptor; a protein vaccine; neuroactive peptides, such as nerve growth factor (NGF) or oxytocin; thrombolytic agents; tissue factors; macrophage activating factors; tissue inhibitors of metalloproteinases; or IL-1 receptor antagonists.

The disclosure is further directed, in part, to a method of delivering a payload to a subject, e.g., an animal or human subject. In some embodiments, a method of delivering a payload to a subject comprises administering to the subject a virus particle, such as but not limited to a dependoparvovirus particle, comprising a variant polypeptide (e.g., described herein) comprising the payload, e.g., in a quantity and for a time sufficient to deliver the payload. In some embodiments, the dependoparvovirus particle is a dependoparvovirus particle described herein and comprises a payload described herein. In some embodiments, the particle delivers the payload to the eye. In some embodiments, the delivery to the eye is increased as compared to a particle without the variant capsid polypeptide or as compared to a wild-type capsid polypeptide.

### Methods of Treatment

In some embodiments, the methods of the invention are not methods for treatment of the human or animal body by therapy. In other embodiments, the methods of the invention are used for therapy.

The disclosure is directed, in part, to a method of treating a disease or condition in a subject, e.g., an animal or human subject. As used herein, the term “treating a disease or condition” refers to treating a manifest disease or condition, for example, where the subject is already suffering from one or more symptoms of the disease or condition, or refers to treating a pre-manifest disease or condition, for example, where the subject is identified as having a disease or condition but is not yet exhibiting one or more symptoms of the disease or condition. Pre-manifest conditions may be identified by, for example, genetic testing. In some embodiments, a method of treating a disease or condition in a subject comprises administering to the subject a virus particle, such as but not limited to a dependoparvovirus particle comprising a variant polypeptide described herein, e.g., comprising a payload described herein. In some embodiments, the dependoparvovirus particle, which comprises a variant polypeptide, comprising a payload described herein is administered in an amount and/or time effective to treat the disease or condition. In some embodiments, the payload is a therapeutic product. In some embodiments, the payload is a nucleic acid, e.g., encoding an exogenous polypeptide.

The viral particles, such as but not limited to, the dependoparvovirus particles comprising a variant polypeptide described herein or produced by the methods described herein can be used to express one or more therapeutic proteins to treat various diseases or disorders. In some embodiments, the disease or disorder is a cancer, e.g., a cancer such as carcinoma, sarcoma, leukemia, lymphoma; or an autoimmune disease, e.g., multiple sclerosis. Non-limiting examples of carcinomas include esophageal carcinoma; bronchogenic carcinoma; colon carcinoma; colorectal carcinoma; gastric carcinoma; hepatocellular carcinoma; basal cell carcinoma, squamous cell carcinoma (various tissues); bladder carcinoma, including transitional cell carcinoma; lung carcinoma, including small cell carcinoma and non-small cell carcinoma of the lung; adrenocortical carcinoma; sweat gland carcinoma; sebaceous gland carcinoma; thyroid carcinoma; pancreatic carcinoma; breast carcinoma; ovarian carcinoma; prostate carcinoma; adenocarcinoma; papillary carcinoma; papillary adenocarcinoma; cystadenocarcinoma; medullary carcinoma; renal cell carcinoma; uterine carcinoma; testicular carcinoma; osteogenic carcinoma; ductal carcinoma in situ or bile duct carcinoma; choriocarcinoma; seminoma; embryonal carcinoma; Wilm's tumor; cervical carcinoma; epithelieal carcinoma; and nasopharyngeal carcinoma. Non-limiting examples of sarcomas include fibrosarcoma, myxosarcoma, liposarcoma, angiosarcoma, endotheliosarcoma, lymphangiosarcoma, chondrosarcoma, chordoma, osteogenic sarcoma, osteosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include ependymoma, pinealoma, hemangioblastoma, acoustic neuroma, oligodendroglioma, glioma, astrocytoma, medulloblastoma, craniopharyngioma, menangioma, melanoma, neuroblastoma, and retinoblastoma. Non-limiting examples of leukemias include chronic myeloproliferative syndromes; T-cell CLL prolymphocytic leukemia, acute myelogenous leukemias; chronic lymphocytic leukemias, including B-cell CLL, hairy cell leukemia; and acute lymphoblastic leukemias. Examples of lymphomas include, but are not limited to, B-cell lymphomas, such as Burkitt's lymphoma; and Hodgkin's lymphoma.

In some embodiments, the disease or disorder is a genetic disorder. In some embodiments, the genetic disorder is sickle cell anemia, Glycogen storage diseases (GSD, e.g., GSD types I, II, III, IV, V, VI, VII, VIII, IX, X, XI, XII, XIII, and XIV), cystic fibrosis, lysosomal acid lipase (LAL) deficiency 1, Tay-Sachs disease, Phenylketonuria, Mucopolysaccharidoses, Galactosemia, muscular dystrophy (e.g., Duchenne muscular dystrophy), hemophilia such as hemophilia A (classic hemophilia) or hemophilia B (Christmas Disease), Wilson's disease, Fabry Disease, Gaucher Disease hereditary angioedema (HAE), and alpha 1 antitrypsin deficiency. Examples of other diseases or disorders are provided above in the “Methods of delivering a payload” section.

The viral particles, such as but not limited to, the dependoparvovirus particles comprising a variant polypeptide described herein or produced by the methods described herein can be used to express one or more therapeutic proteins to treat various diseases or disorders. In some embodiments, the disease or disorder is a disease or disorder of the eye, for example, retinitis pigmentosa; macular degeneration (e.g.; wet age-related macular degeneration), optic neuritis; Leber's congenital amaurosis; Leber's hereditary optic neuropathy; achromatopsia; X-linked retinoschisis; optic neuritis; choroideremia; optic atrophy; retinal cone dystrophy; retinopathy; retinoblastoma; glaucoma; Bardet-Biedl syndrome; Usher syndrome; aniridia; Friedreich's ataxia; vitelliform macular dystrophy; retinoblastoma; Stargardt disease; Charcot-Marie-Tooth disease; Fuch's dystrophy; propionic acidemia; or color blindness; corneal dystrophy; keratoconus; night blindness; dry eye; Bardet-Biedl syndrome; Batten's Disease; Bietti's Crystalline Dystrophy; chorioretinal atrophy; chorioretinal degeneration; cone or cone-rod dystrophies (autosomal dominant, autosomal recessive, and X-linked), congenital stationary night blindness (autosomal dominant, autosomal recessive, and X-linked); disorders of color vision, including achromatopsia (including ACHM2, ACHM3, ACHM4, and ACHM5), protanopia, deuteranopia, and tritanopia; Friedreich's ataxia; Leber's congenital amaurosis (autosomal dominant and autosomal recessive), including, but not limited to, LCA1, LCA2, LCA3, LCA4, LCA6, LCA7, LCA8, LCA12, and LCA15; Leber's Hereditary Optic Neuropathy; macular dystrophy (autosomal dominant and autosomal recessive), including, but not limited to, acute macular degeneration, Best vitelliform macular dystrophy, pattern dystrophy, North Carolina Macular Dystrophy, inherited drusen, Sorsby's fundus dystrophy, malattia levantanese, and genetically-determined retinopathy of prematurity; ocular-retinal developmental disease; ocular albinism; optic atrophies (autosomal dominant, autosomal recessive, and X-linked); retinitis pigmentosa (autosomal dominant, autosomal recessive, X-linked, and mitochondrially-inherited traits), examples of which include RP1, RP2, RP3, RP10, RP20, RP38, RP40, and RP43; X-linked retinoschisis; Stargardt disease; and Usher syndrome, including, but not limited to, USH1B, USH1C, USH1D, USH1F, USH1G, USH2A, USH2C, USH2D, AND USH3. Examples of complex genetic diseases include, but are not limited to, glaucoma (open angle, angle-closure, low-tension, normal-tension, congenital, neovascular, pigmentary, pseudoexfoliation); age-related and other forms of macular degeneration, both exudative and non-exudative forms (autosomal dominant and autosomal recessive), such as acute macular degeneration, vitelliform macular degeneration; retinopathy of prematurity; and Vogt Koyanagi-Harada (VKH) syndrome. Examples of acquired diseases include, but are not limited to, acute macular neuroretinopathy; anterior ischemic optic neuropathy and posterior ischemic optic neuropathy; Behcet's disease; branch retinal vein occlusion; choroidal neovascularization; diabetic retinopathy, including proliferative diabetic retinopathy and associated complications; diabetic uveitis; edema, such as macular edema, cystoid macular edema and diabetic macular edema; epiretinal membrane disorders; macular telangiectasia; multifocal choroiditis; non-retinopathy diabetic retinal dysfunction; ocular tumors; optic atrophies; retinal detachment; retinal disorders, such as central retinal vein occlusion, proliferative vitreoretinopathy (PVR), retinal arterial and venous occlusive disease, vascular occlusion, uveitic retinal disease; uveal effusion; retinal infective and infiltrative disease; optic nerve diseases such as acquired optic atrophy. Examples of traumatic injuries include, but are not limited to, histoplasmosis; optic nerve trauma; ocular trauma which affects a posterior ocular site or location; retinal trauma; viral infection of the eye; viral infection of the optic nerve; a posterior ocular condition caused by or influenced by an ocular laser treatment; posterior ocular conditions caused by or influenced by a photodynamic therapy; photocoagulation, radiation retinopathy; and sympathetic ophthalmia.

In some embodiments, administration of a viral particle, such as but not limited to, a dependoparvovirus particle comprising a variant polypeptide and comprising a payload (e.g., a transgene) to a subject induces expression of the payload (e.g., transgene) in a subject. In some embodiments, the expression is induced in the eye. In some embodiments, the production is increased in the eye as compared to an analogous particle with the wild-type capsid protein. The amount of a payload, e.g., transgene, e.g., heterologous protein, e.g., therapeutic polypeptide, expressed in a subject (e.g., the serum of the subject) can vary. For example, in some embodiments the payload, e.g., protein or RNA product of a transgene, can be expressed in the serum of the subject in the amount of less than about 5 μg/ml. For example, in some embodiments the payload, e.g., protein or RNA product of a transgene, can be expressed in the serum of the subject in the amount of at least about 9 g/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 ag/ml, at least about 700 μg/ml, at least about 800 μg/ml, at least about 900 μg/ml, or at least about 1000 μg/ml. In some embodiments, the payload, e.g., protein or RNA product of a transgene, is expressed in the serum of the subject in the amount of about 9 ag/ml, about 10 μg/ml, about 50 ag/ml, about 100 ag/ml, about 200 μg/ml, about 300 g/ml, about 400 g/ml, about 500 g/ml, about 600 μg/ml, about 700 g/ml, about 800 g/ml, about 900 g/ml, about 1000 g/ml, about 1500 μg/ml, about 2000 g/ml, about 2500 g/ml, or a range between any two of these values.

In some embodiments, the viral particle, such as but not limited to, the dependoparvovirus particle comprising a variant polypeptide and comprising a payload (e.g., a transgene) is administered to a subject via an injection. In some embodiments, the injection is a systemic injection, for example, intravenous, intraarterial, intramuscular, or subcutaneous injection. In some embodiments, the injection is an injection to the eye. In some embodiments, the injection is an intravitreal injection, intraorbital injection, retro-orbital injection, suprachoroidal injection, subretinal injection, subconjunctivital injection, or intracameral injection. In some embodiments, the injection is an intravitreal injection. In some embodiments, the injection is an intraorbital injection. In some embodiments, the injection is a retro-orbital injection. In some embodiments, the injection is a suprachoroidal injection. In some embodiments, the injection is a subretinal injection. In some embodiments, the injection is a subconjunctivital injection. In some embodiments, the injection is an intracameral injection.

Sequences disclosed herein may be described in terms of percent identity. A person of skill will understand that such characteristics involve alignment of two or more sequences. Alignments may be performed using any of a variety of publicly or commercially available Multiple Sequence Alignment Programs, such as “Clustal W”, accessible via the Internet. As another example, nucleic acid sequences may be compared using FASTA, a program in GCG Version 6.1. FASTA provides alignments and percent sequence identity of the regions of the best overlap between the query and search sequences. For instance, percent identity between nucleic acid sequences may be determined using FASTA with its default parameters as provided in GCG Version 6.1, herein incorporated by reference. Similar programs are available for amino acid sequences, e.g., the “Clustal X” program. Additional sequence alignment tools that may be used are provided by (protein sequence alignment; (http://www.ebi.ac.uk/Tools/psa/emboss_needle/)) and (nucleic acid alignment; http://www.ebi.ac.uk/Tools/psa/emboss_needle/nucleotide.html)). Generally, any of these programs may be used at default settings, although one of skill in the art can alter these settings as needed. Alternatively, one of skill in the art can utilize another algorithm or computer program which provides at least the level of identity or alignment as that provided by the referenced algorithms and programs. Sequences disclosed herein may further be described in terms of edit distance. The minimum number of sequence edits (i.e., additions, substitutions, or deletions of a single base or nucleotide) which change one sequence into another sequence is the edit distance between the two sequences. In some embodiments, the distance between two sequences is calculated as the Levenshtein distance.

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 21, 2020. When one gene or protein references a plurality of sequence accession numbers, all of the sequence variants are encompassed.

The invention is further illustrated by the following examples. The examples are provided for illustrative purposes only and are not to be construed as limiting the scope or content of the invention in any way.

### EXAMPLES

### Example 1

A library of 2.5E5 capsid variants of wild-type AAV2 were designed and cloned into plasmids to create a library of plasmids encoding the capsid variants (library for Library Experiment 1). Experimental results from Library Experiment 1 were assessed and machine learning models trained on this and other data, and used to design two separate libraries with 1E8 capsid variants of wild-type AAV2 each (libraries for Library Experiment 2). These libraries are significantly more diverse than the library tested in Library Experiment 1. Variants in one library were designed to maximize posterior eye transduction (including for example retina, macula, non-macular retina, neural retina and choroid/RPE) (posterior eye library) and variants in the other library were designed to maximize anterior eye transduction (including for example tissues of the trabecular meshwork and Schlemm's canal) (anterior eye library). Both libraries were designed to include variants which would produce virus particles. A library of AAV variant genomes encoding each variant's capsid and a unique capsid variant barcode identifier was cloned into three ITR plasmid backbones as described previously (Ogden et al. 2019). Each plasmid backbone contained a unique genomic identifier enabling analysis of biodistribution and transduction efficiencies via different routes of administration. The libraries were produced via transient triple transfection of adherent HEK293T followed by iodixanol gradient purification.

### In Vitro Evaluation of Library

Data was prepared as described below. To measure each variant's packaging efficiency (or “production”), barcodes from vector genomes in the plasmid and produced AAV library were prepared for illumine sequencing using two rounds of PCR. Production efficiency, normalized for presence in the input plasmid library, for each variant is expressed by comparing barcode sequencing levels for each variant in the produced vector pool to the barcode sequence levels for each variant in the input plasmid library used to create the vector pool. The measurements of variant frequency in the vector library also enable downstream normalization of biodistribution and transduction measurements by variant frequency in the input vector library. Production efficiency for variants in Library Experiment 1 is reported in Table 1A, and each reported value is reported as the log 2 production relative to the production of wild-type AAV2. Production efficiency for variants in the posterior eye library and anterior eye library for Library Experiment 2 are reported in Table 1B and 1C, and each reported value is reported as the log 2 production relative to the production of wild-type AAV2.

### In Vivo Evaluation of Library in Non-Human Primate

### Library Experiment 1

All NHP experiments were conducted in accordance with institutional policies and NIH guidelines. One young adult male and one young adult female cynomolgus macaque (Macaca fascicularis) weighing 2.4-2.9 kg seronegative for anti-AAV2 neutralizing antibodies (serum NAb titers <1:20 based on in vitro NAb assay) were selected for the study. Prior to test article administrations samples of blood, aqueous humor (50 L) and vitreous humor (up to 50 μL) were collected. The animals were anesthetized with ketamine and dexmedetomidine and received intravitreal (IVT; 4.8E11 vg/eye in 50 μL), intracameral (IC; 8.5E11 vg/eye in 50 μL) and intravenous (IV; 1.8-2.5E13 vg/kg) injections of the vector libraries. During the in-life period the animals were monitored for signs of ocular inflammation via indirect ophthalmoscopy and slit-lamp biomicroscopy and treated with weekly IM injections of steroids (methylprednisolone, 40-80 mg) and topical steroids (Durezol), and atropine as needed according to the animal facility's SOPs and recommendations from the veterinarian. Serum samples were collected at 1 h, 4 h and 24 h, and weekly after the injections. The animals were sacrificed 4 weeks after the injections and tissues were collected for biodistribution and transduction analyses.

### Library Experiment 2

The evaluations of anterior eye library variants and posterior eye library variants in Library Experiment 2 were carried out in Cynomolgus macaques. Both eyes of two non-naive animals with low serum anti-AAV2 NAb titers (<1:10) received intravitreal injections of the posterior eye library and intracameral injections of the anterior eye library at total doses of 7.3E11 vg/eye (3.9E11 vg for IC, 3.4E11 vg for IVT), with both libraries injected via both routes of administration in each eye. Ophthalmic examinations were performed weekly during the 4 week in-life period to monitor levels of ocular inflammation.

### Tissue Processing and Data Analysis for Library Experiment 1 and Library Experiment 2

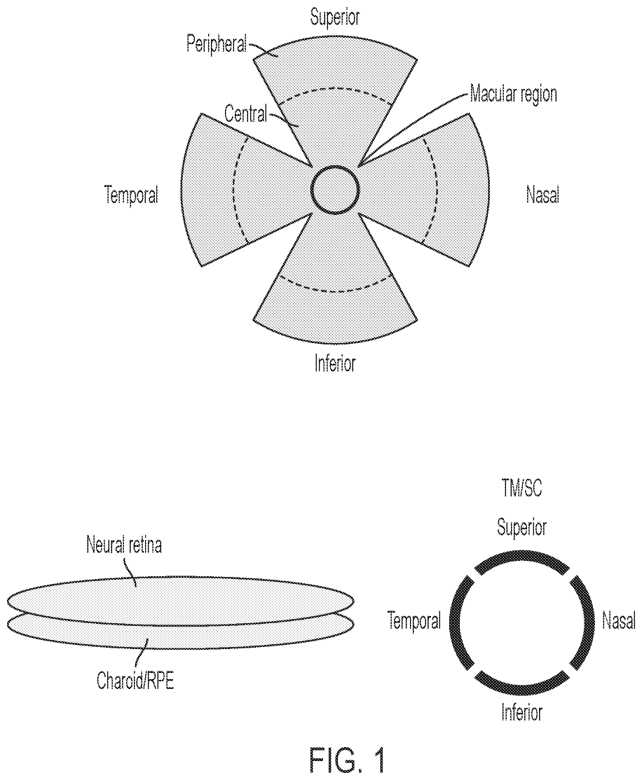

Retinas and trabecular meshwork were dissected as shown in FIG. 1. A list of other tissue samples collected is shown in Table 5. All samples were collected into RNAlater® (Sigma-Aldrich) and incubated overnight at RT, after which the RNAlater® was drained and samples were frozen at −80° C. In addition, samples of aqueous humor, vitreous humor, serum, and cerebrospinal fluid were collected at necropsy and stored at −80° C.

TABLE 5
List of tissues collected.
Tissue

Adrenal gland
brain (cortical slices) coronal axis
dorsal root ganglion (cervical)
dorsal root ganglion (thoracic)
dorsal root ganglion (lumbar)
gonad (testes and ovaries)
heart, basal (left atrium)
heart, apex
heart, right ventricle
kidney
liver
Lung (superior lobe)
lymph nodes, cervical
skeletal muscle, bicep brachii
skeletal muscle, diaphragm
skeletal muscle, quadriceps
spinal cord (cervical)
spinal cord (thoracic)
spinal cord (lumbar)
spleen

For biodistribution and transduction analyses, total DNA and RNA was extracted from tissue samples with Trizol/chloroform and isopropanol precipitation. RNA samples were treated with TURBO DNase (Invitrogen). Reverse transcription was done with Protoscript II Reverse Transcriptase (NEB) with primers that were specific to the vector transgene and included unique molecular identifiers (UMIs). Control reactions lacking the reverse transcriptase enzyme (−RT control) were also prepared. Quantification of biodistribution and transduction was done with Luna Universal Probe qPCR Master Mix (NEB) using primers and probes specific to the transgene construct. Finally, samples were prepared for next-generation sequencing by amplifying the transgene barcode regions with primers compatible with Illumina NGS platform and sequenced with NextSeq 550 (Illumina).

After sequencing, the barcode tags were extracted from reads with the expected amplicon structure, and the abundance (number of reads or number of UMIs) of each barcode was recorded. Analyses were restricted to the set of barcodes that were present in the input plasmid sample and that did not contain errors in the variant sequence, as measured by a separate sequencing assay that targeted the variant regions of the input plasmid sample.

To aggregate packaging replicates, the read counts from replicate virus production samples were summed. To aggregate transduction samples, the UMI counts from samples from the same tissue were summed.

Virus packaging, biodistribution and transduction of tissue were calculated using a Bayesian model with aggregated production, biodistribution and/or transduction samples as the input. Briefly, probabilistic programming and stochastic variational inference were used to model the measurement process and sources of decoupling (e.g., cross-packaging, template switching, and errors in DNA synthesis) between the actual test virus particles and their designed sequences, and to calculate virus production, biodistribution and transduction (in various tissue samples), and error rates. The output was the log 2-transformed mean of the calculated distribution relative to the wild-type (WT) AAV2. Thus, positive values indicate better performance than WT for the measured property, and negative values indicate worse-than-WT performance. Transduction and biodistribution for Library Experiment 1 is reported in Table 1A (transduction) and Table 3 (biodistribution). Transduction and biodistribution for Library Experiment 2 is reported in Tables 1B-1C (transduction) and Table 4A (biodistribution). Where indicated, macula transduction and biodistribution refers to measurements taken from tissues consisting of the neural retina layer of the macula. Retina or non-macula retina transduction and biodistribution refers to measurements taken from tissues consisting of the neural retina layer of the non-macular retina areas of the eye. Measurements including the choroid and/or RIPE are taken from tissues consisting of the choroid layer of the whole retina. Without being bound by theory, because the complexity of the libraries for this experiment were high relative to the total overall dose (1E8 variants with total doses of approximately 7E11 vg/eye), relative transduction and biodistribution values from Library Experiment 2 are compressed and, therefore, represent an underrepresentation of the relative transduction and biodistribution rates. The results demonstrate however, that the relative rank ordering of the variants that were included in both Library Experiment 1 and Library Experiment 2 was consistent (Spearman correlation=0.72), confirming the top variants as having significant transduction (for example, better than wild-type AAV2) improvements. Variants from this experiment are included in a follow-on library experiment of similar complexity to Library Experiment 1 (e.g., 1-2E5 variants per library), and as described in Example 2, and properties are confirmed as described herein for Library Experiment 1 and in Example 2.

### Example 2

The virus particles comprising the variant capsids provided in Table 2 (sequences) are produced individually via transient triple transfection of adherent HEK293T followed by iodixanol gradient purification. Each variant capsid is produced with a genome encoding a unique barcode and a fluorescent reporter gene under the control of a ubiquitous promoter. Production efficiency is assessed as described above. Equivalent amounts (vg) of each virus particle are pooled (approximately 50-100 variants total) in equimolar amounts, and injected into AGM or other non-human primate, for example, Cynomolgus macaque at doses used in Example 1. Virus properties, including biodistribution and tissue transduction are assessed, for example, as described in Example 1.

### REFERENCES

Ogden P J, Kelsic E D, Sinai S, Church G M. Comprehensive AAV capsid fitness landscape reveals a viral gene and enables machine-guided design. Science. 2019 Nov. 29; 366(6469):1139-1143. doi: 10.1126/science.aaw2900. PMID: 31780559; PMCID: PMC7197022.

The results show that variant capsid polypeptides provided for herein produce virus particles that have increased packaging, increased biodistribution, increased transduction and/or increased expression of a transgene (payload) in various regions of the eye relative to a wild-type AAV2 upon intravitreal or intracameral injection. In addition, the variant capsid polypeptides described herein provide selective biodistribution and/or expression in regions of the eye that include target cell populations for gene therapy (for example, macula-selectivity, non-macula retina selectivity, macula/retina selectivity and/or trabecular meshwork selectivity). Without limitation, the capsid polypeptides, nucleic acids and virus particles described herein are used to deliver therapeutics to the eye, e.g., to certain cell types of the eye, and are used to treat disorders of the eye as described herein, with higher efficiency.

### Example 3

### In Vivo Evaluation of Medium-Throughput Library in Non-Human Primate (Library Experiment 3)

Variants in this study were selected from internal data sets acquired from ocular non-human primate (NHP) experiments based on an algorithm that optimizes capsid performance while balancing diversity and measurement uncertainty. In one arm of the experiment, variants were selected based on their transduction performance in the posterior eye (including, for example, the neural retina and choroid/RPE) and anterior eye (including, for example, tissues of the trabecular meshwork and Schlemm's canal) via intravitreal (IVT) delivery. In the other arm of the experiment, variants were selected based on their transduction performance in the anterior eye via intracameral (IC) delivery. Without being bound by theory, capsids with specificity for one region of the eye or specific tissues and/or cell types within that region could have benefits for gene therapies by providing increased targeting to the tissue or cell type of interest and a better safety profile by de-targeting other ocular tissues or cell types. The study also included variants that contain stop codons in VP1 and VP2 as transduction negative controls (expected to produce virus but not transduce cells) and containing VP3 stop codons as production negative controls (not expected to produce virus). The study also included variants having WT AAV2 capsid polypeptides, as well as variants having the capsid polypeptides of SEQ ID NO: 60.

The virus particles for the study, including those comprising a selection of the variant capsids provided in Table 2, were produced individually via separate transient triple transfection of adherent HEK293T cells followed by co-purification by iodixanol gradient. The representation of each individual variant within the virus pool is measured via NGS. Variants identified with low initial productivity yields were produced again individually in a separate production round and combined with virus from the previous productions to balance the representation of every variant to be within 10-fold range in the final test article. Final test article included each variant at an amount of 1E9-9E9 vg/eye for IVT, and 1E9-1E10 vg/eye for IC test articles, as measured by ddPCR for final titer and NGS analysis for variant representation. Production efficiencies for individual variants, relative to production efficiency of wild-type AAV2, are provided in Tables 1D and 1E.

Each variant capsid was included in a virus particle that included a genome bearing identifying unique barcode sets of 8 as well as diverse random sequence IDs for quantification, providing a measure of biological replicates within the study. Each genome further contains a sequence encoding a fluorescent reporter gene under the control of a ubiquitous Cbh promoter.

All NHP experiments were conducted in accordance with institutional policies and NIH guidelines. Four young adult male and one adult female cynomolgus macaques (Macaca fascicularis) weighing 2.2-2.9 kg, seronegative for anti-AAV2 neutralizing antibodies (NAb) were selected for the study (seronegativity status being serum NAb titers <1:20 based on in vitro NAb assay). Prior to test article administration, samples of blood, aqueous humor (50 L), and vitreous humor (up to 50 L) were collected. The animals were anesthetized with ketamine and dexmedetomidine and received bilateral (IVT; 4.27E11 vg/eye in 100 uL) (IC; 2.00E11 vg/eye in 50 uL) injections of vector libraries. This resulted in approximately 1E9-9E9 vg of each individual variant delivered to each eye by the IVT route of administration, and 1E9-1E10 vg of each individual variant delivered to each eye by the IC route of administration. During the in-life period, the animals were monitored weekly for signs of ocular inflammation via indirect ophthalmoscopy and slit-lamp biomicroscopy and treated with weekly IM injections of steroids (methylprednisolone, 80 mg) and topical steroids (Durezol), and atropine as needed according to the animal facility's SOPs and recommendations from the veterinarian. Confocal scanning laser ophthalmoscopy (cSLO) with green fluorescent protein (GFP) imaging using the Heidelberg Spectralis HRA/OCT system was used to take fluorescent images of each eye prior to necropsy. The animals were sacrificed four weeks after dosing, and tissues were collected for biodistribution and transduction analyses.

Retinas and trabecular meshwork were dissected as shown in FIG. 1. All ocular tissues were flash-frozen on dry ice following dissection. A list of all ocular tissues collected is shown in Tables 6A and 6B.

TABLE 6A
Samples from Medium Throughput Study
Front of the Eye Samples

| Sample | |
|---|---|
| # | Region |
| 1 | Iris/ciliary body (superior |
| 2 | Iris/ciliary body (nasal) |
| 3 | Iris/ciliary body (inferior) |
| 4 | Iris/ciliary body (temporal) |
| 5 | Region containing Trabecula/Schlemm's (superior) |
| 6 | Region containing Trabecula/Schlemm's (nasal) |
| 7 | Region containing Trabecula/Schlemm's (inferior) |
| 8 | Region containing Trabecula/Schlemm's (temporal) |
| 9 | Cornea (combined) |
| 10 | Remaining limbal tissue (combined) |

TABLE 6B
Samples from Medium Throughput Study
Retinal and Choroid/RPE Samples

| Sample # | Retinal region | Retinal cell layer |
|---|---|---|
| 1 | Macular region | neural retina |
| 2 | Superior petal (central) | neural retina |
| 3 | Superior petal (peripheral) | neural retina |
| 4 | Nasal petal (central) | neural retina |
| 5 | Nasal petal (peripheral) | neural retina |
| 6 | Inferior petal (central) | neural retina |
| 7 | Inferior petal (peripheral) | neural retina |
| 8 | Temporal petal (central) | neural retina |
| 9 | Temporal petal (peripheral) | neural retina |
| 10 | Macular region | choroid/RPE |
| 11 | Superior petal (central) | choroid/RPE |
| 12 | Superior petal (peripheral) | choroid/RPE |
| 13 | Nasal petal (central) | choroid/RPE |
| 14 | Nasal petal (peripheral) | choroid/RPE |
| 15 | Inferior petal (central) | choroid/RPE |
| 16 | Inferior petal (peripheral) | choroid/RPE |
| 17 | Temporal petal (central) | choroid/RPE |
| 18 | Temporal petal (peripheral) | choroid/RPE |

No significant deviations were reported during the in-life period of the study. Moderate ocular inflammation was observed in all 5 animals by week 2. In week 3, topical ocular medications (atropine and durezol) were applied onto the eyes of each animal in addition to weekly steroid treatment (methylprednisolone, 80 mg). Inflammation was reduced by 4 weeks but was not fully resolved before termination. 4 weeks following the injection, the animals were sacrificed and transduction was measured by NGS of variant associated barcodes isolated from the cDNA of bulk tissue samples and by single nuclear RNA sequencing (snRNA-seq). Through analysis of this data (see further details below), transduction and biodistribution rates were determined for the variants across major cell types within the eye by each route of administration. Analysis of the data allowed for observing correlations, comparing transduction efficiency between bulk and single-cell measurements, and identifying cell types within each tissue that were transduced by each variant. In addition to identifying highly promising capsids for improved ocular gene therapy delivery, this high-resolution dataset provides validation of our prior data measurements in other library contexts and valuable input data for future machine-guided design directed at improving cell transduction, specificity, and tissue distribution. Results from bulk tissue for variants included in this medium throughput study described in this Example 3 are shown in Tables 1D, 1E, 1F, 4B, and 4C (IVT administration) and Tables 1G and 4D (IC administration). Unless otherwise noted, all values are relative to wild-type AAV2. For instance, values for trabecular transduction (IVT and IC administration) and trabecular biodistribution (IC administration) are relative to that of a virus particle having capsid polypeptides of SEQ ID NO: 60. This was chosen because in these tissues by these routes of administration, virus particles with WT AAV2 capsid polypeptides were not detected.

Details of the tissue processing and NGS analysis for bulk tissue sample experimental workflow are described below:

### Tissue Dissection, Homogenization and Nucleic Acid Extraction

Tissue samples are dissected inside the cold chamber of a cryostat (−20° C.) down to <100 mg pieces using clean disposable scalpels and put into a Nuclease-free Eppendorf tube. Scalpels and forceps were changed in between tissues to avoid cross-contamination. 1 mL of Trizol was added into each tube along with a stainless steel bead (5 mm), and tissues are homogenized using TissueLyser II (QIAGEN) according to the manufacturer's recommendations. 200 uL chloroform was added into each homogenized sample, and samples were vortexed to create phase separation.

RNA was extracted from the upper aqueous phase using an RNeasy (QIAGEN) kit according to the manufacturer's recommendations, and was further treated with Turbo™ DNase (Thermo Fisher Scientific) to remove any vector DNA contamination in the RNA sample. Reverse transcription was performed using a specific RT primer that anneals to the vector transcript to generate the targeted cDNA library. DNA was extracted from the organic layer using DNeasy Blood and Tissue Kit (QIAGEN).

### NGS Library Preparation

Each cDNA and DNA sample was amplified using pre-indexing primers specific to the target region that contains the barcode and ID with Illumina handles (R1 and R2) as overhangs. Each sample amplification was tracked live via qPCR and terminated once the Rn of a sample reached the inflection point. Pre-indexed samples were indexed using custom Illumina indexing primers to index every tissue sample. These indexed libraries are then sequenced using an Illumina NextSeq200 Sequencer.

### NGS Sample Parsing and Analysis

### Bulk NGS Sequencing Data Analysis

After sequencing, the barcode tags were extracted from reads with the expected amplicon structure, and the abundance (number of reads or number of UMIs) of each barcode was recorded. For each route of administration, analyses were restricted to the set of designed barcodes in the corresponding test article. To aggregate biodistribution samples, read counts from samples from the same tissue were summed. To aggregate transduction samples, the number of UMIs from samples from the same tissue were summed.

Biodistribution and transduction of tissue were calculated by normalizing aggregated biodistribution or transduction counts with input virus abundance. For neural retina, the rates were calculated as fold change relative to the wild-type (WT) AAV2. The neural retina measurements are reported as mean and standard deviation of eye replicates (n=8). For trabecular meshwork, the rates were calculated as fold change relative to a virus particle having capsid polypeptides of SEQ ID NO: 60, since WT AAV2 transduction was too low or dropped out, and was unable to be used to establish a reliable baseline. The trabecular meshwork measurements are reported as mean and standard error of barcode replicates (n=8).

Data from different numbers of tissue samples are described herein. To generate the data shown in Tables 1D and 4B, the number of tissue samples for cDNA and vDNA that were processed are shown in Tables 7A and 7B, respectively. Additional tissue samples were collected and used to generate the data shown in Tables 1E, 1F, 1G, 4C, and 4D, and Tables 8A and 8B show the number of tissue samples for cDNA and vDNA, respectively. Tissue samples taken from the whole retina, both the macular and non-macular regions, were used to generate the neural retina transduction and biodistribution results shown in Tables 3D, 1E, and 4C. Tissue samples taken from the macular region of the retina were used to generate the macular transduction and biodistribution results shown in Tables 1F and 4C. Tissue samples from the trabecular meshwork and Schlemm's canal were used to generate the trabecular transduction and biodistribution results shown in Tables 1F, 1G, 4C, and 4D.

TABLE 7A
cDNA Number of Samples

| | | NHP | NHP | NHP | NHP | NHP |
|---|---|---|---|---|---|---|
| Organ | Tissue | 1 | 2 | 3 | 4 | 5 |
| eye | neural retina | 9 | 10 | 11 | 3 | 3 |
| eye | trabecular meshwork; | 0 | 0 | 1 | 0 | 0 |
| | Schlemm's canal | | | | | |

TABLE 7B
vDNA Number of Samples

| | | NHP | NHP | NHP | NHP | NHP |
|---|---|---|---|---|---|---|
| Organ | Tissue | 1 | 2 | 3 | 4 | 5 |
| eye | neural retina | 5 | 6 | 6 | 3 | 3 |
| eye | trabecular meshwork; | 4 | 4 | 4 | 0 | 0 |
| | Schlemm's canal | | | | | |

TABLE 8A
cDNA Number of Samples

| | | NHP | NHP | NHP | NHP | NHP |
|---|---|---|---|---|---|---|
| Organ | Tissue | 1 | 2 | 3 | 4 | 5 |
| eye | neural retina | 10 | 11 | 13 | 3 | 4 |
| eye | trabecular meshwork; | 0 | 1 | 1 | 1 | 1 |
| | Schlemm's canal | | | | | |

TABLE 8B
vDNA Number of Samples

| | | NHP | NHP | NHP | NHP | NHP |
|---|---|---|---|---|---|---|
| Organ | Tissue | 1 | 2 | 3 | 4 | 5 |
| eye | neural retina | 5 | 7 | 8 | 3 | 4 |
| eye | trabecular meshwork; | 4 | 5 | 4 | 1 | 1 |
| | Schlemm's canal | | | | | |

### Details of the Single-Nuclei RNA Sequencing and Analysis Process is Provided Below:

Single-cell RNA sequencing has been previously demonstrated to allow characterization of cell-type specific tropism of barcoded rAAVs (Brown et al., Front. Immunol., 2021, which is incorporated by reference in its entirety). However, obtaining single cell suspension from certain tissue types and/or flash frozen samples from externally-sourced NHP studies can be extremely challenging. We developed an approach that combines single-nuclei RNA sequencing (snRNA-Seq) with targeted amplicon sequencing to reliably detect cell-type specific transduction from up to 50-100 barcoded rAAVs with minimal sequencing depth, with an initial focus on tissues of the eye, and applied these methods to the tissues collected from the experiment described in this example. To implement this approach we have: 1) developed protocols for isolation of high quality single nuclei suspensions from flash frozen NHP ocular tissues (e.g. neural retina and trabecular meshwork), 2) used the 10× Genomics Chromium platform to encapsulate these nuclei and generate gene expression libraries for reliable identification of cell types, and 3) selectively amplified (for sequencing) barcoded viral transcripts that were captured using either the 10× CS1 feature designed into the viral genomes or using the 10× oligo dT capture probes. Using this approach we investigated cell-type specific tropism of multiple rAAVs in Cynomolgus macaque neural retina and the trabecular meshwork. Our snRNA-seq gene expression analysis identified all the major neural retina and trabecular meshwork cell types including therapeutically relevant cells such as rods, cones, retinal ganglion cells (RGCs), and cells responsible for draining of ocular fluids in the anterior eye, such as beam cells and juxtacanalicular (JCT) cells. Viral transduction events, as assessed from our targeted library sequencing, were detected in almost all clusters and we could successfully quantify differences in transduction rates between rAAVs and benchmarks. Overall, we demonstrate that snRNA-seq can be used to both effectively determine cell-type specific tropism of barcoded rAAVs, and quantitate relative transduction between multiple rAAVs in a single experiment. These developments open up opportunities for further designing and validating rAAVs capable of cell-type specific targeting for gene therapy. Additionally, this approach enables a medium throughput identification of rAAVs with desired properties for further study.

Details of the single nuclear experimental workflow that was applied to the medium throughput study described in this example are below:

### Materials

EZ lysis buffer+0.2 U/μl of murine RNAse inhibitor
1×PBS+9% BSA+0.2 U/μl murine RNAse inhibitor
1×PBS+5% BSA+0.2 U/μl of murine RNAse inhibitor/Wash
1×PBS+2% BSA+0.2 U/μl of murine RNAse inhibitor
Two 15 ml tubes pre-coated with 2% BSA+1×PBS+0.2 U/μl RNAse inhibitor
Six 1.5 ml protein lobind tubes pre-coated with 2% BSA+1×PBS+0.2 U/l RNAse inhibitor
One 2 ml dounce homogenizer+Pestle A and Pestle B
2×0.4 μm filters
2×0.7 μm filters
1 ml wide bore pipette tips
Hemocytometer
4-6 0.5 ml protein lobind tube with 15 μl of 1×PBS+5% BSA+
   Single Nuclei Dissociation from Retina and Trabecular Meshwork

Mincing: For the retina, the tissue sample was placed in a tube on ice and 100 μl of EZ lysis buffer+RNAse inhibitor was added. The tissue was minced with a pair of microscissors for about 1 min while holding the tube on ice. 50 μl of the minced sample was transferred to a 2 ml dounce homogenizer. 1-2 ml of Trizol was added to the rest of the sample for paired bulk RNA extraction and sequencing.

For the trabecular meshwork, the tissue was transferred to a new 1.5 ml tube and 50 μl of EZ lysis buffer+RNAse inhibitor was added. The tissue was minced with a pair of microscissors for about 1 min while holding the tube on ice. The minced tissue was transferred to a 2 ml dounce homogenizer.

Dounce homogenization: More EZ lysis buffer+RNAse inhibitor was added to the dounce homogenizer with sample to make up the volume to 2 ml. A loose fitting pestle (Pestle A) was used to dounce the sample with 10 steady strokes (about 1 stroke per second). Only for trabecular meshwork (TM) tissue, any larger pieces of tissue that remained and may clog dounce B were carefully removed and discarded using a 1 ml pipette tip. The sample was allowed to stand on ice for 20 seconds and then was dounced with a tight fitting pestle (Pestle B) with 5 steady strokes. The sample was again allowed to stand on ice for 20 seconds and dounced for another 5 strokes with Pestle B.

Filtration and clean up: Post douce the sample (2 ml) was immediately transferred to a 15 ml falcon tube containing 2 ml of 9.5%+1×PBS+RNAse Inhibitor. The sample was mixed and first filtered through a 70 micron filter and then through a 40 micron filter. A small (5 μl) aliquot of the filtered sample was diluted 1:4 in 1×PBS+2% BSA+propidium iodide (PI) for counting.

The sample (˜4 ml) was centrifuged at 200 RCF for 6 mins at 4° C. The supernatant was discarded and the pellet was resuspended in appropriate volume of 5% BSA+1×PBS+RNAse-Inhibitor+PI.

FACS cleanup: The nuclei were then sorted on a WOLF sorter by gating for intact nuclei that were positively stained for PI and discarding any doublets by gating with the area under the curve for PI as a proxy for doublets. The FACS cleaned nuclei were centrifuged at 200 RFC for 5 mins at 4° C. The pellet was resuspended in 2% BSA+1×PBS+RNAse-inhibitor and counted.

Final nuclei concentration was adjusted as needed for 10× encapsulation. 10× Encapsulation and library preparation: The 10× Chromium platform (10× Genomics) was used for single cell encapsulation as per the manufacturer's standard instructions. Reverse transcription was performed as per 10× protocols. cDNA amplification was performed using the 10× feature barcode cDNA amplification kit which allows amplification of both CS1 and oligo-dT captured transcripts.

Post cDNA amplification a portion of the cDNA library was used to generate a gene expression library as per 10× standard protocol and the library was quality controlled and sequenced as per standard 10× protocols. A small portion of the same cDNA library was used to generate targeted libraries by PCR amplifying the Dyno barcode region. For targeted amplification of viral transcripts captured by the CS1 oligo, primers binding to the Nextera Handle in combination with a viral transcript specific primer were used. For targeted amplification of viral transcripts captured by oligo-dT, primers binding to either the TruSeq Handle in combination with a viral transcript specific primer were used. When amplifying using the TruSeq-Handle primer, a gel extraction step was performed right after the targeted amplification to select out the product of interest from the larger linearly amplified background.

Once the targeted amplification product was purified we performed pre-indexing and indexing PCRs and sequenced the libraries using an Illumina NextSeq2000 sequencer.

### Single Nuclei RNA Sequencing Data Analysis:

Gene expression libraries were demultiplexed using Illumina bcl-convert with default settings, then aligned to Macaca fascicularis reference genome (v6.0, assembly GCA_011100615.1) and quantified using the CellRanger pipeline v7.1.0 with intron mode activated. Doublet detection and filtering was performed using Scrublet package v0.2.3. Dimensionality reduction, batch effect removal, clustering, and identification of marker genes were carried out using Scanpy v1.9.3. Identification of cell types was performed with an in-house algorithm that projects cell type labels from reference datasets, which we curated from published literature (Swamy et al., GigaScience, Vol. 10, 2021; van Zyl et al., PNAS, Vol. 117, 2020, each of which is incorporated herein by reference in its entirety). Targeted libraries were processed using an in-house pipeline to obtain the identities of transducing variants and the 10× feature barcode. To completely remove chimeric molecules, we performed transcript per transcript (TPT) filtering (Dixit, bioRxiv 093237, 2021, which is incorporated herein by reference in its entirety) with a threshold of 0.5. Targeted libraries were then filtered against gene expression libraries to associate cell type information and limit the analysis to valid cell barcodes. The data was further filtered with a cut-off of 10 reads per molecule to remove any remaining sequencing artifacts. Finally, cells with more than 20 observed transduction events, which likely represent clumping artifacts, were excluded from downstream analysis.

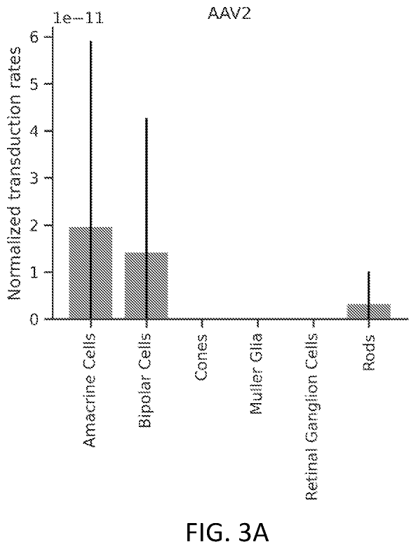

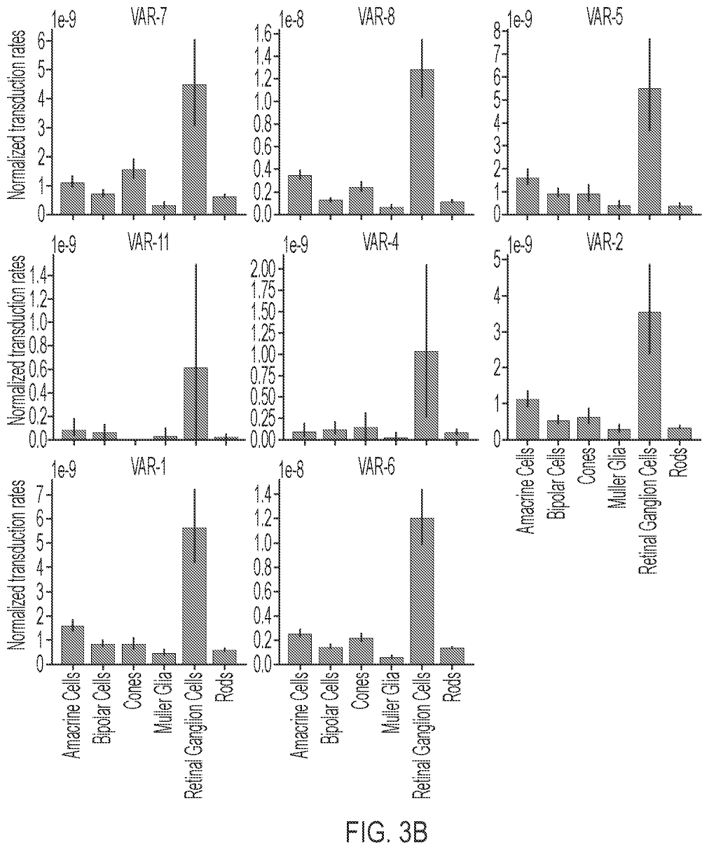

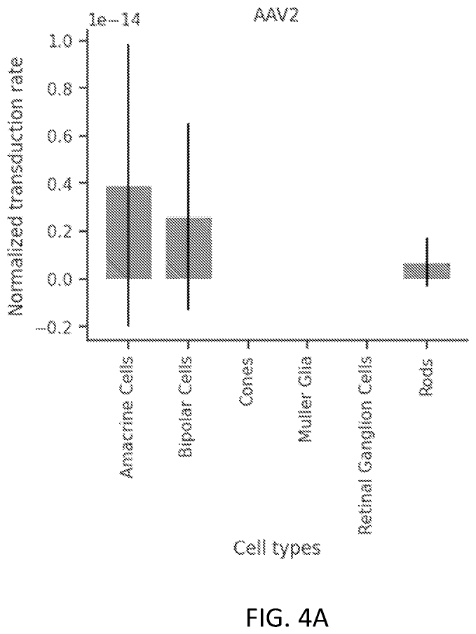

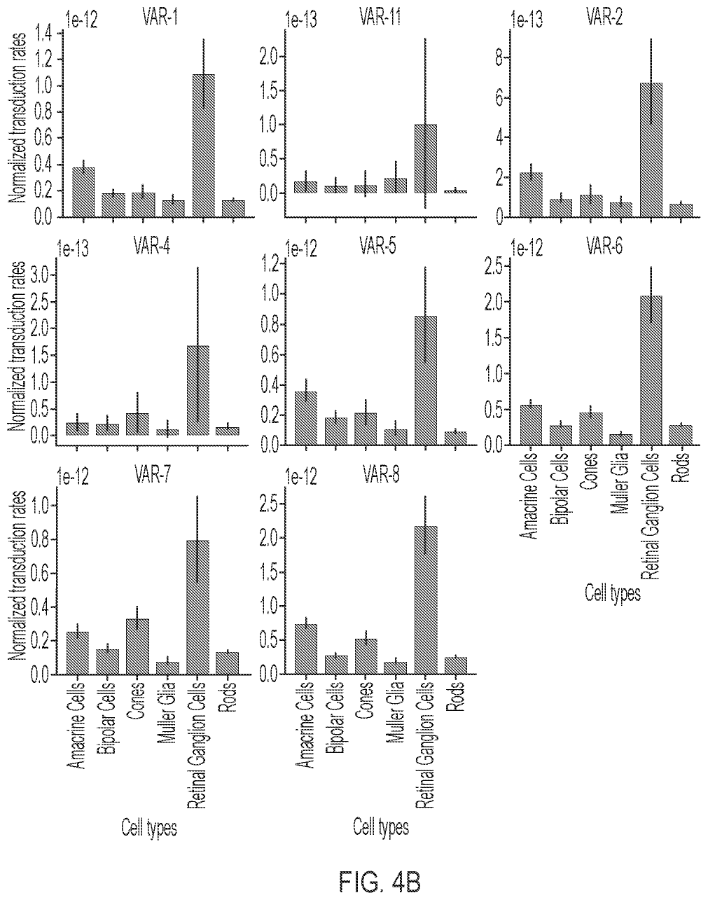

To calculate the normalized transduction rate of variant i in cell type j, the number of transduction events for variant i observed in cell type j was divided by the population count of cell type j. This value was then further normalized by the amount of virus reads for the cell type j to generate the data shown in FIGS. 3A-3B. To generate the data shown in FIGS. 4A-7B, this value was then further normalized by the amount of vector genome (vg) dosed, which is defined as the fraction of sequenced test article DNA reads belonging to variant i multiplied by total vg dosed into the eye (transduction efficiency of variant i in cell type j=(transduction events i/number of cells j)/#of dose vector genomes for variant i). The error bars were computed by bootstrapping cell barcodes (N=2000), calculating the resulting resampled rates, and identifying the 5th and 95th percentiles of the sampled distributions.

Control experiments comprising a mixture of 5% HEK293 cells transduced with AAV2 wild-type containing a barcoded genome and 95% non-transduced NHP liver cells were performed to determine the detection efficiency of the snRNA-seq assay. After processing and data analysis, we were able to determine that 6% of all nuclei processed were HEK-293 nuclei and detected viral transcripts from AAV2 wild-type in approximately 20% of the HEK-293 nuclei. This sensitivity is sufficient to identify and characterize viral vectors comprising variant capsid polypeptides from the medium throughput study described herein.

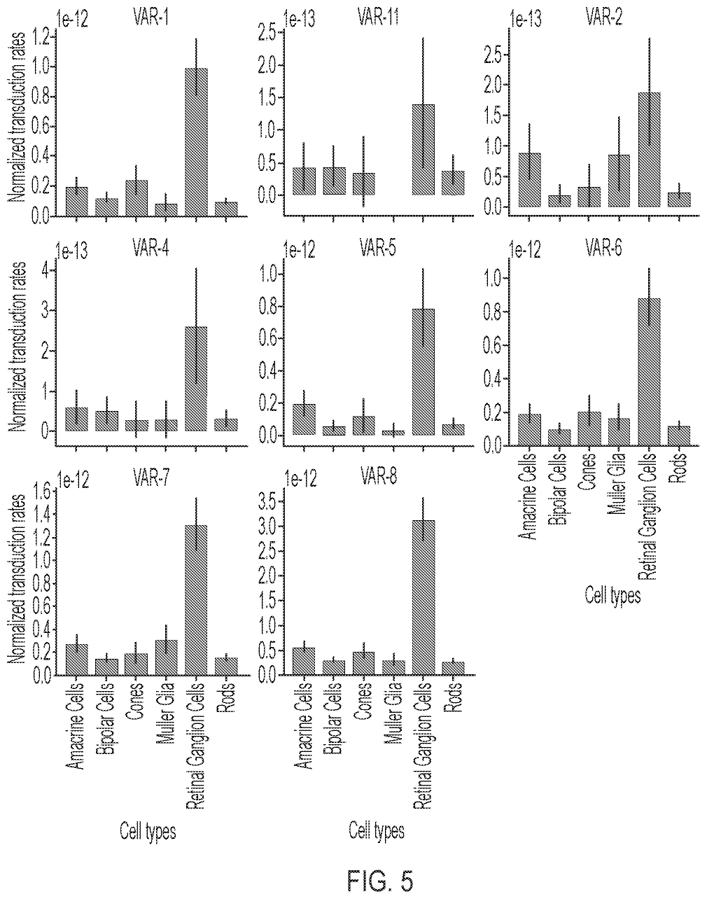

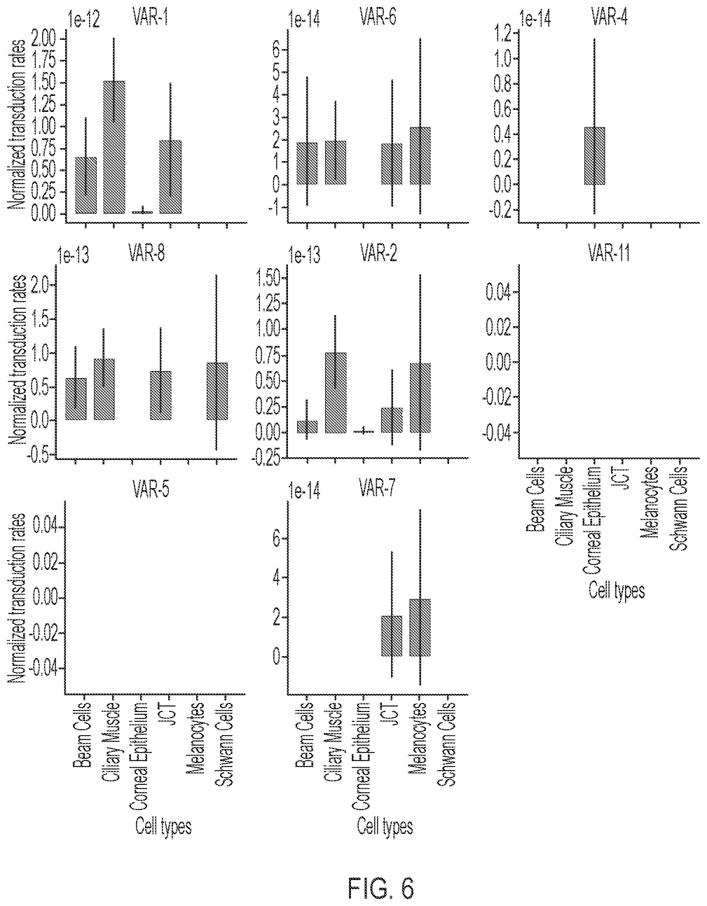

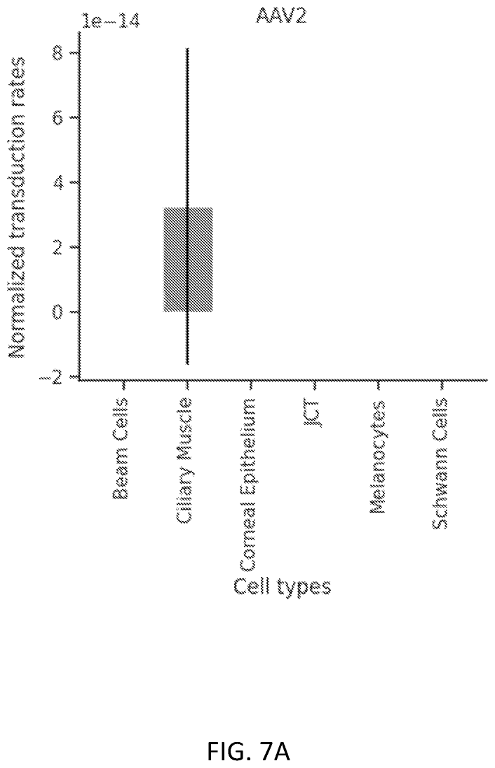

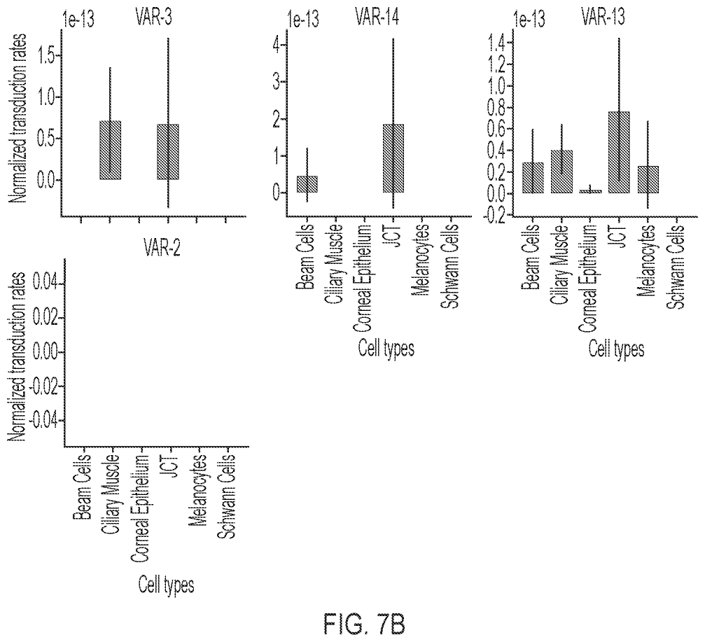

Next, we sequenced data from nuclei isolated from NHP posterior eye and anterior eye tissue. We plotted RNA transcript data on a UMAP plot (Leiden clustering) to show major cell types when projected onto an annotated eye reference dataset using maximum-likelihood (Swamy et al., GigaScience, Vol. 10, 2021, which is incorporated herein by reference in its entirety). This allowed us to annotate major cell types of the retina, including amacrine cells, bipolar cells, cones, horizontal cells, microglia, Müller glia, retinal ganglion cells, and rods. For trabecular meshwork tissues, we used a different reference dataset (van Zyl et al., PNAS 2020, which is incorporated herein by reference in its entirety) to annotate major cell types, which includes corneal epithelium, ciliary muscle, melanocytes, Schlemm's canal, Schwann cells, beam cells, juxtacanalicular tissue (JCT), fibroblasts, and pericytes. Using cell type specific markers from the literature (Menon et al., Nature Comm. 2019; Peng Y et al., Cell, 2019; Patel et al., PNAS, 2020, each of which is incorporated herein by reference in its entirety) we confirmed expected expression patterns from these cell clusters. FIGS. 3A and 3B show the cell type specific transduction for AAV2 (FIG. 3A) and variants (FIG. 3B) from retina tissue samples from intravitreal (IVT) administration of the medium throughput study, where the results are normalized to the amount of virus reads. FIGS. 4A and 4B show the cell type specific transduction for AAV2 (FIG. 4A) and variants (FIG. 4B) from retina tissue samples from IVT administration of the medium throughput study, where the results are normalized to the amount of vector genome (vg) dosed. FIG. 5 shows the cell type specific transduction for variants from macula tissue samples from IVT administration of the medium throughput study, where the results are normalized to the amount of vector genome (vg) dosed. The retina tissue samples used for generating the data shown in FIGS. 3A, 3B, 4A, 4B were from regions of the neural retina layer outside the macular region and, in this way, the data may be considered to show transduction of cell types in the non-macular region of the retina. In contrast, FIG. 5 shows transduction of cell types in the macular region of the retina. FIG. 6 shows the cell type specific transduction for variants from trabecular meshwork tissue samples from the IVT administration of the medium throughput study, where the results are normalized to the amount of vector genome (vg) dosed. No transduction of AAV2 wild-type for any of the listed cell types in FIGS. 5 and 6 were detected in macula and trabecular meshwork tissue samples from the IVT administration of the medium throughput study. FIGS. 7A and 7B show the cell type specific transduction for AAV2 (FIG. 7A) and variants (FIG. 7B) from trabecular meshwork tissue samples from intracameral (IC) administration of the medium throughput study, where the results are normalized to the amount of vector genome (vg) dosed. The error bars show 95% confidence interval, estimated by randomly resampling the cells 2000 times. For each of the above-described figures, lack of a data bar for a specific cell type indicates that there was no transduction of that cell type detected for AAV2 wild-type or the particular variant. For example, there is no data bar for cones in the data plot for AAV2 wild-type in FIG. 4A, which means that there was zero transduction of AAV2 wild-type detected in cones as part of the IVT administration of the medium throughput study.

### Results

Production results for the variants described herein are summarized in Tables 1D, 1E, and 1G, transduction results from bulk tissue are summarized in Tables 1D, 1E, 1F, and 1G, biodistribution results from bulk tissue are summarized in Tables 4B, 4C, and 4D, and single cell results are shown in FIGS. 3A-7B. VAR-1, VAR-2, VAR-3, VAR-4, VAR-5, VAR-6, VAR-7, VAR-8, VAR-11, VAR-13, and VAR-14 each display comparable or improved productivity as compared to WT AAV2. Similarly, as described herein and as shown in the Tables and Figures, variants described herein show improved biodistribution and/or transduction in the neural retina as compared to WT AAV2 by bulk and/or single-cell sequencing analyses. For example, VAR-8 shows greater than 100-fold and 1000-fold improvements in transduction measured in bulk neural retina (aggregated macular and non-macular tissue samples) and macular retina, respectively. Single-cell analyses of VAR-8 in these tissues showed improved transduction in all major neural retina cell types, with highest rate of improvement in retinal ganglion cells. Biodistribution and transduction in the anterior eye samples containing trabecular meshwork and Schlemm's canal were compared to a virus particle having capsid polypeptides of SEQ ID NO: 60. Some of these variants (e.g., VAR-1, VAR-2, VAR-6, and VAR-8) showed an increase in trabecular biodistribution and transduction over the virus particle comprising capsid polypeptides of SEQ ID NO: 60. Single-cell analyses revealed variants (e.g., VAR-1, VAR-2, VAR-6, VAR-8) with improved transduction in beam and juxtacanalicular cells, the cell types responsible for proper function of the trabecular meshwork. Without being bound by theory, altogether these findings indicate that the variants described herein are suitable for gene therapies where targeting the eye is important, for example, as described herein.

We next analyzed the structure of the variants described herein, for example the variants with improved transduction and/or biodistribution to one or more regions of the eye. Several variants comprise a sequence (e.g., a peptide insertion, one or more substitutions) in the surface exposed loop containing the heparin-binding domain relative to wild-type AAV2 capsid polypeptides. The insertion site originates after glutamine (Q) at position 584, arginine (R) at position 585, glycine (G) at position 586, or asparagine (N) at position 587 (WT AAV2 VP1 numbering; SEQ ID NO: 1). The insertions were between 6 and 11 amino acids in length. In addition, many of the variants described herein with insertion peptides originating at a position N-terminal to N587 (according to WT AAV2; SEQ ID NO: 1), for example those variants with an insertion peptide after G586 (according to WT AAV2; SEQ ID NO: 1) comprise a mutation at position N587 (according to SEQ ID NO: 1) to alanine (e.g., VAR-3, VAR-4, VAR-5, VAR-6, VAR-7, VAR-8, VAR-10, VAR-12, VAR-13, VAR-14, VAR-15 and VAR-16). VAR-5, VAR-7, VAR-11, and VAR-14 further comprise additional substitutions outside the insertion, as shown in Tables 1A-1G. VAR-1, VAR-2, VAR-3, VAR-5, VAR-6, VAR-8, VAR-11, VAR-13, and VAR-14 each comprise a “TRPA” motif, such as formed by an insertion having a C-terminal TRPA motif or an insertion peptide C-terminal TRP motif followed by an alanine substitution (e.g., N587A substitution). Without being bound by theory, these results suggest that variant capsid polypeptides comprising a N587 substitution mutation, for example a N587A substitution mutation, in combination with a N-terminally juxtaposed insertion peptide (e.g., a peptide comprising at its C-terminal end threonine, arginine, proline (“TRP”), have increased ocular retinal transduction and/or biodistribution. In some embodiments, the peptide insertion is fewer than 7 amino acids. In other embodiments, the peptide insertion is 7 or more amino acids, for example, 7, 8, 9, 10, or 11 amino acids.

In the neural retina, variants described herein have increased biodistribution in aggregated neural retina and macular retina relative to WT AAV2 by ˜2.1-21 and ˜3.9-181-fold, respectively, in bulk sequencing experiments. Increased macula biodistribution may arise from the inner limiting membrane (ILM) being thinner in the macula, and thus a variant with improved ILM penetrance could therefore present with improved increase in biodistribution in the macular retina in comparison to the aggregated neural retina. Variants described herein have greater transduction in the macula (˜28.1-1037-fold relative to WT AAV2) than in the aggregated neural retina (˜4.8-106-fold relative to WT AAV2). In particular, VAR-8 has transduction in the macula greater than 1000-fold relative to WT AAV2 and transduction in the aggregated neural retina greater than 100-fold relative to WT AAV2. For variants described herein, increases in relative biodistribution between macula and aggregated neural retina was ˜1.9-23.6-fold and increases in relative transduction between macula and aggregated neural retina was ˜5.9-9.8-fold. In general, increases in relative transduction were higher than in relative biodistribution in macula and aggregated neural retina. However, for some variants described herein the opposite was observed. This may imply that improved transduction properties may not solely be caused by increased ILM penetrance, and that the designed capsid modifications may have an impact on other functional properties of the capsid. As an example, VAR-8 showed a significant increase in transduction between aggregated neural retina and macula (˜9.8-fold over WT AAV2), but its increase in biodistribution was modest (˜4.1-fold), particularly as compared to VAR-4, which had the largest increase in biodistribution between aggregated neural retina and macular retina (˜23.6-fold over WT AAV2) of the variants described herein included in Library Experiment 3. In addition, variants described herein transduce macular retina more efficiently than aggregated neural retina, indicating that none of these variants specifically de-targeted the macular retina.

Relative transduction efficiencies of the variants described herein were also measured in single-nucleus sequencing experiments. Of the variants included in Library Experiment 3, VAR-8 has the highest transduction efficiency in macular and non-macular retina cells, corresponding to bulk sequencing measurements for VAR-8. For each of VAR-1, VAR-2, VAR-4, VAR-5, VAR-6, VAR-7, VAR-8, and VAR-11, retinal ganglion cells were the most efficiently transduced cell type in both macular and non-macular retina. For VAR-1, VAR-2, VAR-5, VAR-6, VAR-8, and VAR-9, amacrine cells were the second highest transduced cell type. VAR-4 and VAR-7 each showed comparable or higher transduction of cones than amacrine cells in non-macular retina. VAR-4 and VAR-7 each comprise a “TARPA” sequence (including both inserted and substituted amino acids, relative to WT AAV2; SEQ ID NO: 1, in both cases), without being bound by theory, we hypothesize that this motif may contribute to the observed increase in transduction of cones. However, the same trend was not observed in the macula for VAR-4 and VAR-7, but this may be attributable to an overall lower number of measured transduction events due to the smaller size of the macula tissue sample, which may result in higher uncertainty for these measurements. Thus, without being bound by theory, described herein are capsid polypeptides comprising a sequence threonine-alanine-proline-alanine (“TARPA”), including formed, for example as a combination of insertion and substitution mutations relative to WT AAV2 (SEQ ID NO: 1), with enhanced photoreceptor transduction.

In the anterior eye, transduction was measured in samples containing the trabecular meshwork and the Schlemm's canal by vectors delivered via intravitreal or intracameral routes of administration. Both biodistribution and transduction were increased over a virus particle comprising capsid polypeptides of SEQ ID NO: 60 by VAR-1, VAR-2, VAR-6 and VAR-8 after intravitreal delivery (˜1.6-3.1-fold and ˜1.5-2.8-fold for biodistribution and transduction, respectively). VAR-2 was delivered by both intravitreal and intracameral routes, and showed improved transduction as compared to the virus particle comprising capsid polypeptides of SEQ ID NO: 60 following intravitreal delivery only (˜0.1-fold via intracameral administration vs. ˜1.6-fold via intravitreal administration relative to the virus particle comprising capsid polypeptides of SEQ ID NO: 60). Improved transduction of VAR-1, VAR-2, VAR-6 and VAR-8 following intravitreal delivery was also detected in single-nucleus sequencing experiments. Transduction was detected in beam and juxtacanalicular cells, which are components of the trabecular meshwork.

Variants described herein (e.g., VAR-1, VAR-2, VAR-4, VAR-5, VAR-6, VAR-7, VAR-8 and VAR-11) are efficient in transducing several types of retinal cells via intravitreal delivery, particularly retinal ganglion cells, both in macular and non-macular retina. Therefore, these variants have potential to be used in gene therapy applications for neuroprotective treatments to prevent retinal ganglion cell degeneration associated with several ocular disorders, such as glaucoma, diabetic retinopathy, and Retinitis pigmentosa. Transduction of both rod and cone photoreceptors was also detected by the variants described herein (e.g., VAR-1, VAR-2, VAR-4, VAR-5, VAR-6, VAR-7, VAR-8 and VAR-11), indicating their potential as vectors for retinal diseases where photoreceptors are affected, such as achromatopsia, Retinitis pigmentosa, and forms of Leber congenital amaurosis. For ocular diseases where photoreceptors may no longer be present, transduction of the remaining retinal cells such as bipolar cells and Müller glia may enable optogenetic treatment strategies. The variants described herein (e.g., VAR-1, VAR-2, VAR-4, VAR-5, VAR-6, VAR-7, VAR-8 and VAR-11) may be used in developing optogenetic gene therapies by transducing bipolar cells and Müller glia. Finally, overall high transduction efficiency in the retina suggests that variants described herein may have potential in treating wet age-related macular degeneration.

Variants described herein (e.g., VAR-1, VAR-2, VAR-6 and VAR-8) have increased efficiency in transducing beam and juxtacanalicular cells in the trabecular meshwork following intravitreal delivery. Therefore, they have potential to be used in gene therapies aimed at influencing intraocular fluid balances and therefore intraocular pressure and to provide treatment options for related diseases, such as glaucoma.

### Example 4

To enable transduction of deeper retinal cell layers, current AAV-mediated ocular gene therapies require high doses to achieve clinically relevant efficacy which increases the risk of adverse inflammatory responses, or employ subretinal injections that must be performed by trained surgeons. More efficient cell-type targeted pan-retinal transduction via intravitreal (IVT) administration would allow for more effective therapies at lower and safer doses, broadening the reach of ocular gene therapies.

Using machine-guided design we generated variant capsids with up to 80 times more efficient transduction of the neural retina in non-human primates (NHP) following IVT delivery as compared to AAV2. We then evaluated cell-type specific transduction patterns in the neural retina by single-nucleus RNA-seq. Relative to an AAV2-derived literature variant reported to have improved retinal transduction, these capsids demonstrated 2-6× improvement in retinal cell types including rods, cones, microglia and retinal ganglion cells. Extrapolating to a conservative dose of 8.0e10 vg/eye, we infer that these variants are capable of transducing a high proportion of cells across retinal cell types, for example up to 17% in retinal ganglion cells. The true proportion of transduced cells is likely even higher due to incomplete capture of transduction events in single nucleus sequencing. We selected one top capsid for further validation and injected it intravitreally into NHP eyes. Qualitative and quantitative in-life and histological readouts for transduction properties were performed based on reporter transgene expression. Histological readouts confirmed the variant's significantly improved transduction efficiency compared to AAV2-derived literature variants. These results show the utility of single-molecule based NGS-based readouts in both library and validation contexts and demonstrate the power of applying machine learning to capsid design.

### Example 5

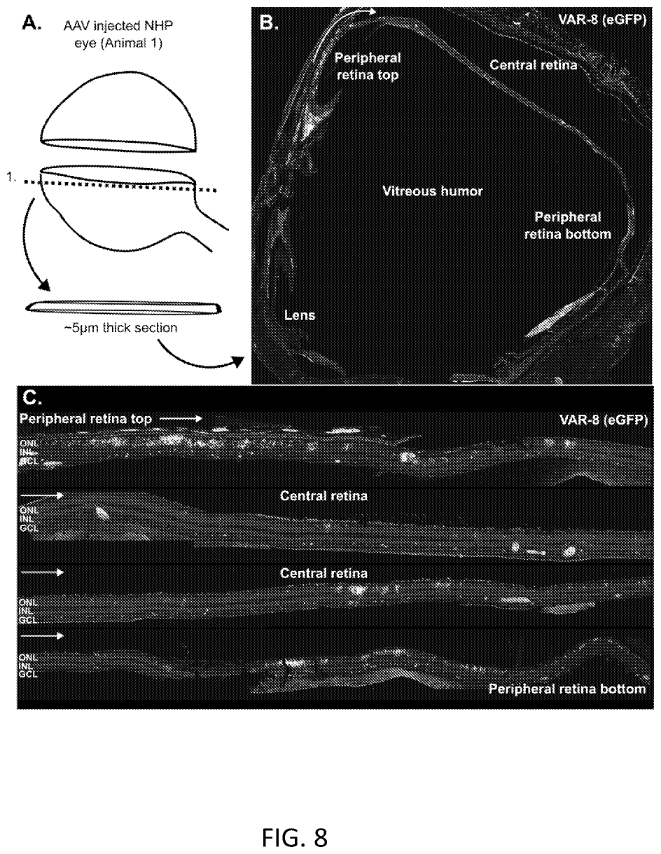

FIG. 8. Retinal distribution of AAV VAR-8-eGFP from a higher dose (2.26e11vg) intravitreal ocular injection in cynomolgus monkey. The genome packaged in the VAR-8 capsid is a self-complementary genome, composed of modified AAV2 ITRs, and an eGFP reporter gene under the control of a CBH promoter. The animals were sacrificed 4 weeks post-dosing. The eyes were enucleated, fixed, halved into two pieces (top and bottom) and embedded in paraffin to enable sectioning for histology. (A) Schematic illustrating where the cross-section of the retina was collected. Briefly a 5 μm section from the top surface of the bottom half of the eye was collected and stained for eGFP. (B) Representative image of a whole cross section of the eye collected. eGFP is shown as white dots in the retina. The arrow represents the starting position of the close up retinal image shown in (C). (C) Close up of the retinal images from each end of the eye shown in (B) stitched together linearly. White dots represent eGFP expression delivered from VAR-8. ONL=outer nuclear layer/photoreceptor layer, INL=inner nuclear layer, GCL=ganglion cell layer.

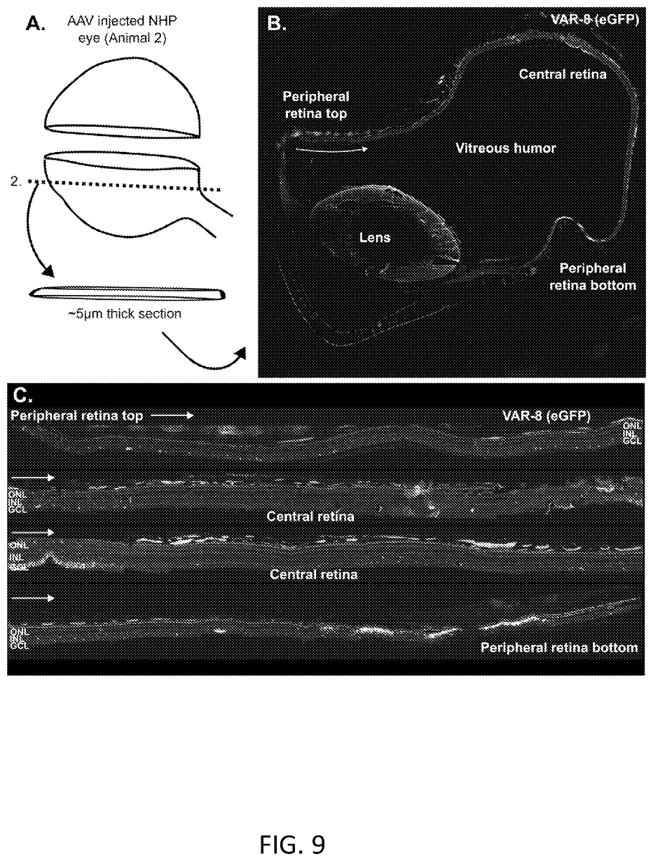

FIG. 9. Retinal distribution of AAV VAR-8-eGFP from a lower dose (8.08e10vg) intravitreal ocular injection in cynomolgus monkey. The genome packaged in the VAR-8 capsid is a self-complementary genome, composed of modified AAV2 ITRs, and an eGFP reporter gene under the control of a CBH promoter. The animals were sacrificed 4 weeks post-dosing. The eyes were enucleated, fixed, halved into two pieces (top and bottom) and embedded in paraffin to enable sectioning for histology. (A) Schematic illustrating where the cross-section of the retina was collected. Briefly a 5 μm section from ˜⅓ into the bottom half of the eye was collected and stained for eGFP. (B) Representative image of a whole cross section of the eye collected. eGFP is shown as white dots in the retina. The arrow represents the starting position of the close up retinal image shown in (C). (C) Close up of the retinal image from each end of the eye shown in (B) stitched together linearly. White dots represent eGFP expression delivered from VAR-8. ONL=outer nuclear layer/photoreceptor layer, INL=inner nuclear layer, GCL=ganglion cell layer.

Throughout the text of this application, should there be a discrepancy between the text of the specification (e.g., Table 2) and the sequence listing, the text of the specification shall prevail.

1. A variant capsid polypeptide comprising a polypeptide that has at least 85, 90, 91, 92, 93, 94, 95, 96, 97, 98, 99%, or 100% identity to a VP1, VP2, or VP3 sequence of SEQ ID NO: 19, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

2. The variant capsid polypeptide of claim 1, wherein the polypeptide comprises:
a mutation selected from a mutation associated with any of VAR-1 to VAR-16.

3. The variant capsid polypeptide of claim 2, wherein:
the mutation associated with any of VAR-1 to VAR-16 comprises mutations at positions corresponding to residues 550-597 as compared to SEQ ID NO: 1.

4. The variant capsid polypeptide of any of the preceding claims, wherein the polypeptide comprises a sequence having at least 85, 90, 91, 92, 93, 94, 95, 96, 97, 98, 99% identity to SEQ ID NO: 1 and comprises a mutation selected from a mutation associated with any of VAR-1 to VAR-16.

5. The variant capsid polypeptide of any of the preceding claims, wherein the polypeptide comprises a sequence having at least 85, 90, 91, 92, 93, 94, 95, 96, 97, 98, 99% identity to SEQ ID NO: 1 and wherein the variant capsid polypeptide comprises a mutation that corresponds to a mutation at position 550, 559, 561, 586, 587, 592, 593, 597, or any combination thereof, an insertion between positions 584 and 585, 586 and 587, or 587 and 588, or any combination thereof according to SEQ ID NO: 1, optionally wherein the mutation comprises an insertion, a deletion or a substitution.

6. The variant capsid polypeptide of any of the preceding claims, wherein the capsid polypeptide comprises:
a mutation that corresponds to an insertion at position between position 587 and 588, as compared to SEQ ID NO: 1;
a mutation that corresponds to an insertion at position between position 586 and 587, as compared to SEQ ID NO: 1;
a mutation that corresponds to a mutation at position 587, as compared to SEQ ID NO: 1, and an insertion at position between positions 586 and 587, as compared to SEQ ID NO: 1;
a mutation that corresponds to a mutation at position 587 and 593, as compared to SEQ ID NO: 1, and an insertion between positions 586 and 587, as compared to SEQ ID NO: 1;
a mutation that corresponds to a mutation at position 586 and 587, as compared to SEQ ID NO: 1, and an insertion between positions 584 and 585, as compared to SEQ ID NO: 1;
a mutation that corresponds to a mutation at position 592 and 597, as compared to SEQ ID NO: 1, and an insertion between positions 587 and 588, as compared to SEQ ID NO: 1;
a mutation that corresponds to a mutation at position 561, 587 and 597, as compared to SEQ ID NO: 1, and an insertion between positions 586 and 587, as compared to SEQ ID NO: 1;
a mutation that corresponds to a mutation at position 550, 586 and 587, as compared to SEQ ID NO: 1, and an insertion between positions 584 and 585, as compared to SEQ ID NO: 1;
a mutation that corresponds to a mutation at position 559 and 587, as compared to SEQ ID NO: 1, and an insertion between positions 586 and 587, as compared to SEQ ID NO: 1;
an insertion, e.g., an insertion of 1 or more amino acids, e.g., 1, 2 amino acids that corresponds to an insertion between positions 584 and 585, 586 and 587, and 587 and 588, as compared to SEQ ID NO: 1;
an insertion, e.g., an insertion of 6 or fewer amino acids, e.g., 6 amino acids, that corresponds to an insertion between positions 586 and 587 wherein the insertion sequence comprises threonine-arginine-proline at its C-terminus, and a substitution mutation at a position corresponding to N587, e.g., an alanine at a position corresponding to N587, all numbering as compared to SEQ ID NO: 1;
an insertion, e.g., an insertion of 7 or more amino acids, e.g., 10 or 11 amino acids, between positions 586 and 587 wherein the insertion sequence comprises threonine-arginine-proline at its C-terminus, and a substitution mutation at a position corresponding to N587, e.g., an alanine at a position corresponding to N587, all numbering as compared to SEQ ID NO: 1;
a mutation comprising the sequence threonine-alanine-arginine-proline-alanine, optionally wherein the sequence is at a location C-terminal to a position corresponding to 584 and N-terminal to a position corresponding to 590, as compared to SEQ ID NO: 1, and optionally wherein the sequence threonine-alanine-arginine-proline-alanine comprises insertion and substitution mutations as compared to SEQ ID NO: 1; or
a combination of any of the foregoing.

7. The variant capsid polypeptide of any of the preceding claims, wherein the capsid polypeptide comprises:
(a) an insertion at a position between positions 586 and 587 as compared to SEQ ID NO: 1, wherein the insertion comprises a polypeptide of LALGEQTRPA (SEQ ID NO: 44), or a fragment of at least 5, at least 6, at least 7, at least 8, or at least 9 amino acids thereof,
(b) an insertion at a position between positions 587 and 588 as compared to SEQ ID NO: 1, wherein the insertion comprises a polypeptide of LAIEQTRPA (SEQ ID NO: 45), or a fragment of at least 5, at least 6, at least 7, or at least 8 amino acids thereof;
(c) a mutation of N587A, and an insertion at a position between positions 586 and 587 as compared to SEQ ID NO: 1, wherein the insertion comprises a polypeptide of LALAEITRP (SEQ ID NO: 46), or a fragment of at least 5, at least 6, at least 7, or at least 8 amino acids thereof;
(d) a mutation of N587A, and an insertion at a position between positions 586 and 587 as compared to SEQ ID NO: 1, wherein the insertion comprises a polypeptide of LKNAETARP (SEQ ID NO: 47), or a fragment of at least 5, at least 6, at least 7, or at least 8 amino acids thereof;
(e) a mutation of N587A and A593T, and an insertion at a position between positions 586 and 587 as compared to SEQ ID NO: 1, wherein the insertion comprises a polypeptide of LNLAIEQTRP (SEQ ID NO: 48), or a fragment of at least 5, at least 6, at least 7, or at least 8 amino acids thereof;
(f) a mutation of N587A, and an insertion at a position between positions 586 and 587 as compared to SEQ ID NO: 1, wherein the insertion comprises a polypeptide of MLNEQTRP (SEQ ID NO: 49), or a fragment of at least 4, at least 5, at least 6, or at least 7 amino acids thereof,
(g) a mutation of G586P and N587A, and an insertion at a position between positions 584 and 585 as compared to SEQ ID NO: 1, wherein the insertion comprises a polypeptide of RSGNRADSETA (SEQ ID NO: 50), or a fragment of at least 5, at least 6, at least 7, at least 8, at least 9, or at least 10 amino acids thereof;
(h) a mutation of N587A, and an insertion at a position between positions 586 and 587 as compared to SEQ ID NO: 1, wherein the insertion comprises a polypeptide of TGDTRP (SEQ ID NO: 51), or a fragment of at least 3, at least 4, or at least 5 amino acids thereof;
(i) an insertion at a position between positions 587 and 588 as compared to SEQ ID NO: 1, wherein the insertion comprises a polypeptide of LQGETIRPA (SEQ ID NO: 52), or a fragment of at least 5, at least 6, at least 7, at least 8, at least 9, or at least 10 amino acids thereof;
(j) a mutation of N587A, and an insertion at a position between positions 586 and 587 as compared to SEQ ID NO: 1, wherein the insertion comprises a polypeptide of QNLANPETTRP (SEQ ID NO: 53), or a fragment of at least 5, at least 6, at least 7, at least 8, at least 9, or at least 10 amino acids thereof;
(k) a mutation of T592A and T597W, and an insertion at a position between positions 587 and 588 as compared to SEQ ID NO: 1, wherein the insertion comprises a polypeptide of RAPQETTRPA (SEQ ID NO: 54), or a fragment of at least 5, at least 6, at least 7, at least 8, or at least 9 amino acids thereof;
(l) a mutation of N587A, and an insertion at a position between positions 586 and 587 as compared to SEQ ID NO: 1, wherein the insertion comprises a polypeptide of ANLTTTRP (SEQ ID NO: 55), or a fragment of at least 4, at least 5, at least 6, or at least 7 amino acids thereof;
(m) a mutation of N587A, and an insertion at a position between positions 586 and 587 as compared to SEQ ID NO: 1, wherein the insertion comprises a polypeptide of ALLAGEQTRP (SEQ ID NO: 56), or a fragment of at least 5, at least 6, at least 7, at least 8, or at least 9 amino acids thereof;
(n) a mutation of N587A, and an insertion at a position between positions 586 and 587 as compared to SEQ ID NO: 1, wherein the insertion comprises a polypeptide of ALLAGEQTRP (SEQ ID NO: 56), or a fragment of at least 5, at least 6, at least 7, at least 8, or at least 9 amino acids thereof;
(o) a mutation of D561C, N587A, and T597N, and an insertion at a position between positions 586 and 587 as compared to SEQ ID NO: 1, wherein the insertion comprises a polypeptide of GLRAEQTRP (SEQ ID NO: 57), or a fragment of at least 5, at least 6, at least 7, or at least 8 amino acids thereof;
(p) a mutation of T550N, G586P, and N587A, and an insertion at a position between positions 584 and 585 as compared to SEQ ID NO: 1, wherein the insertion comprises a polypeptide of RARLDETA (SEQ ID NO: 58), or a fragment of at least 4, at least 5, at least 6, or at least 7 amino acids thereof; or
(q) a mutation of I559L, and N587A, and an insertion at a position between positions 586 and 587 as compared to SEQ ID NO: 1, wherein the insertion comprises a polypeptide of TNLARGETARP (SEQ ID NO: 59), or a fragment of at least 5, at least 6, at least 7, at least 8, at least 9, or at least 10 amino acids thereof.

8. A variant capsid polypeptide, comprising (a) a polypeptide of any one of SEQ ID NO: 19, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27, (b) the VP2 or VP3 sequence of any one of SEQ ID NO: 19, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27, (c) a polypeptide comprising a sequence having at least 90%, at least 95%, at least 96%, at least 97%, at least 98%, or at least 99% identity thereto, wherein said sequence comprises at least one (e.g., one, two, three or more, e.g., all) of the mutation differences associated with any of SEQ ID NO: 12 through SEQ ID NO: 27, relative to SEQ ID NO: 1; or (d) a polypeptide having at least 1, but no more than 20, no more than 19, no more than 18, no more than 17, no more than 16, no more than 15, no more than 14, no more than 13, no more than 12, no more than 10, no more than 9, no more than 8, no more than 7, no more than 6, no more than 5, no more than 3, or no more than 2 amino acid mutations relative to the polypeptide of (a) or (b), wherein said polypeptide comprises at least one (e.g., one, two, three or more, e.g., all) of the mutation differences associated with any of SEQ ID NO: 12 through SEQ ID NO: 27, relative to SEQ ID NO: 1.

9. A variant capsid polypeptide comprising:
an amino acid sequence that has 95% or more amino acid sequence identity to an amino acid sequence of one of SEQ ID NOs: 12-27 and;
has at least 80% of the mutations in said amino acid sequence of one of SEQ ID NO: 12-27 as compared to SEQ ID NO: 1.

10. A variant capsid polypeptide comprising:
an amino acid sequence that has less than 95% amino acid sequence identity to an amino acid sequence of one of SEQ ID NOs: 12-27 and;
has at least 80% of the mutations in said amino acid sequence of one of SEQ ID NO: 12-27 as compared to SEQ ID NO: 1.

11. A variant capsid polypeptide comprising:
an amino acid sequence that has 95% or more amino acid sequence identity to an amino acid sequence of one of SEQ ID NOs: 12-27 and;
has less than 80% of the mutations in said amino acid sequence of one of SEQ ID NO: 12-27 as compared to SEQ ID NO: 1.

12. A nucleic acid molecule comprising a sequence encoding a variant capsid polypeptide of any one of claims 1-11.

13. The nucleic acid molecule of claim 12, comprising one or more regulatory elements operably linked to the sequence encoding the variant capsid polypeptide.

14. The nucleic acid molecule of any of claims 12-13, comprising SEQ ID NO: 35, 28, 29, 30, 31, 32, 33, 34, 36, 37, 38, 39, 40, 41, 42, or 43, or a fragment thereof, or a variant thereof having at least 85, 90, 91, 92, 93, 94, 95, 96, 97, 98, 99, or 100% sequence identity thereto.

15. A virus particle (e.g., adeno-associated virus (“AAV”) particle) comprising the variant capsid polypeptide of any one of claims 1-11 or comprising a variant capsid polypeptide encoded by the nucleic acid molecule of any one of claims 12-14.

16. The virus particle of claim 15, comprising a nucleic acid comprising a heterologous transgene and one or more regulatory elements.

17. A virus particle of any of claims 15-16 comprising the variant capsid polypeptide of any one of claims 1-11, wherein said virus particle, or a virus particle comprising said variant capsid polypeptide or a virus particle comprising a variant capsid polypeptide encoded by a nucleic acid molecule of any one of claims 12-14 exhibits increased ocular transduction, e.g., as measured in a mouse or in NHP, e.g., as described herein, relative to wild-type AAV2 (e.g., a virus particle comprising capsid polypeptides of SEQ ID NO: 1 or encoded by SEQ ID NO: 2).

18. A method of producing a virus particle comprising a variant AAV2 capsid polypeptide, said method comprising introducing a nucleic acid molecule of any one of claims 12-14 into a cell (e.g., a HEK293 cell), and harvesting said virus particle therefrom.

19. A method of delivering a payload (e.g., a nucleic acid) to a cell comprising contacting the cell with a dependoparvovirus particle comprising a variant capsid polypeptide of any one of claims 1-11 or the virus particle of any of claims 15-17 and a payload.

20. The method of claim 19, wherein the cell is an ocular cell.

21. The method of claim 20, wherein the ocular cell is in the retina, the macula, or the trabecular meshwork.

22. A method of delivering a payload (e.g., a nucleic acid) to a subject comprising administering to the subject a dependoparvovirus particle comprising a variant capsid polypeptide of any one of claims 1-11 and the payload, or administering to the subject the virus particle of any one of claims 15-17.

23. The method of claim 22, wherein the particle delivers the payload to the eye.

24. The method of claim 22, wherein the particle delivers the payload to the retina, the macular, or the trabecular meshwork.

25. The method of any one of claims 22-24, wherein the particle delivers the payload to the eye with increased transduction in one or more regions of the eye as compared to a virus particle comprising capsid polypeptides of SEQ ID NO: 1, wherein the one or more regions of the eye is selected from the retina, the macula, the trabecular meshwork, or any combination thereof.

26. The method of claim 25, wherein the retina comprises non-macular retina.

27. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 64-times, 100-times, 128-times or 1000-times as compared to a virus particle comprising capsid polypeptides of SEQ ID NO: 1.

28. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to non-macular retina tissue relative to macular tissue.

29. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to macular tissue relative to non-macular retina tissue.

30. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to macular tissue relative to trabecular meshwork tissue.

31. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, or 64-times as compared to a virus particle comprising capsid polypeptides of SEQ ID NO: 1, and wherein the increase in transduction is specific to non-macular retina tissue relative to trabecular meshwork tissue.

32. The variant capsid polypeptide of any of claims 1-11, the virus particle of any of claims 15-17 or the method of any one of claims 18-26, wherein the (e.g., particle comprising the variant capsid polypeptide) particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, or 128-times as compared to a virus particle comprising capsid polypeptides of SEQ ID NO: 1, and wherein the increase in transduction is specific to macular tissue and non-macular retina tissue relative to trabecular meshwork tissue.

33. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue relative to macular tissue and non-macular retina tissue.

34. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue relative to macular tissue.

35. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue relative to non-macular retina tissue.

36. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, or 8-times as compared to a virus particle comprising capsid polypeptides of SEQ ID NO: 1, and wherein the increase in transduction is specific to trabecular meshwork tissue, macular tissue, and non-macular retina tissue.

37. The variant capsid polypeptide of any of claims 1-11, the virus particle of any of claims 15-17 or the method of any one of claims 18-26, wherein the particle (e.g., particle comprising the variant capsid polypeptide) delivers the payload to the eye with increased transduction specificity in one or more regions of the eye as compared to a virus particle comprising capsid polypeptides of SEQ ID NO: 1 without increased biodistribution in one or more regions of the eye as compared to a virus particle comprising capsid polypeptides of SEQ ID NO: 1.

38. The method of any one of claims 19-26, wherein the administration to the subject is via an intravitreal injection, or an intracameral injection.

39. A method of treating a disease or condition in a subject, comprising administering to the subject a dependoparvovirus particle in an amount effective to treat the disease or condition, wherein the dependoparvovirus particle is a particle comprising a capsid polypeptide of any one of claims 1-11 and 27-37, or encoded by the nucleic acid of anyone of claims 12-14, or is a virus particle of any one of claims 15-17.

40. A cell, cell-free system, or other translation system, comprising the capsid polypeptide, nucleic acid molecule, or virus particle of any one of claims 1-17 or 27-37.

41. A method of making a dependoparvovirus (e.g., an adeno-associated dependoparvovirus (AAV) particle, comprising:
providing a cell, cell-free system, or other translation system, comprising a nucleic acid of any of claims 12-14; and
cultivating the cell, cell-free system, or other translation system, under conditions suitable for the production of the dependoparvovirus particle,
thereby making the dependoparvovirus particle.

42. The method of claim 41, wherein the cell, cell-free system, or other translation system comprises a second nucleic acid molecule and said second nucleic acid molecule is packaged in the dependoparvovirus particle.

43. The method of claim 42, wherein the second nucleic acid comprises a payload, e.g., a heterologous nucleic acid sequence encoding a therapeutic product.

44. The method of any one of claims 41-43, wherein the nucleic acid of any of claims 12-14 mediates the production of a dependoparvovirus particle which does not include said nucleic acid of any of claims 12-14.

45. The method of any one of claims 41-44, wherein the nucleic acid of any of claims 12-14 mediates the production of a dependoparvovirus particle at a level at least 10%, at least 20%, at least 50%, at least 100%, at least 200% or greater than the production level mediated by the nucleic acid of SEQ ID NO: 2.

46. A composition, e.g., a pharmaceutical composition, comprising a virus particle of any one of claims 15-17 or a virus particle produced by the method of any one of claims 18 or 41-45, and a pharmaceutically acceptable carrier.

47. The variant capsid polypeptide of any of claims 1-11 and 27-37, the nucleic acid molecule of any of claims 12-14, or the virus particle of any of claims 15-17 for use in treating a disease or condition in a subject.

48. The variant capsid polypeptide of any of claims 1-11 and 27-37, the nucleic acid molecule of any of claims 12-14, or the virus particle of any of claims 15-17 for use in the manufacture of a medicament for use in treating a disease or condition in a subject.
